(19) United States
(12) Patent Application Publication
(10) Pub. No.: US 2024/0122063 A1
(43) Pub. Date: Apr. 11, 2024

(54) COMPOSITION, POWDER, ORGANIC ELECTROLUMINESCENT ELEMENT, METHOD FOR MANUFACTURING ORGANIC ELECTROLUMINESCENT ELEMENT, AND ELECTRONIC DEVICE

(71) Applicant: IDEMITSU KOSAN CO.,LTD., Chiyoda ku, Tokyo (JP)

(72) Inventors: Tetsuya MASUDA, Sodegaura-shi, Chiba (JP); Hiroaki ITOI, Sodegaura-shi, Chiba (JP)

(73) Assignee: IDEMITSU KOSAN CO.,LTD., Chiyoda ku, Tokyo (JP)

(21) Appl. No.: 18/262,564

(22) PCT Filed: Jan. 25, 2022
(86) PCT No.: PCT/JP2022/002596

§ 371 Date: Jul. 21, 2023

(30) Foreign Application Priority Data
Jan. 26, 2021 (JP) 2021-010478
May 17, 2021 (JP) 2021-083488
Jun. 25, 2021 (JP) 2021-105322

(51) Int. Cl. H10K 85/60; C07C 15/28; C07D 307/77; C07D 307/91; C07D 311/78; C09K 11/06

(52) U.S. Cl. CPC H10K 85/615; C07C 15/28; C07D 307/77; C07D 307/91; C07D 311/78; C09K 11/06; H10K 85/626; H10K 85/6574; H10K 50/11

(57) ABSTRACT

A composition including a compound represented by the formula (A1) (X_{A1} is O or S, Z_{A1} is a single bond or C) and a compound represented by the formula (B1) (X_{B1} is O, S, N, C) (provided that the compound represented by the formula (A1) and the compound represented by the formula (B1) are different compounds).

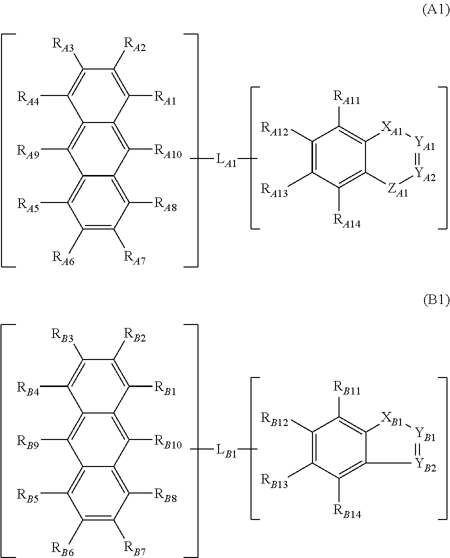

### TECHNICAL FIELD

The present invention relates to a composition, a powder, an organic electroluminescence device, a method for fabricating an organic electroluminescence device and an electronic apparatus.

### BACKGROUND ART

When voltage is applied to an organic electroluminescence device (hereinafter, also referred to as an organic EL device), holes and electrons are injected into an emitting layer from an anode and a cathode, respectively. Then, thus injected holes and electrons are recombined in the emitting layer, and excitons are formed therein.

Conventional organic EL devices have not yet had sufficient device performance. Although materials used for the organic EL device are gradually improved to enhance the device performance, further performance enhancement is required.

Research is being conducted into effectively using the feature of each material in the case where two or more kinds of materials are used in combination in a single organic layer. For example, Patent Documents 1 to 4 disclose a technique that two kinds of materials are used in combination in an emitting layer.

### RELATED ART DOCUMENTS

### Patent Documents

[Patent Document 1] WO 2005/113531 A1
[Patent Document 2] WO 2013/069338 A1
[Patent Document 3] EP 3020782 A1
[Patent Document 4] WO 2019/194615 A1

### SUMMARY OF THE INVENTION

It is an object of the present invention to provide a composition which is capable of improving luminous efficiency of an organic electroluminescence device or reducing driving voltage thereof and which includes two kinds of compounds having the specific structures and an organic electroluminescence device including a single layer composed of the same.

According to the present invention, the following composition, powder, organic electroluminescence device, method for fabricating an organic electroluminescence device, electronic apparatus and the like are provided.
1. A composition comprising a compound represented by the following formula (A1) and a compound represented by the following formula (B1) (provided that the compound represented by the formula (A1) and the compound represented by the formula (B1) are different compounds):

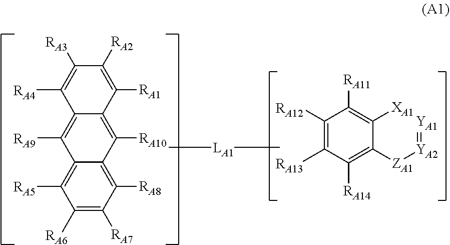

wherein in the formula (A1),
one of R_{A1} to R_{A10} is a single bond bonding with L_{A1};
one or more sets of the adjacent two or more of R_{A1} to R_{A10} which are not a single bond bonding with L_{A1} form a substituted or unsubstituted, saturated or unsaturated ring by bonding with each other, or do not form the ring;
one of R_{A11} to R_{A14} is a single bond bonding with L_{A1};
L_{A1} is a single bond, a substituted or unsubstituted arylene group having 6 to 50 ring carbon atoms, or a substituted or unsubstituted divalent heterocyclic group having 5 to 50 ring atoms;
X_{A1} is O or S.
Y_{A1} and Y_{A2} are independently a single bond, or C(Rₐ);
Z_{A1} is a single bond, or C(Rₐ)(R_{b});
one or more sets of the adjacent two or more of Rₐ, and R_{A11} to R_{A14} which are not a single bond bonding with L_{A1} form a substituted or unsubstituted, saturated or unsaturated ring by bonding with each other, or do not form the ring;
R_{b}, R_{A1} to R_{A10} and R_{A11} to R_{A14} which are not a single bond bonding with L_{A1} and which do not form the ring, and Rₐ which do not form the ring are independently a hydrogen atom, or a substituent R;
   the substituent R is selected from the group consisting of
   a substituted or unsubstituted alkyl group having 1 to 50 carbon atoms.
   a substituted or unsubstituted alkenyl group having 2 to 50 carbon atoms.
   a substituted or unsubstituted alkynyl group having 2 to 50 carbon atoms.
   a substituted or unsubstituted cycloalkyl group having 3 to 50 ring carbon atoms,
   —Si(R₉₀₁)(R₉₀₂)(R₉₀₃),
   —O—(R₉₀₄),
   —S—(R₉₀₅),
   —N(R₉₀₆)(R₉₀₇),
   a halogen atom, a cyano group, a nitro group,
   a substituted or unsubstituted aryl group having 6 to 50 ring carbon atoms, and
a substituted or unsubstituted monovalent heterocyclic group having 5 to 50 ring atoms;
R₉₀₁ to R₉₀₇ are independently
   a hydrogen atom,
   a substituted or unsubstituted alkyl group having 1 to 50 carbon atoms.
   a substituted or unsubstituted cycloalkyl group having 3 to 50 ring carbon atoms.
   a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other:

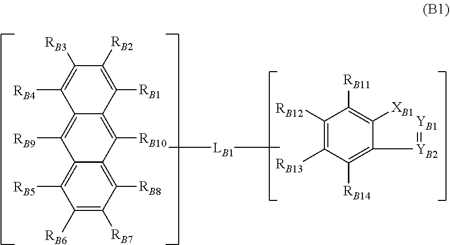

wherein in the formula (B1).
one of R_{B1} to R_{B10} is a single bond bonding with L_{B1};
one or more sets of the adjacent two or more of R_{B1} to R_{B10} which are not a single bond bonding with L_{B1} form a substituted or unsubstituted, saturated or unsaturated ring by bonding with each other, or do not form the ring;
X_{B1} is O, S, N(R_{c}), or C(R_{d})(Rₑ);
one of R_{B11} to R_{B14}, and Rₑ is a single bond bonding with L_{B1};
L_{B1} is a single bond, a substituted or unsubstituted arylene group having 6 to 50 ring carbon atoms, or a substituted or unsubstituted divalent heterocyclic group having 5 to 50 ring atoms;
Y_{B1} and Y_{B2} are independently a single bond, or C(R_{f});
R_{d} and Rₑ, and R_{f}'s form a substituted or unsubstituted, saturated or unsaturated ring by bonding with each other, or do not form the ring;
one or more sets of the adjacent two or more of R_{B11}, to R_{B14} which are not a single bond bonding with L_{B1} form a substituted or unsubstituted, saturated or unsaturated ring by bonding with each other, or do not form the ring; and
R_{B1} to R_{B10} which do not form the ring and which are not a single bond bonding with L_{B1}, R_{B11} to R_{B14} which do not form the ring and which are not a single bond bonding with L_{B1}, Rₑ which are not a single bond bonding with L_{B1}, and R_{d}, Rₑ and R_{f} which do not form the ring are independently a hydrogen atom, or a substituent R; and the substituent R is the same as defined in the formula (A1).
2. A powder composed of the composition according to 1.
3. An organic electroluminescence device comprising the composition according to 1.
4. A method for fabricating an organic electroluminescence device, comprising: forming an emitting layer by depositing the powder according to 2.
5. An organic electroluminescence device comprising
   a cathode,
   an anode, and
   at least one emitting layer arranged between the cathode and the anode,
   wherein the at least one emitting layer comprises a compound represented by the following formula (A1) and a compound represented by the following formula (B1) (provided that the compound represented by the formula (A1) and the compound represented by the formula (B1) are different compounds):

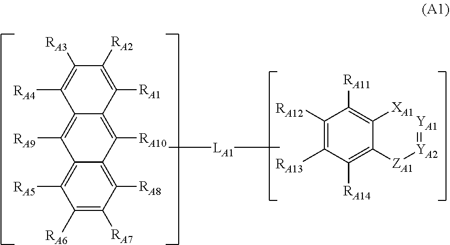

wherein in the formula (A1),
one of R_{A1} to R_{A10} is a single bond bonding with L_{A1};
one or more sets of the adjacent two or more of R_{A1} to R_{A10} which are not a single bond bonding with L_{A1} form a substituted or unsubstituted, saturated or unsaturated ring by bonding with each other, or do not form the ring;
one of R_{A11} to R_{A14} is a single bond bonding with L_{A1};
L_{A1} is a single bond, a substituted or unsubstituted arylene group having 6 to 50 ring carbon atoms, or a substituted or unsubstituted divalent heterocyclic group having 5 to 50 ring atoms;
X_{A1} is O or S;
Y_{A1} and Y_{A2} are independently a single bond, or C(Rₐ);
Z_{A1} is a single bond, or C(Rₐ)(R_{b});
one or more sets of the adjacent two or more of Rₐ, and R_{A11} to R_{A14} which are not a single bond bonding with L_{A1} form a substituted or unsubstituted, saturated or unsaturated ring by bonding with each other, or do not form the ring;
R_{b}, R_{A1} to R_{A10} and R_{A11} to R_{A14} which are not a single bond bonding with L_{A1} and which do not form the ring, and Rₐ which do not form the ring are independently a hydrogen atom, or a substituent R;
   the substituent R is selected from the group consisting of
   a substituted or unsubstituted alkyl group having 1 to 50 carbon atoms.
   a substituted or unsubstituted alkenyl group having 2 to 50 carbon atoms.
   a substituted or unsubstituted alkynyl group having 2 to 50 carbon atoms.
   a substituted or unsubstituted cycloalkyl group having 3 to 50 ring carbon atoms.
   —Si(R₉₀₁)(R₉₀₂)(R₉₀₃),
   —O—(R₉₀₄),
   —S—(R₉₀₅),
   —N(R₉₀₆)(R₉₀₇),
   a halogen atom, a cyano group, a nitro group,
   a substituted or unsubstituted aryl group having 6 to 50 ring carbon atoms, and
a substituted or unsubstituted monovalent heterocyclic group having 5 to 50 ring atoms;
R₉₀₁ to R₉₀₇ are independently
   a hydrogen atom,
   a substituted or unsubstituted alkyl group having 1 to 50 carbon atoms,
   a substituted or unsubstituted cycloalkyl group having 3 to 50 ring carbon atoms.
   a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other:

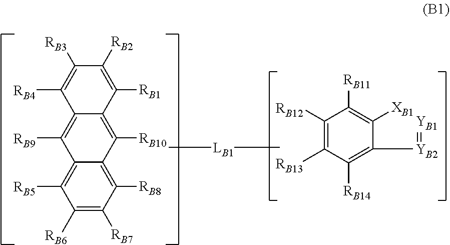

wherein in the formula (B1)
one of R_{B1} to R_{B10} is a single bond bonding with L_{B1};
one or more sets of the adjacent two or more of R_{B1} to R_{B10} which are not a single bond bonding with L_{B1} form a substituted or unsubstituted, saturated or unsaturated ring by bonding with each other, or do not form the ring;
X_{B1} is O, S, N(R_{c}), or C(R_{d})(Rₑ);
one of R_{B11} to R_{B14}, and R_{c} is a single bond bonding with L_{B1};
L_{B1} is a single bond, a substituted or unsubstituted arylene group having 6 to 50 ring carbon atoms, or a substituted or unsubstituted divalent heterocyclic group having 5 to 50 ring atoms;
Y_{B1} and Y_{B2} are independently a single bond, or C(R_{f});
R_{d} and Rₑ, and R_{f}'s form a substituted or unsubstituted, saturated or unsaturated ring by bonding with each other, or do not form the ring;
one or more sets of the adjacent two or more of R_{B11} to R_{B14} which are not a single bond bonding with L_{B1} form a substituted or unsubstituted, saturated or unsaturated ring by bonding with each other, or do not form the ring; and
R_{B1} to R_{B10} which do not form the ring and which are not a single bond bonding with L_{B1}, R_{B11} or, to R_{B14} which do not form the ring and which are not a single bond bonding with L_{B1}, R_{c} which are not a single bond bonding with L_{B1}, and R_{d}, Rₑ and R_{f} which do not form the ring are independently a hydrogen atom, or a substituent R; and the substituent R is the same as defined in the formula (A1).
6. An electronic apparatus comprising the organic electroluminescence device according to 5.

According to the present invention, there can be provided a composition which is capable of improving luminous efficiency of an organic electroluminescence device or reducing driving voltage thereof and which includes two kinds of compounds having the specific structures and an organic electroluminescence device including a single layer composed of the same.

### BRIEF DESCRIPTION OF THE DRAWINGS

FIG. 1 is a diagram showing a schematic configuration of an organic EL device according to one embodiment of the present invention.

FIG. 2 is a diagram showing a schematic configuration of an organic EL device according to another embodiment of the present invention.

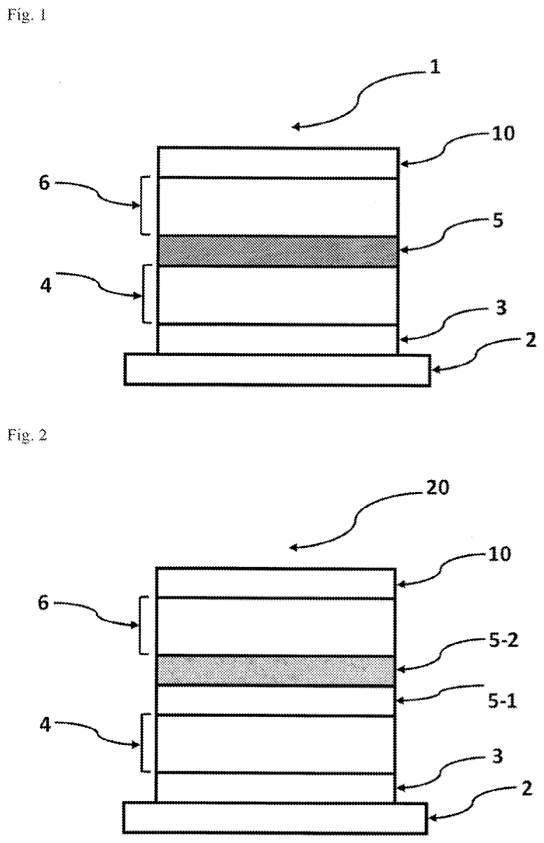

### MODE FOR CARRYING OUT THE INVENTION

### Definition

In this specification, a hydrogen atom includes its isotopes different in the number of neutrons, namely, a protium, a deuterium and a tritium.

In this specification, at a bondable position in a chemical formula where a symbol such as “R”, or “D” representing a deuterium atom is not indicated, a hydrogen atom, that is, a protium atom, a deuterium atom or a tritium atom is bonded.

In this specification, the number of ring carbon atoms represents the number of carbon atoms forming a subject ring itself among the carbon atoms of a compound having a structure in which atoms are bonded in a ring form (for example, a monocyclic compound, a fused ring compound, a cross-linked compound, a carbocyclic compound, or a heterocyclic compound). When the subject ring is substituted by a substituent, the carbon contained in the substituent is not included in the number of ring carbon atoms. The same shall apply to “the number of ring carbon atoms” described below, unless otherwise specified. For example, a benzene ring has 6 ring carbon atoms, a naphthalene ring includes 10 ring carbon atoms, a pyridine ring includes 5 ring carbon atoms, and a furan ring includes 4 ring carbon atoms. Further, for example, a 9,9-diphenylfluorenyl group includes 13 ring carbon atoms, and a 9,9′-spirobifluorenyl group includes 25 ring carbon atoms.

When a benzene ring is substituted by, for example, an alkyl group as a substituent, the number of carbon atoms of the alkyl group is not included in the number of ring carbon atoms of the benzene ring. Therefore, the number of ring carbon atoms of the benzene ring substituted by the alkyl group is 6. When a naphthalene ring is substituted by, for example, an alkyl group as a substituent, the number of carbon atoms of the alkyl group is not included in the number of ring carbon atoms of the naphthalene ring. Therefore, the number of ring carbon atoms of the naphthalene ring substituted by the alkyl group is 10.

In this specification, the number of ring atoms represents the number of atoms forming a subject ring itself among the atoms of a compound having a structure in which atoms are bonded in a ring form (for example, the structure includes a monocyclic ring, a fused ring and a ring assembly) (for example, a monocyclic compound, a fused ring compound, a cross-linked compound, a carbocyclic compound and a heterocyclic compound). The number of ring atoms does not include atoms which do not form the ring (for example, a hydrogen atom which terminates a bond of the atoms forming the ring), or atoms contained in a substituent when the ring is substituted by the substituent. The same shall apply to “the number of ring atoms” described below, unless otherwise specified. For example, the number of atoms of a pyridine ring is 6, the number of atoms of a quinoline ring is 10, and the number of a furan ring is 5. For example, hydrogen atoms bonded to a pyridine ring and atoms constituting a substituent substituted on the pyridine ring are not included in the number of ring atoms of the pyridine ring. Therefore, the number of ring atoms of a pyridine ring with which a hydrogen atom or a substituent is bonded is 6. For example, hydrogen atoms and atoms constituting a substituent which are bonded with a quinoline ring is not included in the number of ring atoms of the quinoline ring. Therefore, the number of ring atoms of a quinoline ring with which a hydrogen atom or a substituent is bonded is 10.

In this specification, “XX to YY carbon atoms” in the expression “a substituted or unsubstituted ZZ group including XX to YY carbon atoms” represents the number of carbon atoms in the case where the ZZ group is unsubstituted by a substituent, and does not include the number of carbon atoms of a substituent in the case where the ZZ group is substituted by the substituent. Here, “YY” is larger than “XX”, and “XX” means an integer of 1 or more and “YY” means an integer of 2 or more.

In this specification, “XX to YY atoms” in the expression “a substituted or unsubstituted ZZ group including XX to YY atoms” represents the number of atoms in the case where the ZZ group is unsubstituted by a substituent, and does not include the number of atoms of a substituent in the case where the 72 group is substituted by the substituent. Here, “YY” is larger than “XX”, and “XX” means an integer of 1 or more and “YY” means an integer of 2 or more.

In this specification, the unsubstituted ZZ group represents the case where the “substituted or unsubstituted ZZ group” is a “ZZ group unsubstituted by a substituent”, and the substituted ZZ group represents the case where the “substituted or unsubstituted ZZ group” is a “ZZ group substituted by a substituent”.

In this specification, a term “unsubstituted” in the case of “a substituted or unsubstituted ZZ group” means that hydrogen atoms in the ZZ group are not substituted by a substituent. Hydrogen atoms in a term “unsubstituted ZZ group” are a protium atom, a deuterium atom, or a tritium atom.

In this specification, a term “substituted” in the case of “a substituted or unsubstituted ZZ group” means that one or more hydrogen atoms in the ZZ group are substituted by a substituent. Similarly, a term “substituted” in the case of “a BB group substituted by an AA group” means that one or more hydrogen atoms in the BB group are substituted by the AA group,

“Substituent as Described in this Specification”

Hereinafter, the substituent described in this specification will be explained.

The number of ring carbon atoms of the “unsubstituted aryl group” described in this specification is 6 to 50, preferably 6 to 30, and more preferably 6 to 18, unless otherwise specified.

The number of ring atoms of the “unsubstituted heterocyclic group” described in this specification is 5 to 50, preferably 5 to 30, and more preferably 5 to 18, unless otherwise specified.

The number of carbon atoms of the “unsubstituted alkyl group” described in this specification is 1 to 50, preferably 1 to 20, and more preferably 1 to 6, unless otherwise specified.

The number of carbon atoms of the “unsubstituted alkenyl group” described in this specification is 2 to 50, preferably 2 to 20, and more preferably 2 to 6, unless otherwise specified.

The number of carbon atoms of the “unsubstituted alkenyl group” described in this specification is 2 to 50, preferably 2 to 20, and more preferably 2 to 6, unless otherwise specified.

The number of ring carbon atoms of the “unsubstituted cycloalkyl group” described in this specification is 3 to 50, preferably 3 to 20, and more preferably 3 to 6, unless otherwise specified.

The number of ring carbon atoms of the “unsubstituted arylene group” described in this specification is 6 to 50, preferably 6 to 30, and more preferably 6 to 18, unless otherwise specified.

The number of ring atoms of the “unsubstituted divalent heterocyclic group” described in this specification is 5 to 50, preferably 5 to 30, and more preferably 5 to 18, unless otherwise specified.

The number of carbon atoms of the “unsubstituted alkylene group” described in this specification is 1 to 50, preferably 1 to 20, and more preferably 1 to 6, unless otherwise specified.

“Substituted or Unsubstituted Aryl Group”

Specific examples of the “substituted or unsubstituted aryl group” described in this specification (specific example group G1) include the following unsubstituted aryl groups (specific example group G1A), substituted aryl groups (specific example group G1B), and the like. (Here, the unsubstituted aryl group refers to the case where the “substituted or unsubstituted aryl group” is an “aryl group unsubstituted by a substituent”, and the substituted aryl group refers to the case where the “substituted or unsubstituted aryl group” is an “aryl group substituted by a substituent”.). In this specification, in the case where simply referred as an “aryl group”, it includes both a “unsubstituted aryl group” and a “substituted aryl group.”

The “substituted aryl group” means a group in which one or more hydrogen atoms of the “unsubstituted aryl group” are substituted by a substituent. Specific examples of the “substituted aryl group” include, for example, groups in which one or more hydrogen atoms of the “unsubstituted aryl group” of the following specific example group G1A are substituted by a substituent, the substituted aryl groups of the following specific example group G1B, and the like. It should be noted that the examples of the “unsubstituted aryl group” and the examples of the “substituted aryl group” enumerated in this specification are mere examples, and the “substituted aryl group” described in this specification also includes a group in which a hydrogen atom bonded with a carbon atom of the aryl group itself in the “substituted aryl group” of the following specific group G1B is further substituted by a substituent, and a group in which a hydrogen atom of a substituent in the “substituted aryl group” of the following specific group G1B is further substituted by a substituent.

Unsubstituted aryl group (specific example group G1A):
a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a bentanthryl group,
a phenanthryl group,
a bentophenanthryl group,
a phenalenyl group,
a pyrenyl group,
a chrysenyl group,
a beniochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group,
a perylenyl group, and
a monovalent aryl group derived by removing one hydrogen atom from the ring structures represented by any of the following general formulas (TEMP-1) to (TEMP-15).

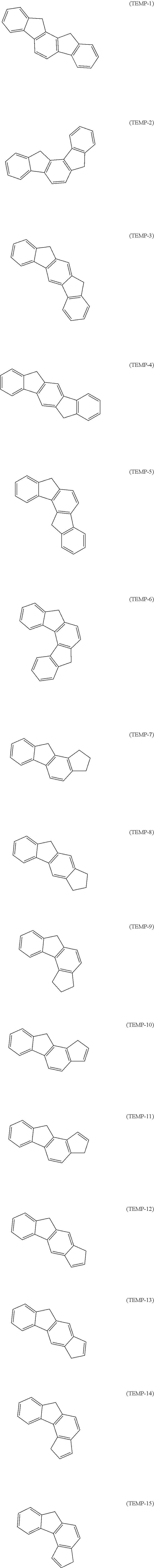

Substituted aryl group (specific example group G1B):
an o-tolyl group,
a m-tolyl group,
a p-tolyl group,
a p-xylyl group,
a m-xylyl group,
an o-xylyl group,
a p-isopropylphenyl group,
a m-isopropylphenyl group,
an o-isopropylphenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group,
a 9,9-bis(4-methylphenyl)fluorenyl group,
a 9,9-bis(4-isopropylphenyl)fluorenyl group,
a 9,9-bis(4-t-butylphenyl)fluorenyl group,
a cyanophenyl group,
a triphenylsilylphenyl group,
a trimethylsilylphenyl group,
a phenylnaphthyl group,
a naphthylphenyl group, and
a group in which one or more hydrogen atoms of a monovalent group derived from the ring structures represented by any of the general formulas (TEMP-1) to (TEMP-15) are substituted by a substituent.

“Substituted or Unsubstituted Heterocyclic Group”

The “heterocyclic group” described in this specification is a ring group having at least one hetero atom in the ring atom. Specific examples of the hetero atom include a nitrogen atom, an oxygen atom, a sulfur atom, a silicon atom, a phosphorus atom, and a boron atom.

The “heterocyclic group” in this specification is a monocyclic group or a fused ring group.

The “heterocyclic group” in this specification is an aromatic heterocyclic group or a non-aromatic heterocyclic group.

Specific examples of the “substituted or unsubstituted heterocyclic group” (specific example group G2) described in this specification include the following unsubstituted heterocyclic group (specific example group G2A), the following substituted heterocyclic group (specific example group G2B), and the like. (Here, the unsubstituted heterocyclic group refers to the case where the “substituted or unsubstituted heterocyclic group” is a “heterocyclic group unsubstituted by a substituent”, and the substituted heterocyclic group refers to the case where the “substituted or unsubstituted heterocyclic group” is a “heterocyclic group substituted by a substituent”.). In this specification, in the case where simply referred as a “heterocyclic group”, it includes both the “unsubstituted heterocyclic group” and the “substituted heterocyclic group.”

The “substituted heterocyclic group” means a group in which one or more hydrogen atom of the “unsubstituted heterocyclic group” are substituted by a substituent. Specific examples of the “substituted heterocyclic group” include a group in which a hydrogen atom of “unsubstituted heterocyclic group” of the following specific example group G2A is substituted by a substituent, the substituted heterocyclic groups of the following specific example group G2B, and the like. It should be noted that the examples of the “unsubstituted heterocyclic group” and the examples of the “substituted heterocyclic group” enumerated in this specification are mere examples, and the “substituted heterocyclic group” described in this specification includes groups in which hydrogen atom bonded with a ring atom of the heterocyclic group itself in the “substituted heterocyclic group” of the specific example group G2B is further substituted by a substituent, and a group in which hydrogen atom of a substituent in the “substituted heterocyclic group” of the specific example group G2B is further substituted by a substituent.

Specific example group G2A includes, for example, the following unsubstituted heterocyclic group containing a nitrogen atom (specific example group G2A1), the following unsubstituted heterocyclic group containing an oxygen atom (specific example group G2A2), the following unsubstituted heterocyclic group containing a sulfur atom (specific example group G2A3), and the monovalent heterocyclic group derived by removing one hydrogen atom from the ring structures represented by any of the following general formulas (TEMP-16) to (TEMP-33) (specific example group G2A4).

Specific example group G2B includes, for example, the following substituted heterocyclic group containing a nitrogen atom (specific example group G2B1), the following substituted heterocyclic group containing an oxygen atom (specific example group G2B2), the following substituted heterocyclic group containing a sulfur atom (specific example group G2B3), and the following group in which one or more hydrogen atoms of the monovalent heterocyclic group derived from the ring structures represented by any of the following general formulas (TEMP-16) to (TEMP-33) are substituted by a substituent (specific example group G2B4).

Unsubstituted heterocyclic group containing a nitrogen atom (specific example group G2A1):
a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthridinyl group,
a phenanthridinyl group,
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

Unsubstituted heterocyclic group containing an oxygen atom (specific example group G2A2):
a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

Unsubstituted heterocyclic group containing a sulfur atom (specific example group G2A3):
a thienyl group,
a thiazolyl group,
an isothiazolyl group,
a thiadiazolyl group,
a benzothiophenyl group (benzothienyl group),
an isobenzothiophenyl group (isobenzothienyl group),
a dibenzothiophenyl group (dibenzothienyl group),
a naphthobenzothiophenyl group (naphthobenzothienyl group),
a benzothiazolyl group,
a benzisothiazolyl group,
a phenothiazinyl group,
a dinaphthothiophenyl group (dinaphthothienyl group),
an azadibenzothiophenyl group (azadibenzothienyl group),
a diazadibenzothiophenyl group (diazadibenzothienyl group),
an azanaphthobenzothiophenyl group (azanaphthobenzothienyl group), and
a diazanaphthobenzothiophenyl group (diazanaphthobenzothienyl group).

Monovalent heterocyclic group derived by removing one hydrogen atom from the ring structures represented by any of the following general formulas (TEMP-16) to (TEMP-33) (specific example group G2A4):

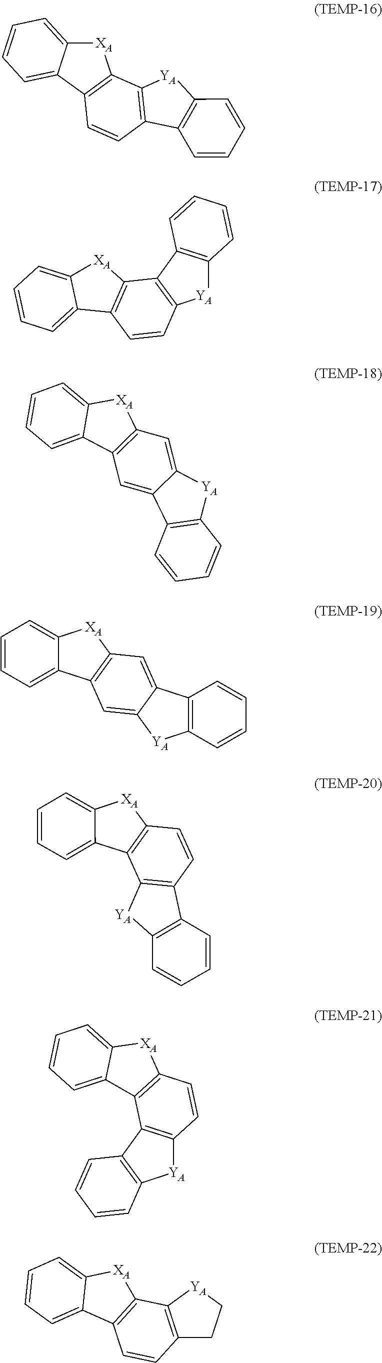

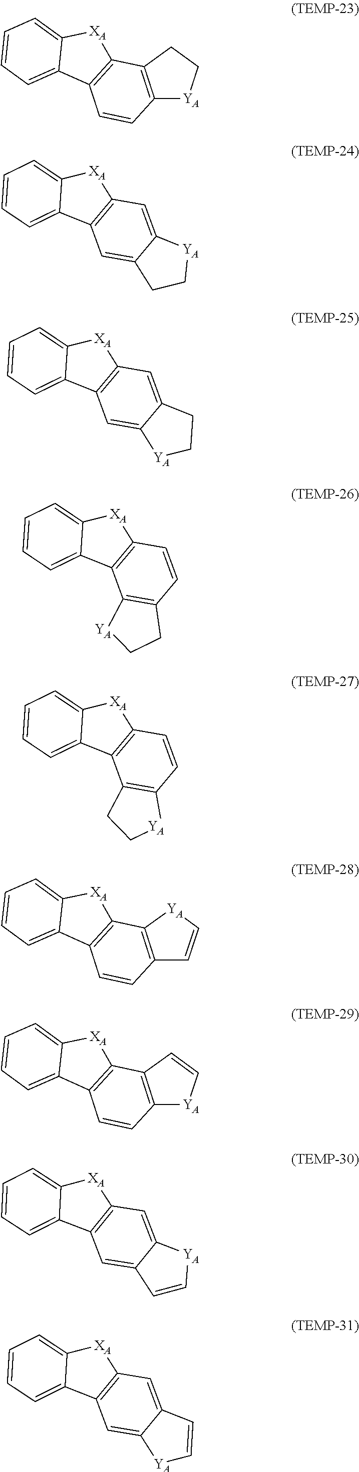

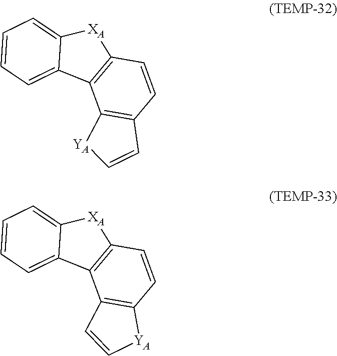

In the general formulas (TEMP-16) to (TEMP-33). X_{A} and Y_{A} are independently an oxygen atom, a sulfur atom, NH, or CH₂. Provided that at least one of X_{A} and Y_{A} is an oxygen atom, a sulfur atom, or NH.

In the general formulas (TEMP-16) to (TEMP-33), when at least one of X_{A} and Y_{A} is NH or CH₂, the monovalent heterocyclic group derived from the ring structures represented by any of the general formulas (TEMP-16) to (TEMP-33) includes a monovalent group derived by removing one hydrogen atom from these NH or CH₂.

Substituted heterocyclic group containing a nitrogen atom (specific example group G2B1):
a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

Substituted heterocyclic group containing an oxygen atom (specific example group G2B2):
a phenyldibenzofuranyl group,
a methyldibenzofuranyl group,
a t-butyldibenzofuranyl group, and
a monovalent residue of spiro[9H-xanthene-9,9′-[9H]fluorene].

Substituted heterocyclic group containing a sulfur atom (specific example group G2B3):
a phenyldibenzothiophenyl group,
a methyldibenzothiophenyl group,
a t-butyldibenzothiophenyl group, and
a monovalent residue of spiro[9H-thioxanthene-9,9′-[9H]fluorene].

Group in which one or more hydrogen atoms of the monovalent heterocyclic group derived from the ring structures represented by any of the following general formulas (TEMP-16) to (TEMP-33) are substituted by a substituent (specific example group G2B4):

The “one or more hydrogen atoms of the monovalent heterocyclic group” means one or more hydrogen atoms selected from hydrogen atoms bonded with ring carbon atoms of the monovalent heterocyclic group, a hydrogen atom bonded with a nitrogen atom when at least one of X_{A} and Y_{A} is NH, and hydrogen atoms of a methylene group when one of X_{A} and Y_{A} is CH₂.

“Substituted or Unsubstituted Alkyl Group”

Specific examples of the “substituted or unsubstituted alkyl group” (specific example group G3) described in this specification include the following unsubstituted alkyl groups (specific example group G3A) and the following substituted alkyl groups (specific example group G3B). (Here, the unsubstituted alkyl group refers to the case where the “substituted or unsubstituted alkyl group” is an “alkyl group unsubstituted by a substituent”, and the substituted alkyl group refers to the case where the “substituted or unsubstituted alkyl group” is an “alkyl group substituted by a substituent”.). In this specification, in the case where simply referred as an “alkyl group” includes both the “unsubstituted alkyl group” and the “substituted alkyl group.”

The “substituted alkyl group” means a group in which one or more hydrogen atoms in the “unsubstituted alkyl group” are substituted by a substituent. Specific examples of the “substituted alkyl group” include groups in which one or more hydrogen atoms in the following “unsubstituted alkyl group” (specific example group G3A) are substituted by a substituent, the following substituted alkyl group (specific example group G3B), and the like. In this specification, the alkyl group in the “unsubstituted alkyl group” means a linear alkyl group. Thus, the “unsubstituted alkyl group” includes a straight-chain “unsubstituted alkyl group” and a branched-chain “unsubstituted alkyl group”. It should be noted that the examples of the “unsubstituted alkyl group” and the examples of the “substituted alkyl group” enumerated in this specification are mere examples, and the “substituted alkyl group” described in this specification includes a group in which hydrogen atom of the alkyl group itself in the “substituted alkyl group” of the specific example group G3B is further substituted by a substituent, and a group in which hydrogen atom of a substituent in the “substituted alkyl group” of the specific example group G3B is further substituted by a substituent.

Unsubstituted alkyl group (specific example group G3A):
a methyl group,
an ethyl group,
a n-propyl group,
an isopropyl group,
a n-butyl group,
an isobutyl group,
a s-butyl group, and
a t-butyl group.

Substituted alkyl group (specific example group G3B):
a heptafluoropropyl group (including isomers),
a pentafluoroethyl group,
a 2.2.2-trifluoroethyl group, and
a trifluoromethyl group,

“Substituted or Unsubstituted Alkenyl Group”

Specific examples of the “substituted or unsubstituted alkenyl group” described in this specification (specific example group 64) include the following unsubstituted alkenyl group (specific example group G4A), the following substituted alkenyl group (specific example group G4B), and the like. (Here, the unsubstituted alkenyl group refers to the case where the “substituted or unsubstituted alkenyl group” is a “alkenyl group unsubstituted by a substituent”, and the “substituted alkenyl group” refers to the case where the “substituted or unsubstituted alkenyl group” is a “alkenyl group substituted by a substituent.”). In this specification, in the case where simply referred as an “alkenyl group” includes both the “unsubstituted alkenyl group” and the “substituted alkenyl group.”

The “substituted alkenyl group” means a group in which one or more hydrogen atoms in the “unsubstituted alkenyl group” are substituted by a substituent. Specific examples of the “substituted alkenyl group” include a group in which the following “unsubstituted alkenyl group” (specific example group G4A) has a substituent, the following substituted alkenyl group (specific example group 64B), and the like. It should be noted that the examples of the “unsubstituted alkenyl group” and the examples of the “substituted alkenyl group” enumerated in this specification are mere examples, and the “substituted alkenyl group” described in this specification includes a group in which a hydrogen atom of the alkenyl group itself in the “substituted alkenyl group” of the specific example group G4B is further substituted by a substituent, and a group in which a hydrogen atom of a substituent in the “substituted alkenyl group” of the specific example group G4B is further substituted by a substituent.

Unsubstituted alkenyl group (specific example group G4A):
a vinyl group,
an ally group,
a 1-butenyl group,
a 2-butenyl group, and
a 3-butenyl group.

Substituted alkenyl group (specific example group G4B):
a 1,3-butanedienyl group,
a 1-methylvinyl group,
a 1-methylallyl group,
a 1,1-dimethylallyl group,
a 2-methylallyl group, and
a 1.2-dimethylallyl group,

“Substituted or Unsubstituted Alkynyl Group”

Specific examples of the “substituted or unsubstituted alkynyl group” described in this specification (specific example group G5) include the following unsubstituted alkynyl group (specific example group G5A) and the like. (Here, the unsubstituted alkynyl group refers to the case where the “substituted or unsubstituted alkynyl group” is an “alkynyl group unsubstituted by a substituent”.). In this specification, in the case where simply referred as an “alkynyl group” includes both the “unsubstituted alkynyl group” and the “substituted alkynyl group.”

The “substituted alkynyl group” means a group in which one or more hydrogen atoms in the “unsubstituted alkynyl group” are substituted by a substituent. Specific examples of the “substituted alkynyl group” include a group in which one or more hydrogen atoms in the following “unsubstituted alkynyl group” (specific example group G5A) are substituted by a substituent, and the like.

Unsubstituted alkynyl group (specific example group G5A):
an ethynyl group,

“Substituted or Unsubstituted Cycloalkyl Group”

Specific examples of the “substituted or unsubstituted cycloalkyl group” described in this specification (specific example group G6) include the following unsubstituted cycloalkyl group (specific example group G6A), the following substituted cycloalkyl group (specific example group G6B), and the like. (Here, the unsubstituted cycloalkyl group refers to the case where the “substituted or unsubstituted cycloalkyl group” is a “cycloalkyl group unsubstituted by a substituent”, and the substituted cycloalkyl group refers to the case where the “substituted or unsubstituted cycloalkyl group” is a “cycloalkyl group substituted by a substituent”.). In this specification, in the case where simply referred as a “cycloalkyl group” includes both the “unsubstituted cycloalkyl group” and the “substituted cycloalkyl group.”

The “substituted cycloalkyl group” means a group in which one or more hydrogen atoms in the “unsubstituted cycloalkyl group” are substituted by a substituent. Specific examples of the “substituted cycloalkyl group” include a group in which one or more hydrogen atoms in the following “unsubstituted cycloalkyl group” (specific example group G6A) are substituted by a substituent, and examples of the following substituted cycloalkyl group (specific example group G6B), and the like. It should be noted that the examples of the “unsubstituted cycloalkyl group” and the examples of the “substituted cycloalkyl group” enumerated in this specification are mere examples, and the “substituted cycloalkyl group” in this specification includes a group in which one or more hydrogen atoms bonded with the carbon atom of the cycloalkyl group itself in the “substituted cycloalkyl group” of the specific example group G6B are substituted by a substituent, and a group in which a hydrogen atom of a substituent in the “substituted cycloalkyl group” of specific example group G6B is further substituted by a substituent.

Unsubstituted cycloalkyl group (specific example group G6A):
a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

Substituted cycloalkyl group (specific example group G6B):
a 4-methylcyclohexyl group.

“Group Represented by —Si(R₉₀₁)(R₉₀₂)(R₉₀₃)”

Specific examples of the group represented by —Si(R₉₀₁)(R₉₀₂)(R₉₀₃) described in this specification (specific example group G7) include:
—Si(G1)(G1)(G1),
—Si(G1)(G2)(G2),
—Si(G1)(G1)(G2),
—Si(G2)(G2)(G2),
—Si(G3)(G3)(G3), and
—Si(G6)(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —Si(G1)(G1)(G1) are the same or different.

Plural G2's in —Si(G1)(G2)(G2) are the same or different.

Plural G1's in —Si(G1)(G1)(G2) are the same or different.

Plural G2's in —Si(G2)(G2)(G2) are be the same or different.

Plural G3's in —Si(G3)(G3)(G3) are the same or different.

Plural G6's in —Si(G6)(G6)(G6) are be the same or different.

“Group Represented by —O—(R₉₀₄)”

Specific examples of the group represented by —O—(R₉₀₄) in this specification (specific example group G8) include:
—O(G1),
—O(G2),
—O(G3), and
—O(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group represented by —S—(R₉₀₅)”

Specific examples of the group represented by —S—(R₉₀₅) in this specification (specific example group G9) include:
—S(G1),
—S(G2),
—S(G3), and
—S(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

“Group Represented by —N(R₉₀₆)(R₉₀₇)”

Specific examples of the group represented by —N(R₉₀₆)(R₉₀₇) in this specification (specific example group G10) include:
—N(G1)(G1).
—N(G2)(G2),
—N(G1)(G2),
—N(G3)(G3), and
—N(G6)(G6).

G1 is the “substituted or unsubstituted aryl group” described in the specific example group G1.

G2 is the “substituted or unsubstituted heterocyclic group” described in the specific example group G2.

G3 is the “substituted or unsubstituted alkyl group” described in the specific example group G3.

G6 is the “substituted or unsubstituted cycloalkyl group” described in the specific example group G6.

Plural G1's in —N(G1)(G1) are the same or different.

Plural G2's in —N(G2)(G2) are the same or different.

Plural G3's in —N(G3)(G3) are the same or different.

Plural G6's in —N(G6)(G6) are the same or different.

“Halogen Atom”

Specific examples of the “halogen atom” described in this specification (specific example group G11) include a fluorine atom, a chlorine atom, a bromine atom, an iodine atom, and the like.

“Substituted or Unsubstituted Fluoroalkyl Group”

The “substituted or unsubstituted fluoroalkyl group” described in this specification is a group in which at least one hydrogen atom bonded with a carbon atom constituting the alkyl group in the “substituted or unsubstituted alkyl group” is substituted by a fluorine atom, and includes a group in which all hydrogen atoms bonded with a carbon atom constituting the alkyl group in the “substituted or unsubstituted alkyl group” are substituted by a fluorine atom (a perfluoro group). The number of carbon atoms of the “unsubstituted fluoroalkyl group” is 1 to 50, preferably 1 to 30, more preferably 1 to 18, unless otherwise specified in this specification. The “substituted fluoroalkyl group” means a group in which one or more hydrogen atoms of the “fluoroalkyl group” are substituted by a substituent. The “substituted fluoroalkyl group” described in this specification also includes a group in which one or more hydrogen atoms bonded with a carbon atom of the alkyl chains in the “substituted fluoroalkyl group” are further substituted by a substituent, and a group in which one or more hydrogen atom of a substituent in the “substituted fluoroalkyl group” are further substituted by a substituent. Specific examples of the “unsubstituted fluoroalkyl group” include a group in which one or more hydrogen atoms in the “alkyl group” (specific group G3) are substituted by a fluorine atom, and the like.

“Substituted or Unsubstituted Haloalkyl Group”

The “substituted or unsubstituted haloalkyl group” described in this specification is a group in which at least one hydrogen atom bonded with a carbon atom constituting the alkyl group in the “substituted or unsubstituted alkyl group” is substituted by a halogen atom, and also includes a group in which all hydrogen atoms bonded with a carbon atom constituting the alkyl group in the “substituted or unsubstituted alkyl group” are substituted by a halogen atom. The number of carbon atoms of the “unsubstituted haloalkyl group” is 1 to 50, preferably 1 to 30, more preferably 1 to 18, unless otherwise specified in this specification. The “substituted haloalkyl group” means a group in which one or more hydrogen atoms of the “haloalkyl group” are substituted by a substituent. The “substituted haloalkyl group” described in this specification also includes a group in which one or more hydrogen atoms bonded with a carbon atom of the alkyl chain in the “substituted haloalkyl group” are further substituted by a substituent, and a group in which one or more hydrogen atoms of a substituent in the “substituted haloalkyl group” are further substituted by a substituent. Specific examples of the “unsubstituted haloalkyl group” include a group in which one or more hydrogen atoms in the “alkyl group” (specific example group G3) are substituted by a halogen atom, and the like. A haloalkyl group is sometimes referred to as an alkyl halide group.

“Substituted or Unsubstituted Alkoxy Group”

Specific examples of the “substituted or unsubstituted alkoxy group” described in this specification include a group represented by −O(G3), wherein G3 is the “substituted or unsubstituted alkyl group” described in the specific example group G3. The number of carbon atoms of the “unsubstituted alkoxy group” is 1 to 50, preferably 1 to 30, more preferably 1 to 18, unless otherwise specified in this specification.

“Substituted or Unsubstituted Alkylthio Group”

Specific examples of the “substituted or unsubstituted alkylthio group” described in this specification include a group represented by —S(G3), wherein G3 is the “substituted or unsubstituted alkyl group” described in the specific example group G3. The number of carbon atoms of the “unsubstituted alkylthio group” is 1 to 50, preferably 1 to 30, more preferably 1 to 18, unless otherwise specified in this specification.

“Substituted or Unsubstituted Aryloxy Group”

Specific examples of the “substituted or unsubstituted aryloxy group” described in this specification include a group represented by −O(G1), wherein G1 is the “substituted or unsubstituted aryl group” described in the specific example group G1. The number of ring carbon atoms of the “unsubstituted aryloxy group” is 6 to 50, preferably 6 to 30, more preferably 6 to 18, unless otherwise specified in this specification.

“Substituted or Unsubstituted Arylthio Group”

Specific examples of the “substituted or unsubstituted arylthio group” described in this specification include a group represented by —S(G1), wherein G1 is a “substituted or unsubstituted aryl group” described in the specific example group G1. The number of ring carbon atoms of the “unsubstituted arylthio group” is 6 to 50, preferably 6 to 30, more preferably 6 to 18, unless otherwise specified in this specification.

“Substituted or Unsubstituted Trialkylsilyl Group”

Specific examples of the “trialkylsilyl group” described in this specification include a group represented by —Si(G3)(G3)(G3), where G3 is the “substituted or unsubstituted alkyl group” described in the specific example group G3. Plural G3's in —Si(G3)(G3)(G3) are the same or different. The number of carbon atoms in each alkyl group of the “trialkylsilyl group” is 1 to 50, preferably 1 to 20, more preferably 1 to 6, unless otherwise specified in this specification.

“Substituted or Unsubstituted Aralkyl Group”

Specific examples of the “substituted or unsubstituted aralkyl group” described in this specification is a group represented by -(G3)-(G1), wherein G3 is the “substituted or unsubstituted alkyl group” described in the specific example group G3, and G1 is the “substituted or unsubstituted aryl group” described in the specific example group G1. Therefore, the “aralkyl group” is a group in which a hydrogen atom of the “alkyl group” is substituted by an “aryl group” as a substituent, and is one form of the “substituted alkyl group.” The “unsubstituted aralkyl group” is the “unsubstituted alkyl group” substituted by the “unsubstituted aryl group”, and the number of carbon atoms of the “unsubstituted aralkyl group” is 7 to 50, preferably 7 to 30, more preferably 7 to 18, unless otherwise specified in this specification.

Specific examples of the “substituted or unsubstituted aralkyl group” include a bent group, a 1-phenylethyl group, a 2-phenylethyl group, a 1-phenylisopropyl group, a 2-phenylisopropyl group, a phenyl-t-butyl group, an α-naphthylmethyl group, a 1-α-naphthylethyl group, a 2-α-naphthylethyl group, a 1-α-naphthylisopropyl group, a 2-α-naphthylisopropyl group, a β-naphthylmethyl group, a 1-β-naphthylethyl group, a 2-β-naphthylethyl group, a 1-β-naphthylisopropyl group, a 2-β-naphthylisopropyl group, and the like.

Unless otherwise specified in this specification, examples of the substituted or unsubstituted aryl group described in this specification preferably include a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, 9,9-dimethylfluorenyl group, 9,9-diphenylfluorenyl group, and the like.

Unless otherwise specified in this specification, examples of the substituted or unsubstituted heterocyclic groups described in this specification preferably include a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenylcarbazolyl group (a (9-phenyl)carbazol-1-yl group, a (9-phenylcarbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazol-9-yl group, a phenylcarbazol-9-yl group, a phenyltriazinyl group, a biphenylyltriazinyl group, a diphenyltriazinyl group, a phenyldibenzofuranyl group, a phenyldibenzothiophenyl group, and the like.

In this specification, the carbazolyl group is specifically any of the following groups, unless otherwise specified in this specification.

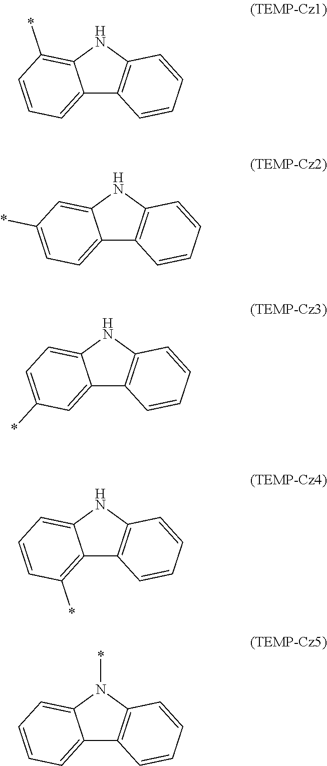 [SMILES: *c1cccc2c1[nH]c1ccccc12 ; *c1ccc2c(c1)[nH]c1ccccc12 ; *c1ccc2[nH]c3ccccc3c2c1 ; *c1cccc2[nH]c3ccccc3c12 ; *n1c2ccccc2c2ccccc21]

In this specification, the (9-phenylcarbazolyl group is specifically any of the following groups, unless otherwise specified in this specification.

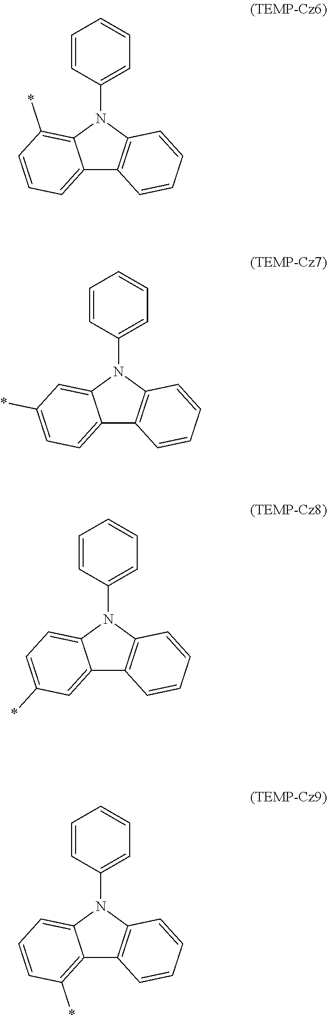 [SMILES: *c1cccc2c3ccccc3n(-c3ccccc3)c12 ; *c1ccc2c3ccccc3n(-c3ccccc3)c2c1 ; *c1ccc2c(c1)c1ccccc1n2-c1ccccc1 ; *c1cccc2c1c1ccccc1n2-c1ccccc1]

In the general formulas (TEMP-Cz1) to (TEMP-Cz9), * represents a bonding site.

In this specification, the dibenzofuranyl group and the dibenzothiophenyl group are specifically any of the following groups, unless otherwise specified in this specification.

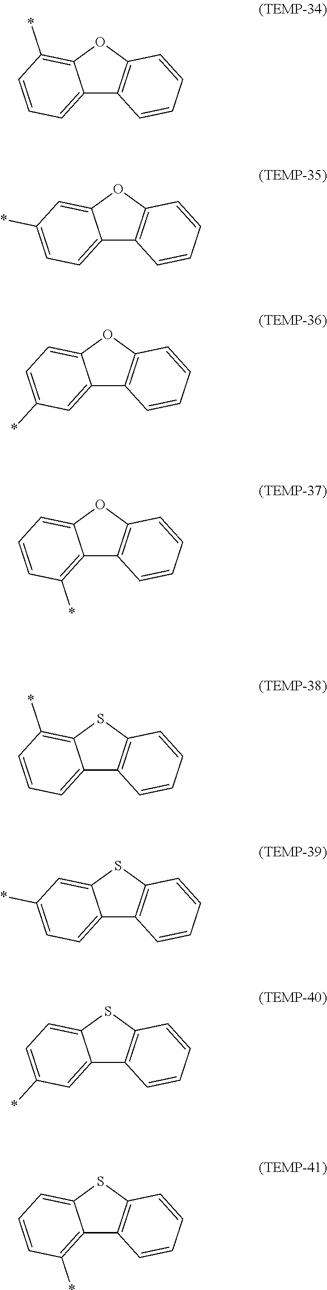

In the general formulas (TEMP-34) to (TEMP-41). * represents a bonding site.

The substituted or unsubstituted alkyl group described in this specification is preferably a methyl group, an ethyl group, a propyl group, an isopropyl group, a n-butyl group, an isobutyl group, a t-butyl group, or the like, unless otherwise specified in this specification.

“Substituted or Unsubstituted Arylene Group”

The “substituted or unsubstituted arylene group” described in this specification is a divalent group derived by removing one hydrogen atom on the aryl ring of the “substituted or unsubstituted aryl group”, unless otherwise specified. Specific examples of the “substituted or unsubstituted arylene group” (specific example group G12) include a divalent group derived by removing one hydrogen atom on the aryl ring of the “substituted or unsubstituted aryl group” described in the specific example group G1, and the like.

“Substituted or Unsubstituted Divalent Heterocyclic Group”

The “substituted or unsubstituted divalent heterocyclic group” described in this specification is a divalent group derived by removing one hydrogen atom on the heterocycle of the “substituted or unsubstituted heterocyclic group”, unless otherwise specified. Specific examples of the “substituted or unsubstituted divalent heterocyclic group” (specific example group G13) include a divalent group derived by removing one hydrogen atom on the heterocycle of the “substituted or unsubstituted heterocyclic group” described in the specific example group G2, and the like.

“Substituted or Unsubstituted Alkylene Group”

The “substituted or unsubstituted alkylene group” described in this specification is a divalent group derived by removing one hydrogen atom on the alkyl chain of the “substituted or unsubstituted alkyl group”, unless otherwise specified. Specific examples of the “substituted or unsubstituted alkylene group” (specific example group G14) include a divalent group derived by removing one hydrogen atom on the alkyl chain of the “substituted or unsubstituted alkyl group” described in the specific example group G3, and the like.

The substituted or unsubstituted arylene group described in this specification is preferably any group of the following general formulas (TEMP-42) to (TEMP-68), unless otherwise specified in this specification.

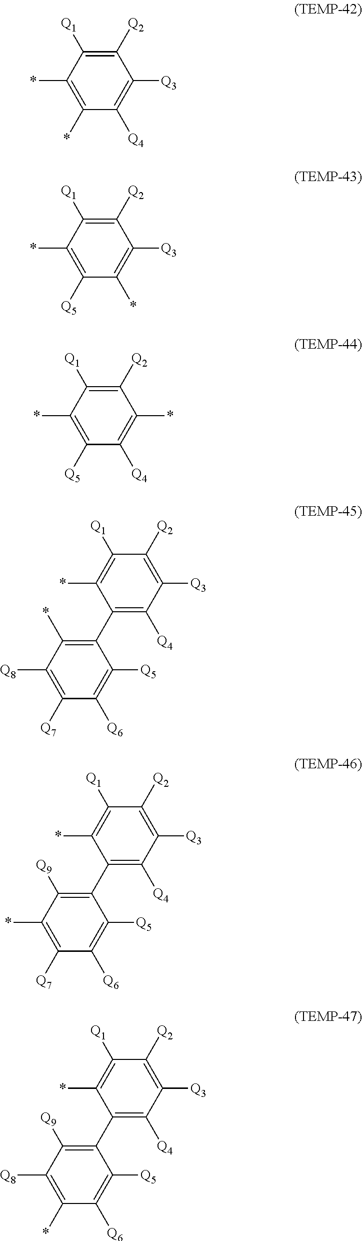

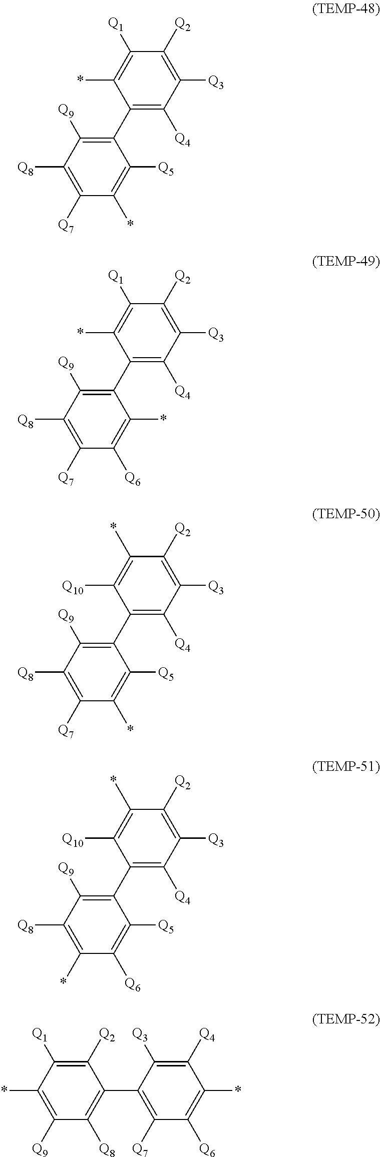

In the general formulas (TEMP-42) to (TEMP-52). Q₁ to Q₁₀ are independently a hydrogen atom or a substituent.

In the general formulas (TEMP-42) to (TEMP-52). * represents a bonding site.

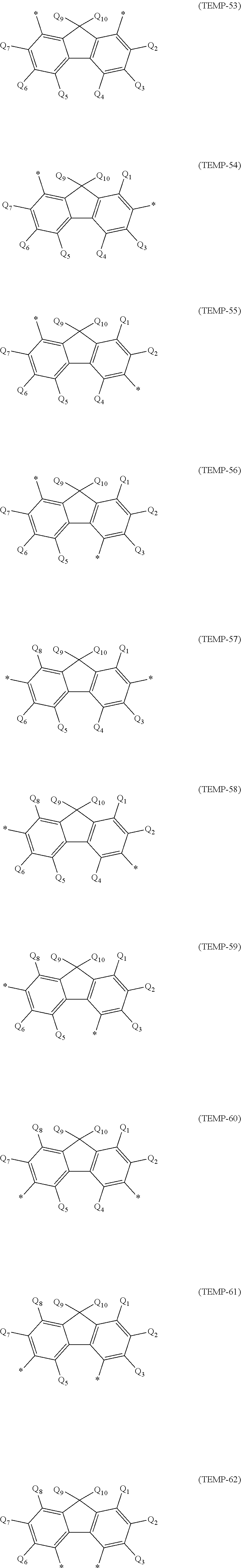

In the general formulas (TEMP-53) to (TEMP-62). Q₁ to Q₁₀ are independently a hydrogen atom or a substituent.

Q₉ and Q₁₀ may be bonded with each other via a single bond to form a ring.

In the general formulas (TEMP-53) to (TEMP-62). * represents a bonding site.

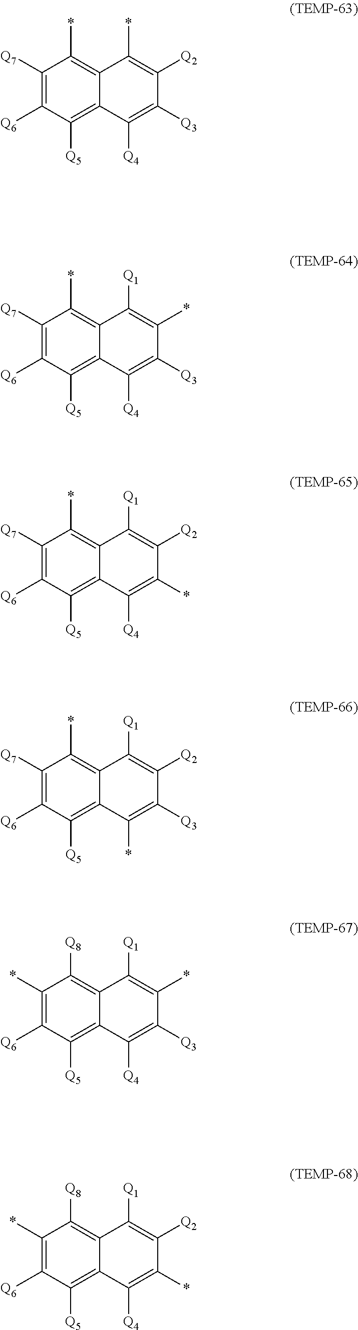

In the general formulas (TEMP-63) to (TEMP-68), Q₁ to Qₓ are independently a hydrogen atom or a substituent.

In the general formulas (TEMP-63) to (TEMP-68). * represents a bonding site.

The substituted or unsubstituted divalent heterocyclic group described in this specification is preferably any group of the following general formulas (TEMP-69) to (TEMP-102), unless otherwise specified in this specification.

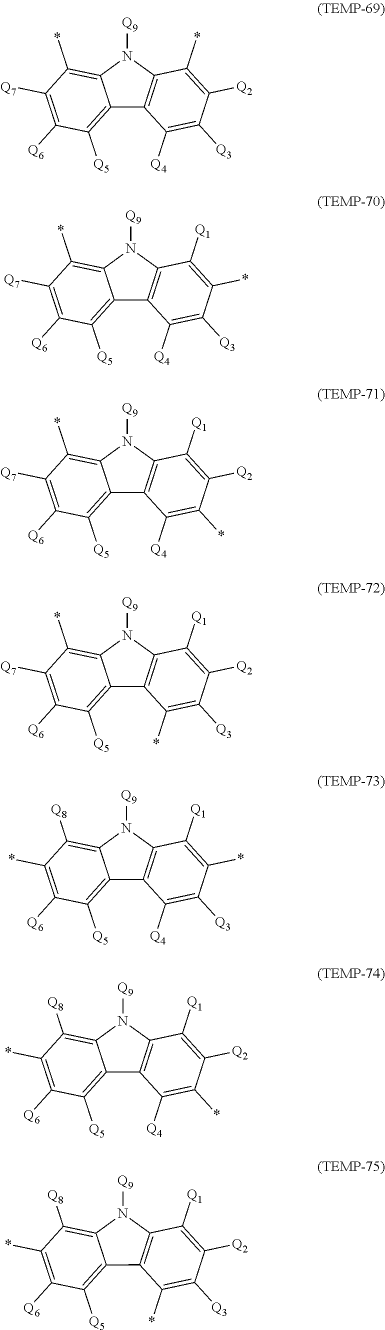

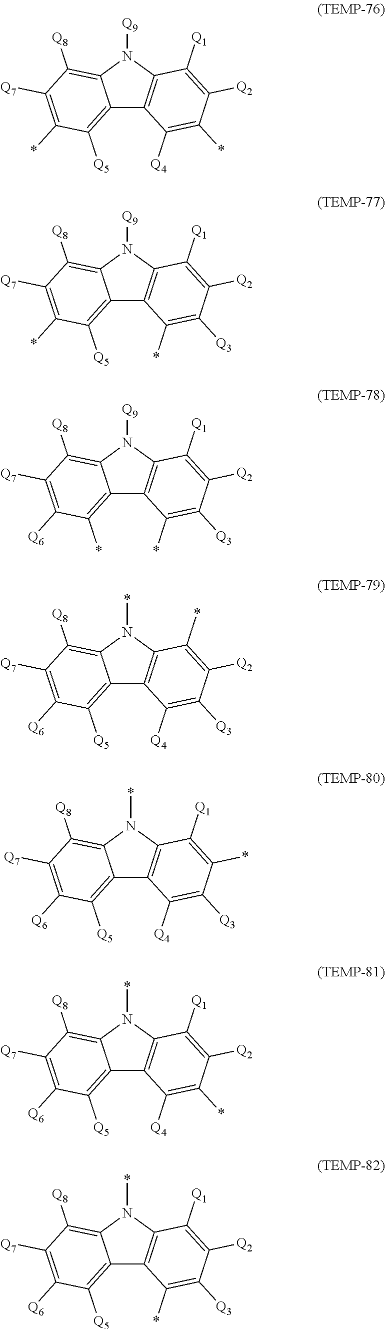

In the general formulas (TEMP-69) to (TEMP-82). Q₁ to Q₉ are independently a hydrogen atom or a substituent.

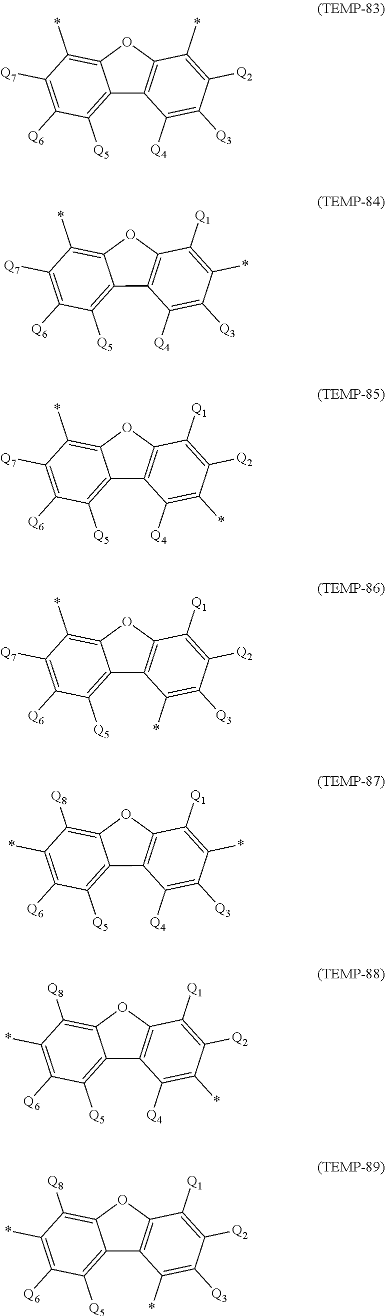

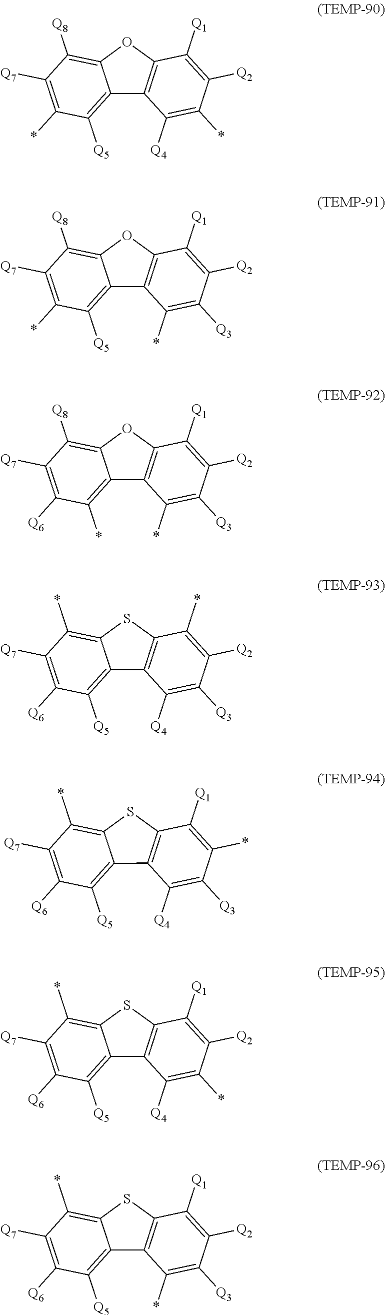

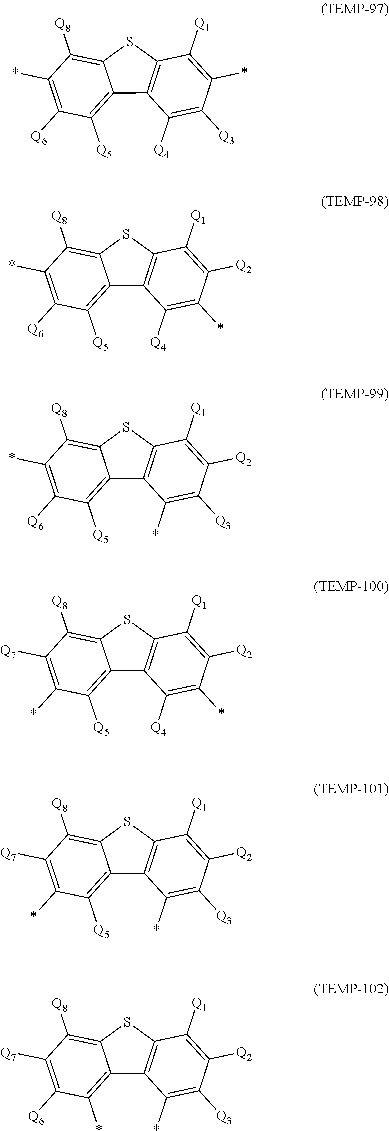

In the general formulas (TEMP-83) to (TEMP-102). Q₁ to Qₓ are independently a hydrogen atom or a substituent.

The above is the explanation of the “Substituent described in this specification.”

“The Case where Bonded with Each Other to Form a Ring”

In this specification, the case where “one or more sets of adjacent two or more form a substituted or unsubstituted monocycle by bonding with each other, form a substituted or unsubstituted fused ring by bonding with each other, or do not bond with each other” means the case where “one or more sets of adjacent two or more form a substituted or unsubstituted monocycle by bonding with each other”; the case where “one or more sets of adjacent two or more form a substituted or unsubstituted fused ring by bonding with each other”; and the case where “one or more sets of adjacent two or more do not bond with each other.”

The case where “one or more sets of adjacent two or more form a substituted or unsubstituted monocycle by bonding with each other” and the case where “one or more sets of adjacent two or more form a substituted or unsubstituted fused ring by bonding with each other” in this specification (these cases may be collectively referred to as “the case where forming a ring by bonding with each other”) will be described below. The case of an anthracene compound represented by the following general formula (TEMP-103) in which the mother skeleton is an anthracene ring will be described as an example.

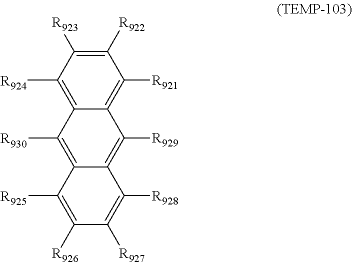

For example, in the case where “one or more sets of adjacent two or more among R₉₂₁ to R₉₃₀ form a ring by bonding with each other”, the one set of adjacent two includes a pair of R₉₂₁ and R₉₂₂, a pair of R₉₂₂ and R₉₂₃, a pair of R₉₂₃ and R₉₂₄, a pair of R₉₂₄ and R₉₃₀, a pair of R₉₃₀ and R₉₂₅, a pair of R₉₂₅ and R₉₂₄, a pair of R₉₂₆ and R₉₂₇, a pair of R₉₂₇ and R₉₂₈, a pair of R₉₂₈ and R₉₂₉, and a pair of R₉₂₉ and R₉₂₁.

The “one or more sets” means that two or more sets of the adjacent two or more sets may form a ring at the same time. For example, R₉₂₁ and R₉₂₂ form a ring Q_{A} by bonding with each other, and at the same, time R₉₂₅ and R₉₂₆, form a ring Q_{B} by bonding with each other, the anthracene compound represented by the general formula (TEMP-103) is represented by the following general formula (TEMP-104).

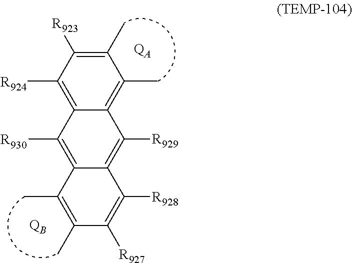

The case where the “set of adjacent two or more” form a ring includes not only the case where the set (pair) of adjacent “two” is bonded with as in the above-mentioned examples, but also the case where the set of adjacent “three or more” are bonded with each other. For example, it means the case where R₉₂₁ and R₉₂₂ form a ring Q_{A} by bonding with each other, and R₉₂₂ and R₉₂₃ form a ring Q_{C} by bonding with each other, and adjacent three (R₉₂₁, R₉₂₂ and R₉₂₃) form rings by bonding with each other and together fused to the anthracene mother skeleton. In this case, the anthracene compound represented by the general formula (TEMP-103) is represented by the following general formula (TEMP-105). In the following general formula (TEMP-105), the ring Q_{A} and the ring Q_{C} share R₉₂₂.

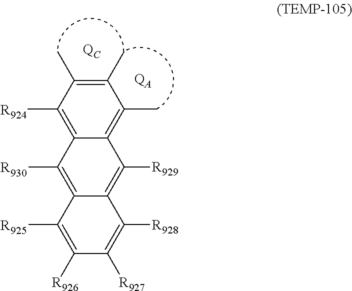

The “monocycle” or “fused ring” formed may be a saturated ring or an unsaturated ring, as a structure of the formed ring alone. Even when the “one pair of adjacent two” forms a “monocycle” or a “fused ring”, the “monocycle” or the “fused ring” may form a saturated ring or an unsaturated ring. For example, the ring Q_{A} and the ring Q_{B} formed in the general formula (TEMP-104) are independently a “monocycle” or a “fused ring.” The ring Q_{A} and the ring Q_{C} formed in the general formula (TEMP-105) are “fused ring.” The ring Q_{A} and ring Q_{C} of the general formula (TEMP-105) are fused ring by fusing the ring Q_{A} and the ring Q_{C} together. When the ring Q_{A} of the general formula (TEMP-104) is a benzene ring, the ring Q_{A} is a monocycle. When the ring Q_{A} of the general formula (TMEP-104) is a naphthalene ring, the ring Q_{A} is a fused ring.

The “unsaturated ring” includes, in addition to an aromatic hydrocarbon ring and an aromatic heterocycle, an aliphatic hydrocarbon ring with an unsaturated bond, i.e., double and or triple bonds in the ring structure (e.g., cyclohexene, cyclohexadiene, etc.), and a non-aromatic heterocycle with an unsaturated bond (e.g., dihydropyran, imidazoline, pyrazoline, quinolizine, indoline, isoindoline, etc.). The “saturated ring” includes an aliphatic hydrocarbon ring without an unsaturated bond and a non-aromatic heterocycle without ab unsaturated bond.

Specific examples of the aromatic hydrocarbon ring include a structure in which the group listed as a specific example in the specific example group G1 is terminated by a hydrogen atom.

Specific examples of the aromatic heterocycle include a structure in which the aromatic heterocyclic group listed as a specific example in the example group G2 is terminated by a hydrogen atom.

Specific examples of the aliphatic hydrocarbon ring include a structure in which the group listed as a specific example in the specific example group G6 is terminated by a hydrogen atom.

The term “to form a ring” means forming a ring only with plural atoms of the mother skeleton, or with plural atoms of the mother skeleton and one or more arbitrary atoms in addition. For example, the ring Q_{A} shown in the general formula (TEMP-104), which is formed by bonding R₉₂₁ and R₉₂₂ with each other, is a ring formed from the carbon atom of the anthracene skeleton with which R₉₂₁ is bonded, the carbon atom of the anthracene skeleton with which R₉₂₂ is bonded, and one or more arbitrary atoms. For example, in the case where the ring Q_{A} is formed with R₉₂₁ and R₉₂₂, when a monocyclic unsaturated ring is formed with the carbon atom of the anthracene skeleton with which R₉₂₁ is bonded, the carbon atom of the anthracene skeleton with which R₉₂₂ is bonded, and four carbon atoms, the ring formed with R₉₂₁ and R₉₂₂ is a benzene ring.

Here, the “arbitrary atom” is preferably at least one atom selected from the group consisting of a carbon atom, a nitrogen atom, an oxygen atom, and a sulfur atom, unless otherwise specified in this specification. In the arbitrary atom (for example, a carbon atom or a nitrogen atom), a bond which does not form a ring may be terminated with a hydrogen atom or the like, or may be substituted with “arbitrary substituent” described below. When an arbitrary atom other than a carbon atom is contained, the ring formed is a heterocycle.

The number of “one or more arbitrary atom(s)” constituting a monocycle or a fused ring is preferably 2 or more and 15 or less, more preferably 3 or more and 12 or less, and still more preferably 3 or more and 5 or less, unless otherwise specified in this specification.

The “monocycle” is preferable among the “monocycle” and the “fused ring”, unless otherwise specified in this specification.

The “unsaturated ring” is preferable among the “saturated ring” and the “unsaturated ring”, unless otherwise specified in this specification.

Unless otherwise specified in this specification, the “monocycle” is preferably a benzene ring.

Unless otherwise specified in this specification, the “unsaturated ring” is preferably a benzene ring.

Unless otherwise specified in this specification, when “one or more sets of adjacent two or more” are “bonded with each other to form a substituted or unsubstituted monocycle” or “bonded with each other to form a substituted or unsubstituted fused ring”, this specification, one or more sets of adjacent two or more are preferably bonded with each other to form a substituted or unsubstituted “unsaturated ring” from plural atoms of the mother skeleton and one or more and 15 or less atoms which is at least one kind selected from a carbon atom, a nitrogen atom, an oxygen atom, and a sulfur atom.

The substituent in the case where the above-mentioned “monocycle” or “fused ring” has a substituent is, for example, an “arbitrary substituent” described below. Specific examples of the substituent which the above-mentioned “monocycle” or “fused ring” has include the substituent described above in the “Substituent described in this specification” section.

The substituent in the case where the above-mentioned “saturated ring” or “unsaturated ring” has a substituent is, for example, an “arbitrary substituent” described below. Specific examples of the substituent which the above-mentioned “monocycle” or “fused ring” has include the substituent described above in the “Substituent described in this specification” section.

The foregoing describes the case where “one or more sets of adjacent two or more form a substituted or unsubstituted monocycle by bonding with each other” and the case where “one or more sets of adjacent two or more form a substituted or unsubstituted fused ring by bonding with each other” (the case where “forming a ring by bonding with each other”).

Substituent in the Case of “Substituted or Unsubstituted”

In one embodiment in this specification, the substituent (in this specification, sometimes referred to as an “arbitrary substituent”) in the case of “substituted or unsubstituted” is, for example, a group selected from the group consisting of:
an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
an unsubstituted aryl group including 6 to 50 ring carbon atoms, and
an unsubstituted heterocyclic group including 5 to 50 ring atoms,
wherein, R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted heterocyclic group including 5 to 50 ring atoms.

When two or more R₉₀₁'s are present, the two or more R₉₀₁'s may be the same or different.

When two or more R₉₀₂'s are present, the two or more R₉₀₂'s may be the same or different.

When two or more R₉₀₃'s are present, the two or more R₉₀₃'s may be the same or different.

When two or more R₉₀₄'s are present, the two or more R₉₀₄'s may be the same or different.

When two or more R₉₀₅'s are present the two or more R₉₀₅'s may be the same or different.

When two or more R₉₀₆'s are present, the two or more R₉₀₆'s may be the same or different.

When two or more R₉₀₇'s are present, the two or more R₉₀₇'s may be the same or different.

In one embodiment, the substituent in the case of “substituted or unsubstituted” is a group selected from the group consisting of
an alkyl group including 1 to 50 carbon atoms,
an aryl group including 6 to 50 ring carbon atoms, and
a heterocyclic group including 5 to 50 ring atoms.

In one embodiment, the substituent in the case of “substituted or unsubstituted” is a group selected from the group consisting of:
an alkyl group including 1 to 18 carbon atoms,
an aryl group including 6 to 18 ring carbon atoms, and
a heterocyclic group including 5 to 18 ring atoms.

Specific examples of each of the arbitrary substituents include specific examples of substituent described in the section “Substituent described in this specification” above.

Unless otherwise specified in this specification, adjacent arbitrary substituents may form a “saturated ring” or an “unsaturated ring”, preferably form a substituted or unsubstituted saturated 5-membered ring, a substituted or unsubstituted saturated 6-membered ring, a substituted or unsubstituted unsaturated 5-membered ring, or a substituted or unsubstituted unsaturated 6-membered ring, more preferably form a benzene ring.

Unless otherwise specified in this specification, the arbitrary substituent may further have a substituent. The substituent which the arbitrary substituent further has is the same as that of the above-mentioned arbitrary substituent.

In this specification, the numerical range represented by “AA to BB” means the range including the numerical value AA described on the front side of “AA to BB” as the lower limit and the numerical value BB described on the rear side of “AA to BB” as the upper limit.

### [Composition]

A composition according to an aspect of the present invention including a compound represented by the formula (A1) and a compound represented by the formula (B1). Provided that the compound represented by the formula (A1) and the compound represented by the formula (B1) are different compounds.

When the compound represented by the formula (A1) and the compound represented by the formula (B1) are used in combination, it can be achieved that the driving voltage of the organic electroluminescence device is reduced, the external quantum efficiency EQE thereof is enhanced and the device lifetime thereof is enhanced.

The form of the composition in an aspect of the present invention is not particularly limited, and for example, solid, powder, solution, and a film (layer) can be given. As the film (layer), for example, an organic layer configuring the organic EL device (for example, an emitting layer) can be used. When it is solid or powder, it may be formed in the shape of pellet.

Further, in terms of the “composition” in the form of the film (layer), the method for forming it is not also particularly limited. For example, when the “composition” is the organic layer configuring the organic EL device, as the organic layer, a single organic layer may be formed using materials in which two compounds are mixed in advance, or a single organic layer may be formed separately using two compounds.

Here, the expression “the compound represented by the formula (A1) and the compound represented by the formula (B1) are different” means that chemical structures (skeletal structures) of two compounds are different. For example, when the two compounds have the same structure, except that hydrogen atoms possessed by them are protium atoms (H) or deuterium atoms (D), the two compounds are not “different compounds”.

Regarding the composition in an aspect of the present invention, the amount ratio of the compound represented by the formula (A1) and the compound represented by the formula (B1) is not particularly limited, and the amount ratio of materials can be appropriately determined depending on the property of each compound and effects desired for the composition.

In one embodiment, the amount of the compound represented by the formula (A1) is 10% by mass or more and 90% by mass or less based on the total amount of the compound represented by the formula (A1) and the compound represented by the formula (B1), and it is preferably 20% by mass or more and 80% by mass or less, more preferably 40% by mass or more and 60% by mass or less.

The composition in an aspect of the present invention may include a component other than the compound represented by the formula (A1) and the compound represented by the formula (B1). Other component is not particularly limited, but an aryl compound, an azine compound, an mole compound, an oxide compound, a metal complex and the like can be given.

### <Compound Represented by the Formula (A1)>

One compound included in the composition according to an aspect of the present invention is a compound represented by the following formula (A1).

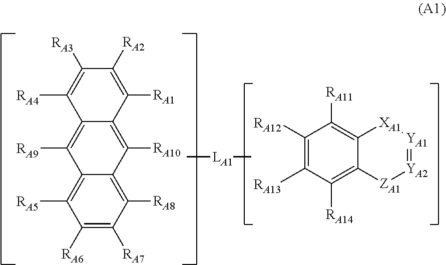

In the formula (A1),
one of R_{A1} to R_{A10} is a single bond bonding with L_{A1};
one or more sets of the adjacent two or more of R_{A1} to R_{A10} which are not a single bond bonding with L_{A1} form a substituted or unsubstituted, saturated or unsaturated ring by bonding with each other, or do not form the ring;
one of R_{A11} to R_{A14} is a single bond bonding with L_{A1};
L_{A1} is a single bond, a substituted or unsubstituted arylene group having 6 to 50 ring carbon atoms, or a substituted or unsubstituted divalent heterocyclic group having 5 to 50 ring atoms;
X_{A1} is O or S;
Y_{A1} and Y_{A2} are independently a single bond, or (Rₐ);
Z_{A1} is a single bond, or C(Rₐ)(R_{b});
one or more sets of the adjacent two or more of Rₐ, and R_{A11} to R_{A14} which are not a single bond bonding with L_{A1} form a substituted or unsubstituted, saturated or unsaturated ring by bonding with each other, or do not form the ring;
R_{b}, R_{A1} to R_{A10} and R_{A11} to R_{A14} which are not a single bond bonding with L_{A1} and which do not form the ring, and Rₐ which do not form the ring are independently a hydrogen atom, or a substituent R;
   the substituent R is selected from the group consisting of
   a substituted or unsubstituted alkyl group having 1 to 50 carbon atoms.
   a substituted or unsubstituted alkenyl group having 2 to 50 carbon atoms.
   a substituted or unsubstituted alkynyl group having 2 to 50 carbon atoms,
   a substituted or unsubstituted cycloalkyl group having 3 to 50 ring carbon atoms.
   —Si(R₉₀₁)(R₉₀₂)(R₉₀₃),
   —O—(R₉₀₄),
   —S—(R₉₀₅),
   —N(R₉₀₆)(R₉₀₇),
   a halogen atom, a cyano group, a nitro group,
   a substituted or unsubstituted aryl group having 6 to 50 ring carbon atoms, and
a substituted or unsubstituted monovalent heterocyclic group having 5 to 50 ring atoms; and
R₉₀₁ to R₉₀₇ are independently
   a hydrogen atom,
   a substituted or unsubstituted alkyl group having 1 to 50 carbon atoms,
   a substituted or unsubstituted cycloalkyl group having 3 to 50 ring carbon atoms.
   a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other.

A structure of the following moiety (p_{A}) in the compound represented by the formula (A1) can be a structure of the following moiety (p_{A-1}) or (p_{A-2}).

When L_{A1} is a single bond, the structure of the following moiety (p_{A}) and the anthracene skeleton are bonded via a single bond in the compound represented by the formula (A1).

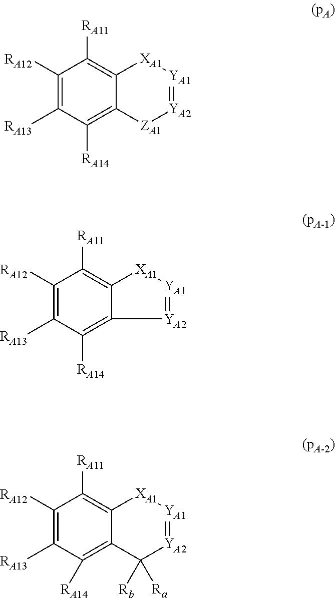

In the formulas (p_{A-1}) and (p_{A-2}), R_{A11} to R_{A14}, X_{A1}, Y_{A1}, Y_{A2}, Rₐ, and R_{b} are the same as defined in the formula (A1).

In one embodiment, the compound represented by the formula (A1) is a compound represented by the following formula (A2).

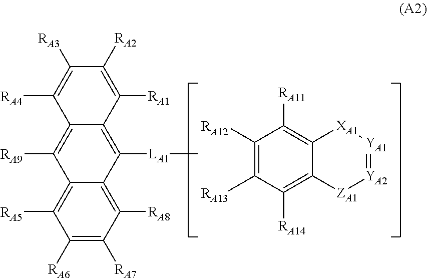

In the formula (A2), R_{A1} to R_{A9}, R_{A11} to R_{A14}, L_{A1}, X_{A1}, Y_{A1}, Y_{A2}, and Z_{A1} are the same as defined in the formula (A1).

In one embodiment, the compound represented by the formula (A1) is a compound represented by the following formula (A2-1).

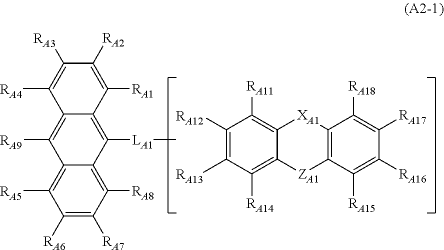

In the formula (A2-1), R_{A1} to R_{A9}, R_{A11} to R_{A14}, L_{A1}, X_{A1}, and Z_{A1} are the same as defined in the formula (A1);
one or more sets of the adjacent two or more of Rₐ and R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

In one embodiment, the compound represented by the formula (A1) is a compound represented by the following formula (A3).

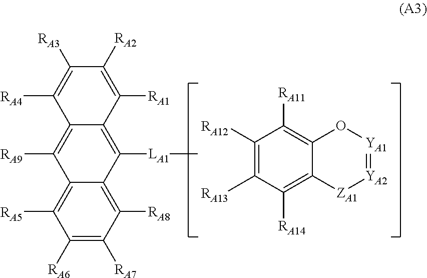

In the formula (A3), R_{A1} to R_{A9}, R_{A11} to R_{A14}, L_{A1}, Y_{A1}, Y_{A2}, and Z_{A1} are the same as defined in the formula (A1).

In one embodiment, the compound represented by the formula (A1) is a compound represented by the following formula (A4).

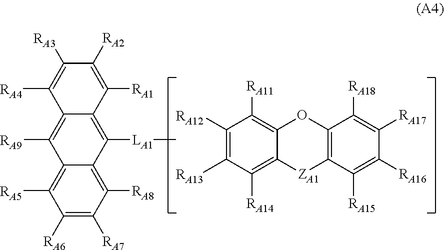

In the formula (A4), R_{A1} to R_{A9}, R_{A11} to R_{A14}, L_{A1}, and Z_{A1} are the same as defined in the formula (A1);
one or more sets of the adjacent two or more of Rₐ, and R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

When Z_{A1} in the formula (A4) is C(Rₐ)(R_{b}), as defined in the formula (A1), Rₐ and R_{b} do not form the ring by bonding with each other and a spiroxanthene is not formed.

In one embodiment, the compound represented by the formula (A1) is a compound represented by the following formula (A5-1).

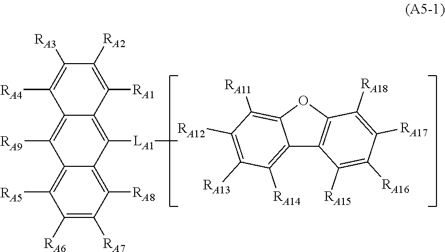

In the formula (A5-1), R_{A1} to R_{A9}, R_{A11} to R_{A14}, and L_{A1} are the same as defined in the formula (A1);
one or more sets of the adjacent two or more of R_{A14} to R_{A18} form a substituted or unsubstituted, saturated or unsaturated ring by bonding with each other, or do not form the ring; and
R_{A15} to R_{A18} is which do not form the ring are independently a hydrogen atom, or a substituent R; and the substituent R is the same as defined in the formula (A1).

In one embodiment, the compound represented by the formula (A1) is selected from the group consisting of a compound represented by the following formula (A5-1-1), and a compound represented by the following formula (A5-1-2).

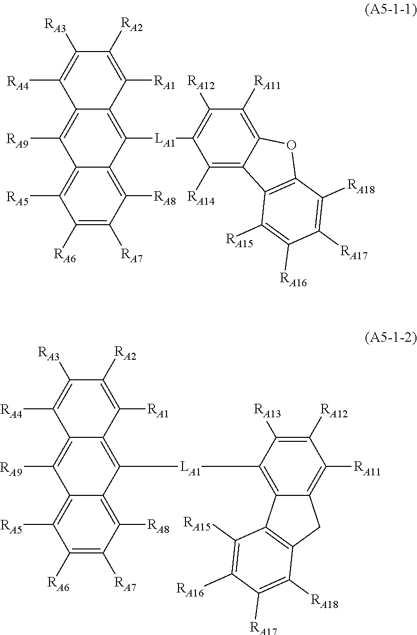

In the formulas (A5-1-1) and (A5-1-2), R_{A1} to R_{A9}, R_{A11} to R_{A14}, and L_{A1} are the same as defined in the formula (A1
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

In one embodiment, the compound represented by the formula (A1) is selected from the group consisting of a compound represented by the following formula (A5-1-1b), and a compound represented by the following formula (A5-1-2b).

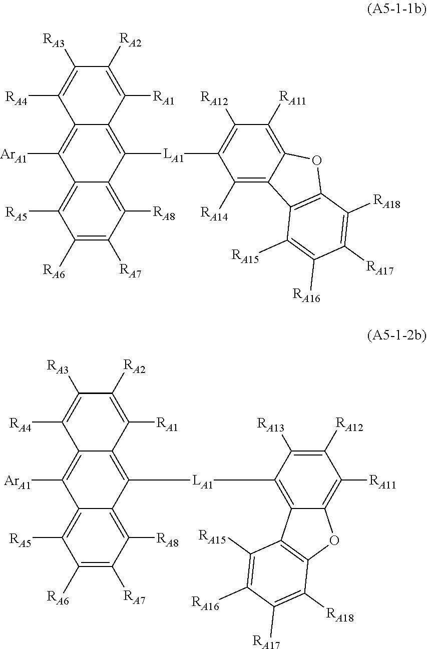

In the formulas (A5-1-1b) and (A5-1-2b), R_{A1} to R_{A8}, R_{A11} to R_{A14}, and L_{A1} are the same as defined in the formula (A1):

Ar_{A1} is a substituted or unsubstituted aryl group having 6 to 50 ring carbon atoms:
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

In one embodiment, the compound represented by the formula (A1) is selected from the group consisting of a compound represented by the following formula (A6-1), a compound represented by the following formula (A6-2), and a compound represented by the following formula (A6-3).

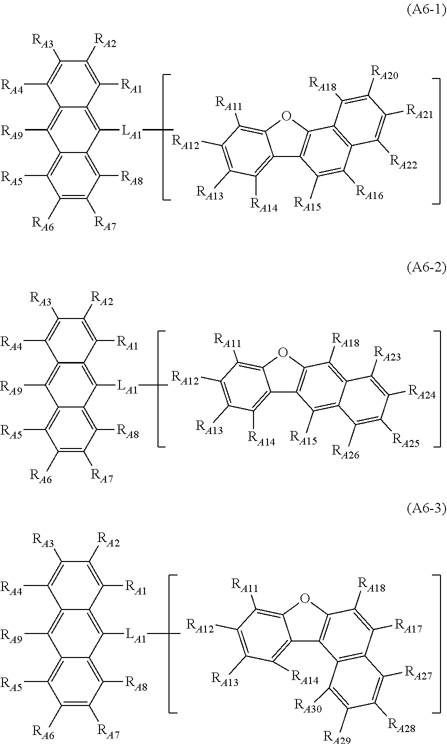

In the formulas (A6-1) to (A6-3), R_{A1} to R_{A9}, R_{A11} to R_{A14}, and L_{A1} are the same as defined in the formula (A1);
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; and the substituent R is the same as defined in the formula (A1).

In one embodiment, the compound represented by the formula (A1) is selected from the group consisting of a compound represented by the following formula (A6-1a), a compound represented by the following formula (A6-3a), and a compound represented by the following formula (A6-3a).

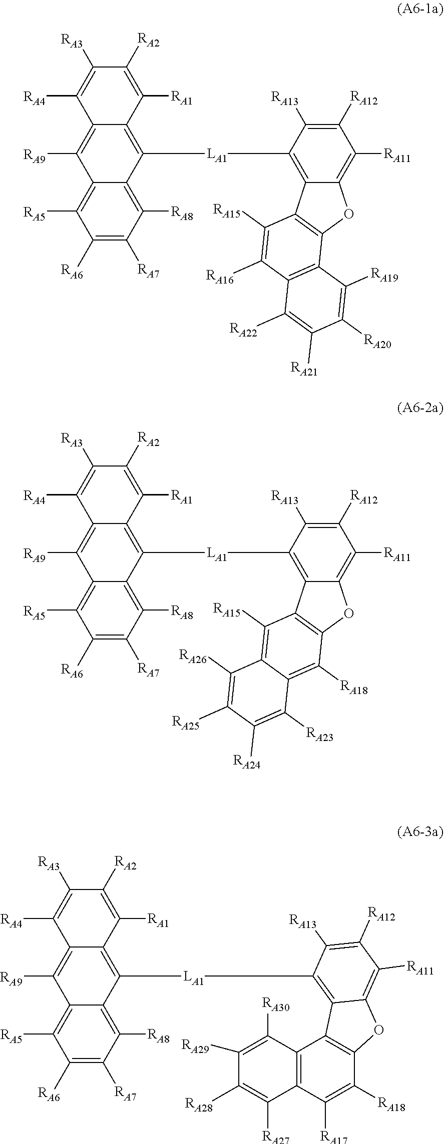

In the formulas (A6-1a) to (A6-3a), R_{A1} to R_{A9}, R_{A11} to R_{A14}, and L_{A1} are the same as defined in the formula (A1);
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; and the substituent R is the same as defined in the formula (A1).

In one embodiment, the compound represented by the formula (A1 is selected from the group consisting of a compound represented by the following formula (A6-4a), a compound represented by the following formula (A5-5a), and a compound represented by the following formula (A6-6a).

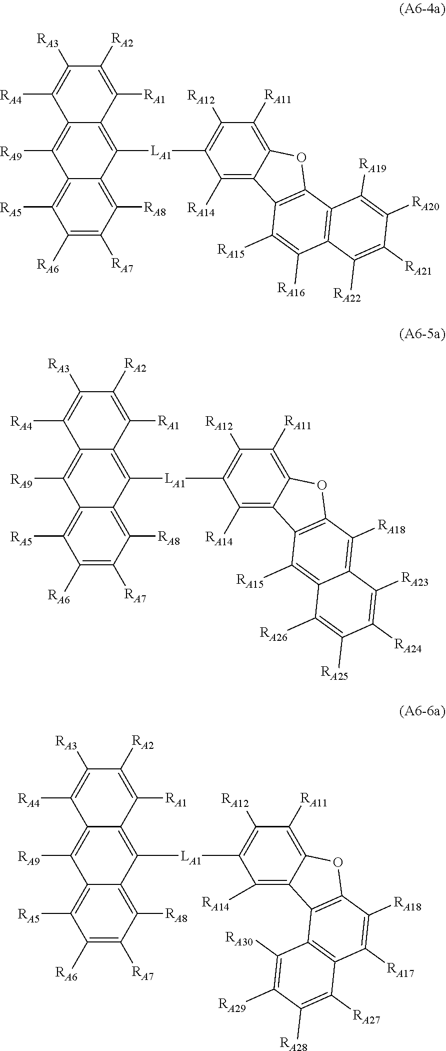

In the formulas (A6-4a) to (A6-6a), R_{A1} to R_{A9}, R_{A11}, R_{A12}, R_{A14}, and L_{A1} are the same as defined in the formula (A1):
one or more sets of the adjacent two or more of R_{A15} to R_{A30} form a substituted or unsubstituted, saturated or unsaturated ring by bonding with each other, or do not form the ring; and
R_{A15} to R_{A30} go which do not form the ring are independently a hydrogen atom, or a substituent R; and the substituent R is the same as defined in the formula (A1).

In one embodiment, the compound represented by the formula (A1) is selected from the group consisting of a compound represented by the following formula (A6-1b), a compound represented by the following formula (A6-2b), and a compound represented by the following formula (A6-3b).

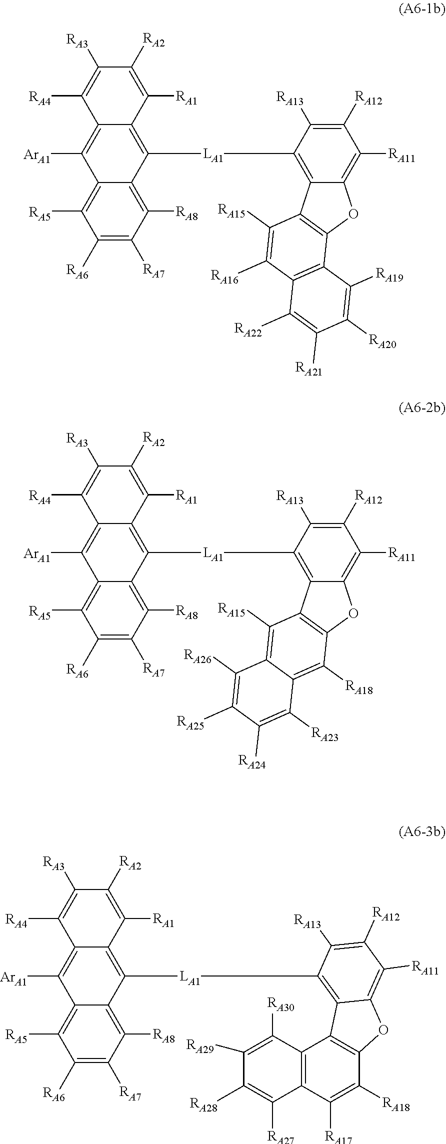

In the formulas (A6-1b) to (A6-3b), R_{A1} to R_{A8}, R_{A11} to R_{A13}, and L_{A1} are the same as defined in the formula (A1):

Arm is a substituted or unsubstituted aryl group having 6 to 50 ring carbon atoms:
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; and the substituent R is the same as defined in the formula (A1).

In one embodiment, the compound represented by the formula (A1) is selected from the group consisting of a compound represented by the following formula (A6-4b), a compound represented by the following formula (A6-5b), and a compound represented by the following formula (A6-6b).

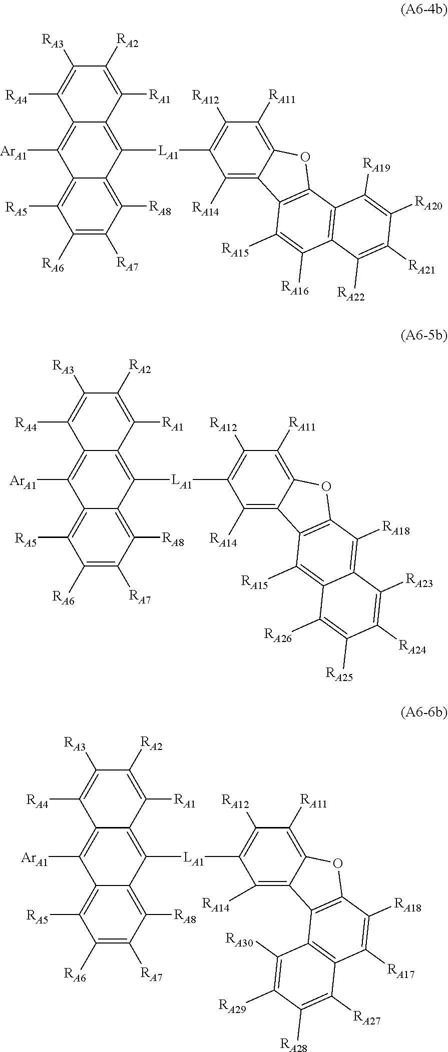

In the formulas (A6-4b) to (A6-6b), R_{A1} to R_{A8}, R_{A11}, R_{A12}, R_{A14}, and L_{A1} are the same as defined in the formula (A1).
R_{A1} is a substituted or unsubstituted aryl group having 6 to 50 ring carbon atoms;
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; and the substituent R is the same as defined in the formula (A1).

In one embodiment, the compound represented by the formula (A1) is a compound represented by the following formula (A5-2).

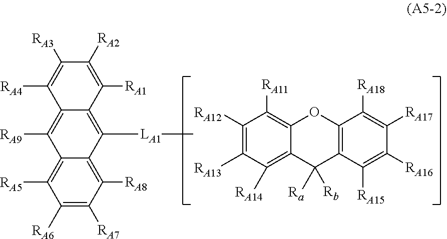

In the formula (A5-2), R_{A1} to R_{A9}, R_{A11} to R_{A14}, L_{A1}, Rₐ, and R_{b} are the same as defined in the formula (A1):
one or more sets of the adjacent two or more of Rₐ, and R_{A14} to R_{A18} form a substituted or unsubstituted, saturated or unsaturated ring by bonding with each other, or do not form the ring; and
Rₐ and R_{b} which do not form the ring, and R_{A15} to R_{A18} which do not form the ring are independently a hydrogen atom, or a substituent R; and the substituent R is the same as defined in the formula (A1).

In one embodiment, the compound represented by the formula (A1) is selected from the group consisting of a compound represented by the following formula (A5-2-1), and a compound represented by the following formula (A5-2-2).

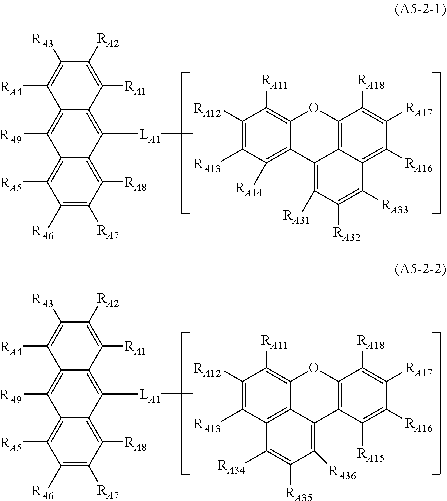

In the formulas (A5-2-1) and (A5-2-2), R_{A1} to R_{A9}, R_{A11} to R_{A14}, and L_{A1} are the same as defined in the formula (A1):
one or more sets of the adjacent two or more of R_{A13} a R_{A18}, and R_{A31} to R_{A34}, form a substituted or unsubstituted, saturated or unsaturated ring by bonding with each other, or do not form the ring; and
R_{A15} to R_{A18} and R_{A31}, to R_{A36}, which do not form the ring are independently a hydrogen atom, or a substituent R; and the substituent R is the same as defined in the formula (A1).

In one embodiment, the compound represented by the formula (A1) is selected from the group consisting of a compound represented by the following formula (A5-2-1a), and a compound represented by the following formula (A5-2-2a).

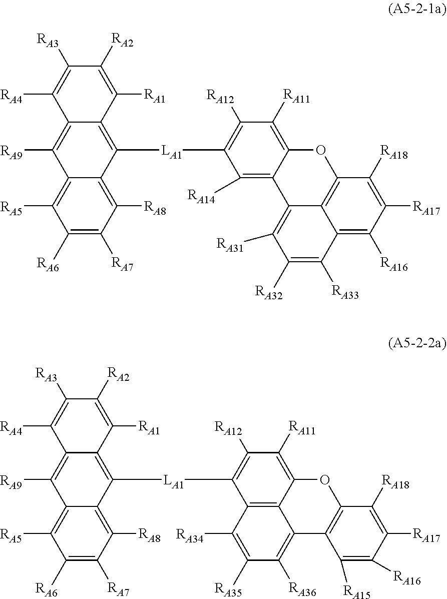

In the formulas (A5-2-1a) and (A5-2-2a), R_{A1} to R_{A9}, R_{A11}, R_{A12}, R_{A14}, and L_{A1} are the same as defined in the formula (A1):
one or more sets of the adjacent two or more of R_{A14} to R_{A18}, and R_{A31} to R_{A36}, form
a substituted or unsubstituted, saturated or unsaturated ring by bonding with each other, or do not form the ring; and
R_{A15} to R_{A18} and R_{A31} to R_{A36}, which do not form the ring are independently a hydrogen atom, or a substituent R; and the substituent R is the same as defined in the formula (A1).

In one embodiment, the compound represented by the formula (A1) is selected from the group consisting of a compound represented by the following formula (A5-2-1b), and a compound represented by the following formula (A5-2-2b).

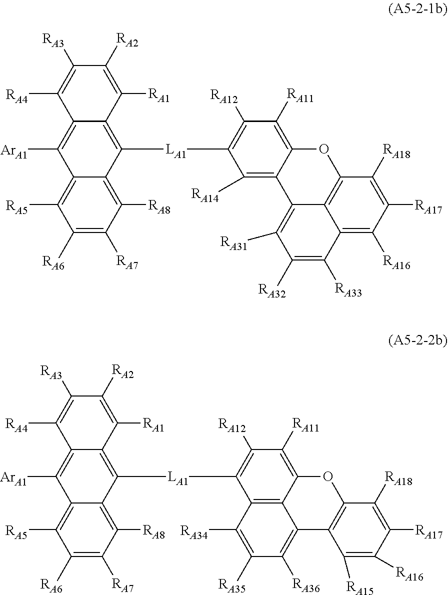

In the formulas (A5-2-1b) and (A5-2-2b), R_{A1} to R_{A8}, R_{A11}, R_{A12}, R_{A14}, and L_{A1} are the same as defined in the formula (A1):
Ar_{A1} is a substituted or unsubstituted aryl group having 6 to 50 ring carbon atoms;
one or more sets of the adjacent two or more of R_{A14} to R_{A18}, and R_{A31} to R_{A36}, form a substituted or unsubstituted, saturated or unsaturated ring by bonding with each other, or do not form the ring; and
R_{A15} to R_{A18} and R_{A31} to R_{A36}, which do not form the ring are independently a hydrogen atom, or a substituent R; and the substituent R is the same as defined in the formula (A1).

In one embodiment, the compound represented by the formula (A1) is a compound represented by the following formula (A5-3).

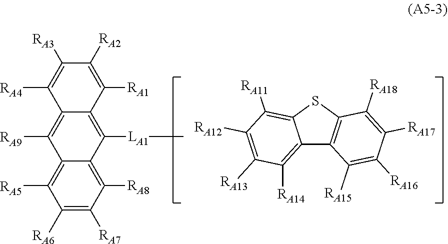

In the formula (A5-3), R_{A1} to R_{A9}, R_{A11} to R_{A14}, and L_{A1} are the same as defined in the formula (A1);
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

In one embodiment, the compound represented by the formula (A1) is selected from the group consisting of a compound represented by the following formula (A5-3-1), and a compound represented by the following formula (A5-3-2).

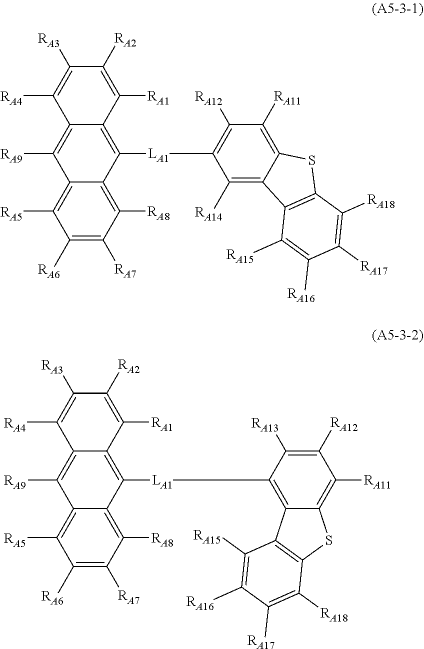

In the formulas (A5-3-1) and (A5-3-2), R_{A1}, to R_{A9}, R_{A11} to R_{A14}, and L_{A1} are the same as defined in the formula (A1):
one or more sets of the adjacent two or more of R_{A14} to R_{A18} form a substituted or unsubstituted, saturated or unsaturated ring by bonding with each other, or do not form the ring; and
R_{A15} to R_{A18}, which do not form the ring are independently a hydrogen atom, or a substituent R; and the substituent R is the same as defined in the formula (A1).

In one embodiment, the compound represented by the formula (A1) is selected from the group consisting of a compound represented by the following formula (A5-3-1b), and a compound represented by the following formula (A5-3-2b).

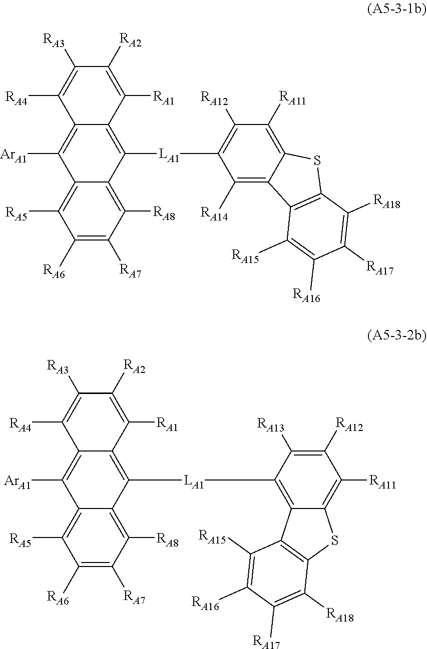

In the formulas (A5-3-1b) and (A5-3-2b), R_{A1} to R_{A8}, R_{A11} to R_{A14}, and L_{A1} are the same as defined in the formula (A1):
Ar_{A1} is a substituted or unsubstituted aryl group having 6 to 50 ring carbon atoms;
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

In one embodiment, R_{A1} to R_{A8} in the formula (A1) are hydrogen atoms.

In one embodiment, R_{A11} to R_{A14} which are not a single bond bonding with L_{A1} and which do not form the ring in the formula (A1) are hydrogen atoms.

In one embodiment, L_{A1} in the formula (A1) is a single bond, or a substituted or unsubstituted arylene group having 6 to 50 ring carbon atoms.

In one embodiment, L_{A1} in the formula (A1) is a single bond, or a substituted or unsubstituted arylene group having 6 to 20 ring carbon atoms.

In one embodiment, L_{A1} in the formula (A1) is a single bond, a phenylene group, or a biphenylene group.

In one embodiment, Ar_{A1} is a substituted or unsubstituted phenyl group, a substituted or unsubstituted biphenyl group, or a substituted or unsubstituted naphthyl group.

### <Compound Represented by the Formula (B1)>

The other compound included in the composition according to an aspect of the present invention is a compound represented by the following formula (B1).

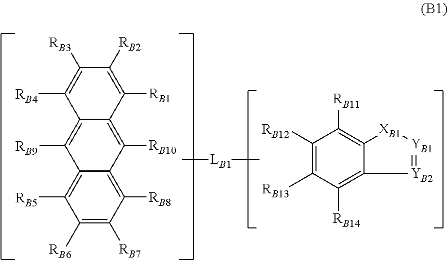

In the formula (B1),
one of R_{B1} to R_{B10} is a single bond bonding with L_{B1};
one or more sets of the adjacent two or more of R_{B1} to R_{B10}, which are not a single bond bonding with L_{B1} form a substituted or unsubstituted, saturated or unsaturated ring by bonding with each other, or do not form the ring;
X_{B1} is O, S, N(R_{c}), or C(R_{d})(Rₑ);
one of R_{B11} to R_{B14}, and R_{c} is a single bond bonding with L_{B1};
L_{B1} is a single bond, a substituted or unsubstituted arylene group having 6 to 50 ring carbon atoms, or a substituted or unsubstituted divalent heterocyclic group having 5 to 50 ring atoms;
Y_{B1} and Y_{B2} are independently a single bond, or C(R_{f});
R_{d} and Rₑ, and R_{f}'s form a substituted or unsubstituted, saturated or unsaturated ring by bonding with each other, or do not form the ring;
one or more sets of the adjacent two or more of R_{B11} to R_{B14} which are not a single bond bonding with L_{B1} form a substituted or unsubstituted, saturated or unsaturated ring by bonding with each other, or do not form the ring; and
R_{B1} to R_{B10} which do not form the ring and which are not a single bond bonding with L_{B1}, R_{B1} to R_{B14} which do not form the ring and which are not a single bond bonding with L_{B1}, R_{c} which are not a single bond bonding with L_{B1}, and R_{d}, Rₑ and R_{f} which do not form the ring are independently a hydrogen atom, or a substituent R; and the substituent R is the same as defined in the formula (A1).

When L_{B1} is a single bond, the structure of the following moiety (p_{B}) and the anthracene skeleton are bonded via a single bond in the compound represented by the formula (B1).

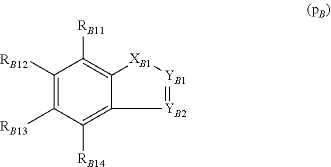

In one embodiment, the compound represented by the formula (B1) is a compound represented by the following formula (B2).

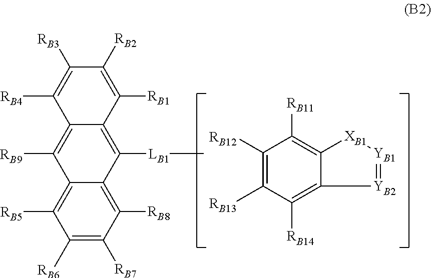

In the formula (B2), R_{B1} to R_{B9}, R_{B11} to R_{B14}, L_{B1}, X_{B1}, Y_{B1}, and Y_{B2} are the same as defined in the formula (B1).

In one embodiment, the compound represented by the formula (B1) is selected from the group consisting of a compound represented by the following formula (B3-1), and a compound represented by the following formula (B3-2).

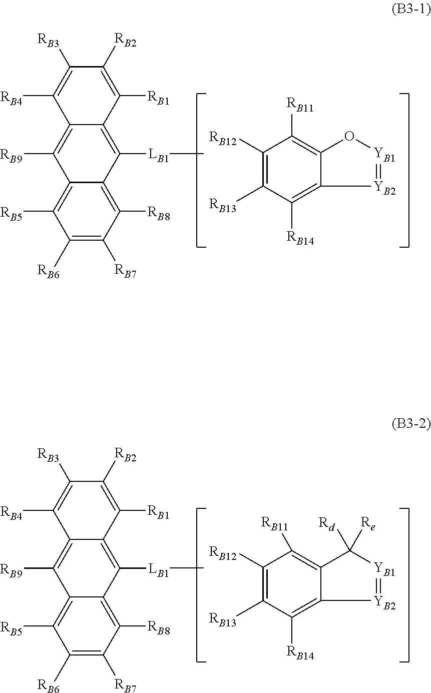

In the formulas (B3-1) and (B3-2), R_{B1} to R_{B9}, R_{B11} to R_{B14}, L_{B1}, Y_{B1}, Y_{B2}, R_{d}, and Rₑ are the same as defined in the formula (B1).

In one embodiment, the compound represented by the formula (B1) is selected from the group consisting of a compound represented by the following formula (B3-3), and a compound represented by the following formula (B3-4).

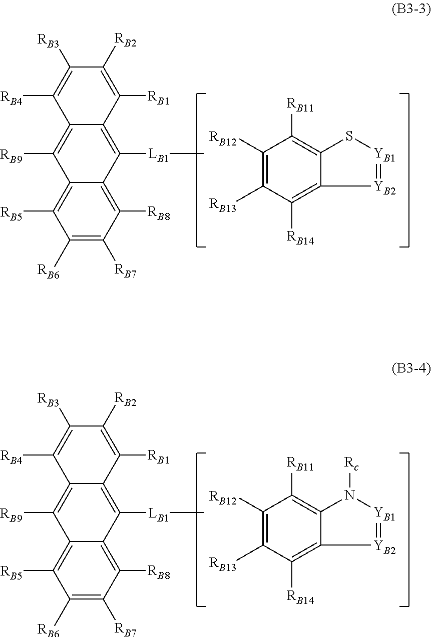

In the formulas (B3-3) and (B3-4), R_{B1} to R_{B9}, R_{B11} to R_{B14}, L_{B1}, Y_{B1}, Y_{B2}, and R_{c} are the same as defined in the formula (B1).

In one embodiment, the compound represented by the formula (B1) is a compound represented by the following formula (B4).

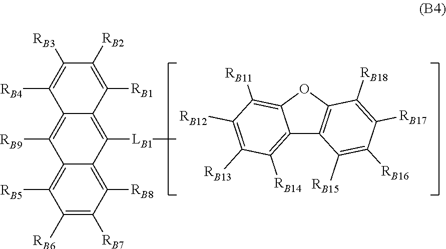

In the formula (B4), R_{B1} to R_{B9}, R_{B11} to R_{B14} and L_{B1} are the same as defined in the formula (B1):
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B4-1), and a compound represented by the following formula (B4-2).

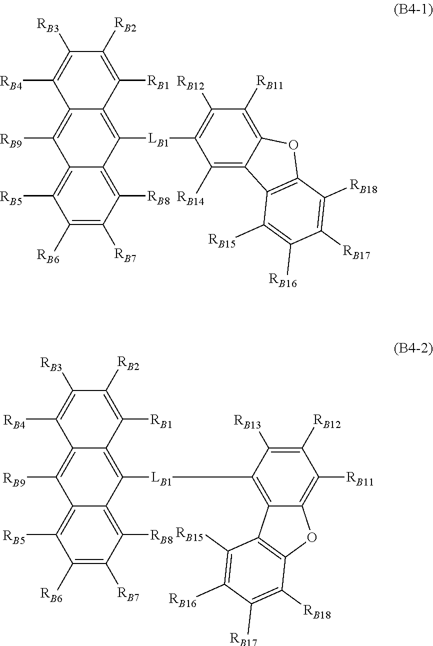

In the formulas (B4-1) and (B4-2), R_{B1} to R_{B9}, R_{B11} to R_{B14}, and L_{B1} are the same as defined in the formula (B1);
one or more sets of the adjacent two or more of R_{B14} to R_{B18}, form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B4-1b), and a compound represented by the following formula (B4-2b).

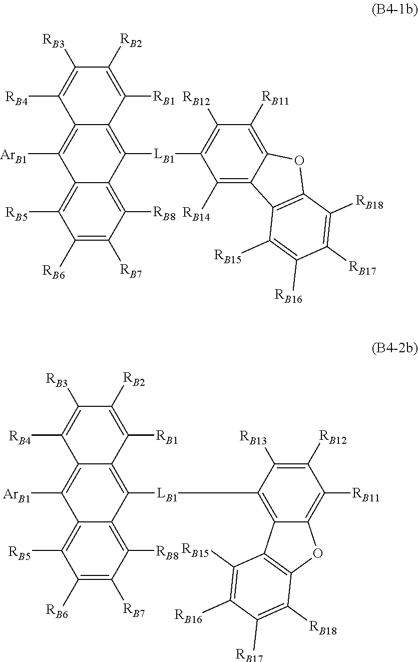

In the formulas (B4-1b) and (B4-2b), R_{B1} to R_{B8}, R_{B11} to R_{B14}, and L_{B1} are the same as defined in the formula (B1):
Ar_{B1} is a substituted or unsubstituted aryl group having 6 to 50 ring carbon atoms;
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B5-1), a compound represented by the following formula (B5-2), and a compound represented by the following formula (B5-3).

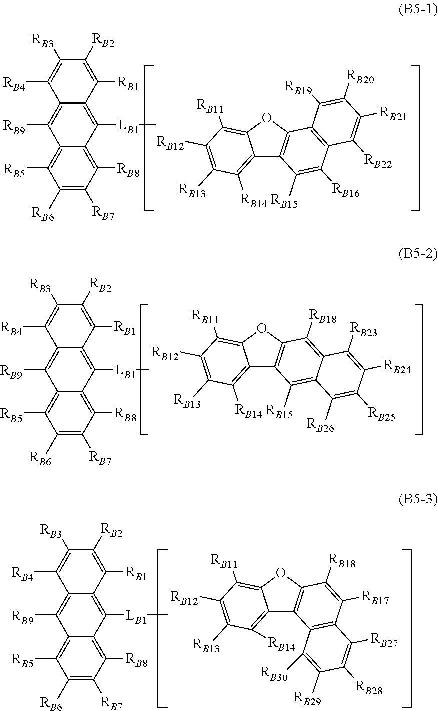

wherein in the formulas (B5-1) to (B5-3), R_{B1} to R_{B9}, R_{B11} to R_{B14}, and L_{B1} are the same as defined in the formula (B1);
one or more sets of the adjacent two or more of R_{B15} to R_{B30} form a substituted or unsubstituted, saturated or unsaturated ring by bonding with each other, or do not form the ring; and
R_{B15} to R_{B30} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B5-1a), a compound represented by the following formula (B5-2a), and a compound represented by the following formula (B5-3a).

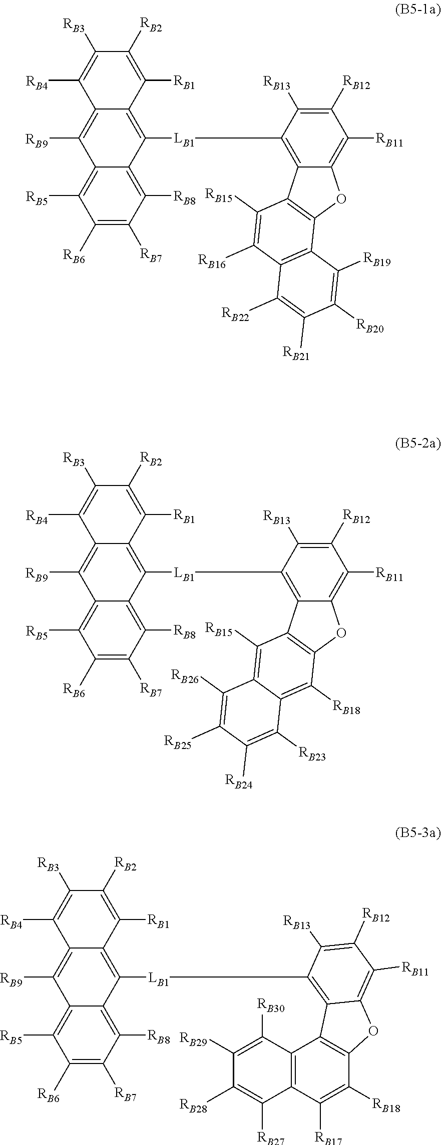

In the formulas (B5-1a) to (B5-3a), R_{B1} to R_{B9}, R_{B11} to R_{B15} and L_{B1} are the same as defined in the formula (B1):
one or more sets of the adjacent two or more of R_{B15} to R_{B30} form a substituted or unsubstituted, saturated or unsaturated ring by bonding with each other, or do not form the ring; and
R_{B15} to R_{B30} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B5-4a), a compound represented by the following formula (B5-5a), and a compound represented by the following formula (B5-6a).

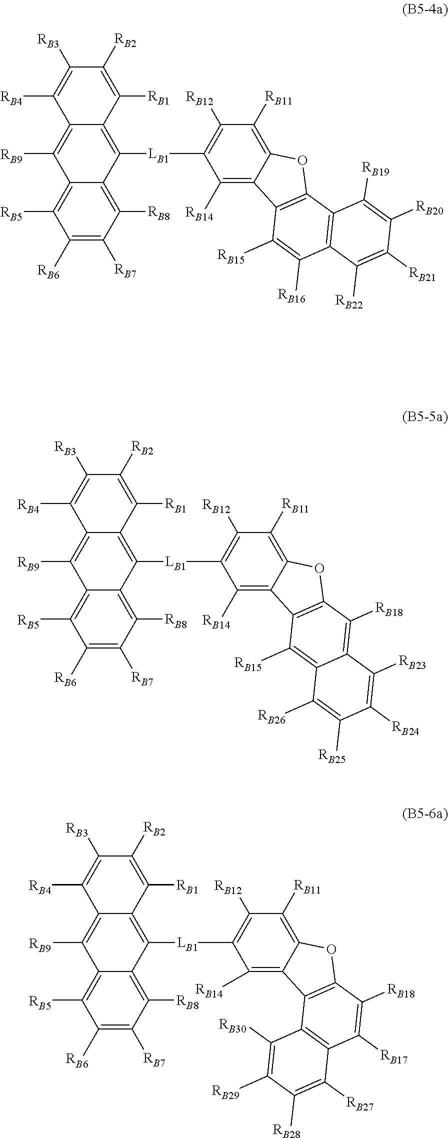

In the formulas (B5-4a) to (B5-6a), R_{B1} to R_{B9}, R_{B11}, R_{B12}, R_{B14}, and L_{B1} are the same as defined in the formula (B1):
one or more sets of the adjacent two or more of R_{B15} to R_{B30} form a substituted or unsubstituted, saturated or unsaturated ring by bonding with each other, or do not form the ring; and
R_{B15} to R_{B30} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B5-1b), a compound represented by the following formula (B5-2b), and a compound represented by the following formula (B5-3b).

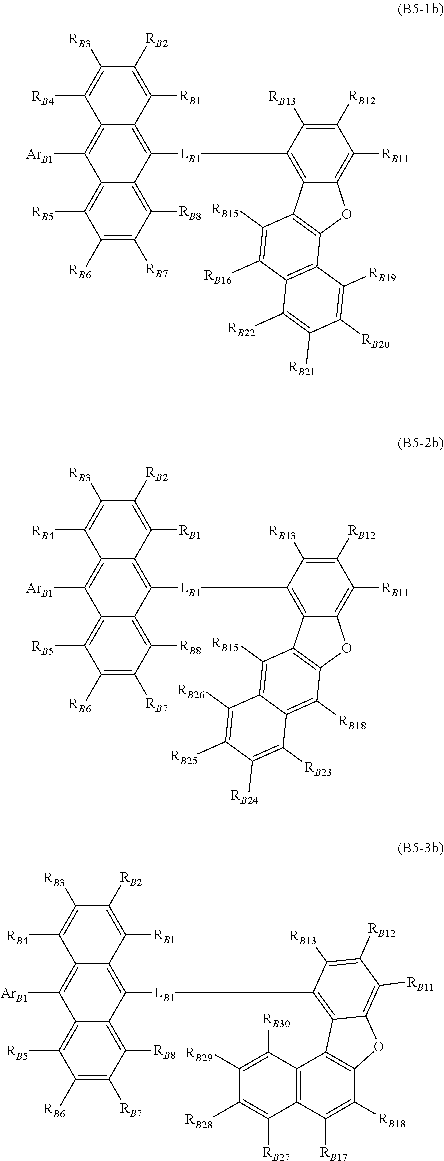

In the formulas (B5-1b) to (B5-3b), R_{B1} to R_{B8}, R_{B11} to R_{B13}, and L_{B1} are the same as defined in the formula (B1):
one or more sets of the adjacent two or more of R_{B15} to R_{B30} form a substituted or unsubstituted, saturated or unsaturated ring by bonding with each other, or do not form the ring;
R_{B15} to R_{B30} which do not form the ring are independently a hydrogen atom, or a substituent R; the substituent K is the same as defined in the formula (B1); and
Ar_{B1} is a substituted or unsubstituted aryl group having 6 to 50 ring carbon atoms.

In one embodiment, the compound represented by the formula (B1) is selected from the group consisting of a compound represented by the following formula (B5-4b), a compound represented by the following formula (B5-5b), and a compound represented by the following formula (B5-6b).

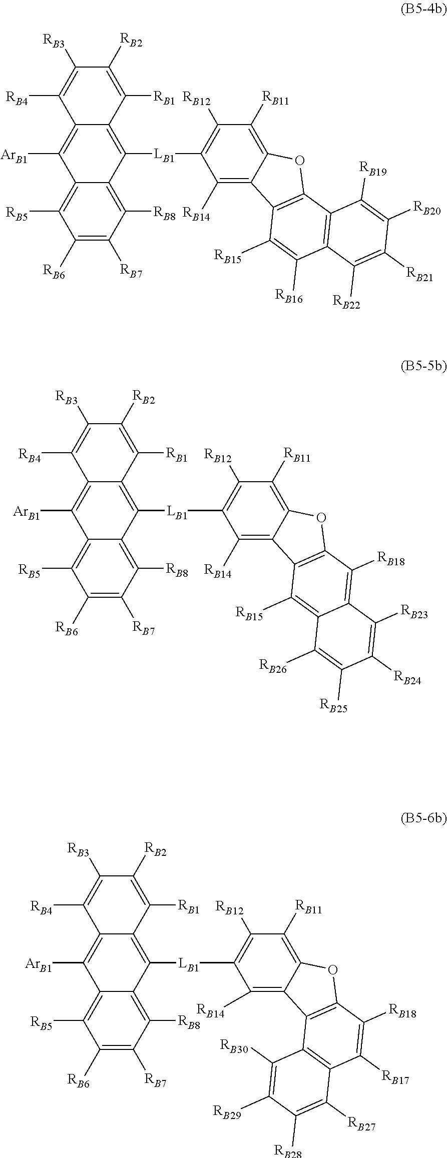

In the formulas (B5-4b) to (B5-6b), R_{B1} to R_{B8}, R_{B11}, R_{B12}, R_{B14}, and L_{b1} are the same as defined in the formula (B1):
one or more sets of the adjacent two or more of R_{B15} to R_{B30} form a substituted or unsubstituted, saturated or unsaturated ring by bonding with each other, or do not form the ring;
R_{B15} to R_{B30} which do not form the ring are independently a hydrogen atom, or a substituent R; and the substituent R is the same as defined in the formula (B1); and
Ar_{B1} is a substituted or unsubstituted aryl group having 6 to 50 ring carbon atoms.

In one embodiment, the compound represented by the formula (B1) is a compound represented by the following formula (B6).

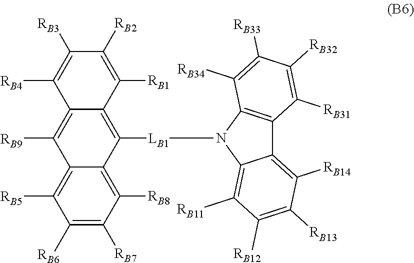

In the formula (B6), R_{B1} to R_{B9}, R_{B11} to R_{B14}, and L_{B1} are the same as defined in the formula (B1):
one or more sets of the adjacent two or more of R_{B31} to R_{B34} form a substituted or unsubstituted, saturated or unsaturated ring by bonding with each other, or do not form the ring; and
R_{B31} to R_{B34} which do not form the ring are independently a hydrogen atom, or a substituent R; and the substituent R is the same as defined in the formula (B1).

In one embodiment, the compound represented by the formula (B1) is a compound represented by the following formula (B6-1 b).

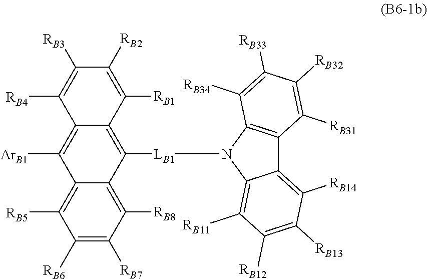

In the formula (B6-1b), R_{B1} to R_{B8}, R_{B11} to R_{B14}, and L_{B1} are the same as defined in the formula (B1):
Ar_{B1} is a substituted or unsubstituted aryl group having 6 to 50 ring carbon atoms;
the adjacent R_{B31} to R_{B34} form a substituted or unsubstituted, saturated or unsaturated ring by bonding with each other, or do not form the ring; and
R_{B31} to R_{B34} which do not form the ring are independently a hydrogen atom, or a substituent R; and the substituent R is the same as defined in the formula (B1).

In one embodiment, the compound represented by the formula (B1) is a compound represented by the following formula (B7).

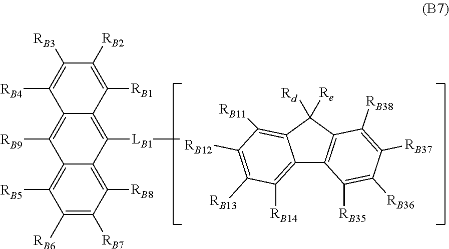

In the formula (B7), R_{B1} to R_{B9}, R_{B11} to R_{B14}, L_{B1}, R_{d}, and Rₑ are the same as defined in the formula (B1):
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

In one embodiment, R_{d} and Rₑ in the formula (B7) form a substituted or unsubstituted spiro ring by bonding with each other.

In one embodiment, the compound represented by the formula (B1) is selected from the group consisting of a compound represented by the following formula (B7-1a), a compound represented by the following formula (B7-2a), and a compound represented by the following formula (B7-3a).

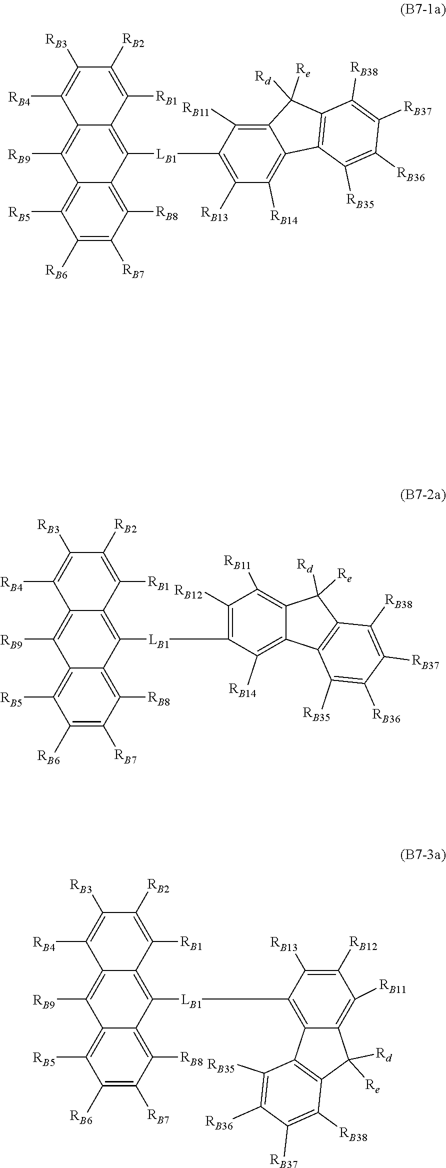

In the formulas (B7-1a) to (B7-3a), R_{B1} to R_{B9}, R_{B11} to R_{B14}, L_{B1}, R_{d}, and Rₑ are the same as defined in the formula (B1):
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B7-1b), a compound represented by the following formula (B7-2b), a compound represented by the following formula (B7-3b), and a compound represented by the following formula (B7-4b).

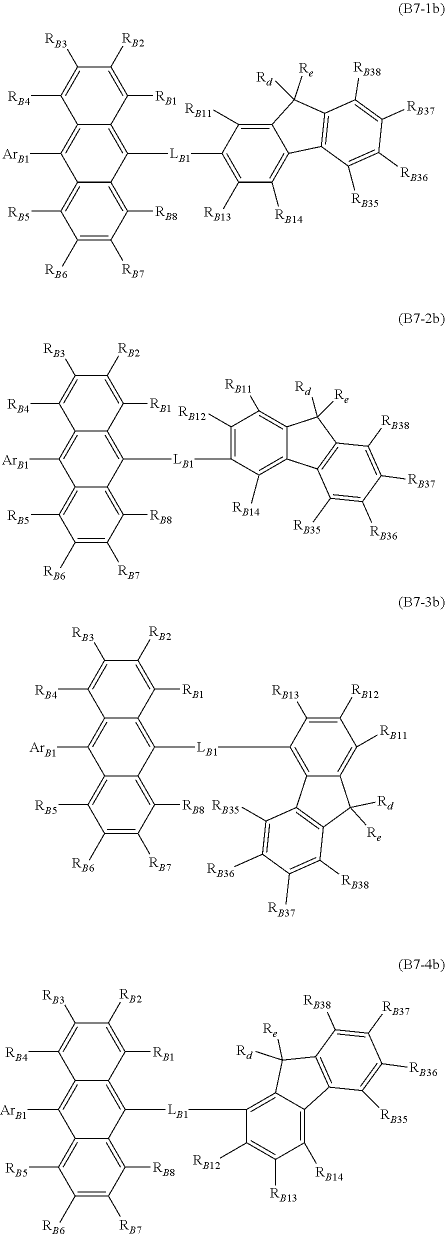

In the formulas (B7-1b) to (B7-4b), R_{B1} to R_{B8}, R_{B11} to R_{B14}, L_{B1}, R_{d}, and Rₑ are the same as defined in the formula (B1):
Ar_{B1} is a substituted or unsubstituted aryl group having 6 to 50 ring carbon atoms;
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

In one embodiment, the compound represented by the formula (B1) is a compound represented by the following formula (B8).

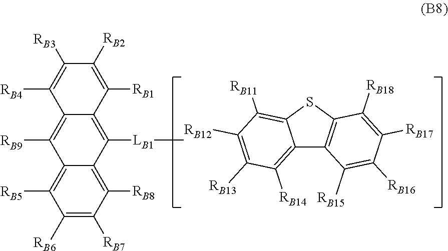

In the formula (B8), R_{B1} to R_{B9}, R_{B11} to R_{B14}, and L_{B1} are the same as defined in the formula (B1):
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

In one embodiment, the compound represented by the formula (B1) is selected from the group consisting of a compound represented by the following formula (B8-1), and a compound represented by the following formula (B8-2).

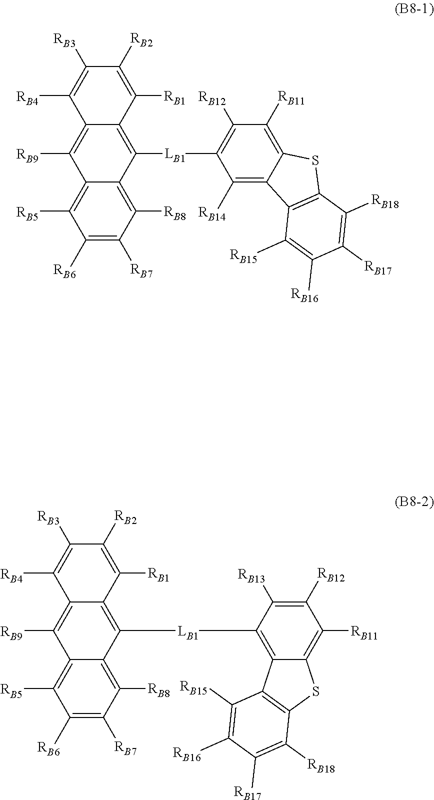

In the formulas (B8-1) and (B8-2), R_{B1} to R_{B9}, R_{B11} to R_{B14}, and L_{B1} are the same as defined in the formula (B1);
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substitute R, and the substituent R is the same as defined in the formula (B1).

In one embodiment, R_{B1} to R_{B8} in the formula (B1) are hydrogen atoms.

In one embodiment, R_{B11} to R_{B14} which are not a single bond bonding with L_{B1} and which do not form the ring in the formula (B1) are hydrogen atoms.

In one embodiment, L_{B1} in the formula (B1) is a single bond, or a substituted or unsubstituted arylene group having 6 to 50 ring carbon atoms.

In one embodiment, L_{B1} in the formula (B1) is a single bond, or a substituted or unsubstituted arylene group having 6 to 20 ring carbon atoms.

In one embodiment, L_{B1} in the formula (B1) is a single bond, a phenylene group, or a biphenylene group.

In one embodiment, Ar_{B1} is a substituted or unsubstituted phenyl group, a substituted or unsubstituted biphenyl group, or a substituted or unsubstituted naphthyl group.

In one embodiment, the compound represented by the formula (A1) is a compound represented by the following formula (A5-1), and
the compound represented by the formula (B1) is a compound represented by the following formula (B4) (provided that the compound represented by the formula (A5-1) and the compound represented by the formula (B4) are different compounds).

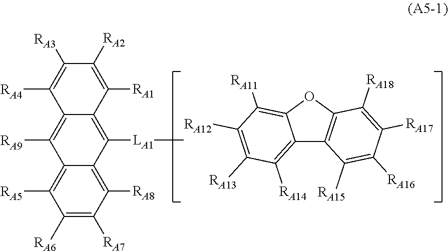

In the formula (A5-1), R_{A1} to R_{A9}, R_{A11} to R_{A14}, and L_{A1} are the same as defined in the formula (A1);
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; the substituent R is the same as defined in the formula (A1).

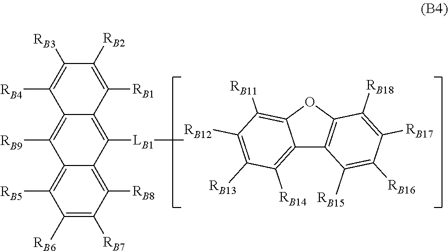

In the formula (B4), R_{B1} to R_{B9}, R_{B11} to R_{B14}, and L_{B1} are the same as defined in the formula (B1):
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

In one embodiment, the compound represented by the formula (A1) is a compound selected from the group consisting of a compound represented by the following formula (A5-1-1b), and a compound represented by the following formula (A5-1-2b), and
the compound represented by the formula (B1) is selected from the group consisting of a compound represented by the following formula (B4-1b), and a compound represented by the following formula (B4-2b).

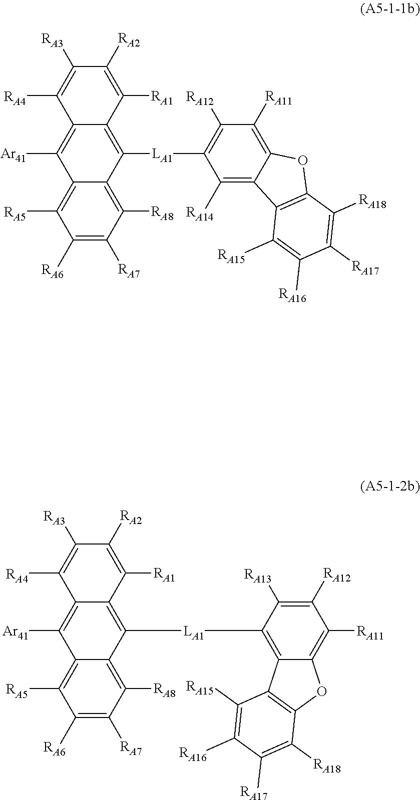

In the formulas (A5-1-1b) and (A5-1-2b), R_{A1} to R_{A8}, R_{A11} to R_{A14}, and L_{A1} are the same as defined in the formula (A1):
Ar_{A1} is a substituted or unsubstituted aryl group having 6 to 50 mg carbon atoms;
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; the substituent R is the same as defined in the formula (A1).

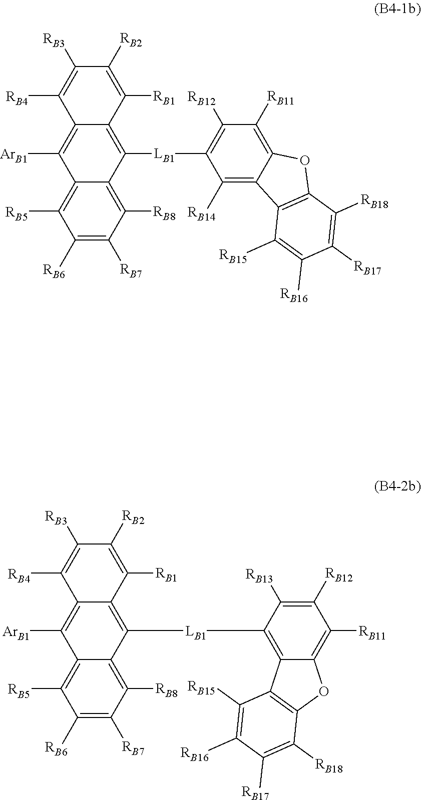

In the formulas (B4-1b) and (B4-2b), R_{B1} to R_{B8}, R_{B11} to R_{B14}, and L_{B1} are the same as defined in the formula (B1):
Ar_{B1} is a substituted or unsubstituted aryl group having 6 to 50 ring carbon atoms;
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

In one embodiment, the compound represented by the formula (A1) is selected from the group consisting of a compound represented by the following formula (A6-1b), a compound represented by the following formula (A6-2b), and a compound represented by the following formula (A6-3b), and
the compound represented by the formula (B1) is selected from the group consisting of a compound represented by the following formula (B5-1b), a compound represented by the following formula (B5-2b), and a compound represented by the following formula (B5-3b).

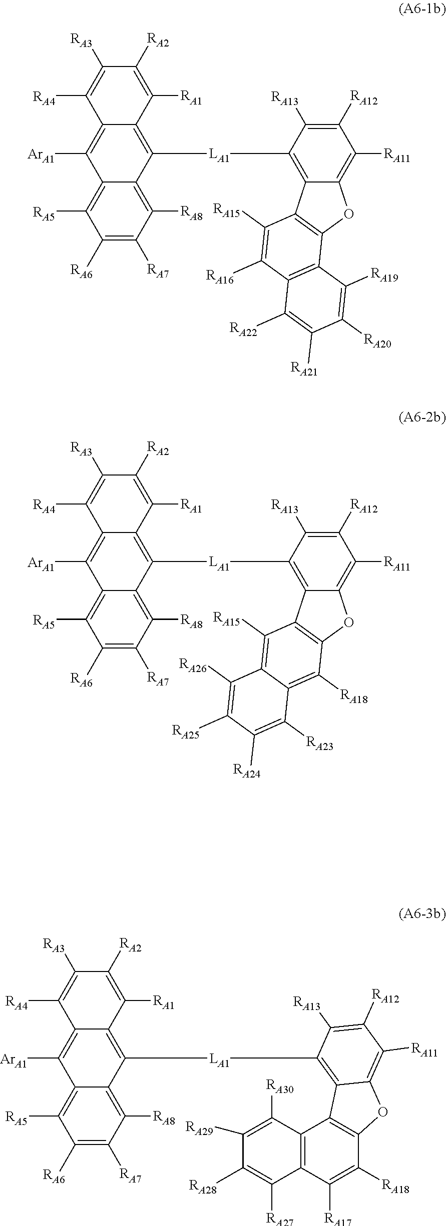

In the formulas (A6-1b) to (A6-3b), R_{A1} to R_{A8}, R_{A11} to R_{A13}, and L_{A1} are the same as defined in the formula (A1):
Ar_{A1} is a substituted or unsubstituted aryl group having 6 to 50 ring carbon atoms;
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; the substituent R is the same as defined in the formula (A1).

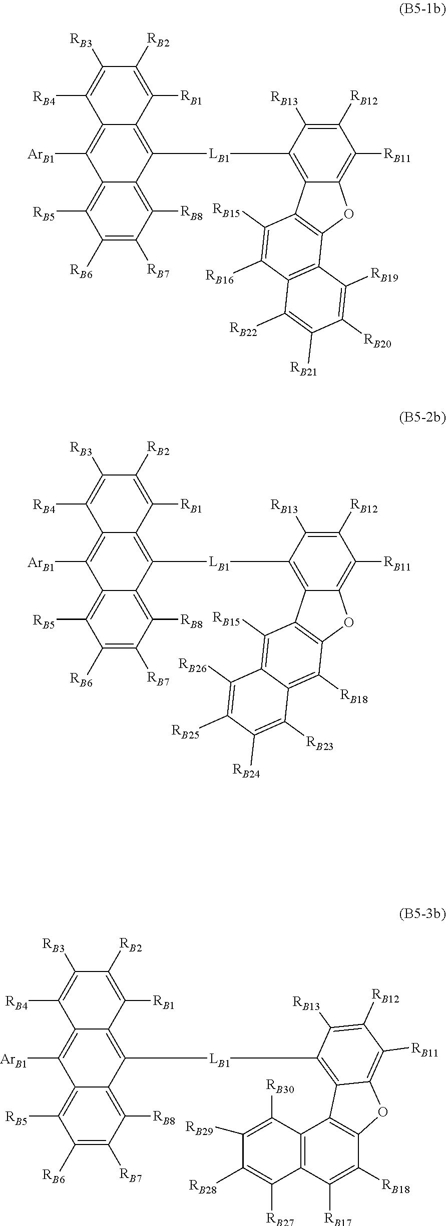

In the formulas (B5-1b) to (B5-3b), R_{B1} to R_{B8}, R_{B11} to R_{B13}, and L_{B1} are the same as defined in the formula (B1):
one or more sets of the adjacent two or more of R_{B15} to R_{B30}, form a substituted or unsubstituted, saturated or unsaturated ring by bonding with each other, or do not form the ring;
R_{B15} to R_{B30} which do not form the ring are independently a hydrogen atom, or a substituent R; the substituent R is the same as defined in the formula (B1); and
Ar_{B1} is a substituted or unsubstituted aryl group having 6 to 50 ring carbon atoms.

In one embodiment, the compound represented by the formula (A1) is a compound represented by the following formula (A5-1), and
the compound represented by the formula (B1) is a compound represented by the following formula (B7).

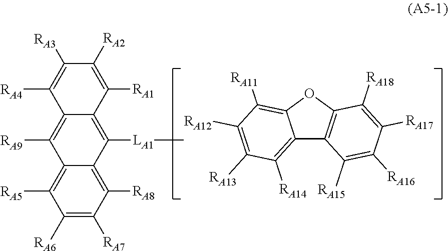

In the formula (A5-1), R_{A1} to R_{A9}, R_{A11} to R_{A14}, and L_{A1} are the same as defined in the formula (A1):
one or more sets of the adjacent two or more of R_{A14} to R_{A18} form a substituted or unsubstituted, saturated or unsaturated ring by bonding with each other, or do not form the ring; and
R_{A15} to R_{A18} is which do not form the ring are independently a hydrogen atom, or a substituent R; the substituent R is the same as defined in the formula (A1).

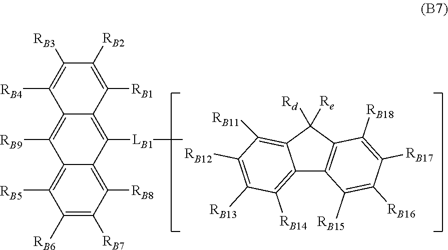

In the formula (B7), R_{B1} to R_{B9}, R_{B11} to R_{B14}, L_{B1}, R_{d}, and Rₑ are the same as defined in the formula (B1):
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

In one embodiment, the compound represented by the formula (A1) is selected from the group consisting of a compound represented by the following formula (A6-1b), a compound represented by the following formula (A6-2b), and a compound represented by the following formula (A6-3b), and
the compound represented by the formula (B1) is selected from the group consisting of a compound represented by the following formula (B7-1b), a compound represented by the following formula (B7-2b), and a compound represented by the following formula (B7-3b).

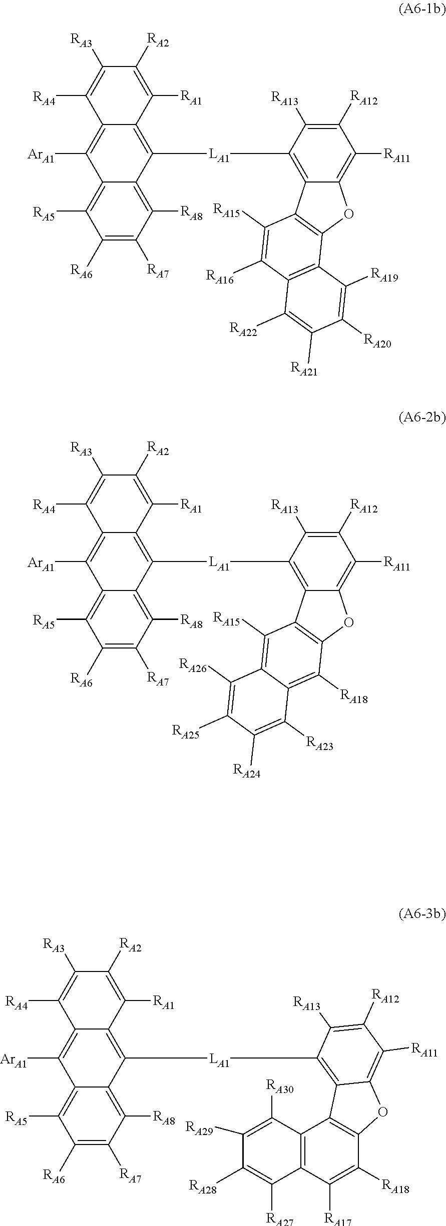

In the formulas (A6-1b) to (A6-3b), R_{A1} to R_{A8}, R_{A11} to R_{A13}, and L_{A1} are the same as defined in the formula (A1):
Ar_{A1} is a substituted or unsubstituted aryl group having 6 to 50 ring carbon atoms;
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substitutent R; the substituent R is the same as defined in the formula (A1).

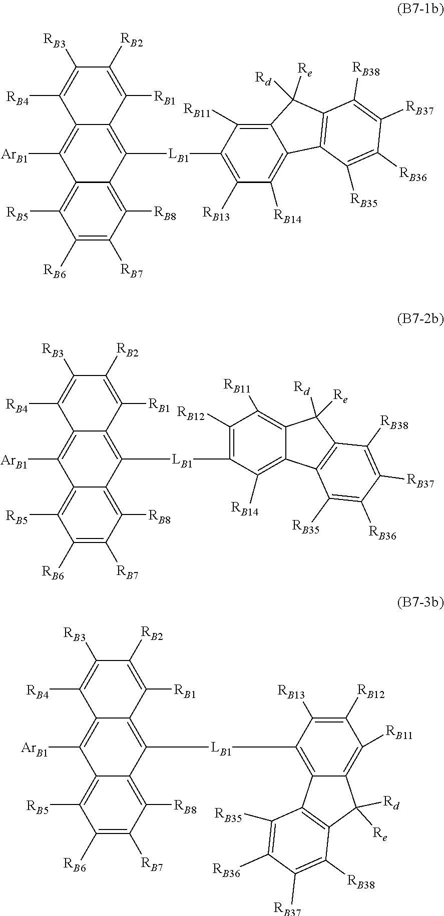

In the formulas (B7-1b) to (B7-3b), R_{B1} to R_{B8}, R_{B11} to R_{B14}, L_{B1}, R_{d}, and Rₑ are the same as defined in the formula (B1):

Ar_{B1} is a substituted or unsubstituted aryl group having 6 to 50 ring carbon atoms:
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

In one embodiment, the compound represented by the formula (A1) is selected from the group consisting of a compound represented by the following formula (A5-2-1), and a compound represented by the following formula (A5-2-2), and
the compound represented by the formula (B1) is selected from the group consisting of a compound represented by the following formula (B5-1), a compound represented by the following formula (B5-2), and a compound represented by the following formula (B5-3).

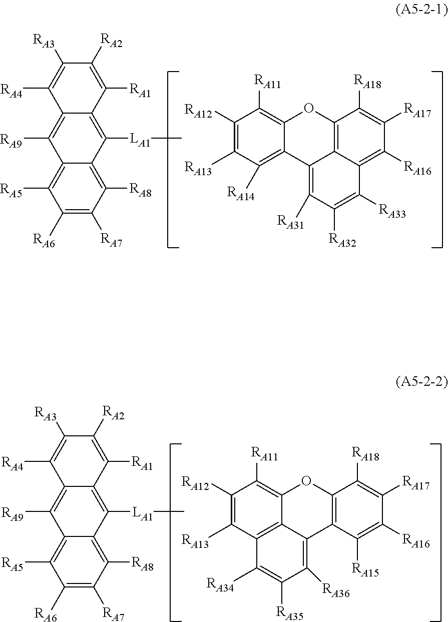

In the formulas (A5-2-1) and (A5-2-2), R_{A1} to R_{A9}, R_{A11} to R_{A14}, and L_{A1} are the same as defined in the formula (A1):
one or more sets of the adjacent two or more of R_{A13} to R_{A18}, and R_{A31} to R_{A36} form a substituted or unsubstituted, saturated or unsaturated ring by bonding with each other, or do not form the ring;
R_{A15} to R_{A18} and R_{A31} to R_{A36}, which do not form the ring are independently a hydrogen atom, or a substituent R; and the substituent R is the same as defined in the formula (A1).

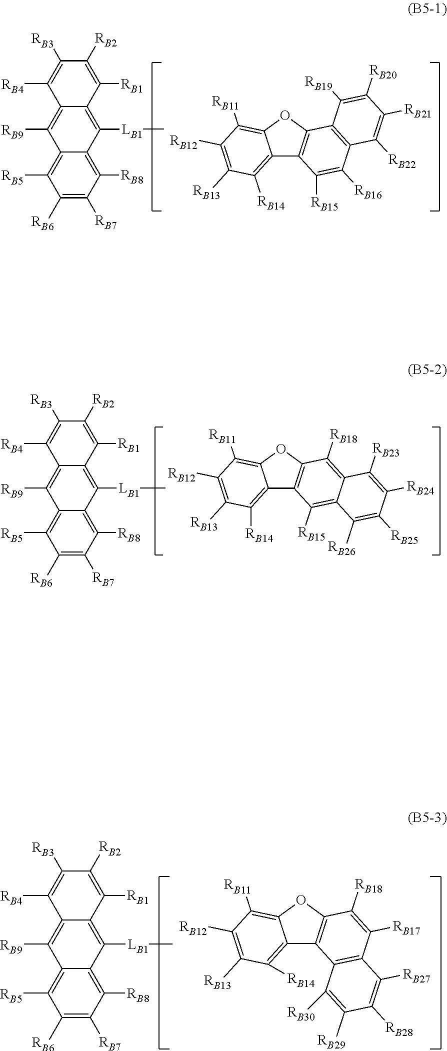

In the formulas (B5-1) to (B5-3), R_{B1} to R_{B9}, R_{B11} to R_{B14}, and L_{B1}, are the same as defined in the formula (B1);
one or more sets of the adjacent two or more of R_{B15} to R_{B30} form a substituted or unsubstituted, saturated or unsaturated ring by bonding with each other, or do not form the ring; and
R_{B15} to R_{B30} which do not form the ring are independently a hydrogen atom, or a substituent R; and the substituent R is the same as defined in the formula (B1).

In one embodiment, the substituent in the case of “substituted or unsubstituted” in the formulas (A1) and (B1) is selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
   —Si(R₉₀₁)(R₉₀₂)(R₉₀₃),
   —O—(R₉₀₄),
   —S—(R₉₀₄),
   —N(R₉₀₆)(R₉₀₇)
   (wherein R₉₀₁ to R₉₀₇ independently
   a hydrogen atom,
   a substituted or unsubstituted alkyl group having 1 to 50 carbon atoms,
   a substituted or unsubstituted cycloalkyl group having 3 to 50 ring carbon atoms,
   a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom a cyano group, a nitro group,
an unsubstituted aryl group having 6 to 50 ring carbon atoms, and
an unsubstituted monovalent heterocyclic group having 5 to 50 ring atoms.

In one embodiment, the substituent in the case of “substituted or unsubstituted” in the formulas (A1) and (B1) is selected from the group consisting of
an unsubstituted alkyl group having 1 to 50 carbon atoms.
an unsubstituted cycloalkyl group having 3 to 50 ring carbon atoms.
   —Si(R₉₀₁)(R₉₀₂)(R₉₀₃),
   —O—(R₉₀₄),
   —S—(R₉₀₅),
   —N(R₉₀₆)(R₉₀₇)
   (wherein R₉₀₁ to R₉₀₇ are independently
   a hydrogen atom,
   a substituted or unsubstituted alkyl group having 1 to 50 carbon atoms,
   a substituted or unsubstituted cycloalkyl group having 3 to 50 ring carbon atoms,
   a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
an unsubstituted aryl group having 6 to 50 ring carbon atoms, and
an unsubstituted monovalent heterocyclic group having 5 to 50 ring atoms.

In one embodiment, the substituent in the case of “substituted or unsubstituted” in the formulas (A1) and (B1) is selected from the group consisting of
an unsubstituted alkyl group having 1 to 10 carbon atoms,
an unsubstituted cycloalkyl group having 3 to 10 ring carbon atoms,
   —Si(R₉₀₁)(R₉₀₂)(R₉₀₃),
   —O—(R₉₀₂),
   —S—(R₉₀₅),
   —N(R₉₀₆)(R₉₀₇)
   (wherein R₉₀₆ to R₉₀₇ are independently
a hydrogen atom,
an unsubstituted alkyl group having 1 to 10 carbon atoms,
an unsubstituted cycloalkyl group having 3 to 10 ring carbon atoms,
an unsubstituted aryl group having 6 to 20 ring carbon atoms, or
an unsubstituted monovalent heterocyclic group having 5 to 20 ring atoms; when two or more of each of R₉₀₁ to R₉₀₇ are present, the two or more of each of R₉₀₁ to R₉₀₇ may be the same as or different from each other).
a halogen atom, a cyano group, a nitro group,
an unsubstituted aryl group having 6 to 20 ring carbon atoms, and
an unsubstituted monovalent heterocyclic group having 3 to 20 ring atoms.

Specific examples of the compound represented by the formula (A1) will be described below, but these are merely examples, and the compound represented by the formula (A1) is not limited to the following specific examples. In the following specific examples. “D” represents a deuterium atom. “Me” represents a methyl group, and “Ph” represents a phenyl group.

(Specific examples of compound in which X_{A1} is O and Z_{A1} is single bond)

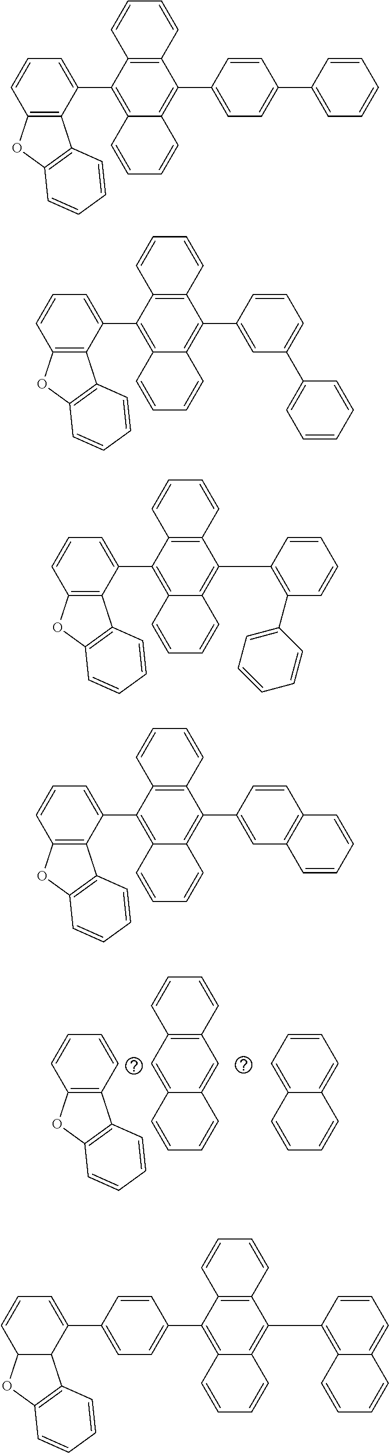

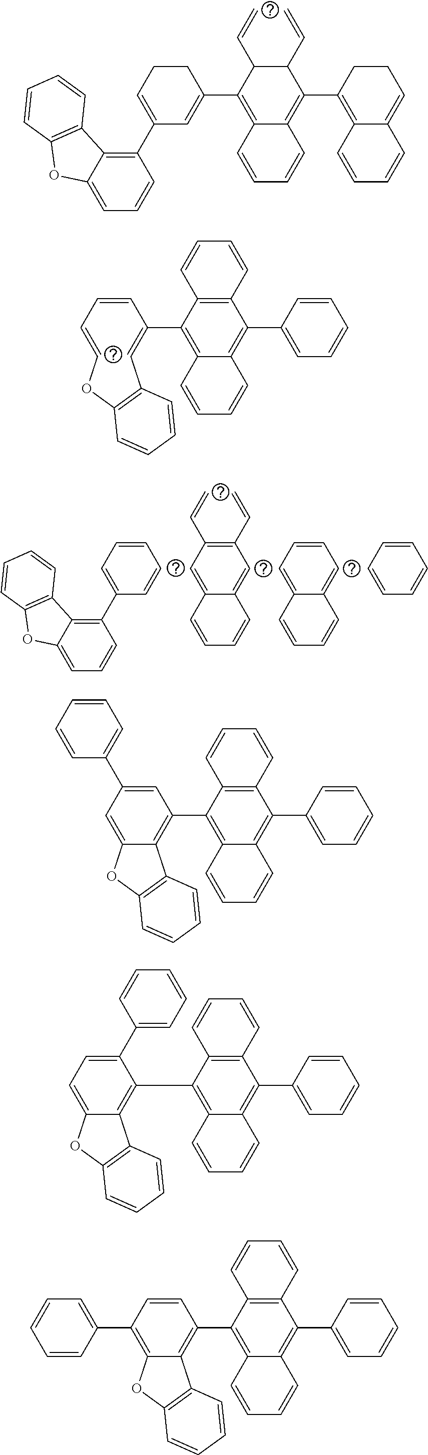

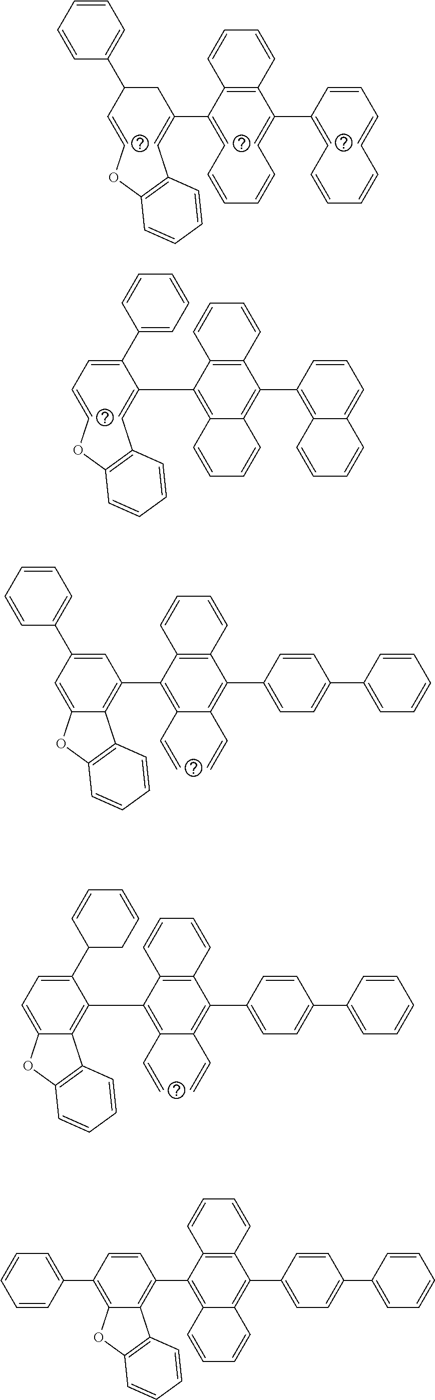

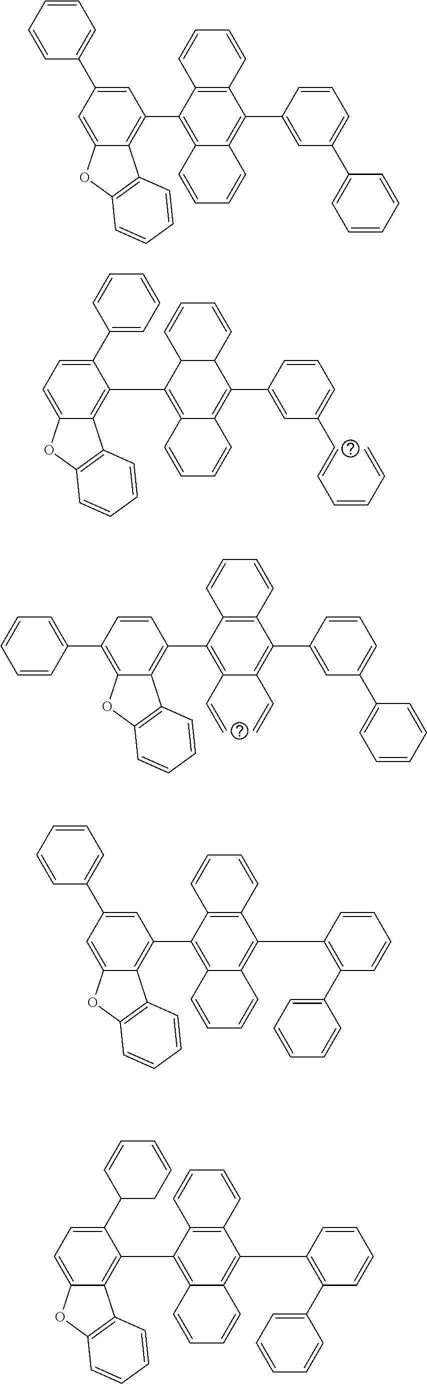

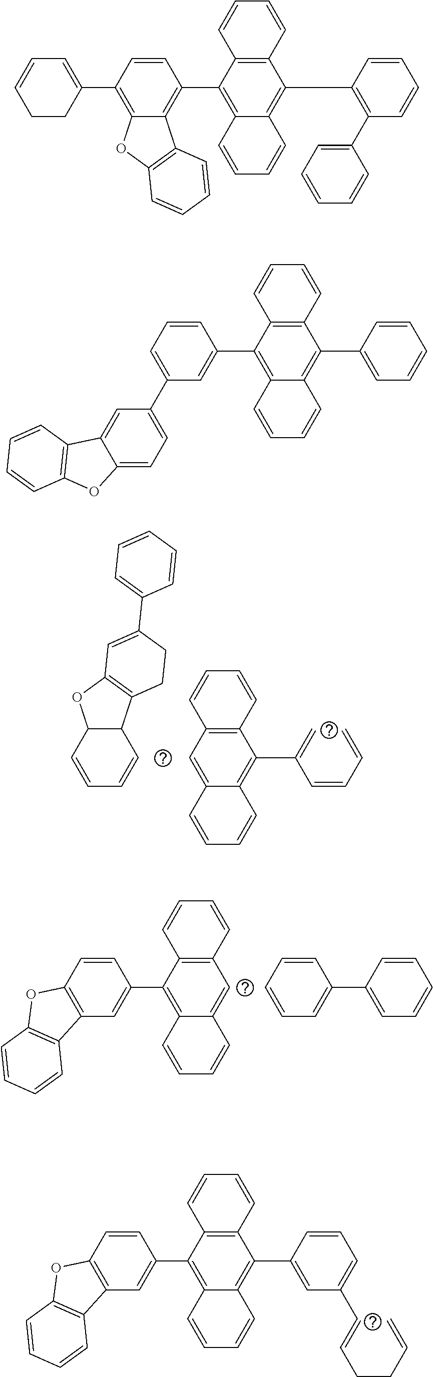

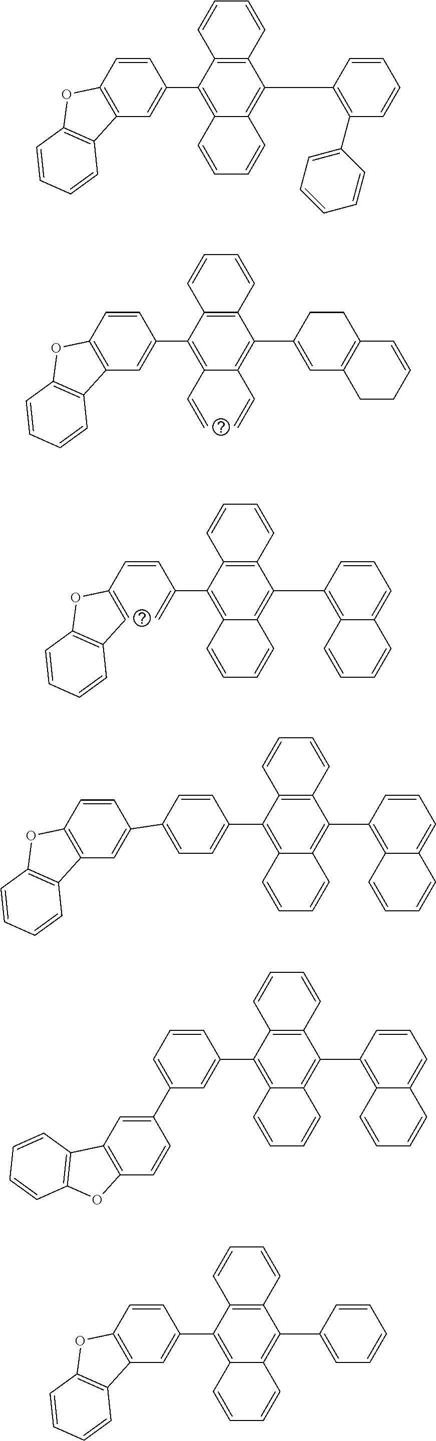 [SMILES: c1ccc(-c2ccccc2-c2c3ccccc3c(-c3ccc4oc5ccccc5c4c3)c3ccccc23)cc1 ; C=Cc1c(C=C)c(-c2ccc3oc4ccccc4c3c2)c2ccccc2c1C1=CC2=C(C=CCC2)CC1 ; C=C(/C=C\c1cc2ccccc2o1)c1c2ccccc2c(-c2cccc3ccccc23)c2ccccc12 ; c1ccc2c(-c3c4ccccc4c(-c4ccc(-c5ccc6oc7ccccc7c6c5)cc4)c4ccccc34)cccc2c1 ; c1cc(-c2ccc3oc4ccccc4c3c2)cc(-c2c3ccccc3c(-c3cccc4ccccc34)c3ccccc23)c1 ; c1ccc(-c2c3ccccc3c(-c3ccc4oc5ccccc5c4c3)c3ccccc23)cc1]

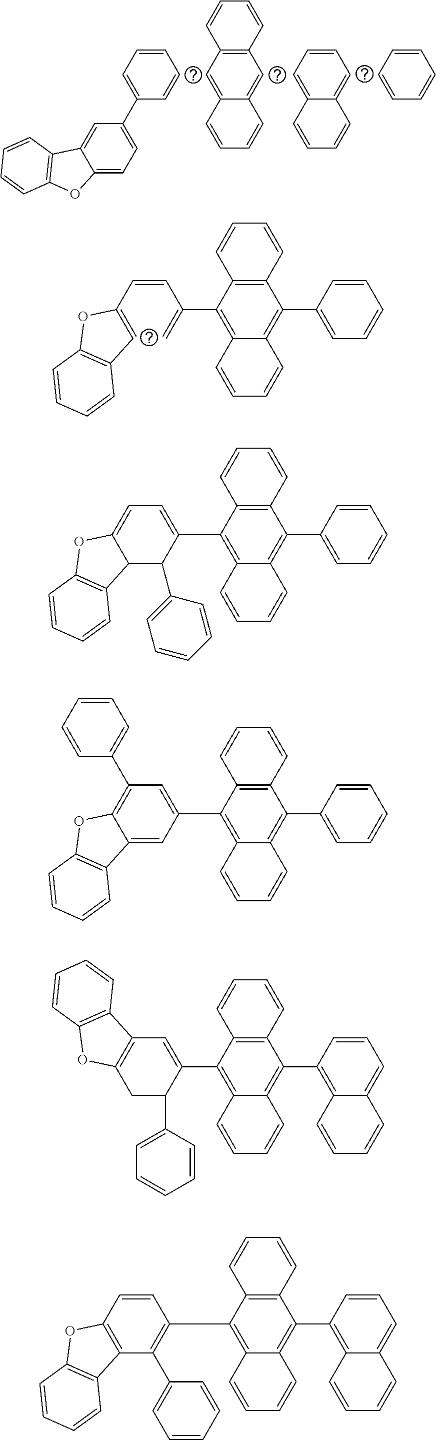

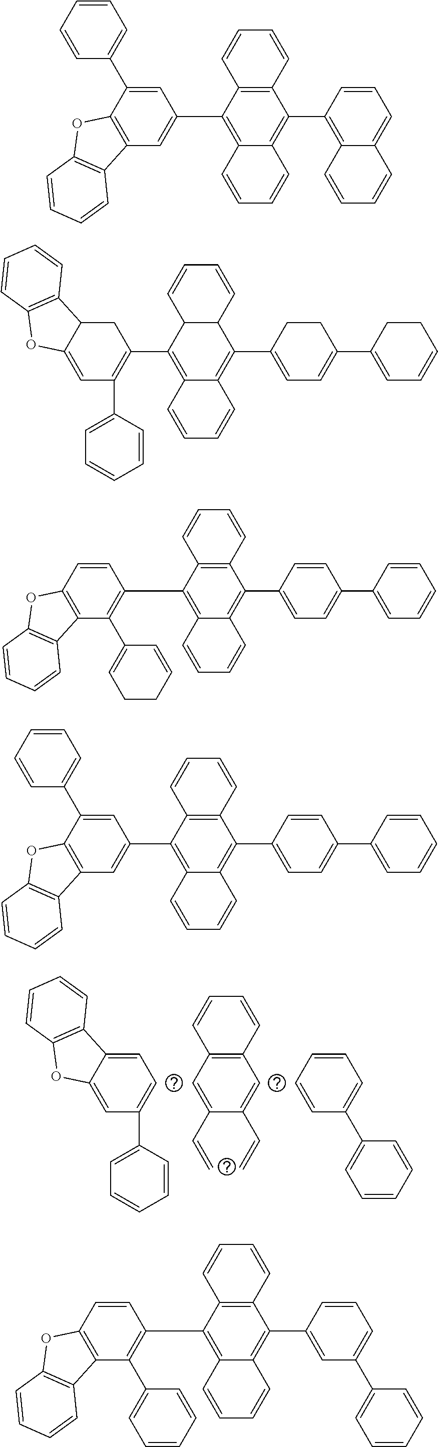

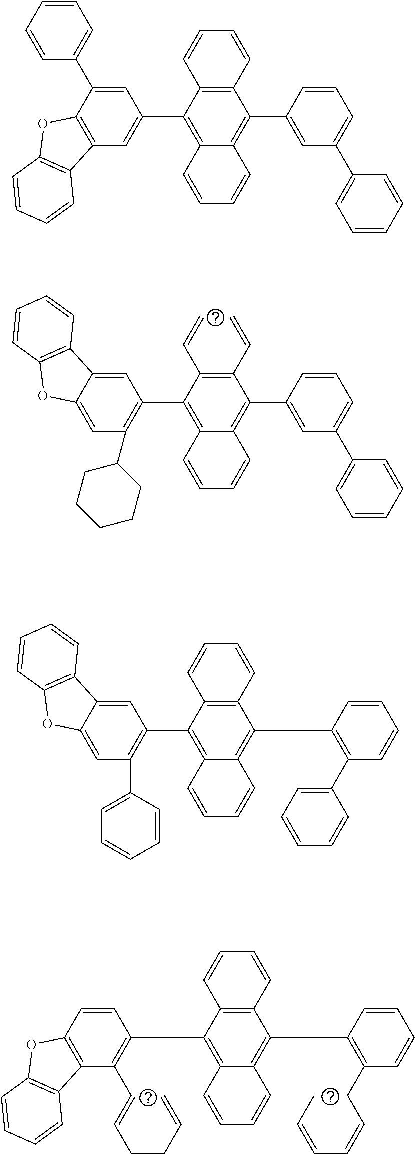 [SMILES: c1ccc(-c2cccc(-c3c4ccccc4c(-c4cc(-c5ccccc5)c5oc6ccccc6c5c4)c4ccccc34)c2)cc1 ; C=Cc1c(C=C)c(-c2cc3c(cc2C2CCCCC2)oc2ccccc23)c2ccccc2c1-c1cccc(-c2ccccc2)c1 ; c1ccc(-c2ccccc2-c2c3ccccc3c(-c3cc4c(cc3-c3ccccc3)oc3ccccc34)c3ccccc23)cc1 ; C=CCC/C=C/c1c(-c2c3ccccc3c(-c3ccccc3C/C=C\C=C/C)c3ccccc23)ccc2oc3ccccc3c12]

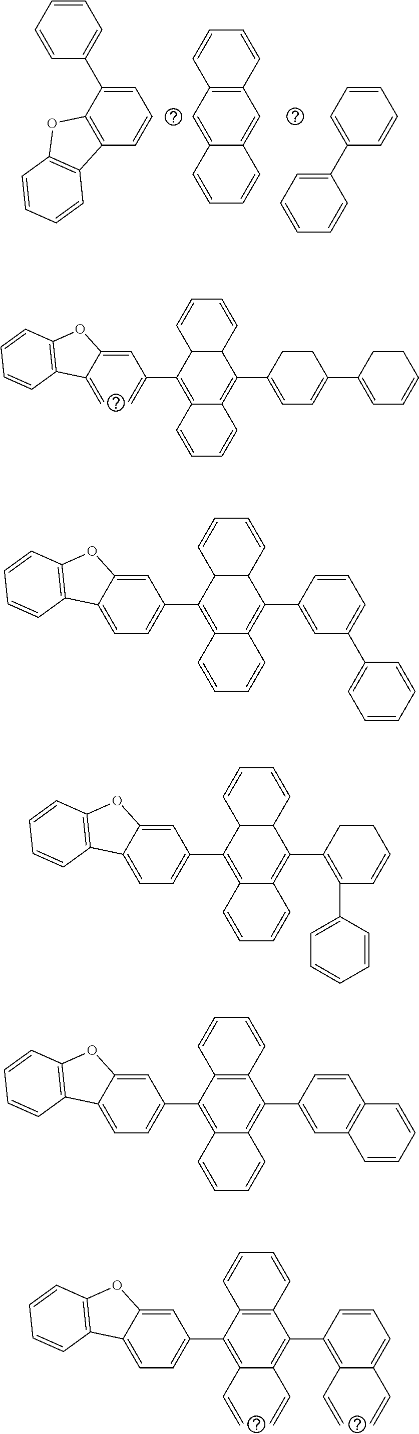

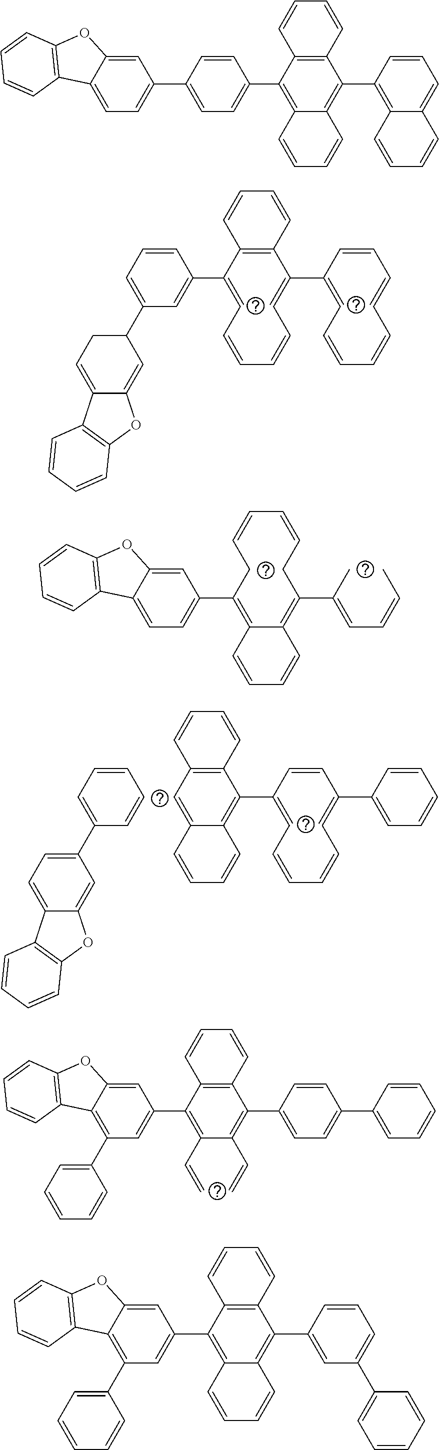

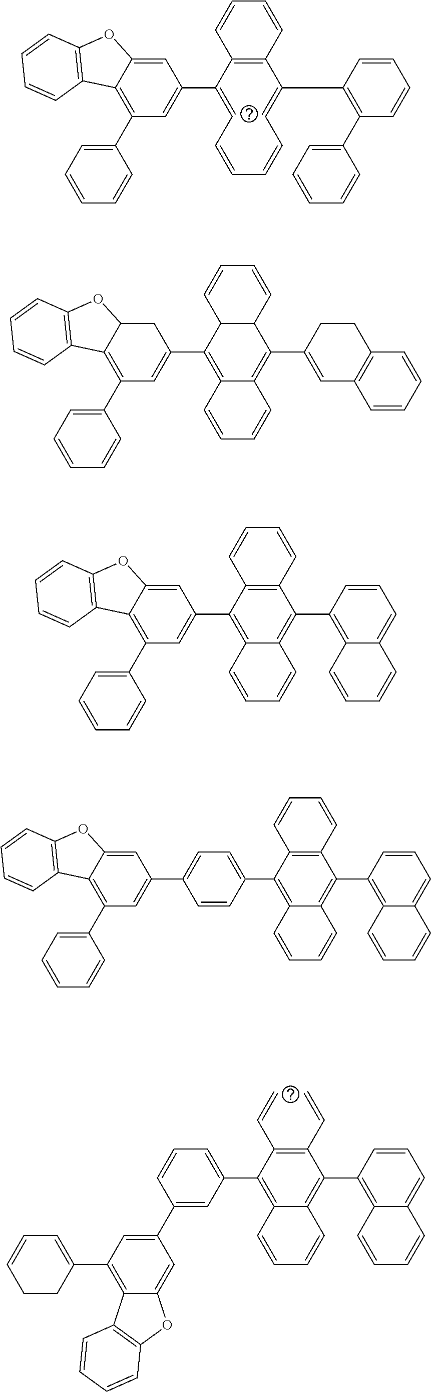 [SMILES: c1ccc(-c2ccccc2-c2ccccccc(-c3cc(-c4ccccc4)c4c(c3)oc3ccccc34)c3ccccc23)cc1 ; C1=CC2C(C3=Cc4ccccc4CC3)=c3ccccc3=C(C3=CC(c4ccccc4)=C4c5ccccc5OC4C3)C2C=C1 ; c1ccc(-c2cc(-c3c4ccccc4c(-c4cccc5ccccc45)c4ccccc34)cc3oc4ccccc4c23)cc1 ; c1ccc(-c2cc(-c3ccc(-c4c5ccccc5c(-c5cccc6ccccc56)c5ccccc45)cc3)cc3oc4ccccc4c23)cc1 ; C=Cc1c(C=C)c(-c2cccc3ccccc23)c2ccccc2c1-c1cccc(-c2cc(C3=CC=CCC3)c3c(c2)oc2ccccc23)c1]

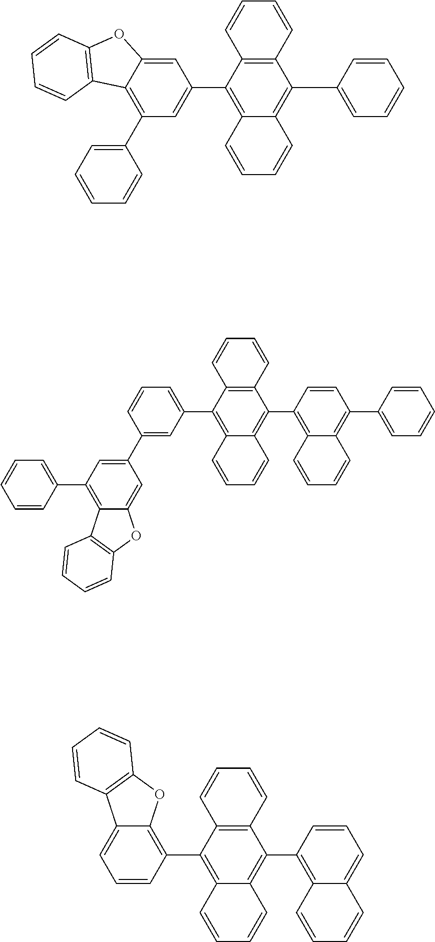 [SMILES: c1ccc(-c2c3ccccc3c(-c3cc(-c4ccccc4)c4c(c3)oc3ccccc34)c3ccccc23)cc1 ; c1ccc(-c2ccc(-c3c4ccccc4c(-c4cccc(-c5cc(-c6ccccc6)c6c(c5)oc5ccccc56)c4)c4ccccc34)c3ccccc23)cc1 ; c1ccc2c(-c3c4ccccc4c(-c4cccc5c4oc4ccccc45)c4ccccc34)cccc2c1]

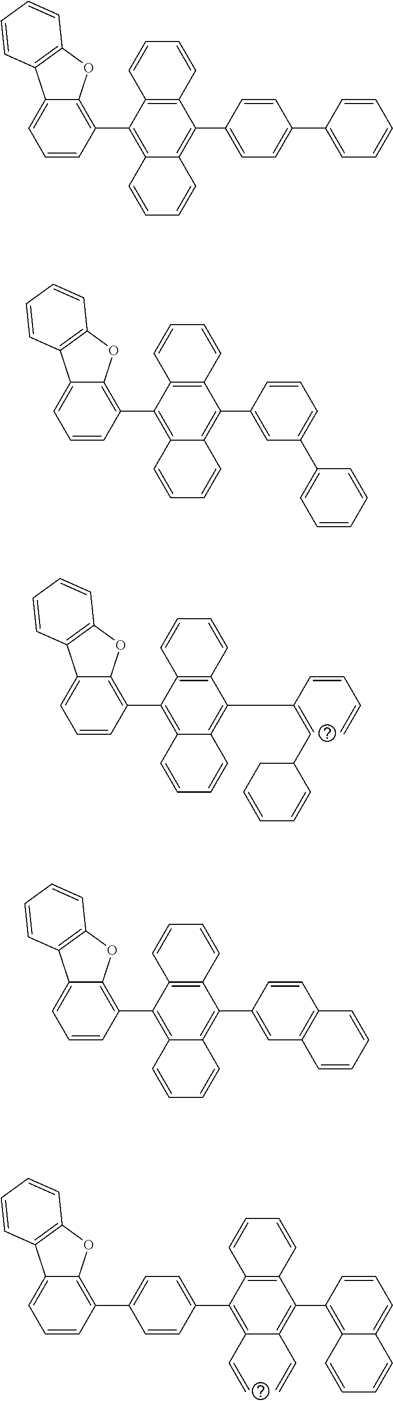 [SMILES: c1ccc(-c2ccc(-c3c4ccccc4c(-c4cccc5c4oc4ccccc45)c4ccccc34)cc2)cc1 ; c1ccc(-c2cccc(-c3c4ccccc4c(-c4cccc5c4oc4ccccc45)c4ccccc34)c2)cc1 ; C=C/C=C\C(=C\C1C=CC=CC1)c1c2ccccc2c(-c2cccc3c2oc2ccccc23)c2ccccc12 ; c1ccc2cc(-c3c4ccccc4c(-c4cccc5c4oc4ccccc45)c4ccccc34)ccc2c1 ; C=Cc1c(C=C)c(-c2cccc3ccccc23)c2ccccc2c1-c1ccc(-c2cccc3c2oc2ccccc23)cc1]

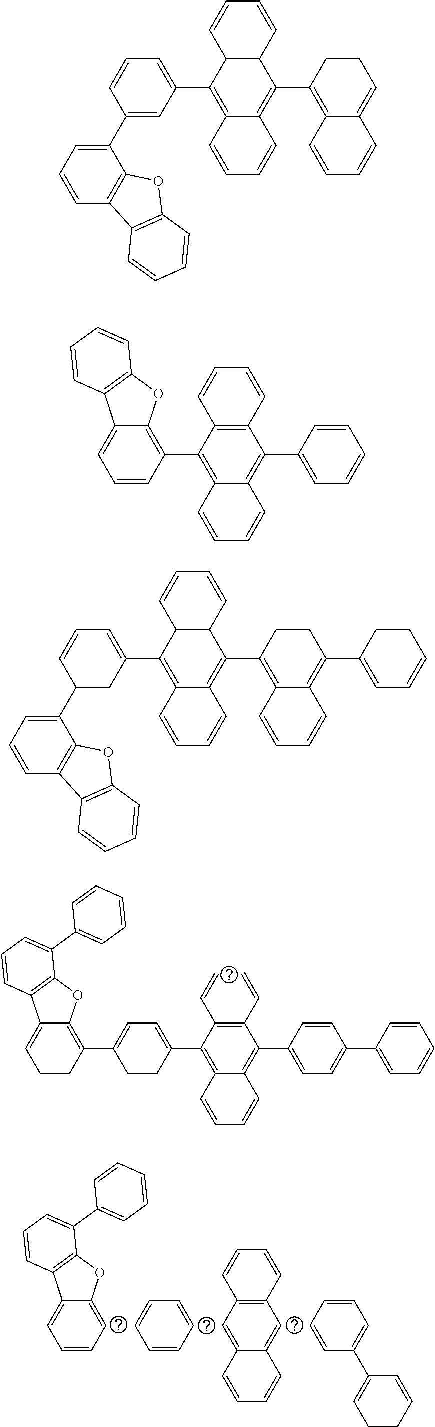

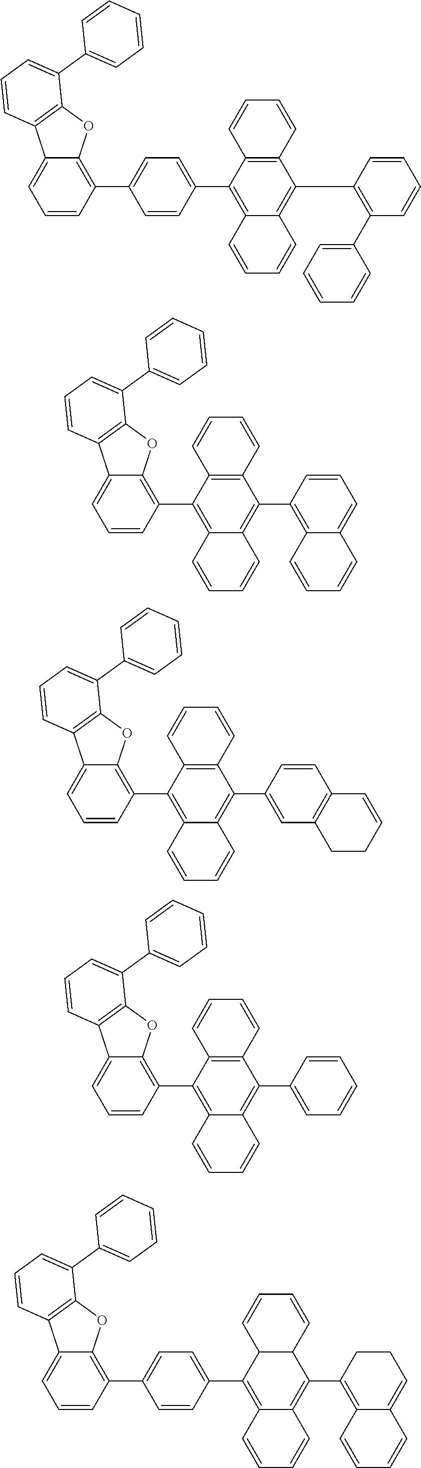

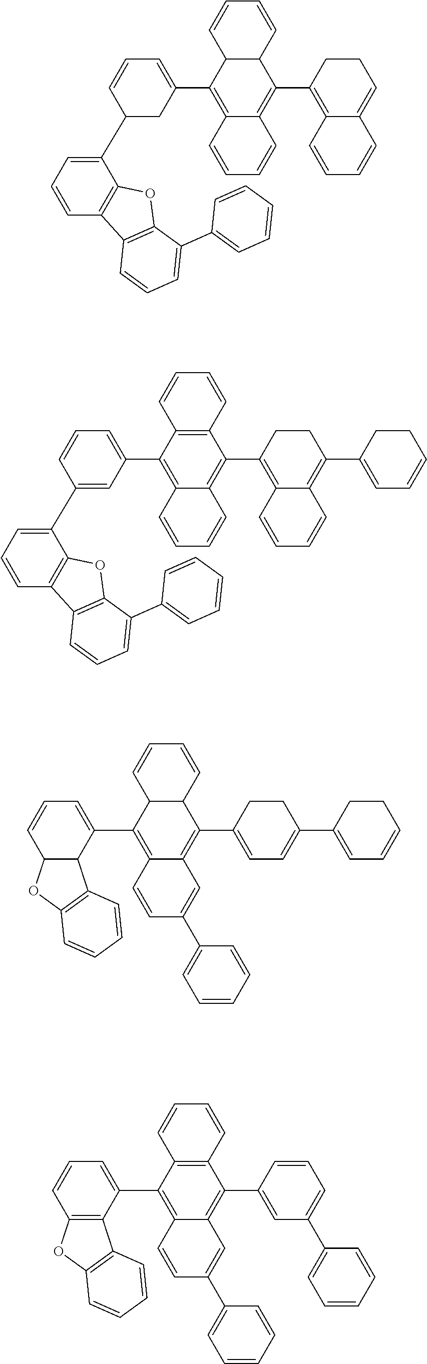 [SMILES: C1=CC(c2cccc3c2oc2c(-c4ccccc4)cccc23)CC(C2=c3ccccc3=C(C3=c4ccccc4=CCC3)C3C=CC=CC23)=C1 ; C1=CCCC(C2=c3ccccc3=C(c3c4ccccc4c(-c4cccc(-c5cccc6c5oc5c(-c7ccccc7)cccc56)c4)c4ccccc34)CC2)=C1 ; C1=CCCC(C2=CC=C(C3=c4cc(-c5ccccc5)ccc4=C(C4=CC=CC5Oc6ccccc6C45)C4C=CC=CC34)CC2)=C1 ; c1ccc(-c2cccc(-c3c4ccccc4c(-c4cccc5oc6ccccc6c45)c4ccc(-c5ccccc5)cc34)c2)cc1]

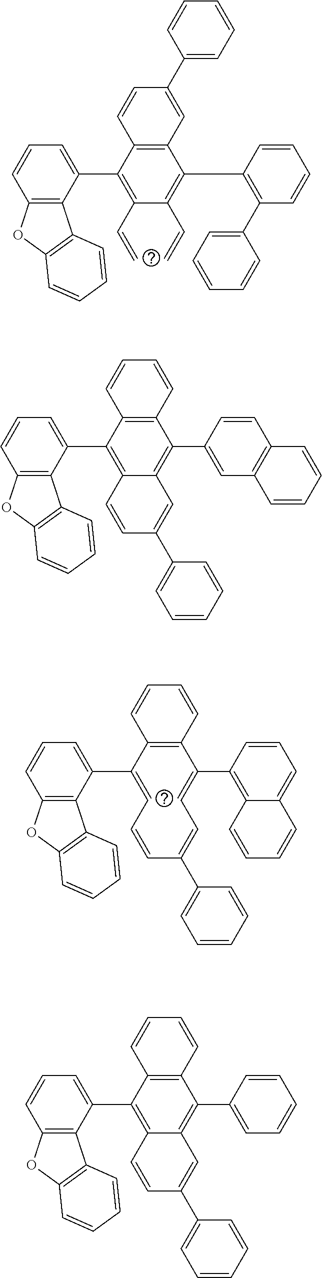 [SMILES: C=Cc1c(C=C)c(-c2cccc3oc4ccccc4c23)c2ccc(-c3ccccc3)cc2c1-c1ccccc1-c1ccccc1 ; c1ccc(-c2ccc3c(-c4cccc5oc6ccccc6c45)c4ccccc4c(-c4ccc5ccccc5c4)c3c2)cc1 ; c1ccc(-c2cccc(-c3cccc4oc5ccccc5c34)c3ccccc3c(-c3cccc4ccccc34)cc2)cc1 ; c1ccc(-c2ccc3c(-c4cccc5oc6ccccc6c45)c4ccccc4c(-c4ccccc4)c3c2)cc1]

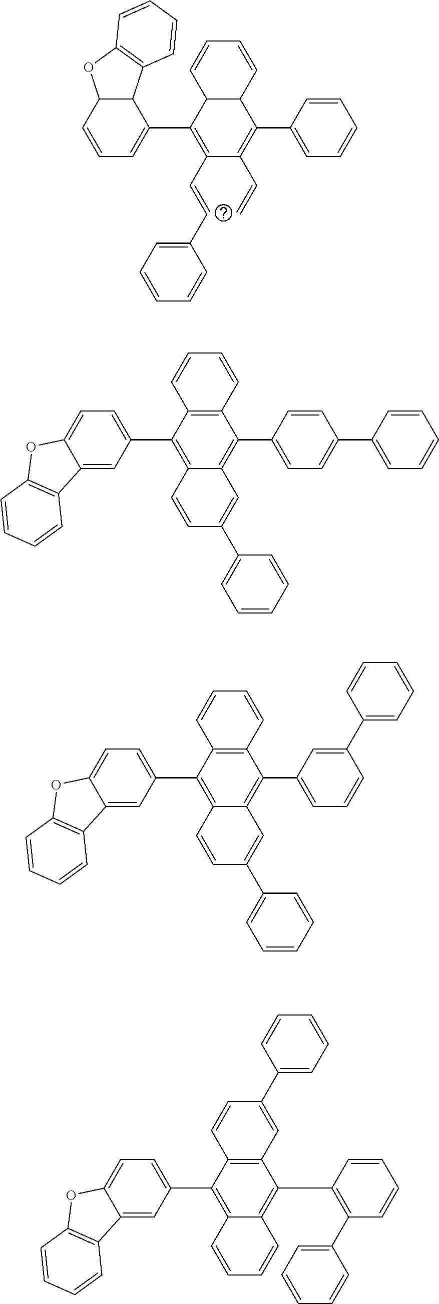 [SMILES: C=CC1=C(c2ccccc2)C2C=CC=CC2C(C2=CC=CC3Oc4ccccc4C23)=C1/C=C/c1ccccc1 ; c1ccc(-c2ccc(-c3c4ccccc4c(-c4ccc5oc6ccccc6c5c4)c4ccc(-c5ccccc5)cc34)cc2)cc1 ; c1ccc(-c2cccc(-c3c4ccccc4c(-c4ccc5oc6ccccc6c5c4)c4ccc(-c5ccccc5)cc34)c2)cc1 ; c1ccc(-c2ccc3c(-c4ccc5oc6ccccc6c5c4)c4ccccc4c(-c4ccccc4-c4ccccc4)c3c2)cc1]

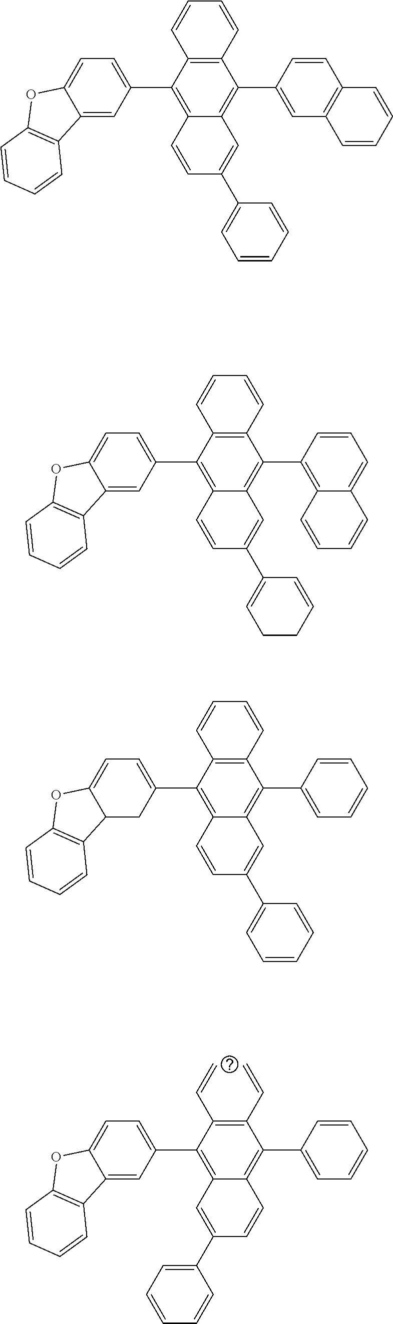 [SMILES: c1ccc(-c2ccc3c(-c4ccc5oc6ccccc6c5c4)c4ccccc4c(-c4ccc5ccccc5c4)c3c2)cc1 ; C1=CC(c2ccc3c(-c4ccc5oc6ccccc6c5c4)c4ccccc4c(-c4cccc5ccccc45)c3c2)=CCC1 ; C1=C(c2c3ccccc3c(-c3ccccc3)c3cc(-c4ccccc4)ccc23)CC2C(=C1)Oc1ccccc12 ; C=Cc1c(C=C)c(-c2ccc3oc4ccccc4c3c2)c2cc(-c3ccccc3)ccc2c1-c1ccccc1]

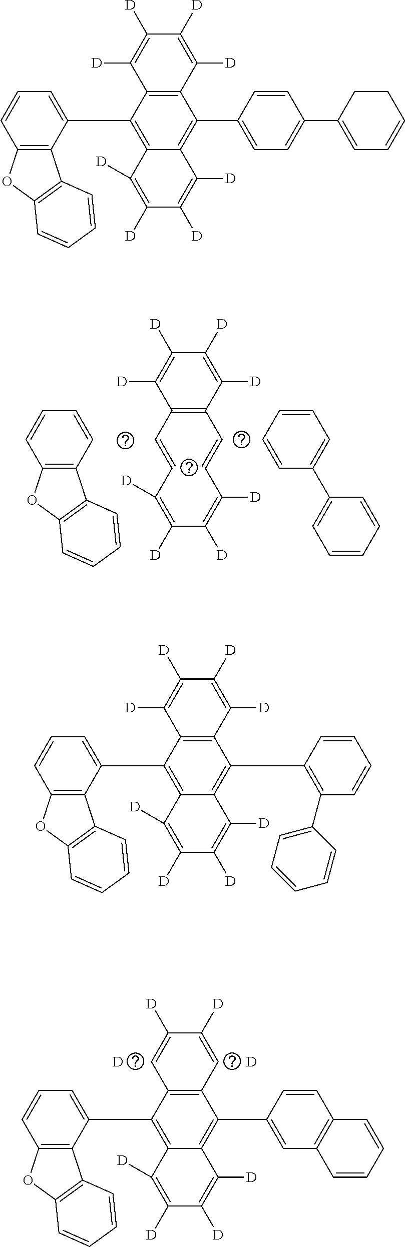

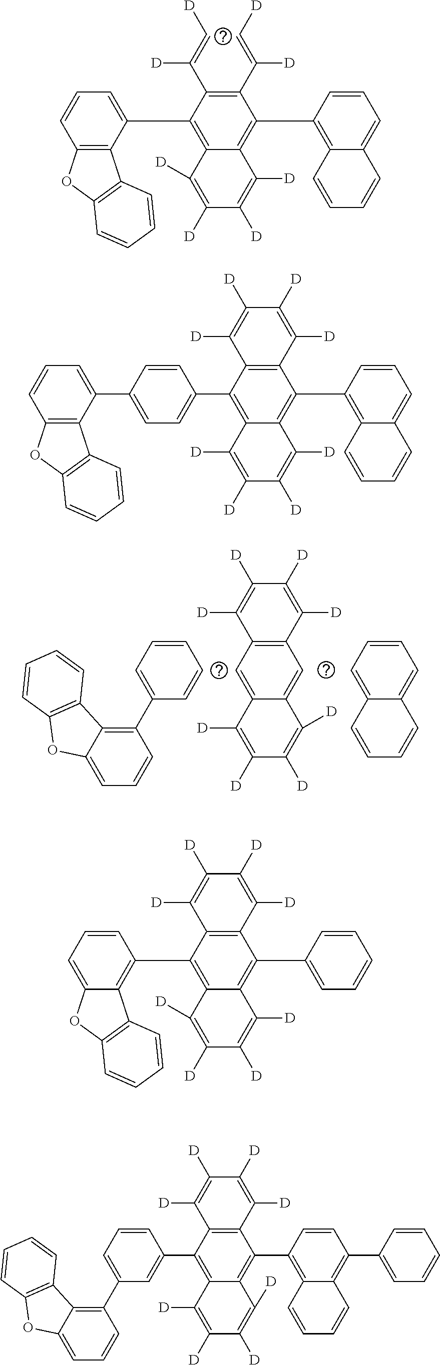

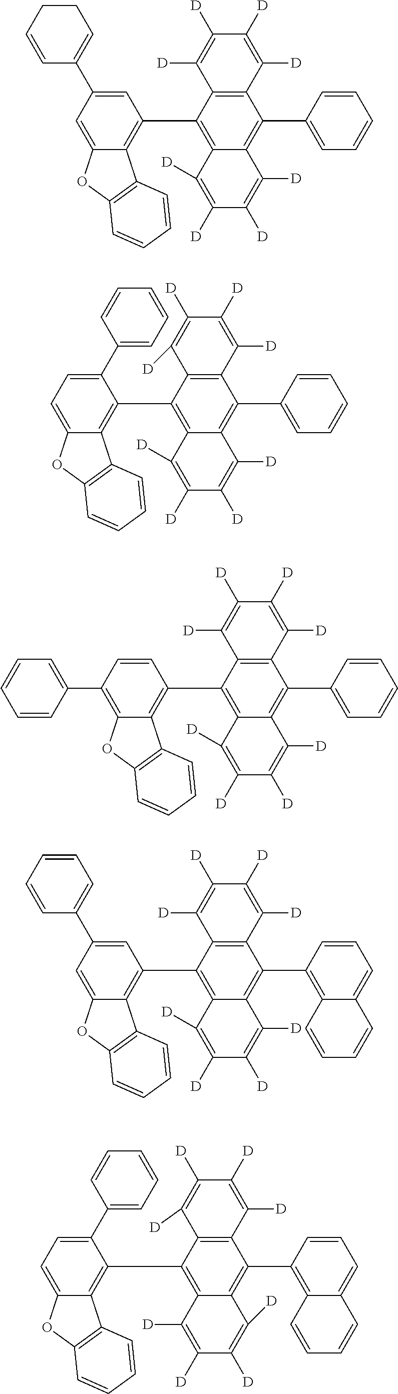

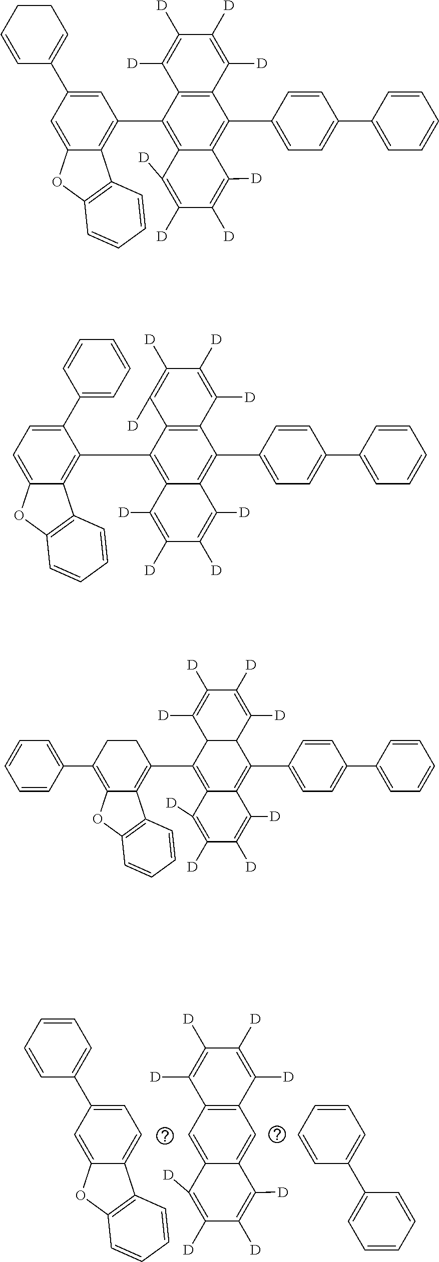

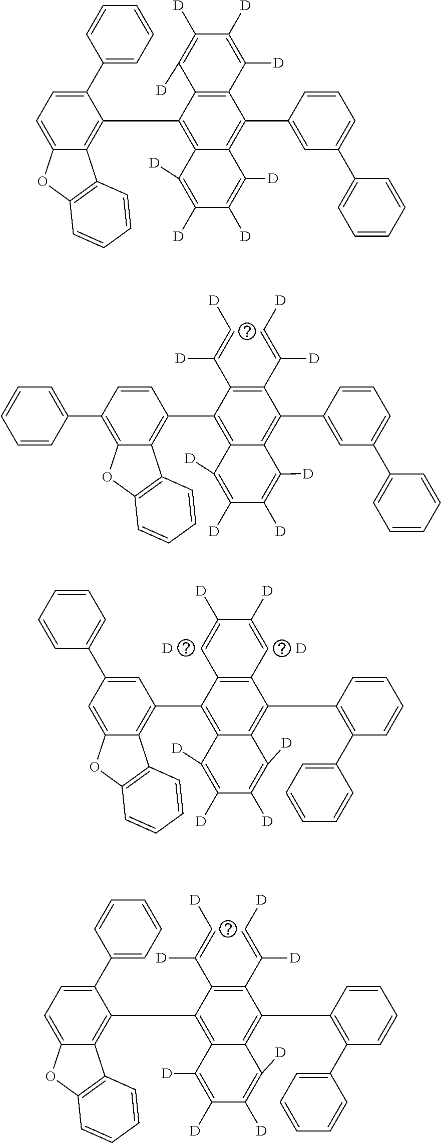

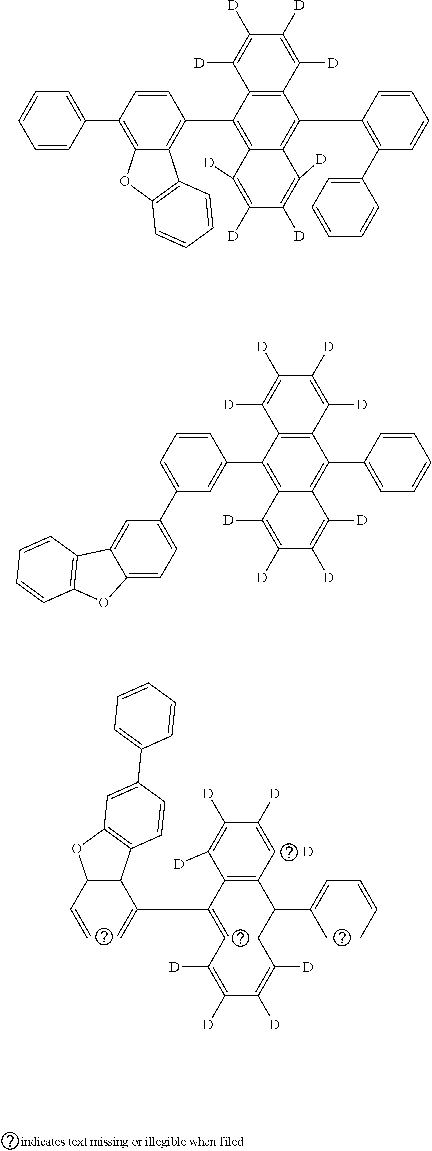

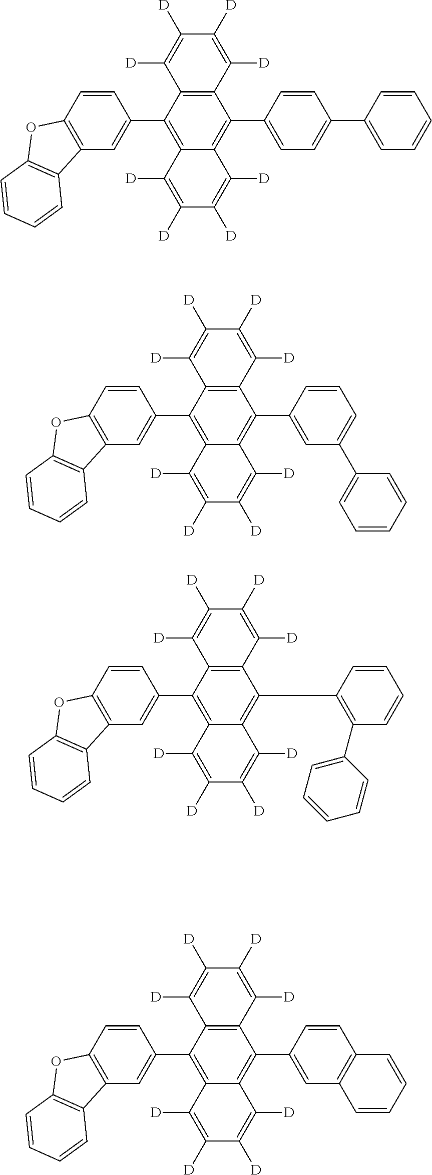 [SMILES: [2H]c1c([2H])c([2H])c2c(-c3ccc4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3ccc4oc5ccccc5c4c3)c3c([2H])c([2H])c([2H])c([2H])c3c(-c3cccc(-c4ccccc4)c3)c2c1[2H] ; [2H]c1c([2H])c([2H])c2c(-c3ccccc3-c3ccccc3)c3c([2H])c([2H])c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c([2H])c([2H])c([2H])c3c(-c3ccc4ccccc4c3)c2c1[2H]]

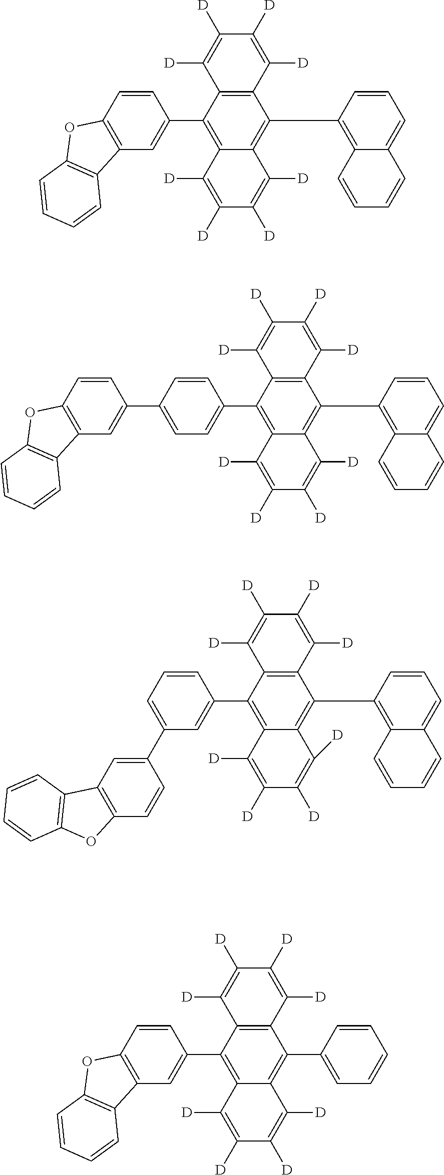 [SMILES: [2H]c1c([2H])c([2H])c2c(-c3cccc4ccccc34)c3c([2H])c([2H])c([2H])c([2H])c3c(-c3ccc4oc5ccccc5c4c3)c2c1[2H] ; [2H]c1c([2H])c([2H])c2c(-c3cccc4ccccc34)c3c([2H])c([2H])c([2H])c([2H])c3c(-c3ccc(-c4ccc5oc6ccccc6c5c4)cc3)c2c1[2H] ; [2H]c1c([2H])c([2H])c2c(-c3cccc4ccccc34)c3c([2H])c([2H])c([2H])c([2H])c3c(-c3cccc(-c4ccc5oc6ccccc6c5c4)c3)c2c1[2H] ; [2H]c1c([2H])c([2H])c2c(-c3ccc4oc5ccccc5c4c3)c3c([2H])c([2H])c([2H])c([2H])c3c(-c3ccccc3)c2c1[2H]]

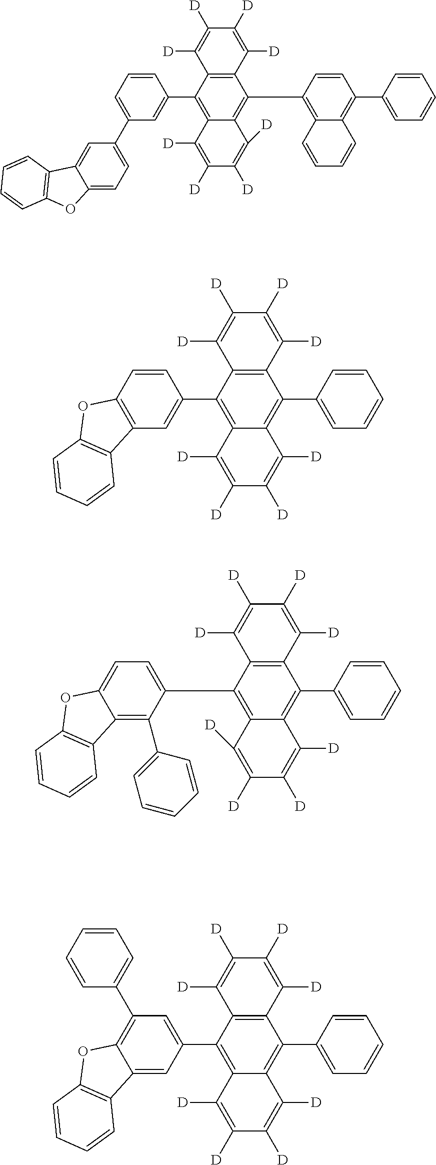 [SMILES: [2H]c1c([2H])c([2H])c2c(-c3ccc(-c4ccccc4)c4ccccc34)c3c([2H])c([2H])c([2H])c([2H])c3c(-c3cccc(-c4ccc5oc6ccccc6c5c4)c3)c2c1[2H] ; [2H]c1c([2H])c([2H])c2c(-c3ccc4oc5ccccc5c4c3)c3c([2H])c([2H])c([2H])c([2H])c3c(-c3ccccc3)c2c1[2H] ; [2H]c1c([2H])c([2H])c2c(-c3ccc4oc5ccccc5c4c3-c3ccccc3)c3c([2H])c([2H])c([2H])c([2H])c3c(-c3ccccc3)c2c1[2H] ; [2H]c1c([2H])c([2H])c2c(-c3cc(-c4ccccc4)c4oc5ccccc5c4c3)c3c([2H])c([2H])c([2H])c([2H])c3c(-c3ccccc3)c2c1[2H]]

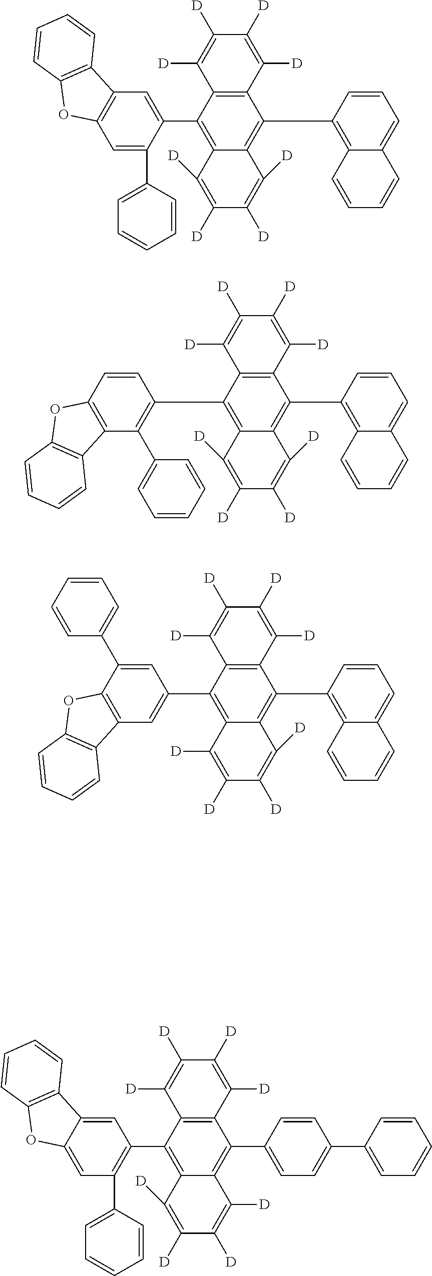 [SMILES: [2H]c1c([2H])c([2H])c2c(-c3cccc4ccccc34)c3c([2H])c([2H])c([2H])c([2H])c3c(-c3cc4c(cc3-c3ccccc3)oc3ccccc34)c2c1[2H] ; [2H]c1c([2H])c([2H])c2c(-c3cccc4ccccc34)c3c([2H])c([2H])c([2H])c([2H])c3c(-c3ccc4oc5ccccc5c4c3-c3ccccc3)c2c1[2H] ; [2H]c1c([2H])c([2H])c2c(-c3cccc4ccccc34)c3c([2H])c([2H])c([2H])c([2H])c3c(-c3cc(-c4ccccc4)c4oc5ccccc5c4c3)c2c1[2H] ; [2H]c1c([2H])c([2H])c2c(-c3cc4c(cc3-c3ccccc3)oc3ccccc34)c3c([2H])c([2H])c([2H])c([2H])c3c(-c3ccc(-c4ccccc4)cc3)c2c1[2H]]

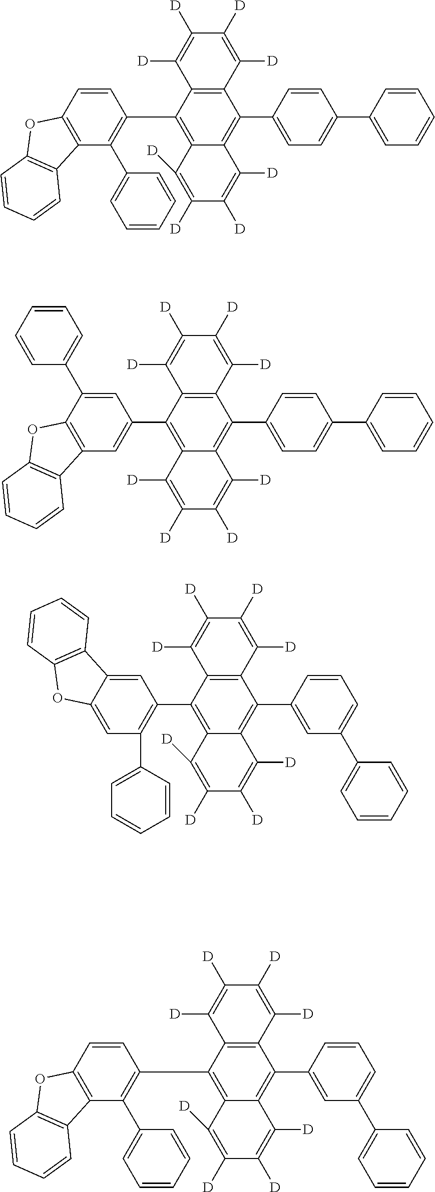 [SMILES: [2H]c1c([2H])c([2H])c2c(-c3ccc4oc5ccccc5c4c3-c3ccccc3)c3c([2H])c([2H])c([2H])c([2H])c3c(-c3ccc(-c4ccccc4)cc3)c2c1[2H] ; [2H]c1c([2H])c([2H])c2c(-c3cc(-c4ccccc4)c4oc5ccccc5c4c3)c3c([2H])c([2H])c([2H])c([2H])c3c(-c3ccc(-c4ccccc4)cc3)c2c1[2H] ; [2H]c1c([2H])c([2H])c2c(-c3cc4c(cc3-c3ccccc3)oc3ccccc34)c3c([2H])c([2H])c([2H])c([2H])c3c(-c3cccc(-c4ccccc4)c3)c2c1[2H] ; [2H]c1c([2H])c([2H])c2c(-c3ccc4oc5ccccc5c4c3-c3ccccc3)c3c([2H])c([2H])c([2H])c([2H])c3c(-c3cccc(-c4ccccc4)c3)c2c1[2H]]

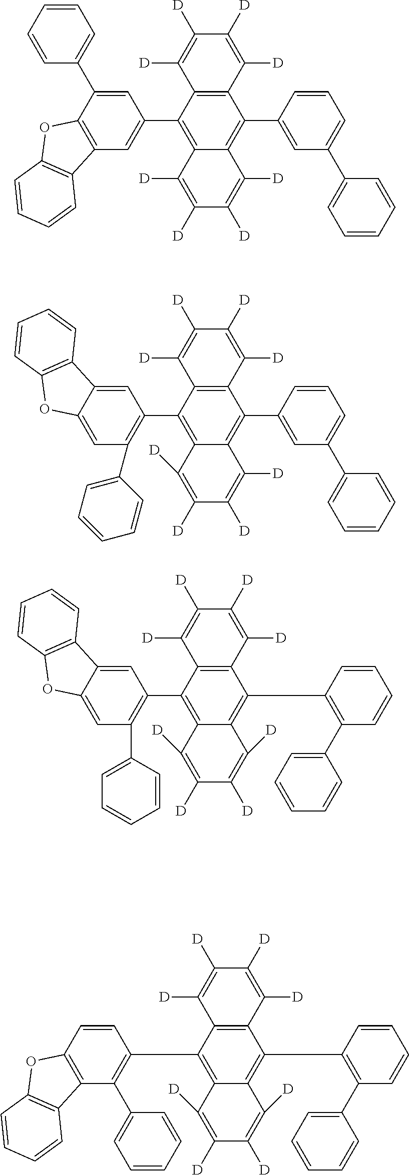 [SMILES: [2H]c1c([2H])c([2H])c2c(-c3cc(-c4ccccc4)c4oc5ccccc5c4c3)c3c([2H])c([2H])c([2H])c([2H])c3c(-c3cccc(-c4ccccc4)c3)c2c1[2H] ; [2H]c1c([2H])c([2H])c2c(-c3cc4c(cc3-c3ccccc3)oc3ccccc34)c3c([2H])c([2H])c([2H])c([2H])c3c(-c3cccc(-c4ccccc4)c3)c2c1[2H] ; [2H]c1c([2H])c([2H])c2c(-c3cc4c(cc3-c3ccccc3)oc3ccccc34)c3c([2H])c([2H])c([2H])c([2H])c3c(-c3ccccc3-c3ccccc3)c2c1[2H] ; [2H]c1c([2H])c([2H])c2c(-c3ccc4oc5ccccc5c4c3-c3ccccc3)c3c([2H])c([2H])c([2H])c([2H])c3c(-c3ccccc3-c3ccccc3)c2c1[2H]]

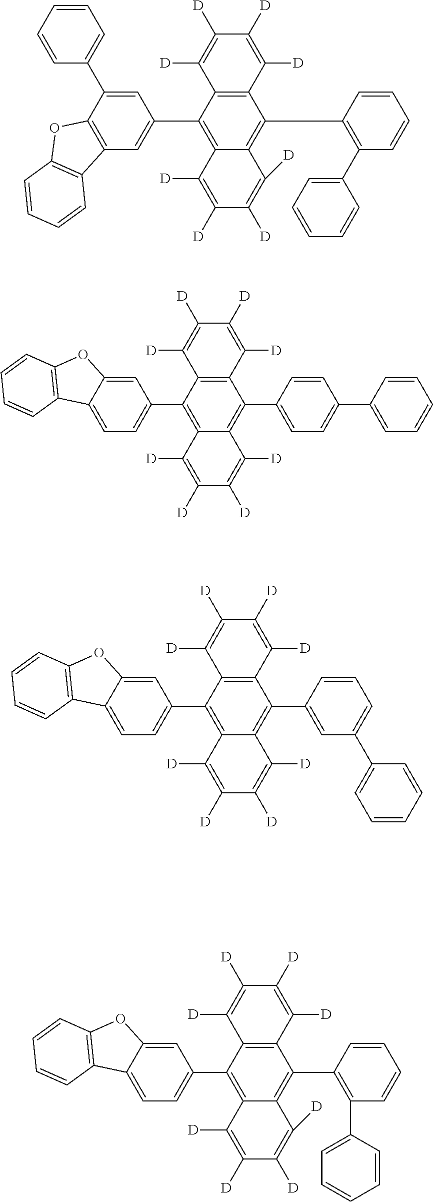 [SMILES: [2H]c1c([2H])c([2H])c2c(-c3ccccc3-c3ccccc3)c3c([2H])c([2H])c([2H])c([2H])c3c(-c3cc(-c4ccccc4)c4oc5ccccc5c4c3)c2c1[2H] ; [2H]c1c([2H])c([2H])c2c(-c3ccc4c(c3)oc3ccccc34)c3c([2H])c([2H])c([2H])c([2H])c3c(-c3ccc(-c4ccccc4)cc3)c2c1[2H] ; [2H]c1c([2H])c([2H])c2c(-c3ccc4c(c3)oc3ccccc34)c3c([2H])c([2H])c([2H])c([2H])c3c(-c3cccc(-c4ccccc4)c3)c2c1[2H] ; [2H]c1c([2H])c([2H])c2c(-c3ccccc3-c3ccccc3)c3c([2H])c([2H])c([2H])c([2H])c3c(-c3ccc4c(c3)oc3ccccc34)c2c1[2H]]

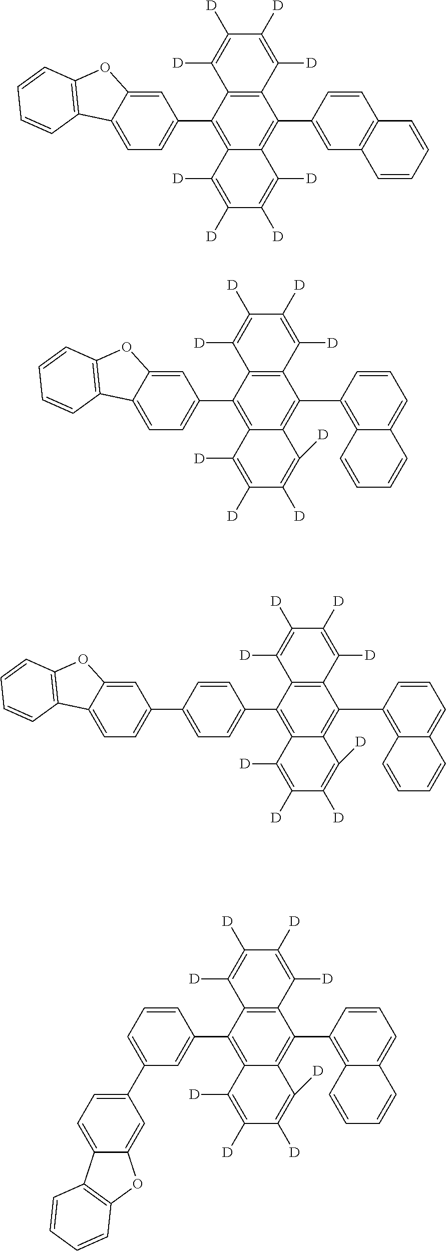 [SMILES: [2H]c1c([2H])c([2H])c2c(-c3ccc4c(c3)oc3ccccc34)c3c([2H])c([2H])c([2H])c([2H])c3c(-c3ccc4ccccc4c3)c2c1[2H] ; [2H]c1c([2H])c([2H])c2c(-c3cccc4ccccc34)c3c([2H])c([2H])c([2H])c([2H])c3c(-c3ccc4c(c3)oc3ccccc34)c2c1[2H] ; [2H]c1c([2H])c([2H])c2c(-c3cccc4ccccc34)c3c([2H])c([2H])c([2H])c([2H])c3c(-c3ccc(-c4ccc5c(c4)oc4ccccc45)cc3)c2c1[2H] ; [2H]c1c([2H])c([2H])c2c(-c3cccc4ccccc34)c3c([2H])c([2H])c([2H])c([2H])c3c(-c3cccc(-c4ccc5c(c4)oc4ccccc45)c3)c2c1[2H]]

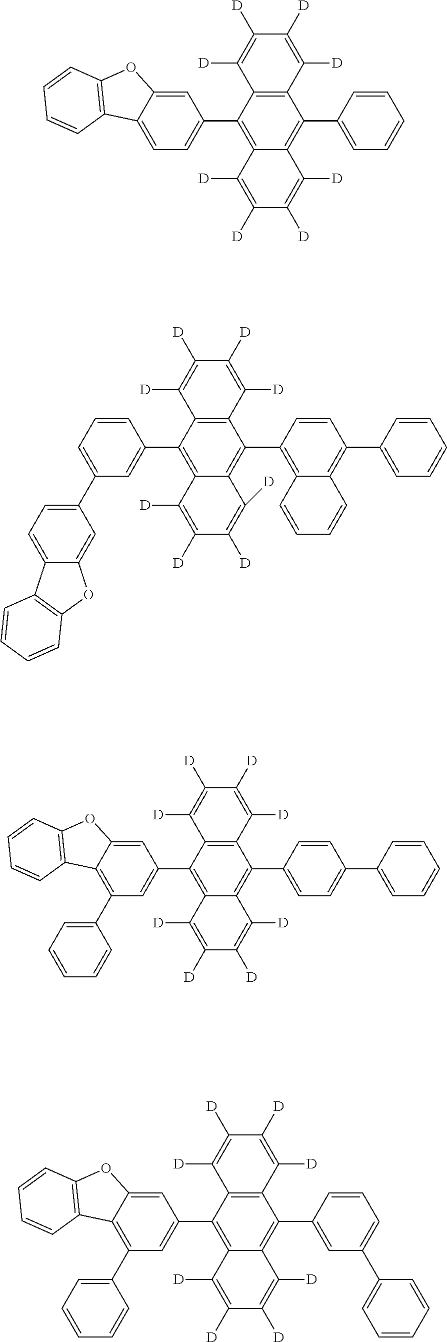 [SMILES: [2H]c1c([2H])c([2H])c2c(-c3ccc4c(c3)oc3ccccc34)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3cccc(-c4ccc5c(c4)oc4ccccc45)c3)c2c1[2H] ; [2H]c1c([2H])c([2H])c2c(-c3cc(-c4ccccc4)c4c(c3)oc3ccccc34)c3c([2H])c([2H])c([2H])c([2H])c3c(-c3ccc(-c4ccccc4)cc3)c2c1[2H] ; [2H]c1c([2H])c([2H])c2c(-c3cc(-c4ccccc4)c4c(c3)oc3ccccc34)c3c([2H])c([2H])c([2H])c([2H])c3c(-c3cccc(-c4ccccc4)c3)c2c1[2H]]

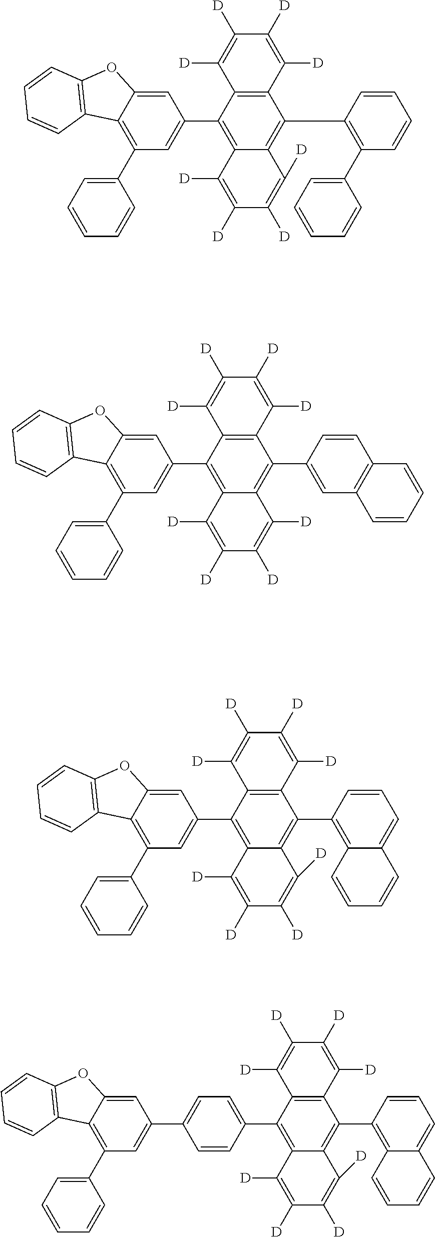 [SMILES: [2H]c1c([2H])c([2H])c2c(-c3ccccc3-c3ccccc3)c3c([2H])c([2H])c([2H])c([2H])c3c(-c3cc(-c4ccccc4)c4c(c3)oc3ccccc34)c2c1[2H] ; [2H]c1c([2H])c([2H])c2c(-c3cc(-c4ccccc4)c4c(c3)oc3ccccc34)c3c([2H])c([2H])c([2H])c([2H])c3c(-c3ccc4ccccc4c3)c2c1[2H] ; [2H]c1c([2H])c([2H])c2c(-c3cccc4ccccc34)c3c([2H])c([2H])c([2H])c([2H])c3c(-c3cc(-c4ccccc4)c4c(c3)oc3ccccc34)c2c1[2H] ; [2H]c1c([2H])c([2H])c2c(-c3cccc4ccccc34)c3c([2H])c([2H])c([2H])c([2H])c3c(-c3ccc(-c4cc(-c5ccccc5)c5c(c4)oc4ccccc45)cc3)c2c1[2H]]

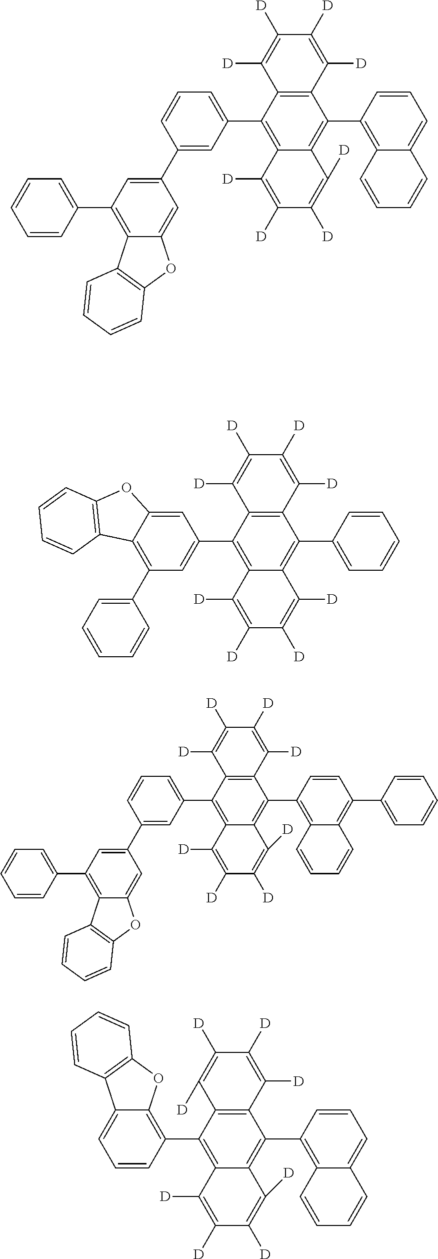

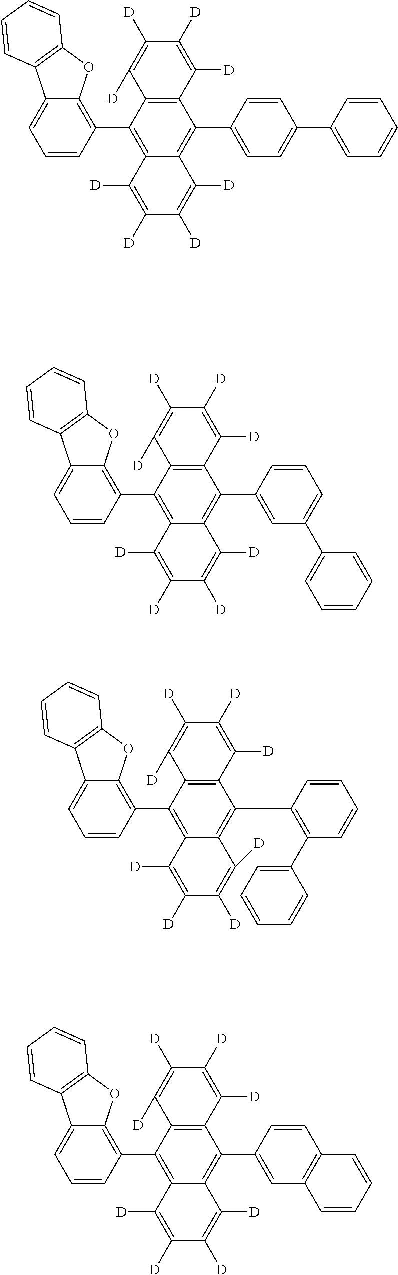 [SMILES: [2H]c1c([2H])c([2H])c2c(-c3cccc4c3oc3ccccc34)c3c([2H])c([2H])c([2H])c([2H])c3c(-c3ccc(-c4ccccc4)cc3)c2c1[2H] ; [2H]c1c([2H])c([2H])c2c(-c3cccc4c3oc3ccccc34)c3c([2H])c([2H])c([2H])c([2H])c3c(-c3cccc(-c4ccccc4)c3)c2c1[2H] ; [2H]c1c([2H])c([2H])c2c(-c3cccc4c3oc3ccccc34)c3c([2H])c([2H])c([2H])c([2H])c3c(-c3ccccc3-c3ccccc3)c2c1[2H] ; [2H]c1c([2H])c([2H])c2c(-c3cccc4c3oc3ccccc34)c3c([2H])c([2H])c([2H])c([2H])c3c(-c3ccc4ccccc4c3)c2c1[2H]]

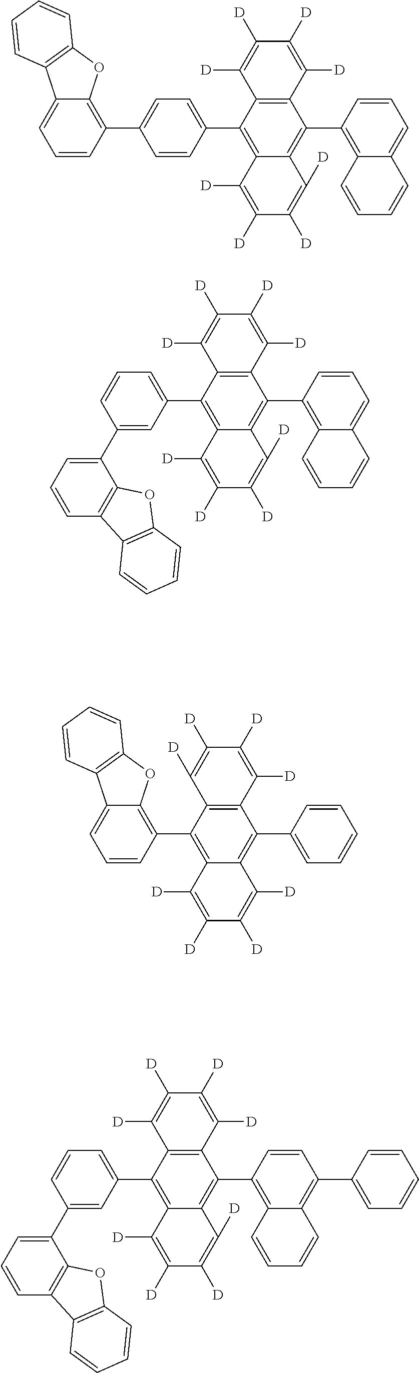 [SMILES: [2H]c1c([2H])c([2H])c2c(-c3cccc4ccccc34)c3c([2H])c([2H])c([2H])c([2H])c3c(-c3ccc(-c4cccc5c4oc4ccccc45)cc3)c2c1[2H] ; [2H]c1c([2H])c([2H])c2c(-c3cccc4ccccc34)c3c([2H])c([2H])c([2H])c([2H])c3c(-c3cccc(-c4cccc5c4oc4ccccc45)c3)c2c1[2H] ; [2H]c1c([2H])c([2H])c2c(-c3cccc4c3oc3ccccc34)c3c([2H])c([2H])c([2H])c([2H])c3c(-c3ccccc3)c2c1[2H] ; [2H]c1c([2H])c([2H])c2c(-c3ccc(-c4ccccc4)c4ccccc34)c3c([2H])c([2H])c([2H])c([2H])c3c(-c3cccc(-c4cccc5c4oc4ccccc45)c3)c2c1[2H]]

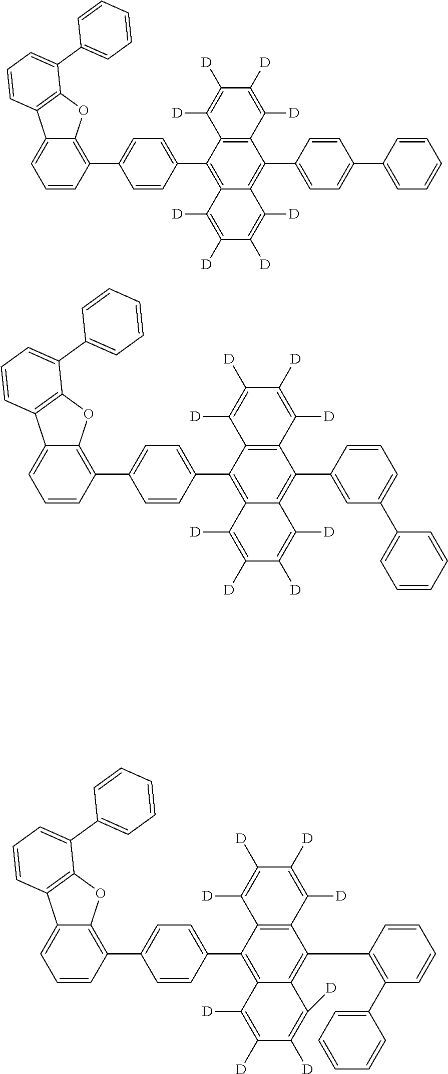 [SMILES: [2H]c1c([2H])c([2H])c2c(-c3ccc(-c4cccc5c4oc4c(-c6ccccc6)cccc45)cc3)c3c([2H])c([2H])c([2H])c([2H])c3c(-c3ccc(-c4ccccc4)cc3)c2c1[2H] ; [2H]c1c([2H])c([2H])c2c(-c3cccc(-c4ccccc4)c3)c3c([2H])c([2H])c([2H])c([2H])c3c(-c3ccc(-c4cccc5c4oc4c(-c6ccccc6)cccc45)cc3)c2c1[2H] ; [2H]c1c([2H])c([2H])c2c(-c3ccccc3-c3ccccc3)c3c([2H])c([2H])c([2H])c([2H])c3c(-c3ccc(-c4cccc5c4oc4c(-c6ccccc6)cccc45)cc3)c2c1[2H]]

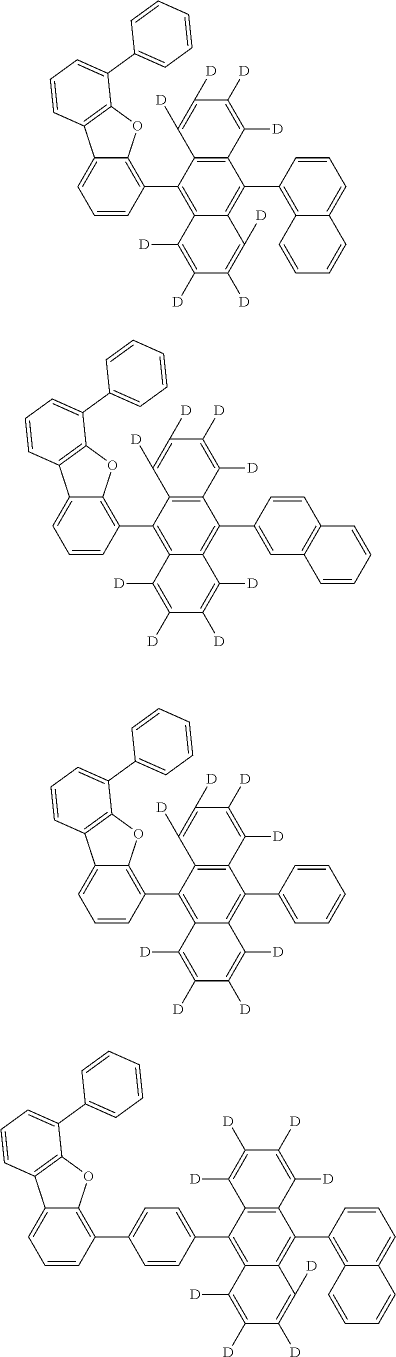 [SMILES: [2H]c1c([2H])c([2H])c2c(-c3cccc4c3oc3c(-c5ccccc5)cccc34)c3c([2H])c([2H])c([2H])c([2H])c3c(-c3cccc4ccccc34)c2c1[2H] ; [2H]c1c([2H])c([2H])c2c(-c3cccc4c3oc3c(-c5ccccc5)cccc34)c3c([2H])c([2H])c([2H])c([2H])c3c(-c3ccc4ccccc4c3)c2c1[2H] ; [2H]c1c([2H])c([2H])c2c(-c3cccc4c3oc3c(-c5ccccc5)cccc34)c3c([2H])c([2H])c([2H])c([2H])c3c(-c3ccccc3)c2c1[2H] ; [2H]c1c([2H])c([2H])c2c(-c3cccc4ccccc34)c3c([2H])c([2H])c([2H])c([2H])c3c(-c3ccc(-c4cccc5c4oc4c(-c6ccccc6)cccc45)cc3)c2c1[2H]]

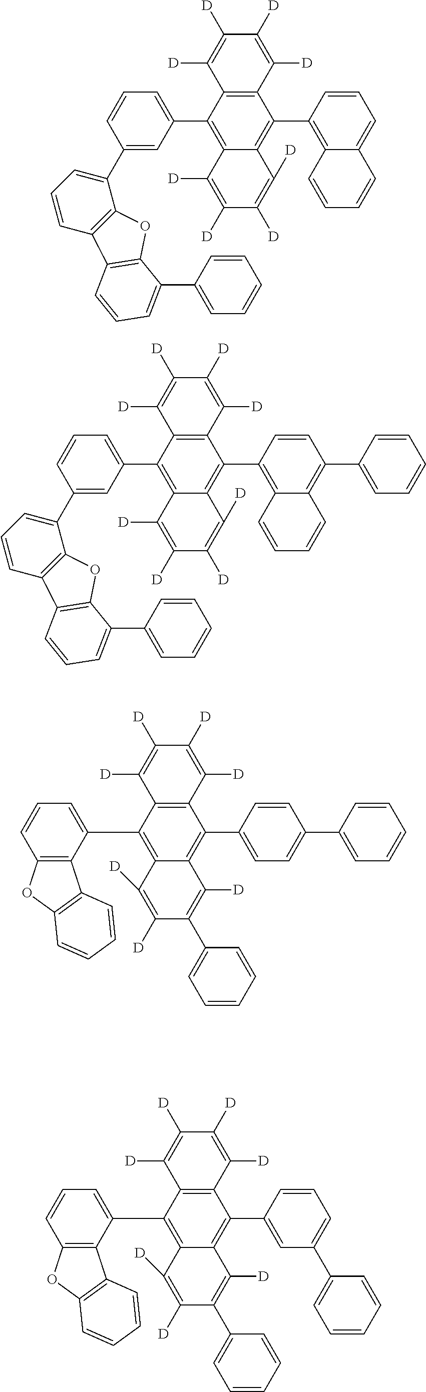

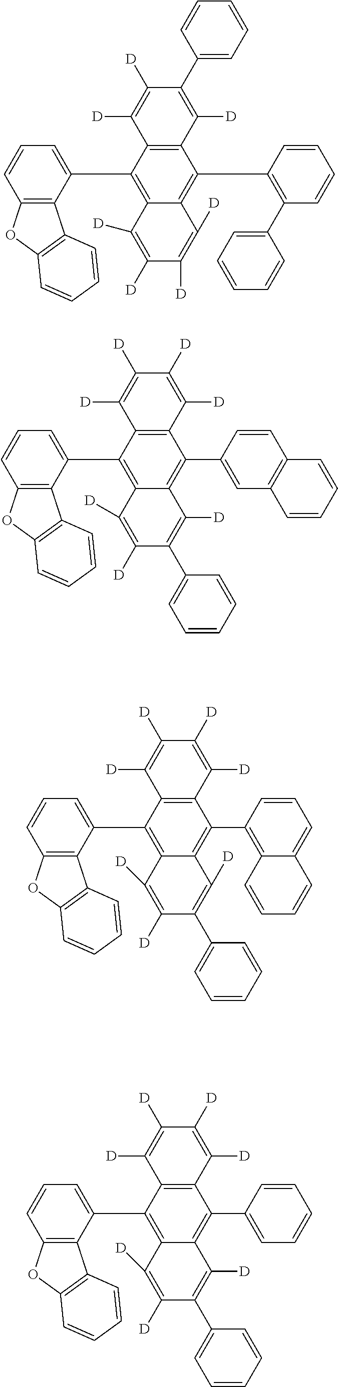 [SMILES: [2H]c1c([2H])c([2H])c2c(-c3cccc4oc5ccccc5c34)c3c([2H])c([2H])c(-c4ccccc4)c([2H])c3c(-c3ccccc3-c3ccccc3)c2c1[2H] ; [2H]c1c([2H])c([2H])c2c(-c3cccc4oc5ccccc5c34)c3c([2H])c([2H])c(-c4ccccc4)c([2H])c3c(-c3ccc4ccccc4c3)c2c1[2H] ; [2H]c1c([2H])c([2H])c2c(-c3cccc4oc5ccccc5c34)c3c([2H])c([2H])c(-c4ccccc4)c([2H])c3c(-c3cccc4ccccc34)c2c1[2H] ; [2H]c1c([2H])c([2H])c2c(-c3cccc4oc5ccccc5c34)c3c([2H])c([2H])c(-c4ccccc4)c([2H])c3c(-c3ccccc3)c2c1[2H]]

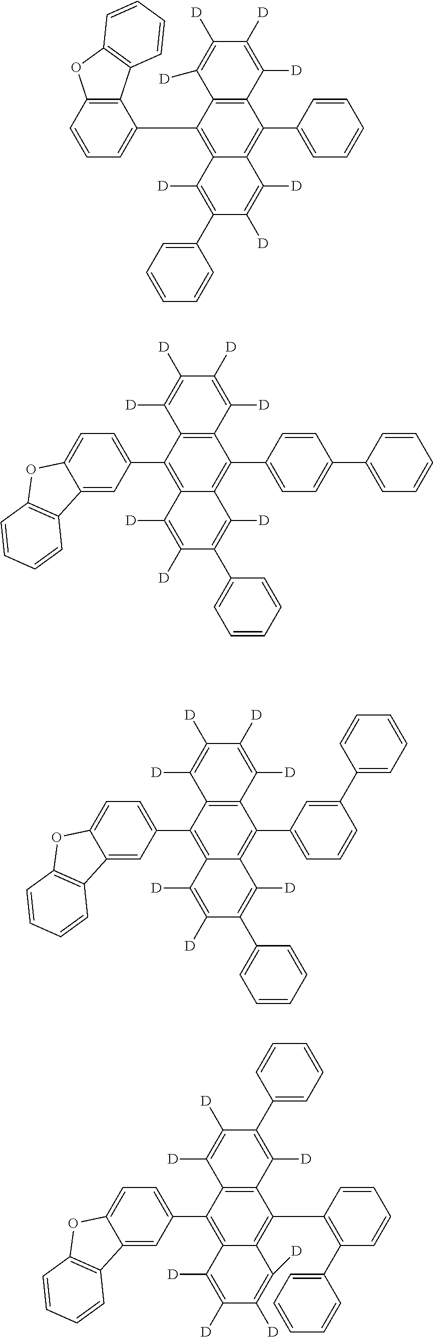

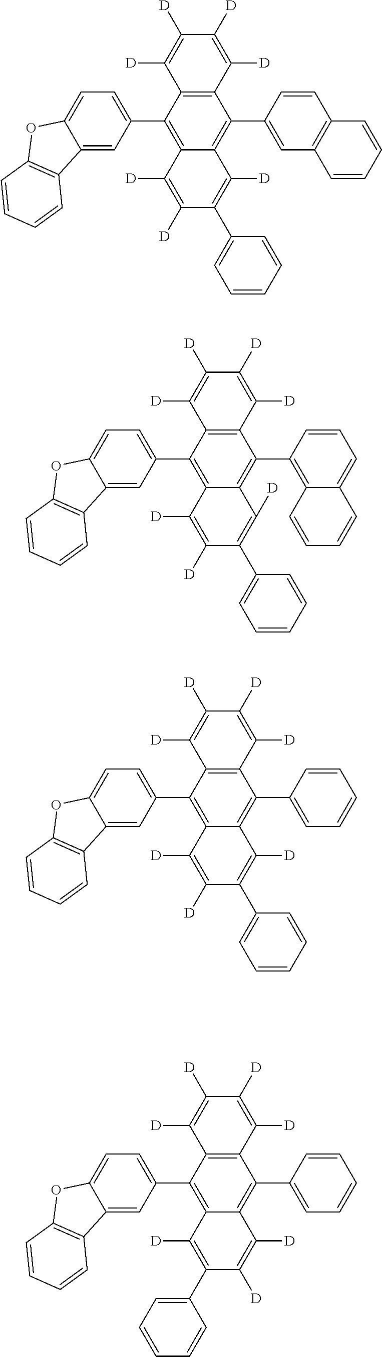 [SMILES: [2H]c1c([2H])c([2H])c2c(-c3ccc4ccccc4c3)c3c([2H])c(-c4ccccc4)c([2H])c([2H])c3c(-c3ccc4oc5ccccc5c4c3)c2c1[2H] ; [2H]c1c([2H])c([2H])c2c(-c3cccc4ccccc34)c3c([2H])c(-c4ccccc4)c([2H])c([2H])c3c(-c3ccc4oc5ccccc5c4c3)c2c1[2H] ; [2H]c1c([2H])c([2H])c2c(-c3ccccc3)c3c([2H])c(-c4ccccc4)c([2H])c([2H])c3c(-c3ccc4oc5ccccc5c4c3)c2c1[2H] ; [2H]c1c([2H])c([2H])c2c(-c3ccc4oc5ccccc5c4c3)c3c([2H])c(-c4ccccc4)c([2H])c([2H])c3c(-c3ccccc3)c2c1[2H]]

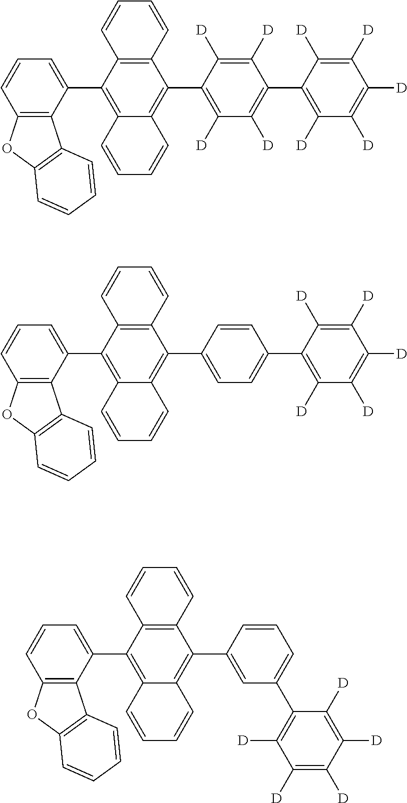 [SMILES: [2H]c1c([2H])c([2H])c(-c2c([2H])c([2H])c(-c3c4ccccc4c(-c4cccc5oc6ccccc6c45)c4ccccc34)c([2H])c2[2H])c([2H])c1[2H] ; [2H]c1c([2H])c([2H])c(-c2ccc(-c3c4ccccc4c(-c4cccc5oc6ccccc6c45)c4ccccc34)cc2)c([2H])c1[2H] ; [2H]c1c([2H])c([2H])c(-c2cccc(-c3c4ccccc4c(-c4cccc5oc6ccccc6c45)c4ccccc34)c2)c([2H])c1[2H]]

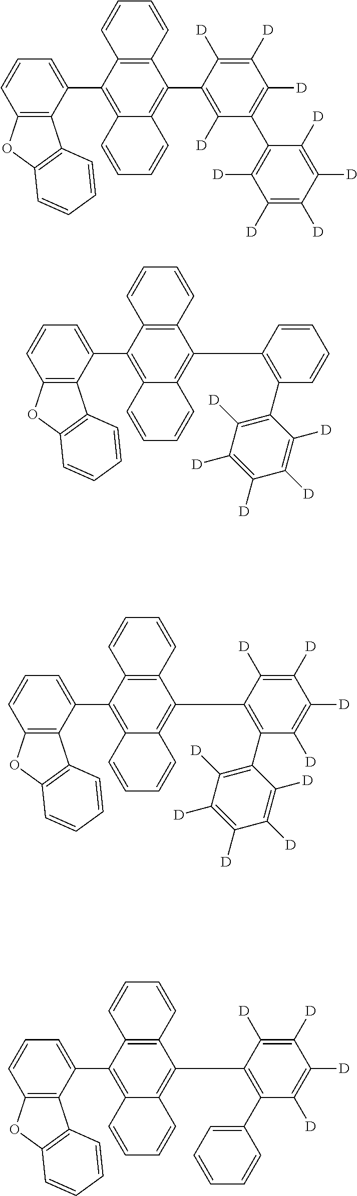 [SMILES: [2H]c1c([2H])c([2H])c(-c2c([2H])c([2H])c([2H])c(-c3c4ccccc4c(-c4cccc5oc6ccccc6c45)c4ccccc34)c2[2H])c([2H])c1[2H] ; [2H]c1c([2H])c([2H])c(-c2ccccc2-c2c3ccccc3c(-c3cccc4oc5ccccc5c34)c3ccccc23)c([2H])c1[2H] ; [2H]c1c([2H])c([2H])c(-c2c([2H])c([2H])c([2H])c([2H])c2-c2c3ccccc3c(-c3cccc4oc5ccccc5c34)c3ccccc23)c([2H])c1[2H] ; [2H]c1c([2H])c([2H])c(-c2c3ccccc3c(-c3cccc4oc5ccccc5c34)c3ccccc23)c(-c2ccccc2)c1[2H]]

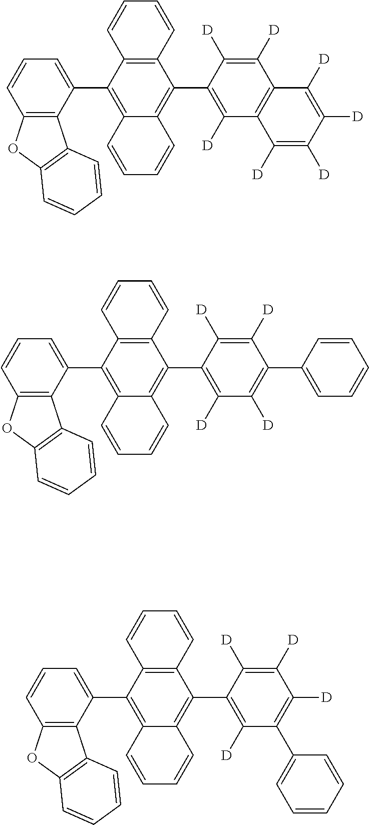 [SMILES: [2H]c1c([2H])c([2H])c2c([2H])c(-c3c4ccccc4c(-c4cccc5oc6ccccc6c45)c4ccccc34)c([2H])c([2H])c2c1[2H] ; [2H]c1c([2H])c(-c2c3ccccc3c(-c3cccc4oc5ccccc5c34)c3ccccc23)c([2H])c([2H])c1-c1ccccc1 ; [2H]c1c([2H])c(-c2ccccc2)c([2H])c(-c2c3ccccc3c(-c3cccc4oc5ccccc5c34)c3ccccc23)c1[2H]]

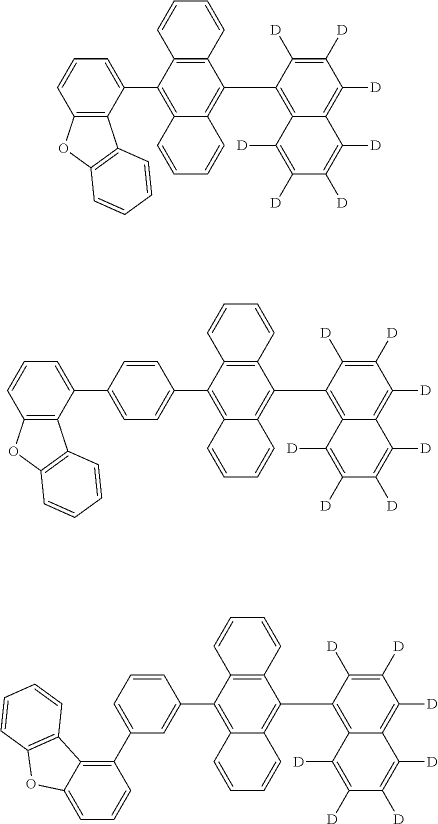 [SMILES: [2H]c1c([2H])c([2H])c2c(-c3c4ccccc4c(-c4cccc5oc6ccccc6c45)c4ccccc34)c([2H])c([2H])c([2H])c2c1[2H] ; [2H]c1c([2H])c([2H])c2c(-c3c4ccccc4c(-c4ccc(-c5cccc6oc7ccccc7c56)cc4)c4ccccc34)c([2H])c([2H])c([2H])c2c1[2H] ; [2H]c1c([2H])c([2H])c2c(-c3c4ccccc4c(-c4cccc(-c5cccc6oc7ccccc7c56)c4)c4ccccc34)c([2H])c([2H])c([2H])c2c1[2H]]

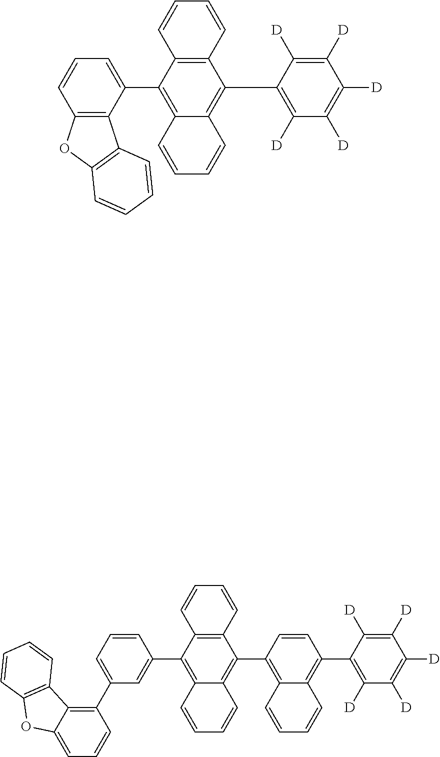 [SMILES: [2H]c1c([2H])c([2H])c(-c2c3ccccc3c(-c3cccc4oc5ccccc5c34)c3ccccc23)c([2H])c1[2H] ; [2H]c1c([2H])c([2H])c(-c2ccc(-c3c4ccccc4c(-c4cccc(-c5cccc6oc7ccccc7c56)c4)c4ccccc34)c3ccccc23)c([2H])c1[2H]]

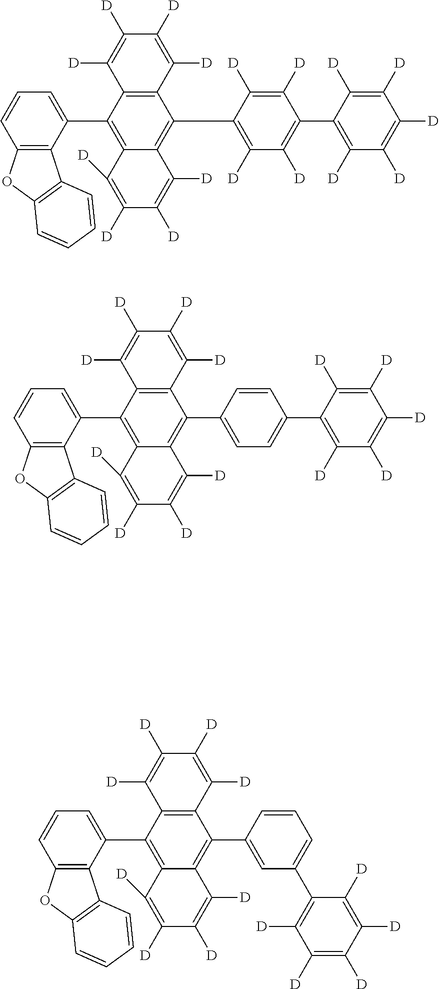 [SMILES: [2H]c1c([2H])c([2H])c(-c2c([2H])c([2H])c(-c3c4c([2H])c([2H])c([2H])c([2H])c4c(-c4cccc5oc6ccccc6c45)c4c([2H])c([2H])c([2H])c([2H])c34)c([2H])c2[2H])c([2H])c1[2H] ; [2H]c1c([2H])c([2H])c(-c2ccc(-c3c4c([2H])c([2H])c([2H])c([2H])c4c(-c4cccc5oc6ccccc6c45)c4c([2H])c([2H])c([2H])c([2H])c34)cc2)c([2H])c1[2H] ; [2H]c1c([2H])c([2H])c(-c2cccc(-c3c4c([2H])c([2H])c([2H])c([2H])c4c(-c4cccc5oc6ccccc6c45)c4c([2H])c([2H])c([2H])c([2H])c34)c2)c([2H])c1[2H]]

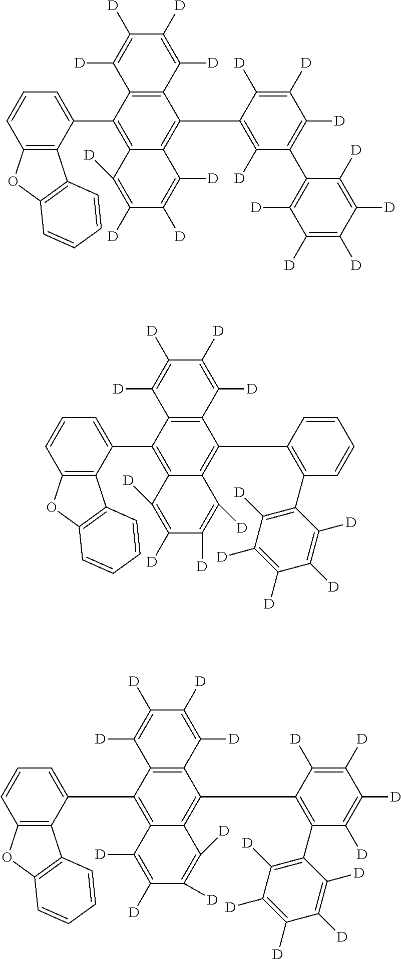 [SMILES: [2H]c1c([2H])c([2H])c(-c2c([2H])c([2H])c([2H])c(-c3c4c([2H])c([2H])c([2H])c([2H])c4c(-c4cccc5oc6ccccc6c45)c4c([2H])c([2H])c([2H])c([2H])c34)c2[2H])c([2H])c1[2H] ; [2H]c1c([2H])c([2H])c(-c2ccccc2-c2c3c([2H])c([2H])c([2H])c([2H])c3c(-c3cccc4oc5ccccc5c34)c3c([2H])c([2H])c([2H])c([2H])c23)c([2H])c1[2H] ; [2H]c1c([2H])c([2H])c(-c2c([2H])c([2H])c([2H])c([2H])c2-c2c3c([2H])c([2H])c([2H])c([2H])c3c(-c3cccc4oc5ccccc5c34)c3c([2H])c([2H])c([2H])c([2H])c23)c([2H])c1[2H]]

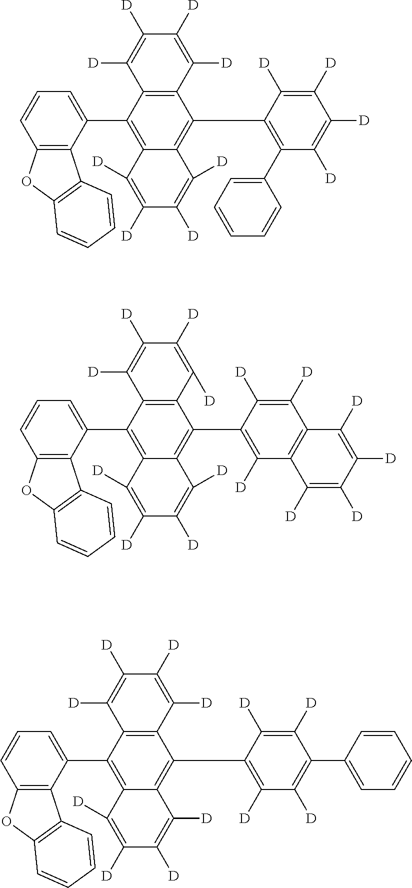 [SMILES: [2H]c1c([2H])c([2H])c(-c2c3c([2H])c([2H])c([2H])c([2H])c3c(-c3cccc4oc5ccccc5c34)c3c([2H])c([2H])c([2H])c([2H])c23)c(-c2ccccc2)c1[2H] ; [2H]c1c([2H])c([2H])c2c([2H])c(-c3c4c([2H])c([2H])c([2H])c([2H])c4c(-c4cccc5oc6ccccc6c45)c4c([2H])c([2H])c([2H])c([2H])c34)c([2H])c([2H])c2c1[2H] ; [2H]c1c([2H])c(-c2c3c([2H])c([2H])c([2H])c([2H])c3c(-c3cccc4oc5ccccc5c34)c3c([2H])c([2H])c([2H])c([2H])c23)c([2H])c([2H])c1-c1ccccc1]

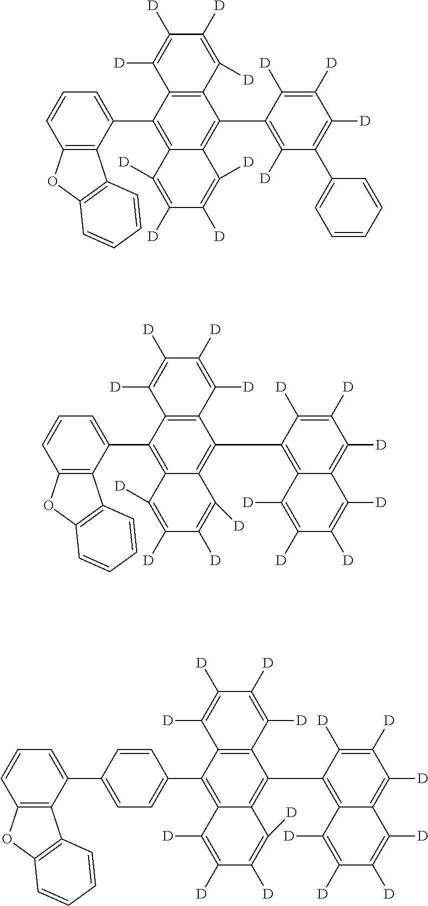 [SMILES: [2H]c1c([2H])c(-c2ccccc2)c([2H])c(-c2c3c([2H])c([2H])c([2H])c([2H])c3c(-c3cccc4oc5ccccc5c34)c3c([2H])c([2H])c([2H])c([2H])c23)c1[2H] ; [2H]c1c([2H])c([2H])c2c(-c3c4c([2H])c([2H])c([2H])c([2H])c4c(-c4cccc5oc6ccccc6c45)c4c([2H])c([2H])c([2H])c([2H])c34)c([2H])c([2H])c([2H])c2c1[2H] ; [2H]c1c([2H])c([2H])c2c(-c3c4c([2H])c([2H])c([2H])c([2H])c4c(-c4ccc(-c5cccc6oc7ccccc7c56)cc4)c4c([2H])c([2H])c([2H])c([2H])c34)c([2H])c([2H])c([2H])c2c1[2H]]

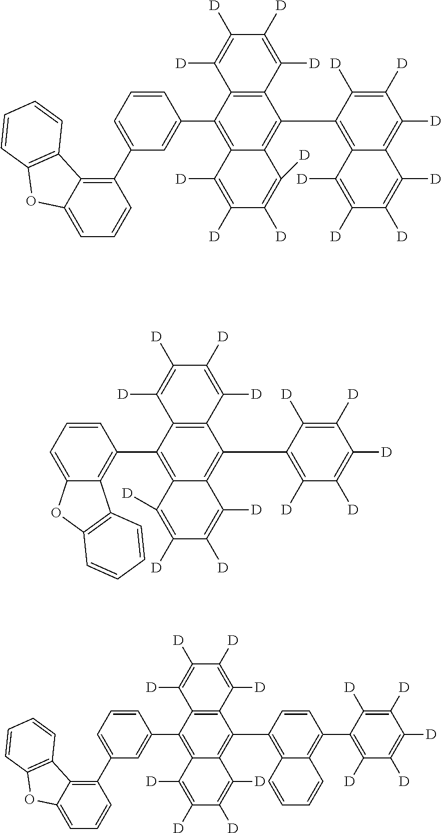 [SMILES: [2H]c1c([2H])c([2H])c2c(-c3c4c([2H])c([2H])c([2H])c([2H])c4c(-c4cccc(-c5cccc6oc7ccccc7c56)c4)c4c([2H])c([2H])c([2H])c([2H])c34)c([2H])c([2H])c([2H])c2c1[2H] ; [2H]c1c([2H])c([2H])c(-c2c3c([2H])c([2H])c([2H])c([2H])c3c(-c3cccc4oc5ccccc5c34)c3c([2H])c([2H])c([2H])c([2H])c23)c([2H])c1[2H] ; [2H]c1c([2H])c([2H])c(-c2ccc(-c3c4c([2H])c([2H])c([2H])c([2H])c4c(-c4cccc(-c5cccc6oc7ccccc7c56)c4)c4c([2H])c([2H])c([2H])c([2H])c34)c3ccccc23)c([2H])c1[2H]]

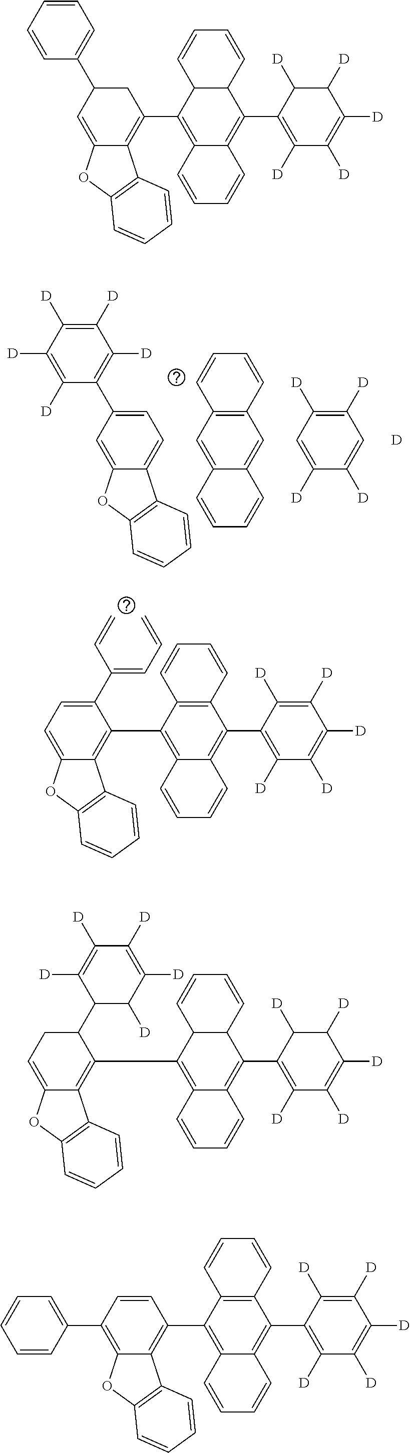

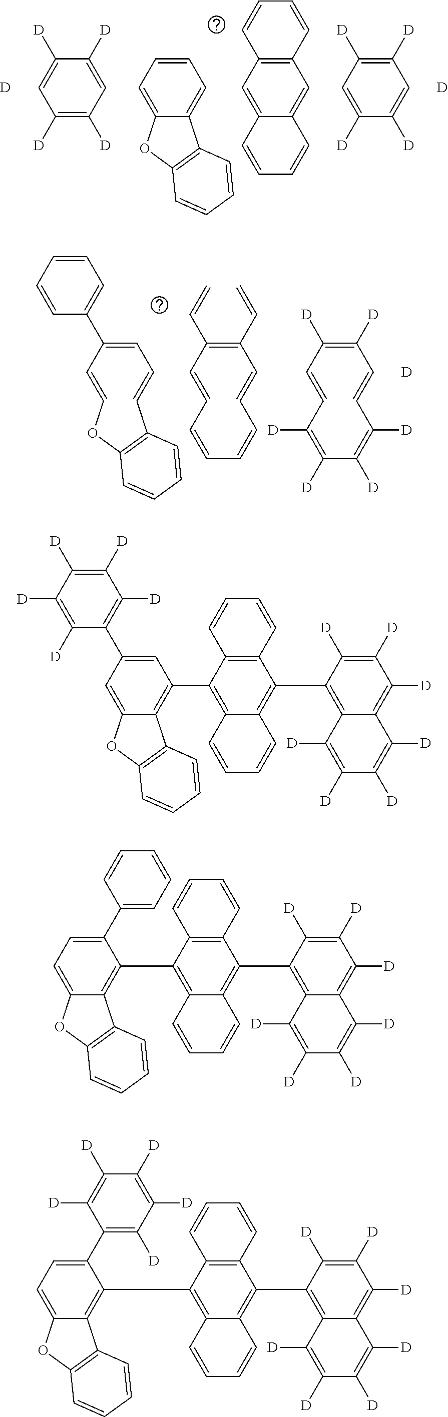

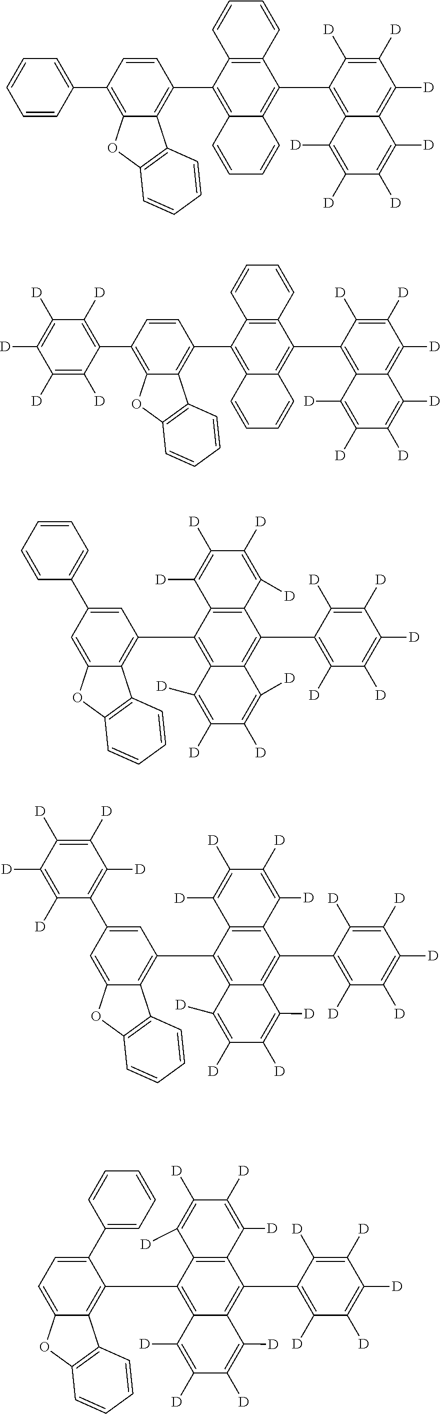

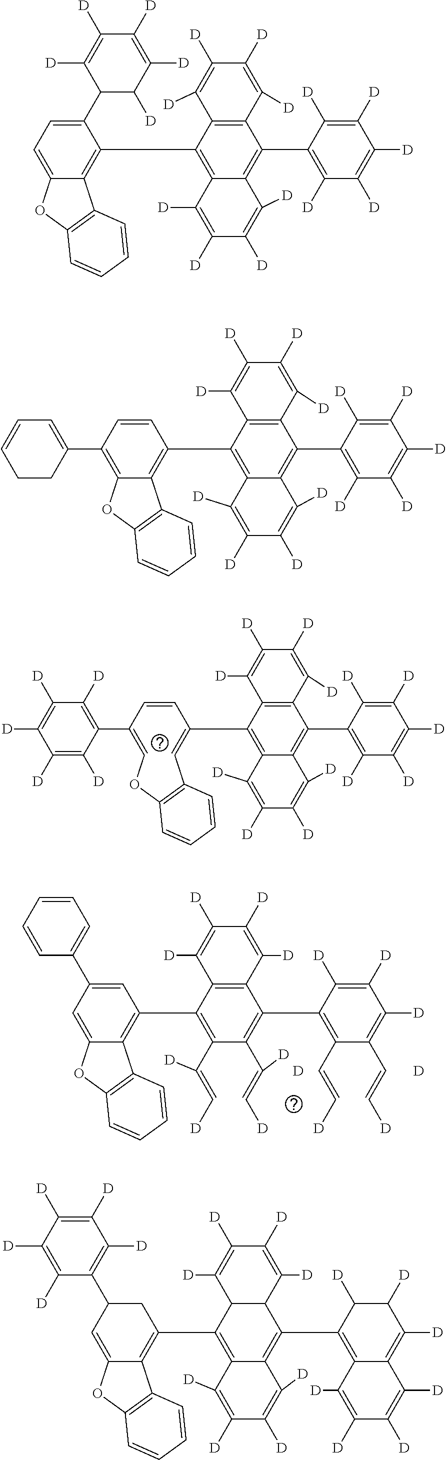

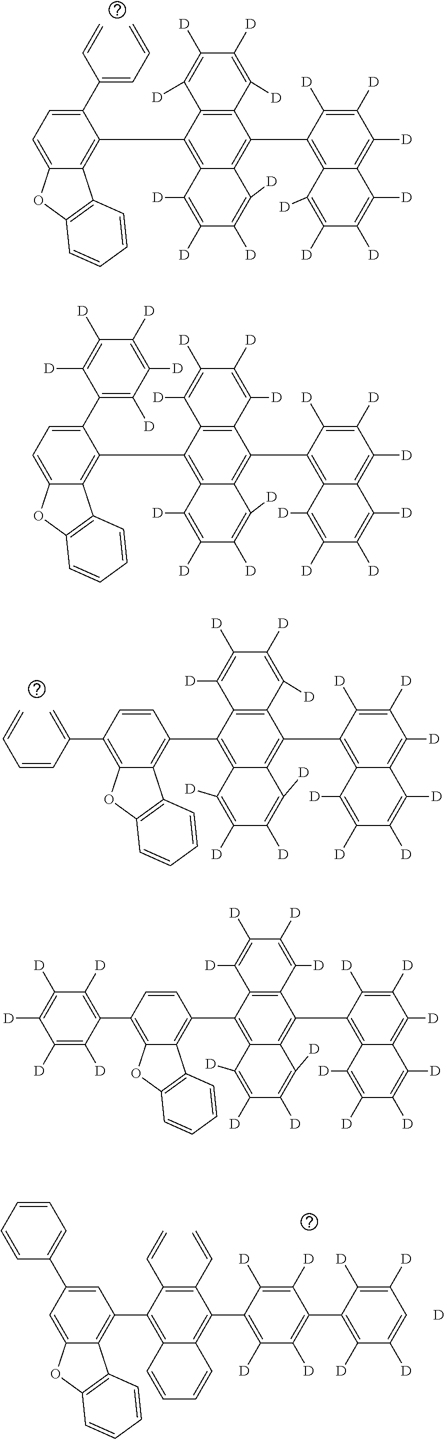

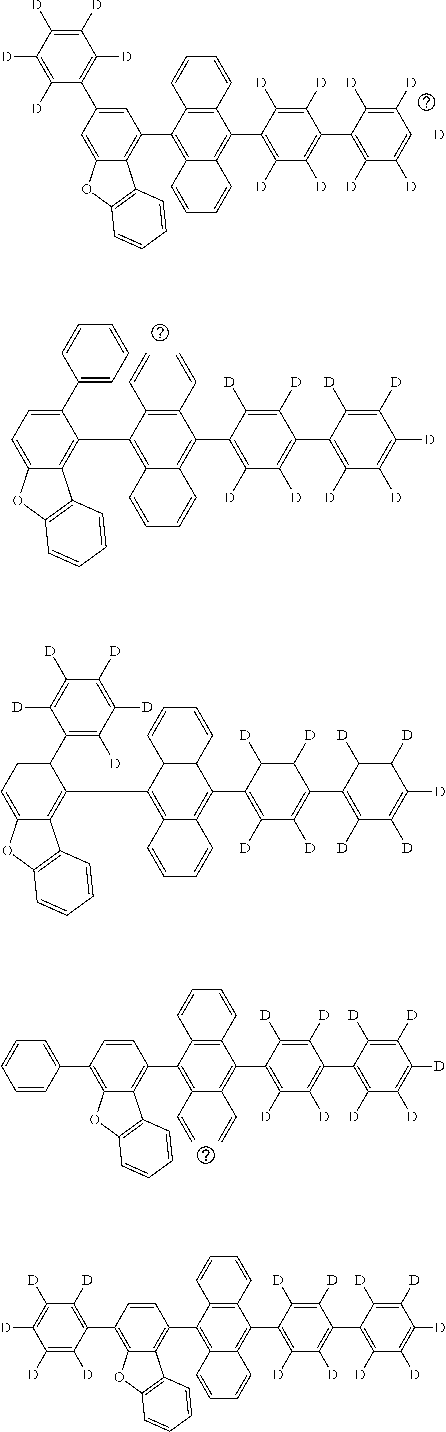

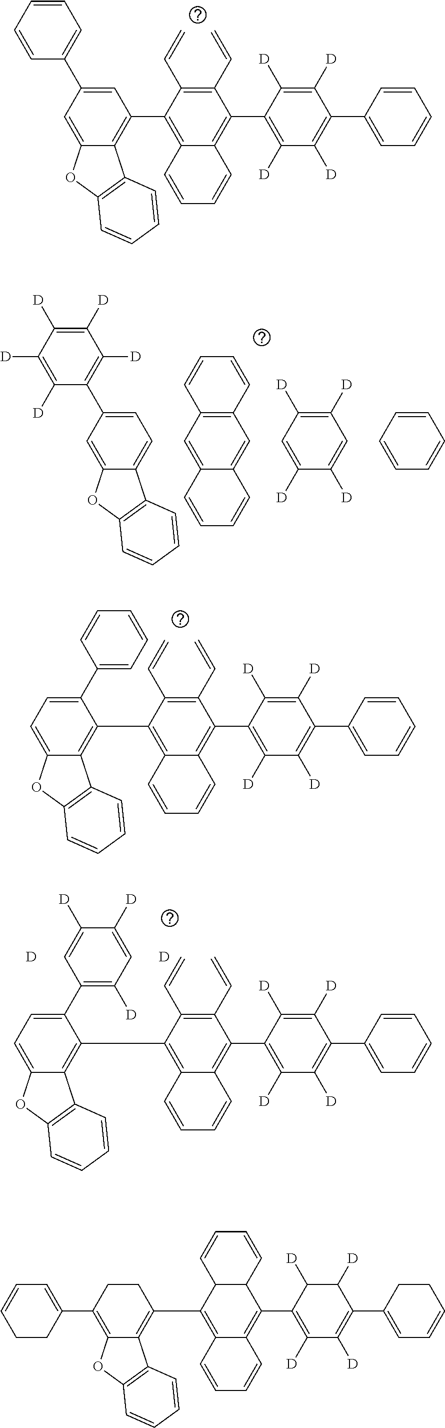

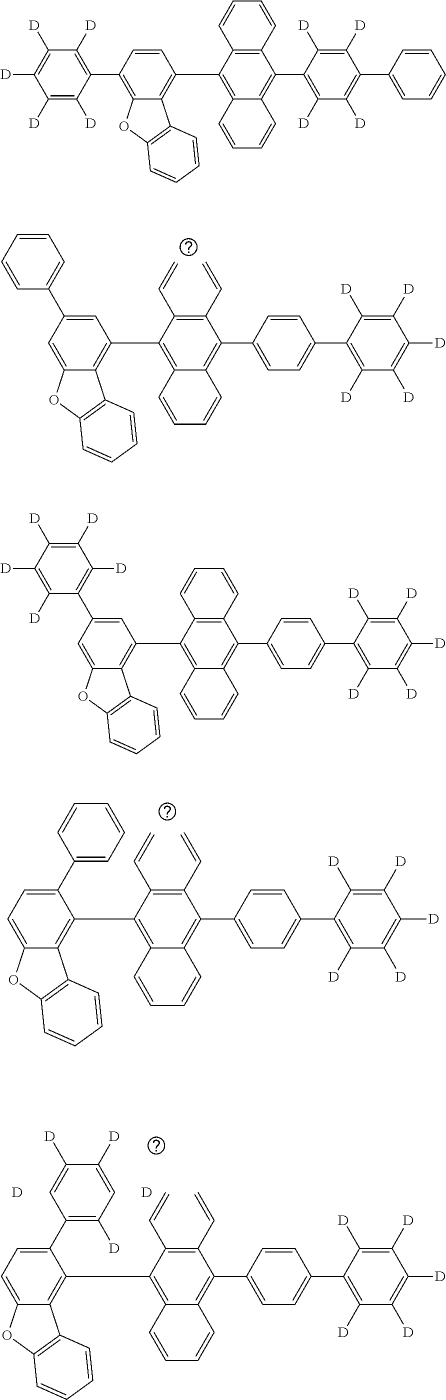

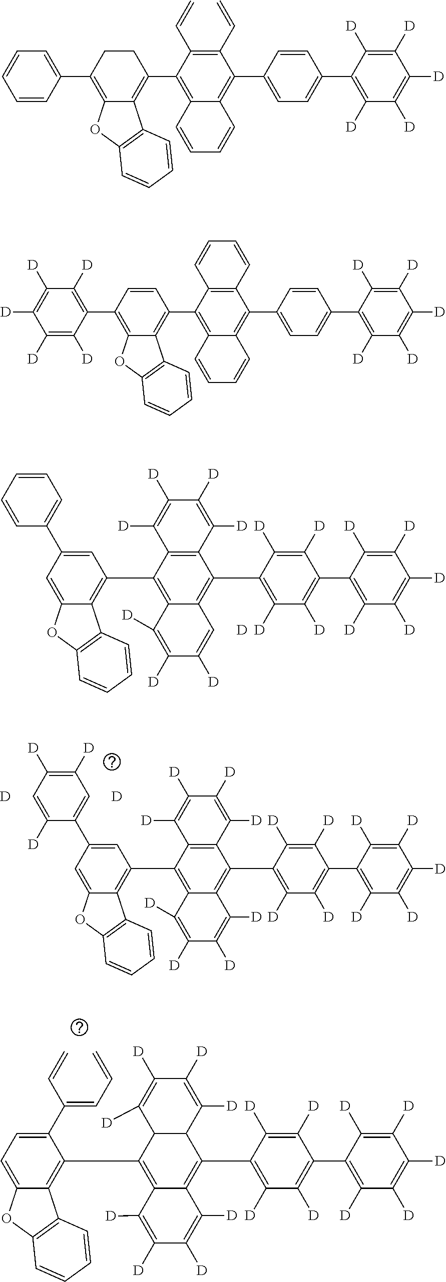

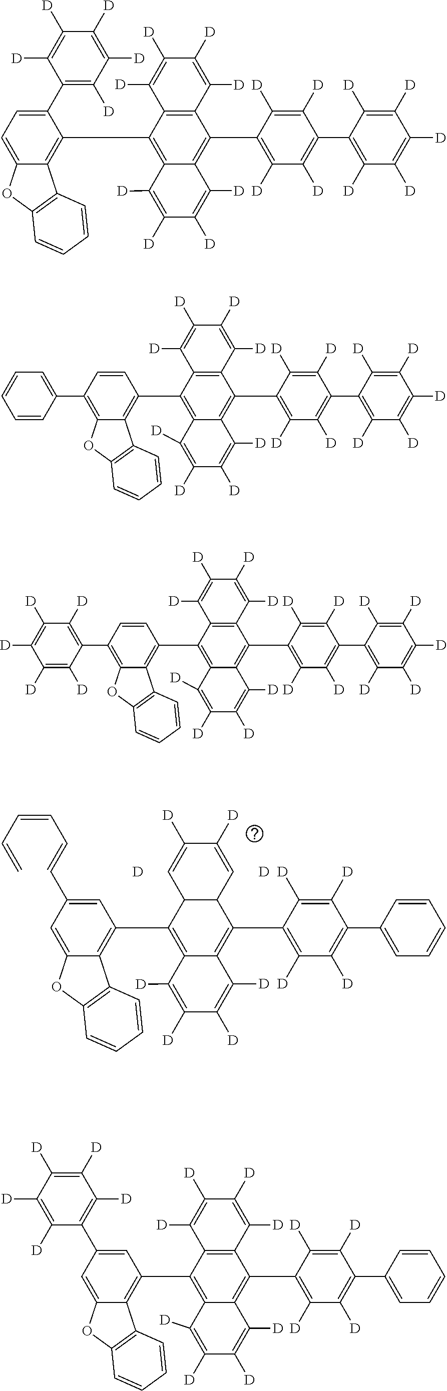

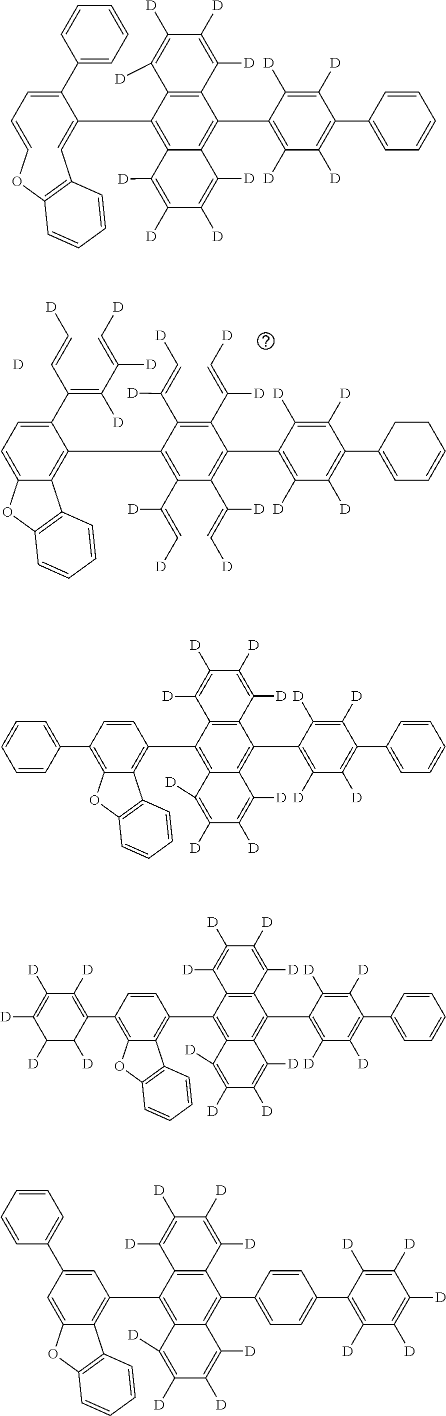

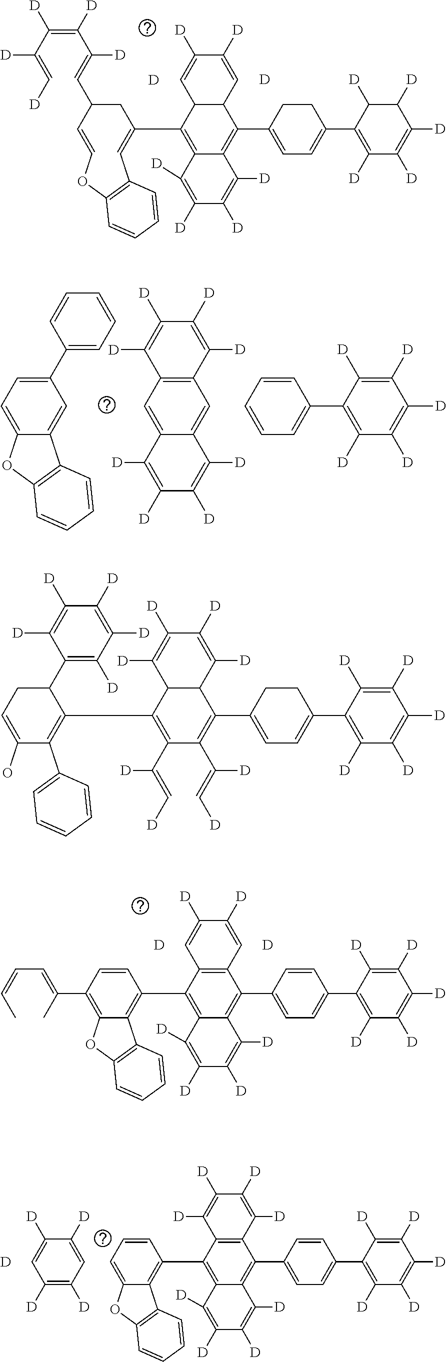

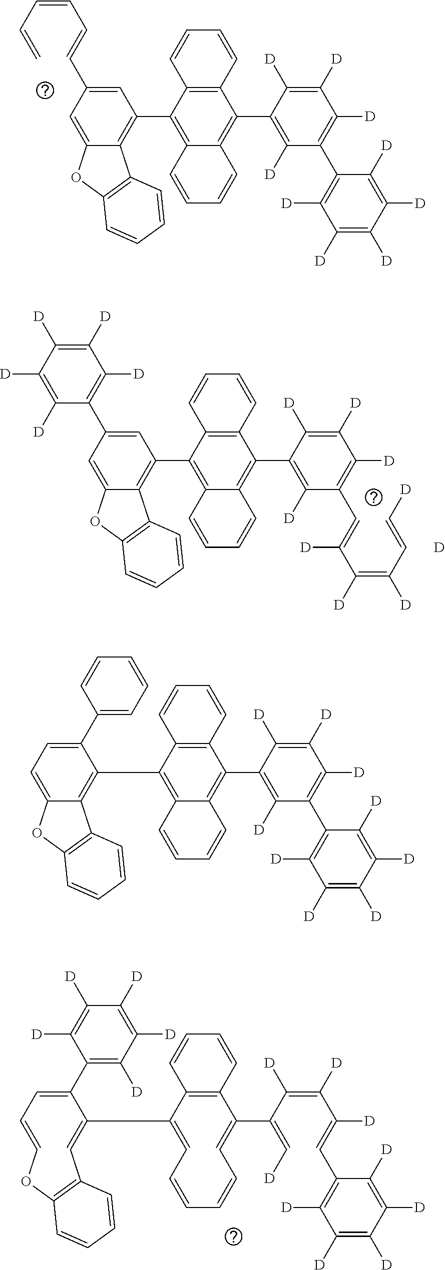

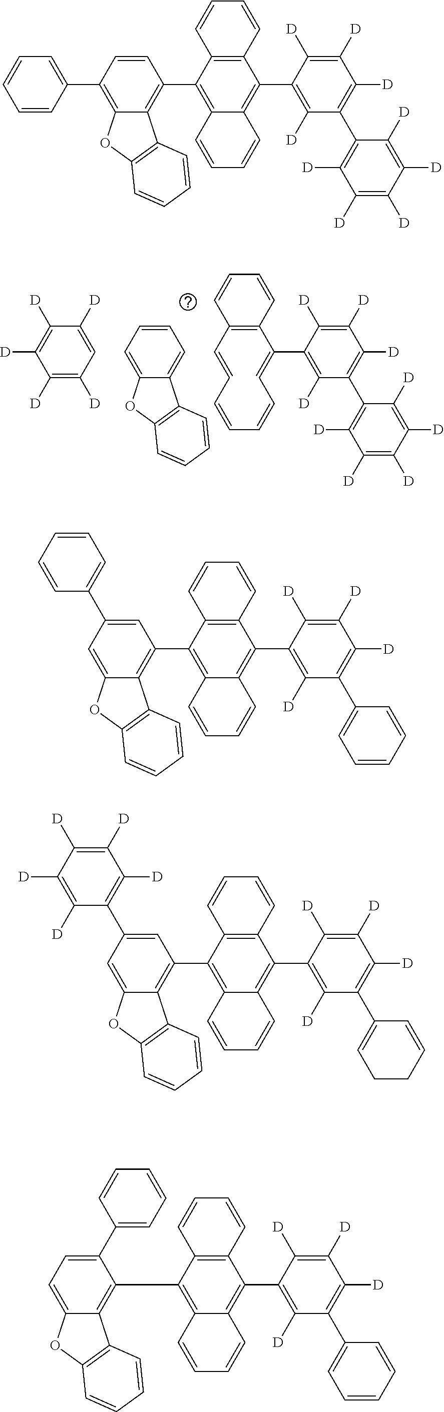

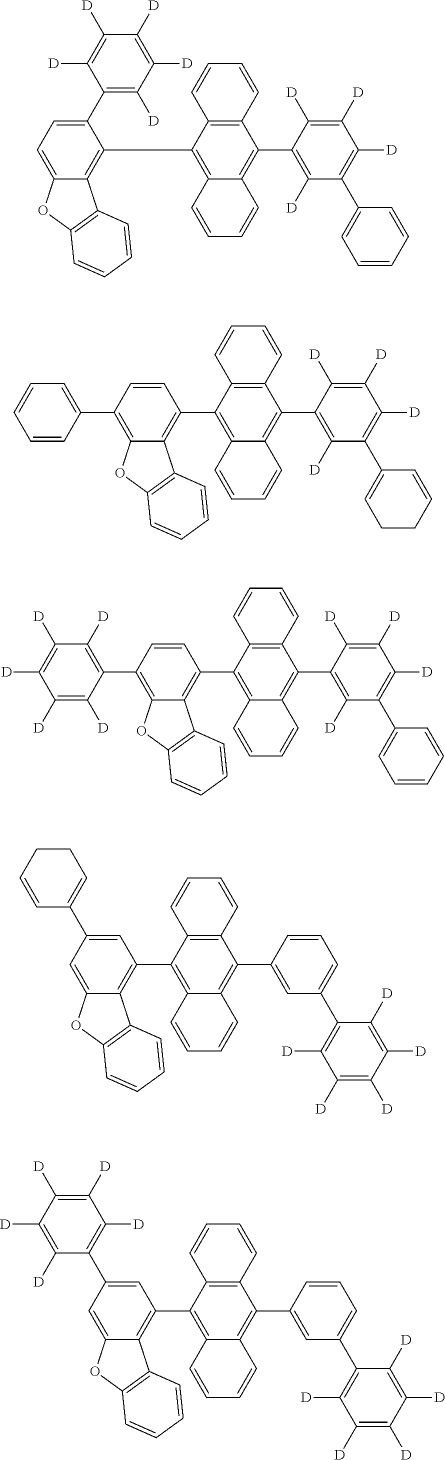

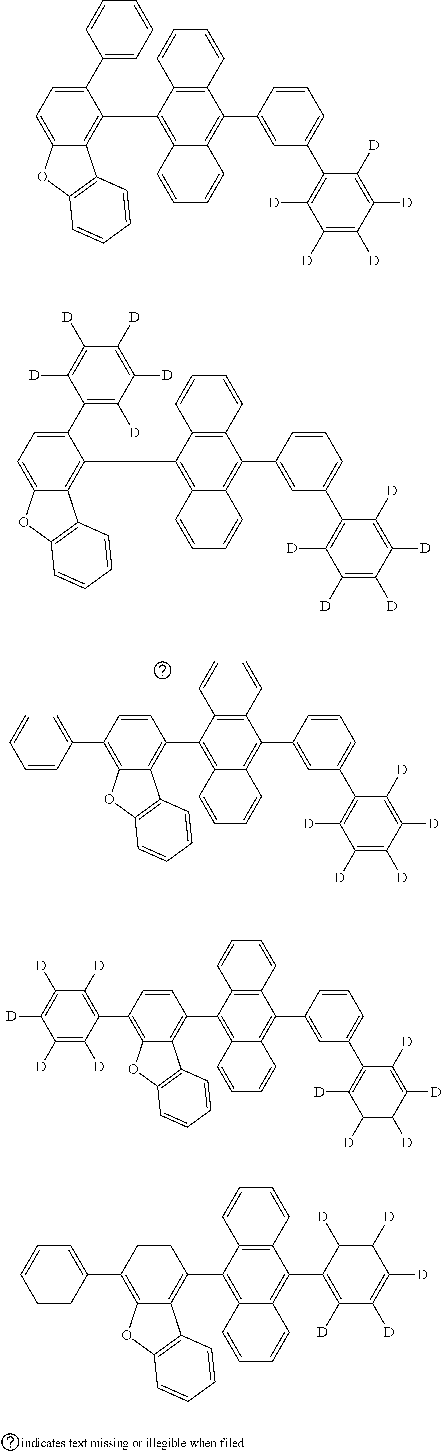

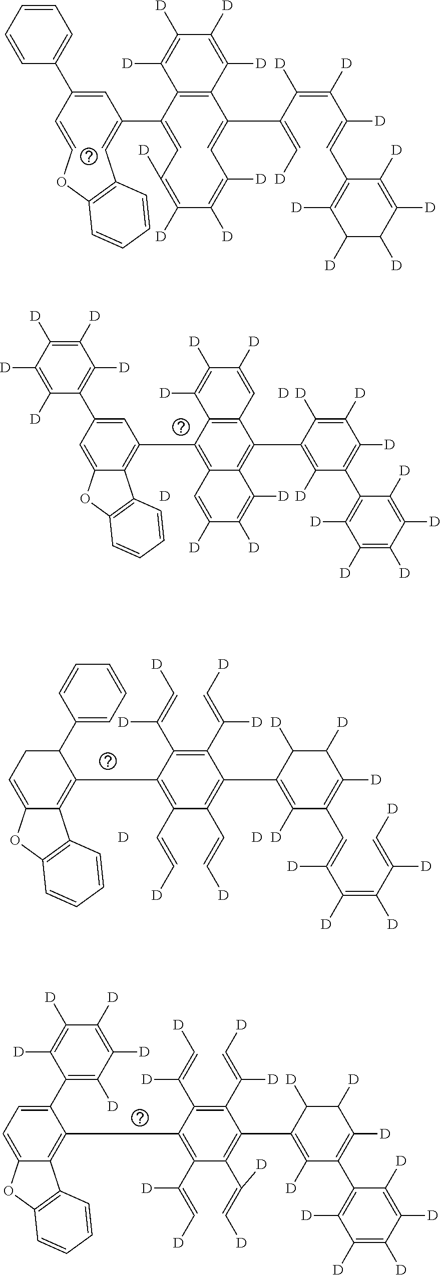

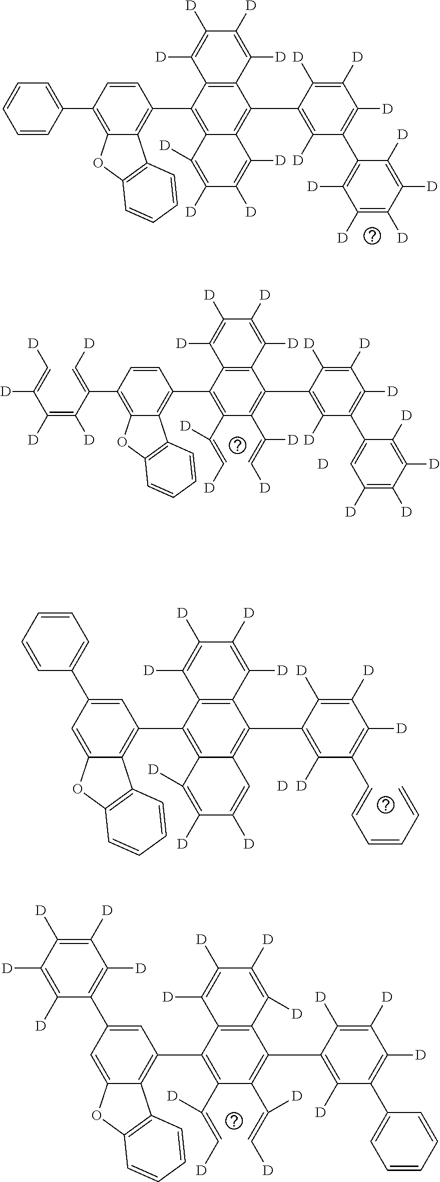

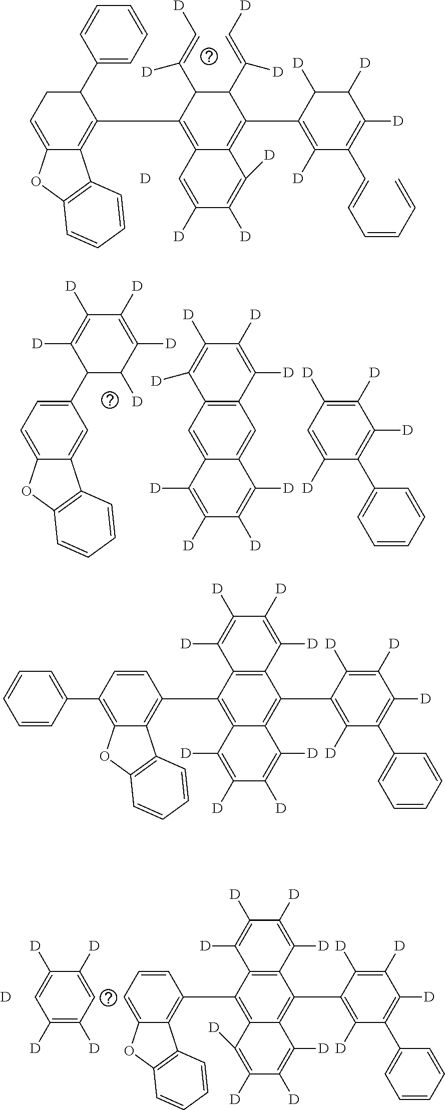

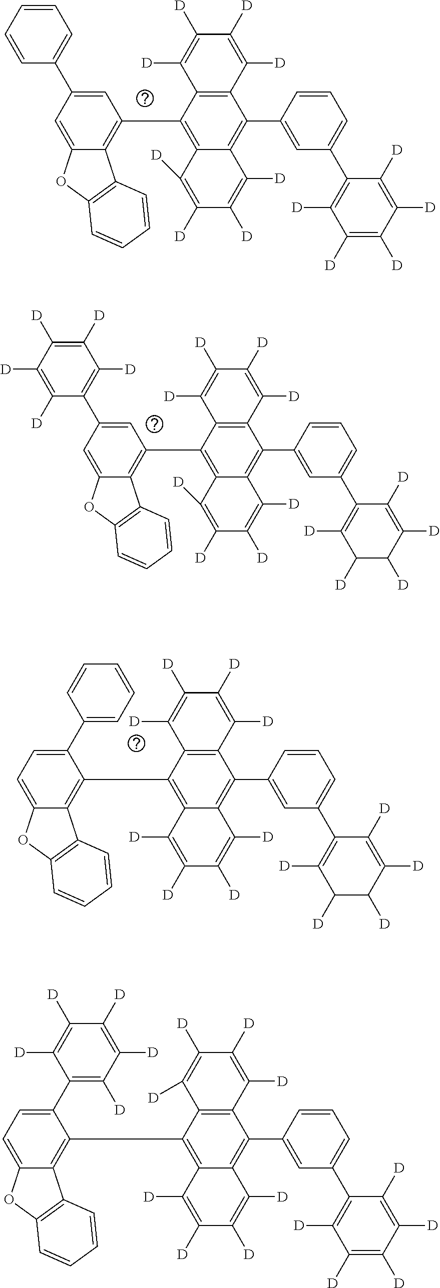

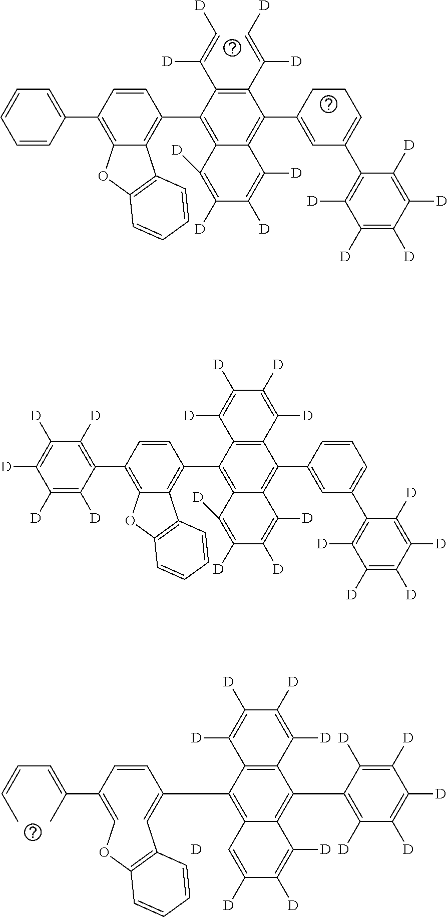

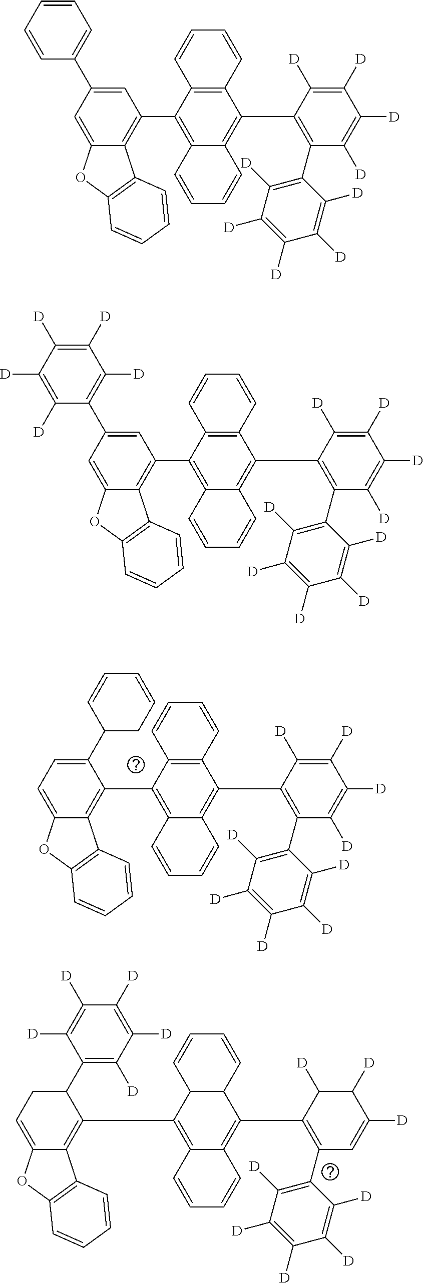

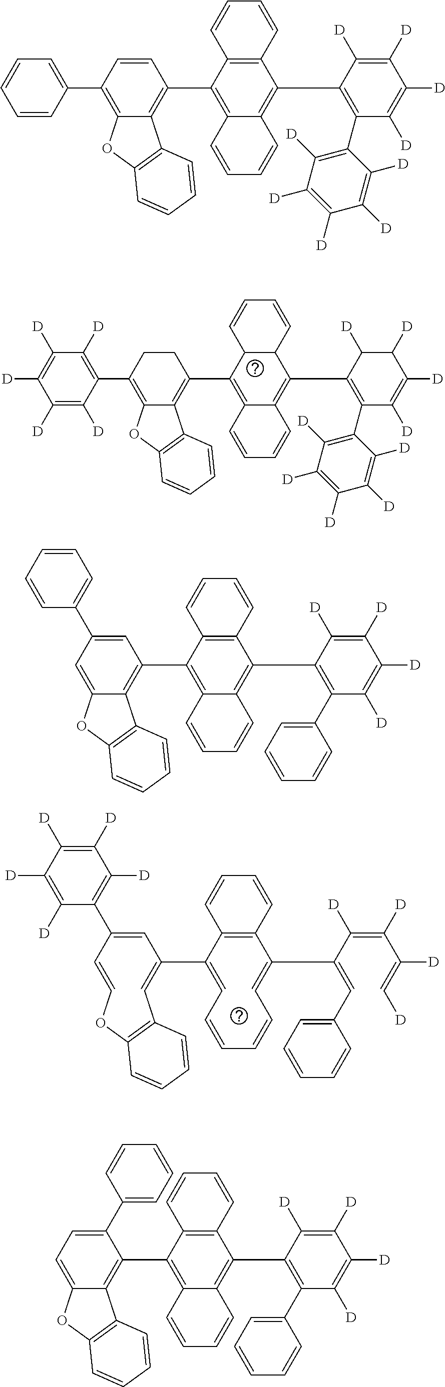

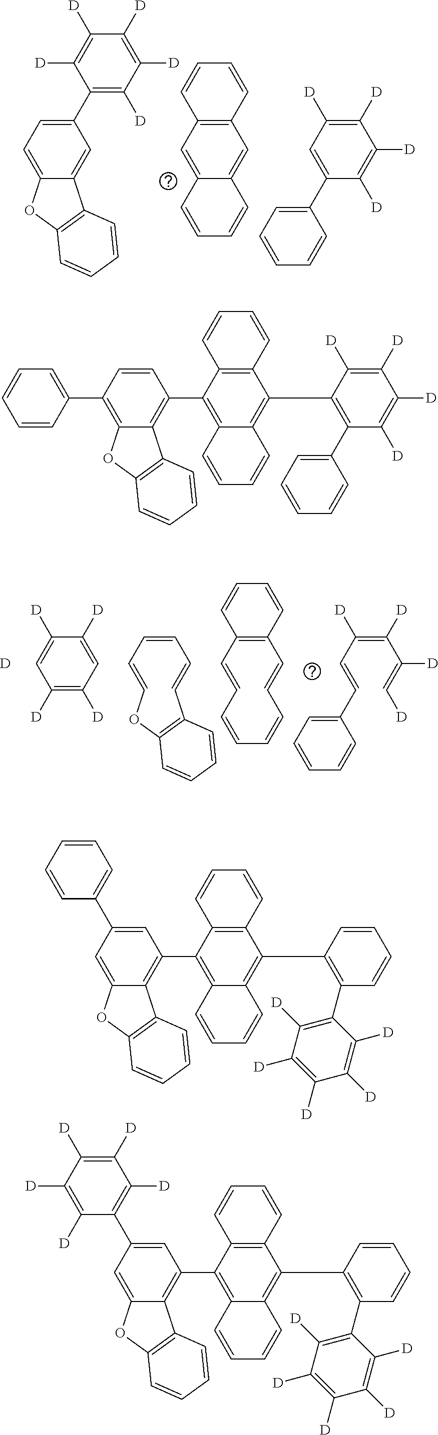

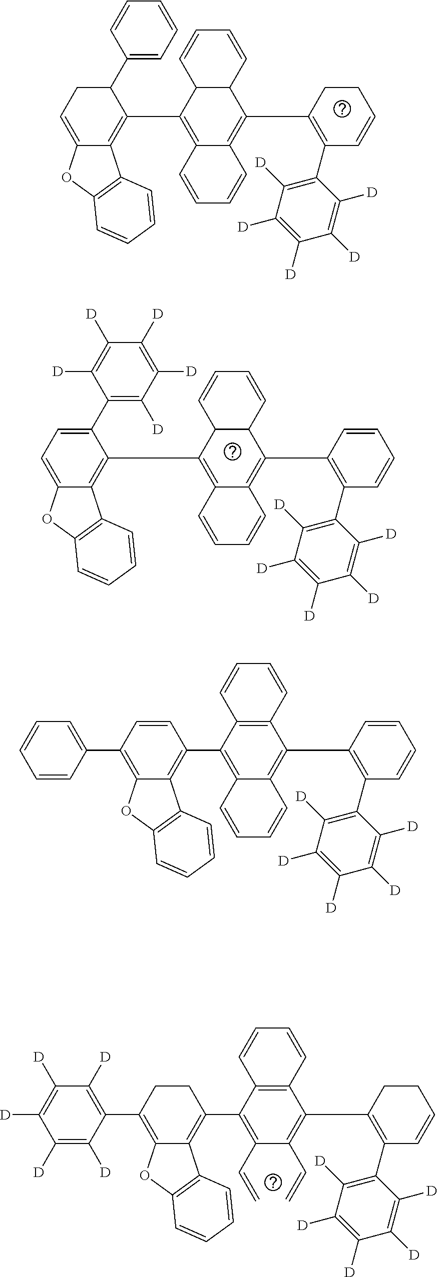 [SMILES: [2H]c1c([2H])c([2H])c(C2=C(C3=c4ccccc4=C(C4=c5c(oc6ccccc56)=CCC4c4ccccc4)C4C=CC=CC34)CCC=C2)c([2H])c1[2H] ; [2H]c1c([2H])c([2H])c(-c2ccccc2C2=c3ccccc3=C(c3c(-c4c([2H])c([2H])c([2H])c([2H])c4[2H])ccc4oc5ccccc5c34)C3C=CC=CC23)c([2H])c1[2H] ; [2H]c1c([2H])c([2H])c(-c2ccccc2-c2c3ccccc3c(-c3ccc(-c4ccccc4)c4oc5ccccc5c34)c3ccccc23)c([2H])c1[2H] ; [2H]c1c([2H])c([2H])c(C2=C(c3c(C=C)c(C=C)c(C4=c5c(oc6ccccc56)=C(c5c([2H])c([2H])c([2H])c([2H])c5[2H])CC4)c4ccccc34)CCC=C2)c([2H])c1[2H]]

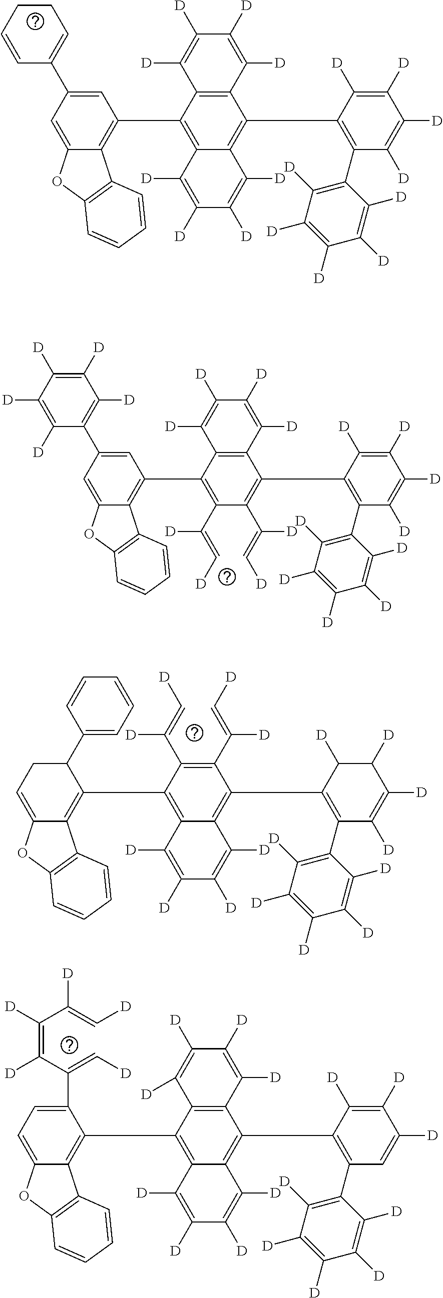

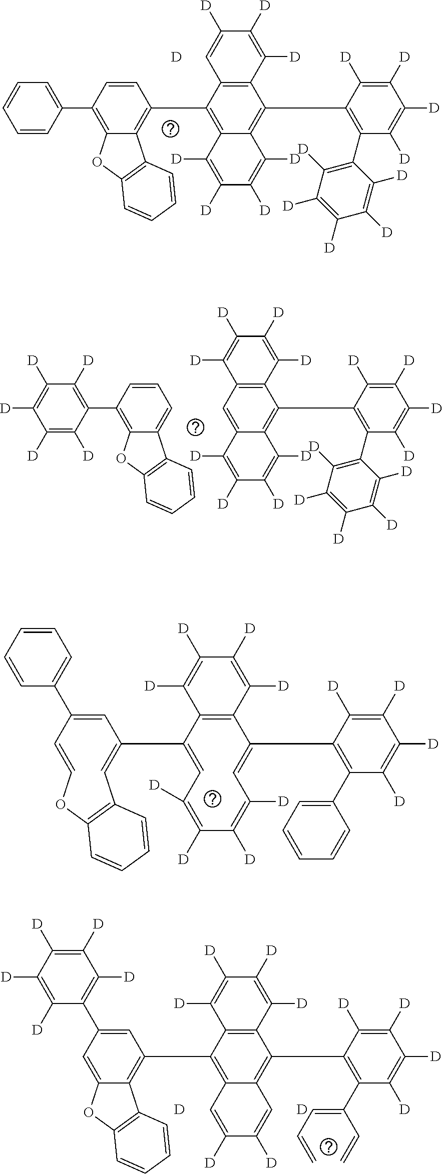

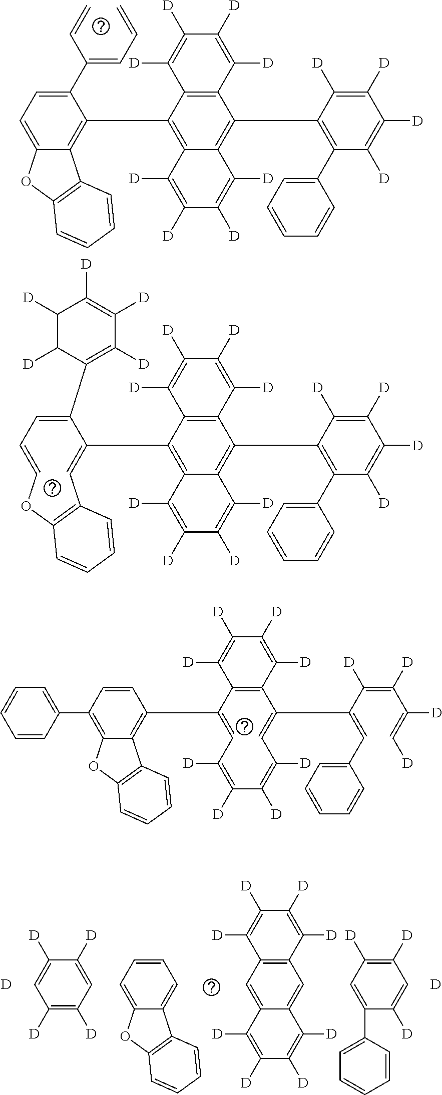

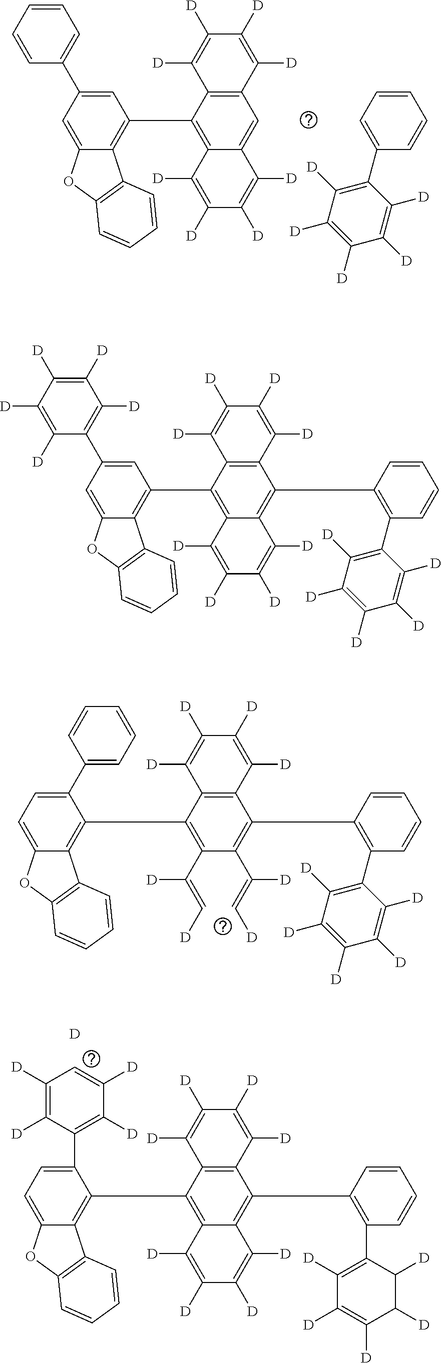

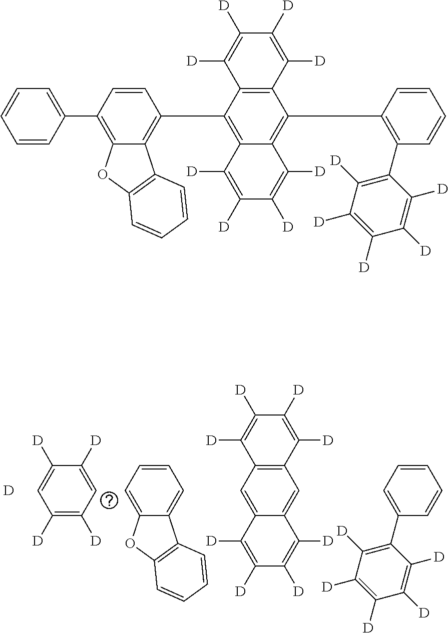

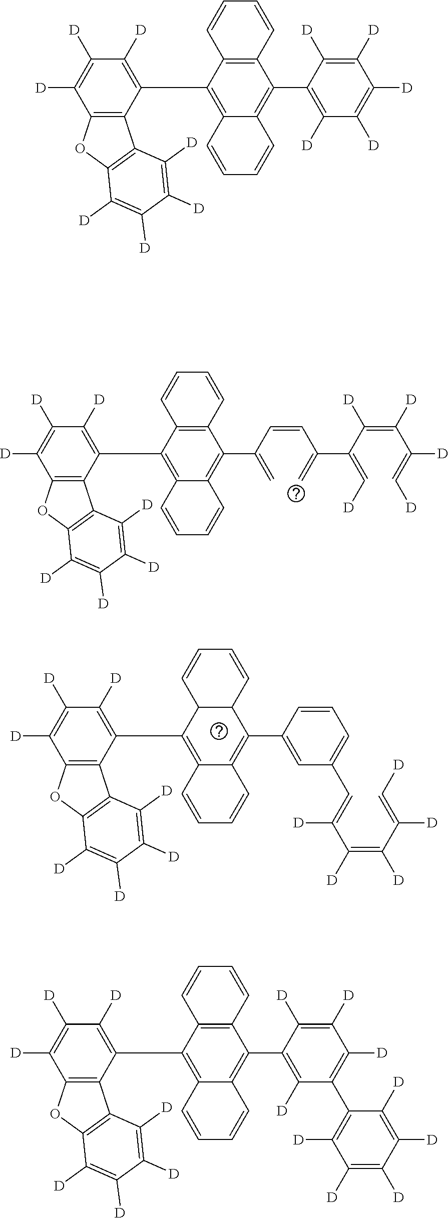

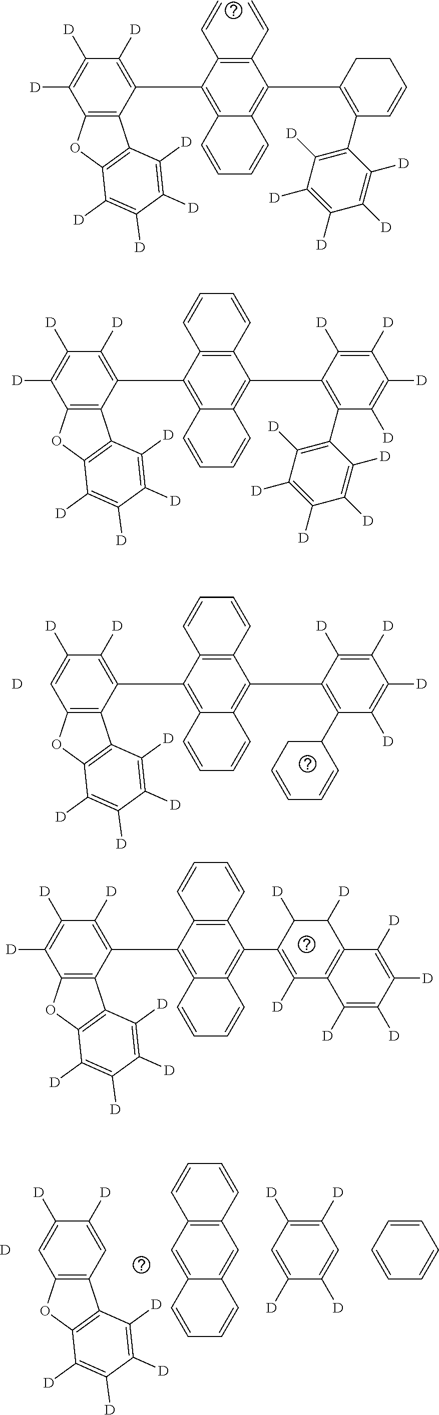

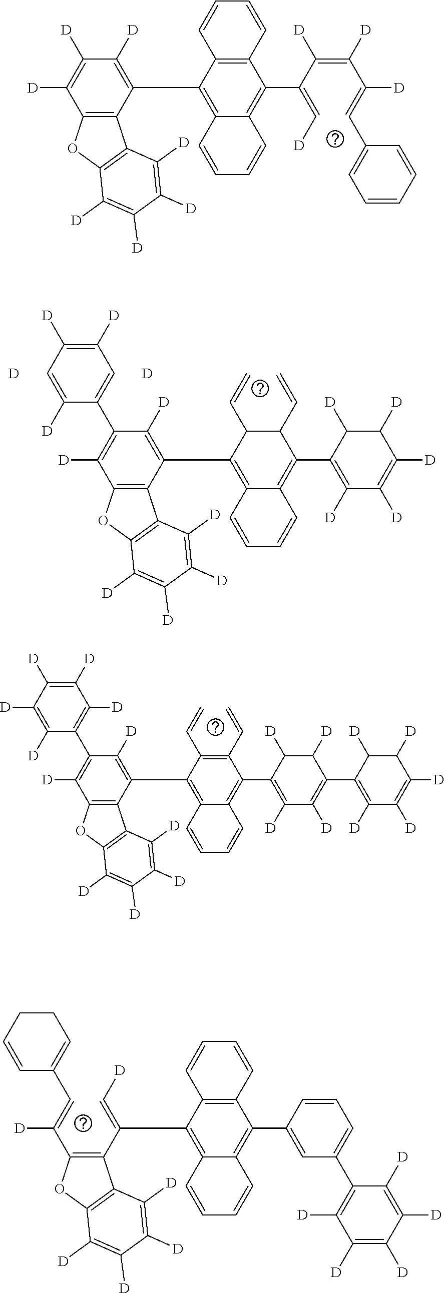

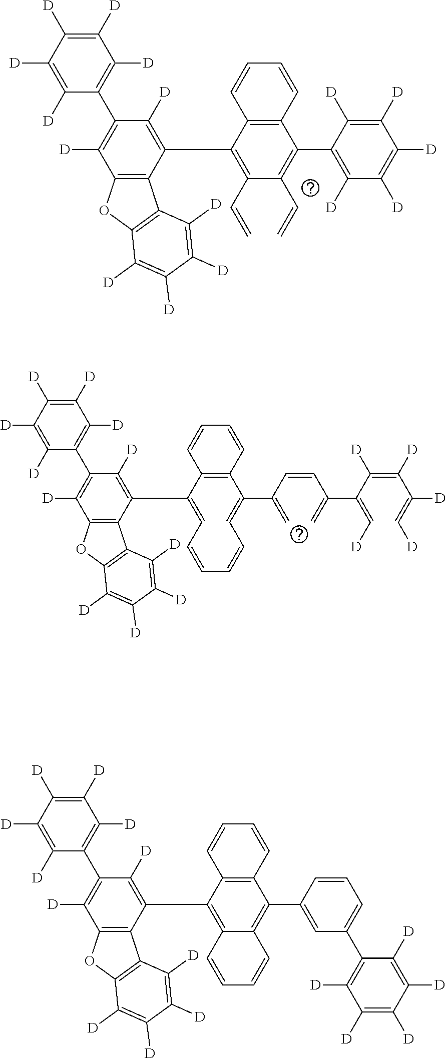

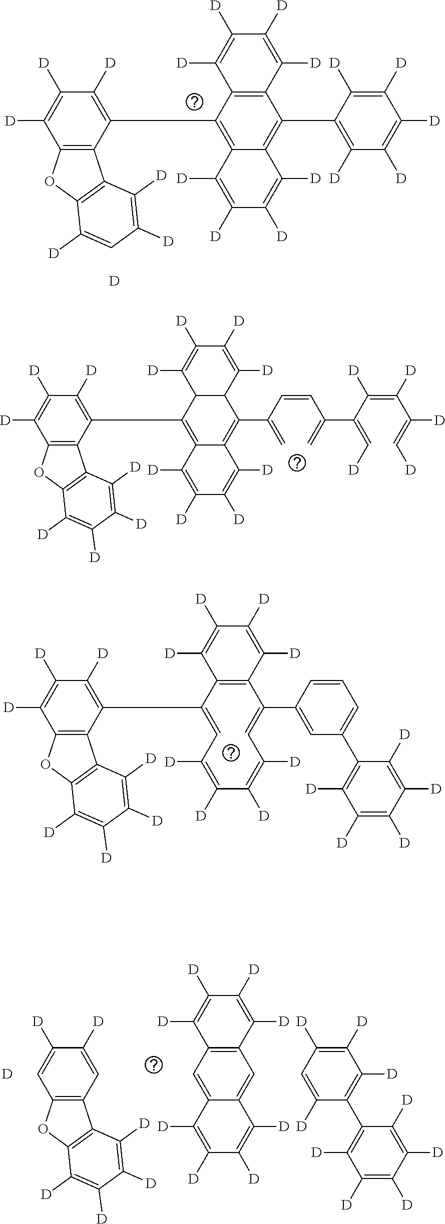

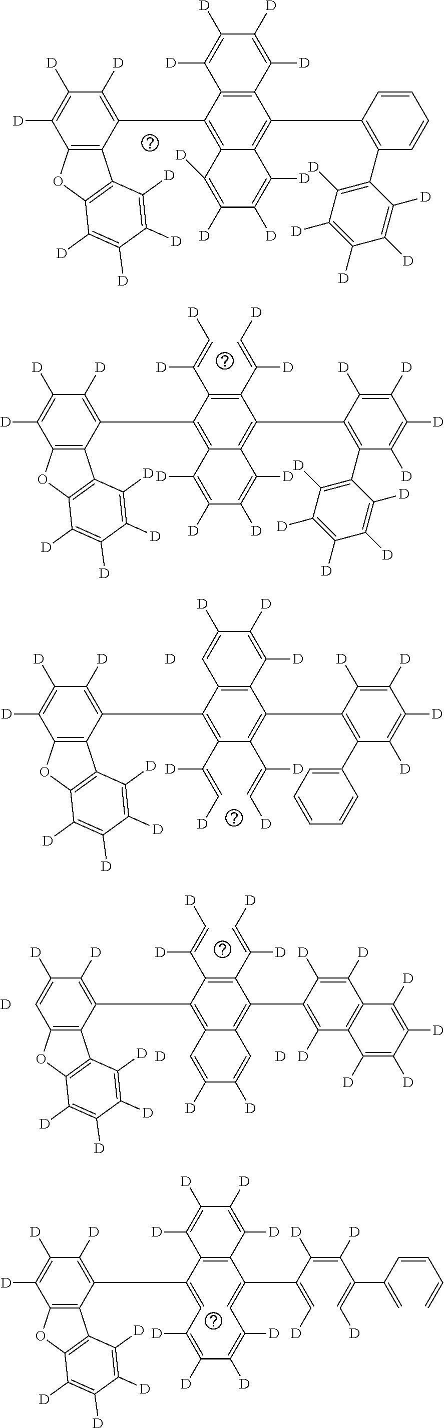

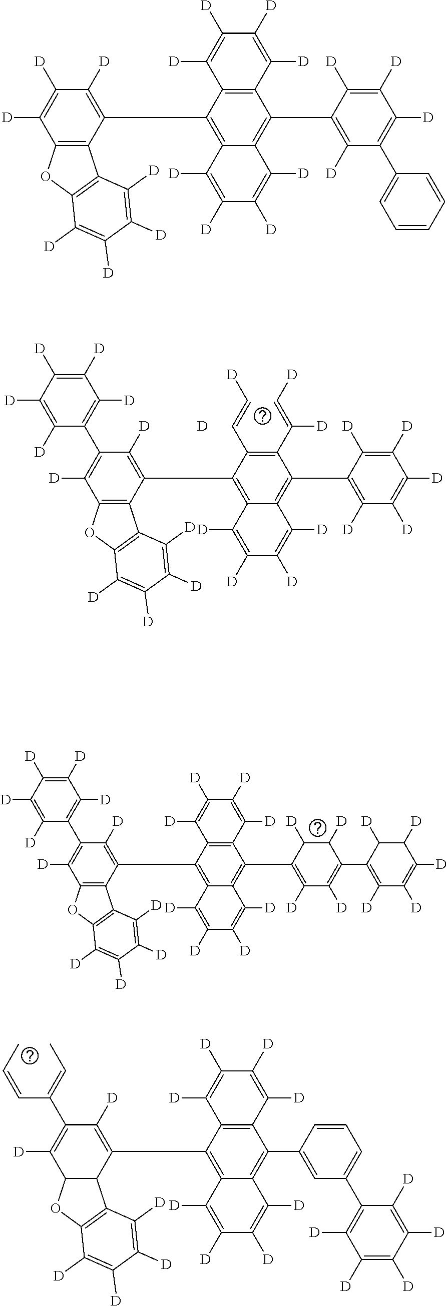

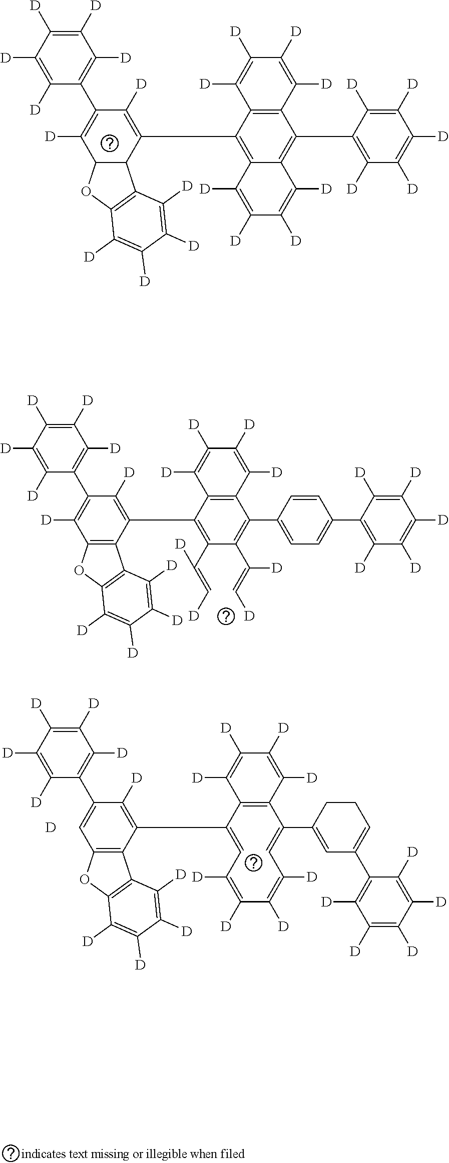

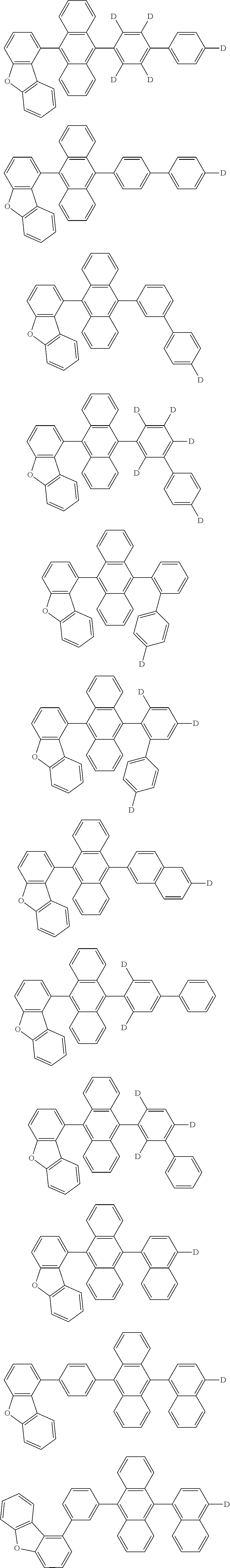

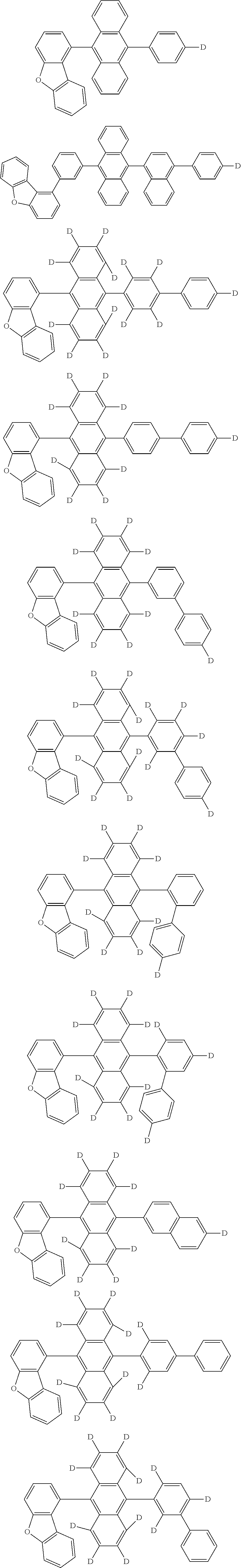

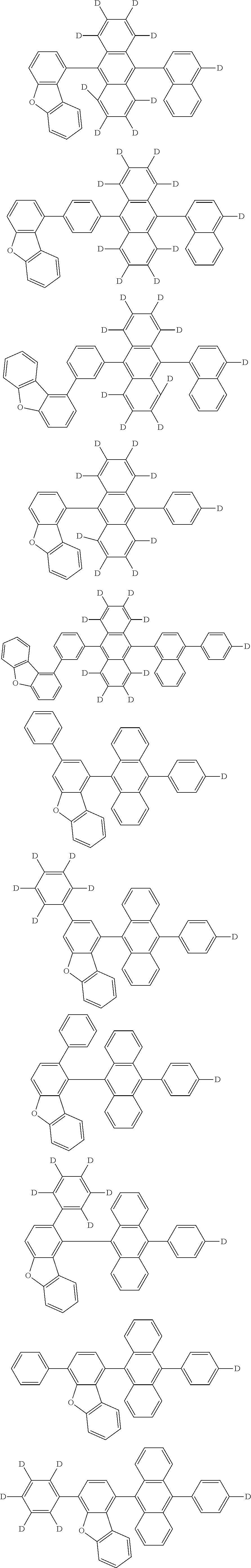

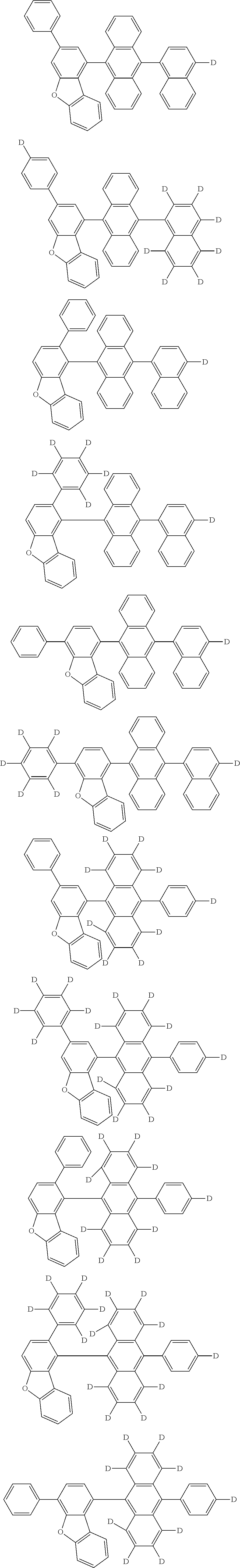

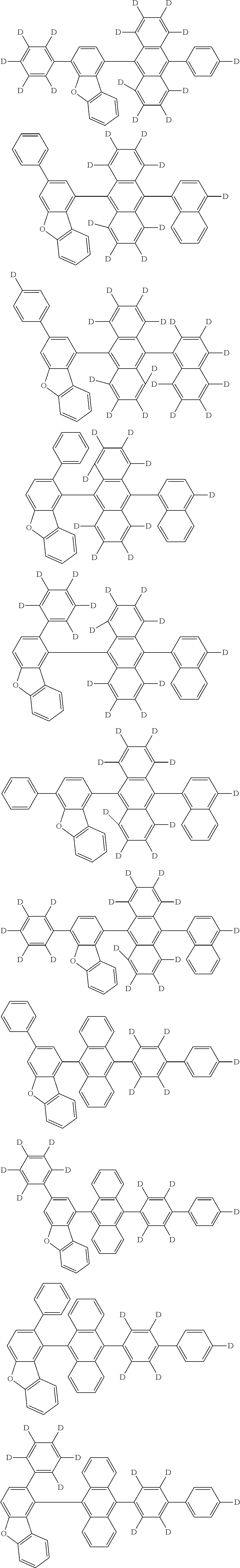

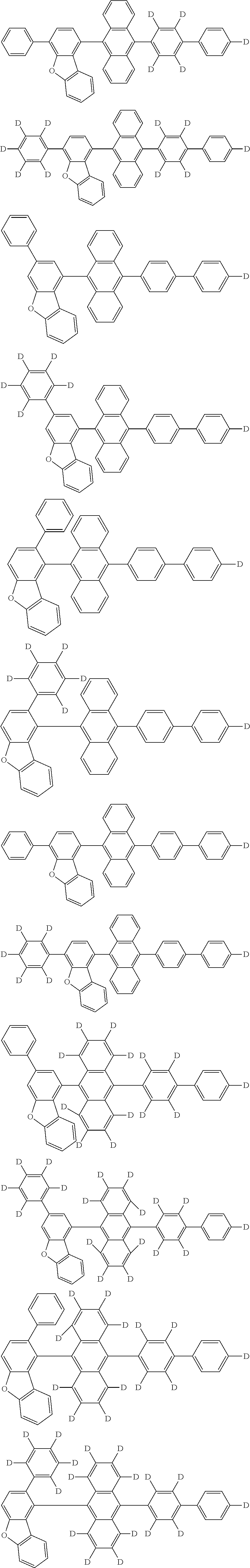

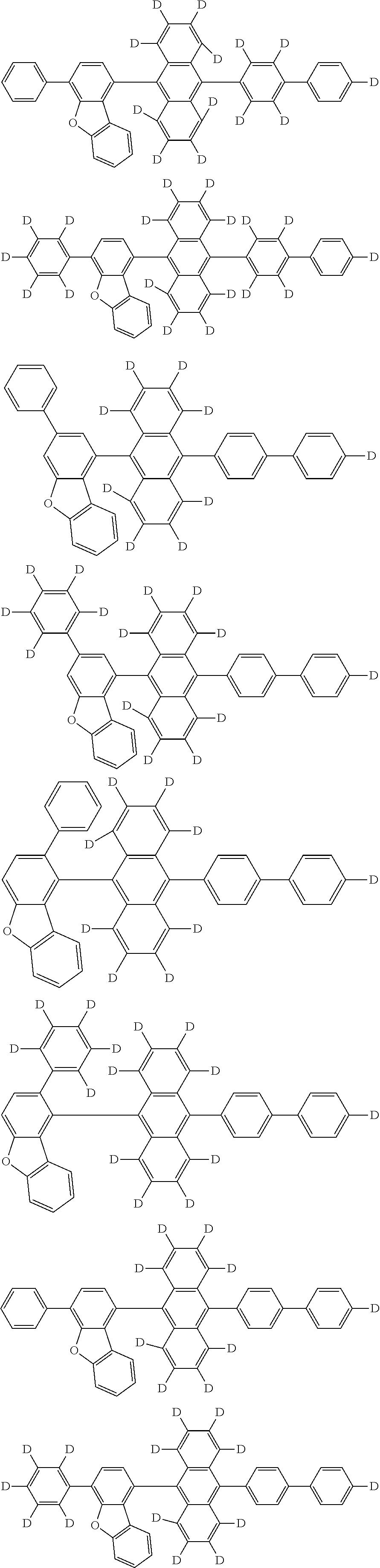

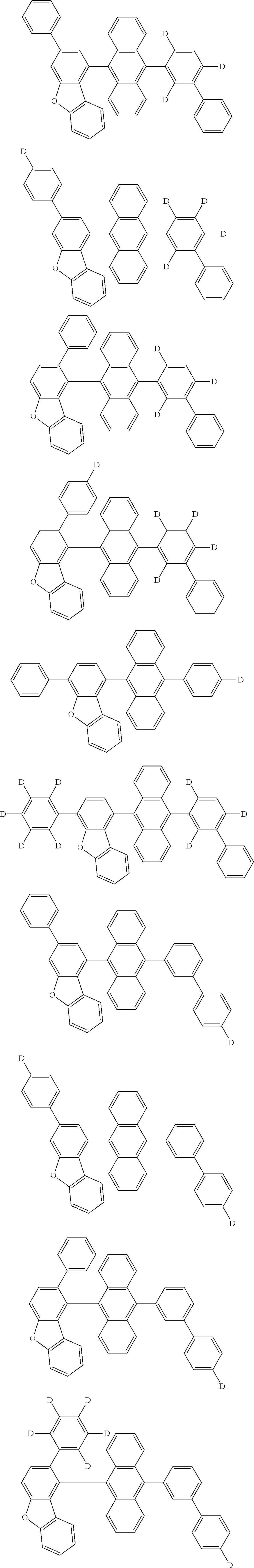

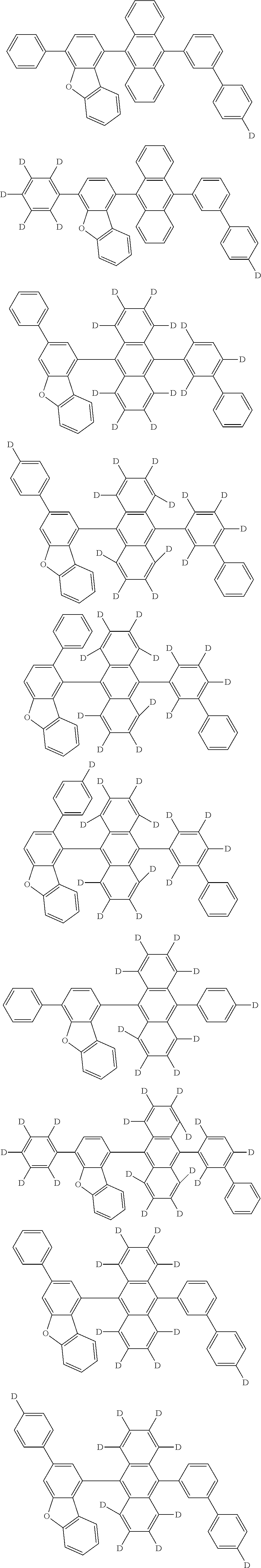

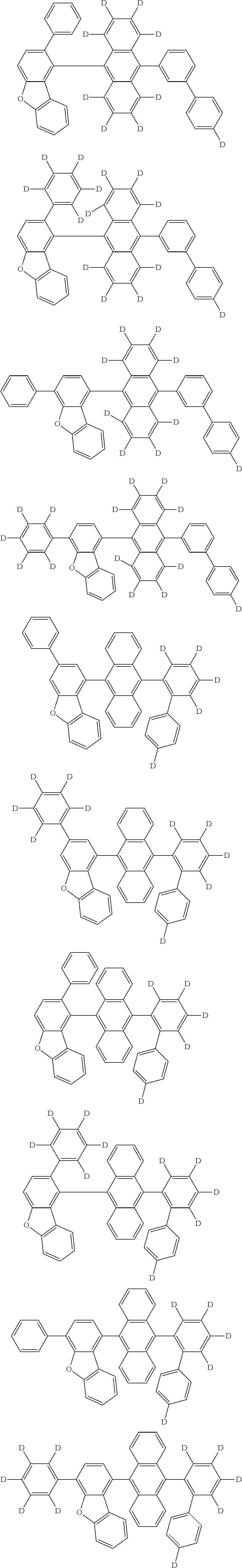

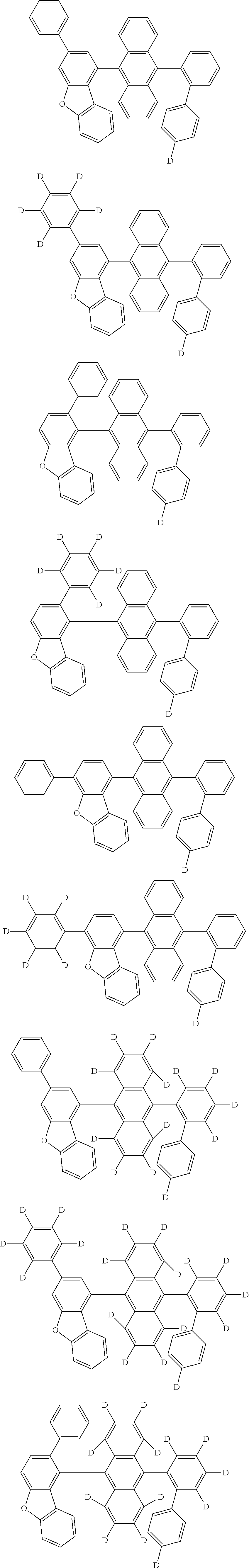

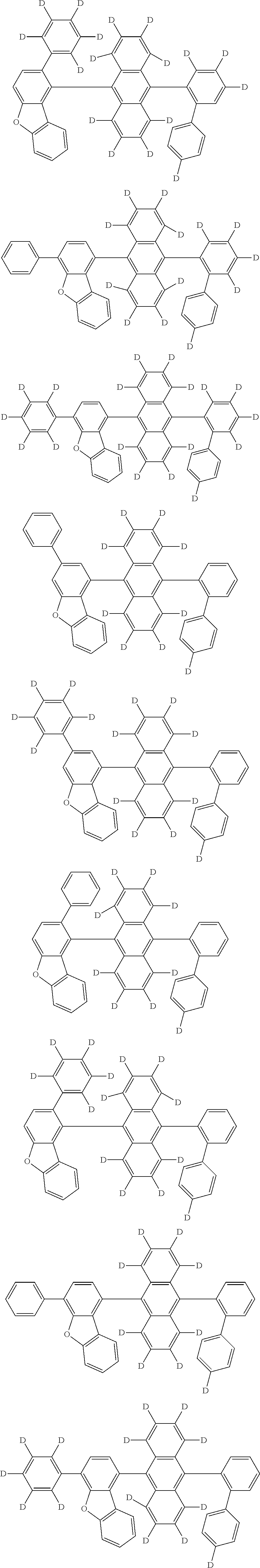

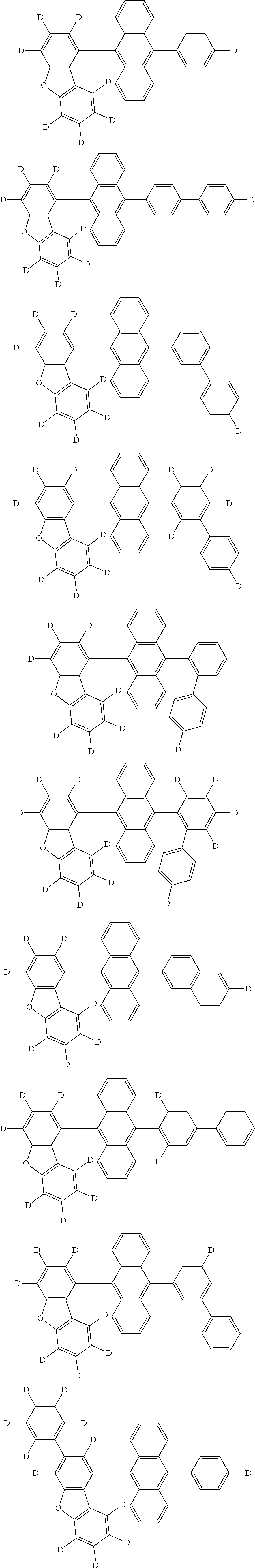

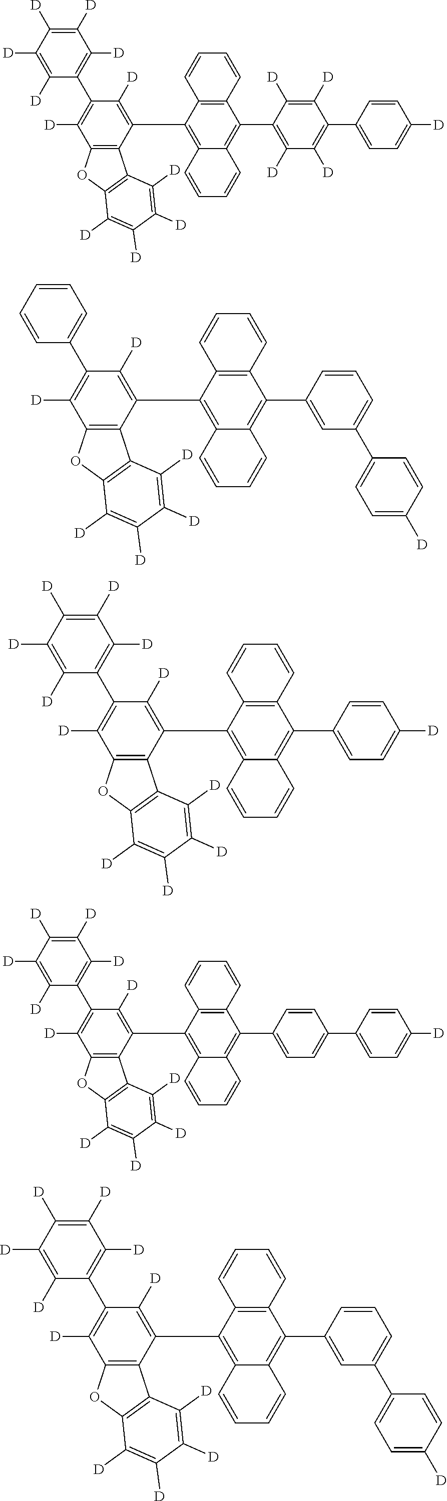

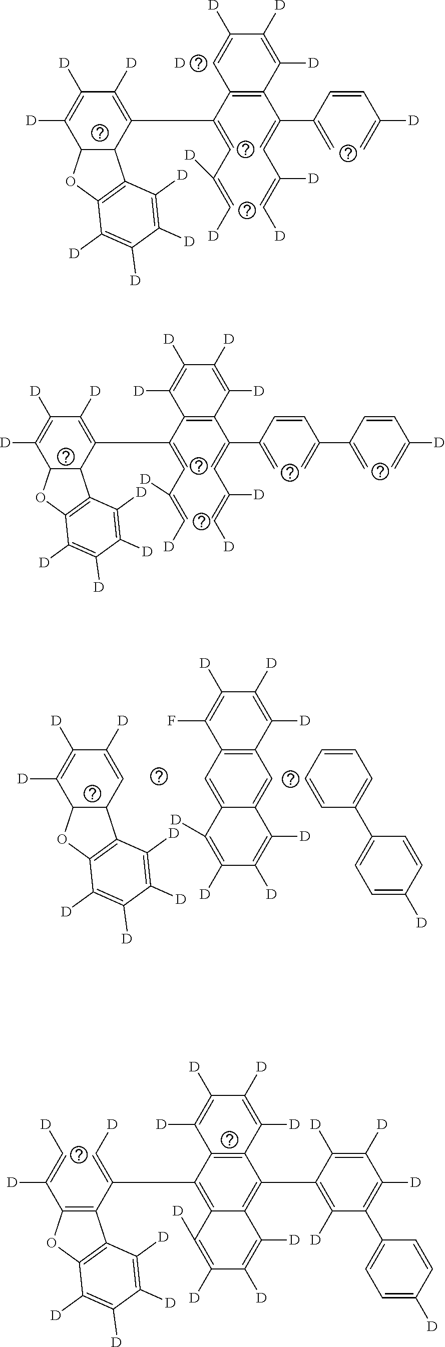

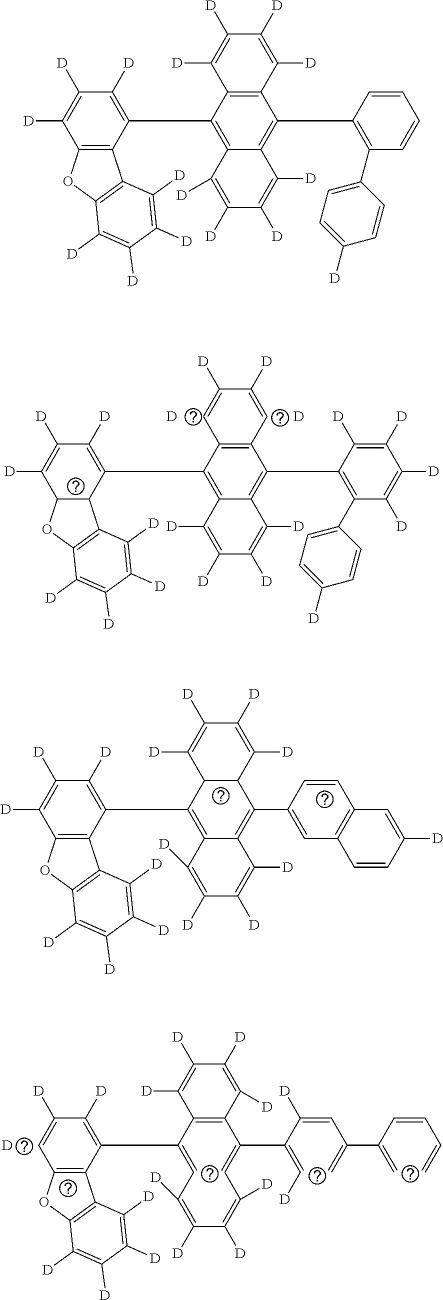

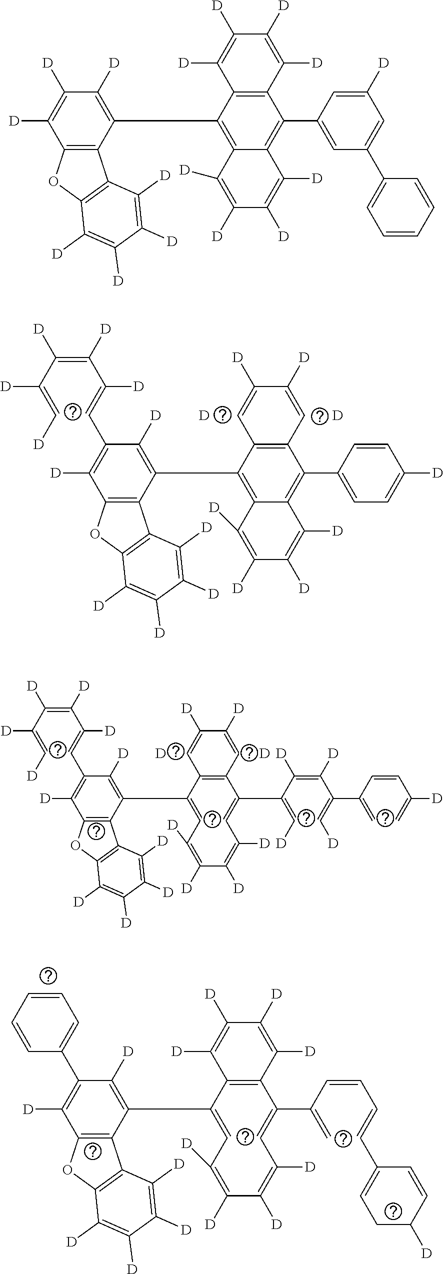

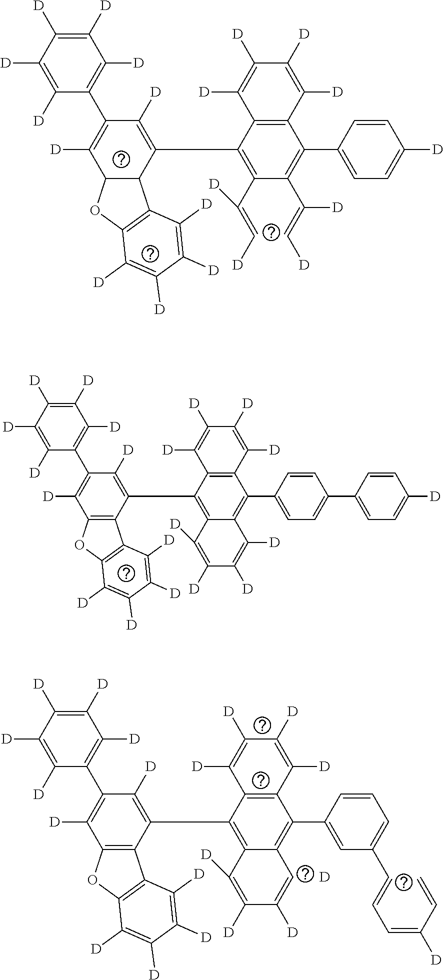

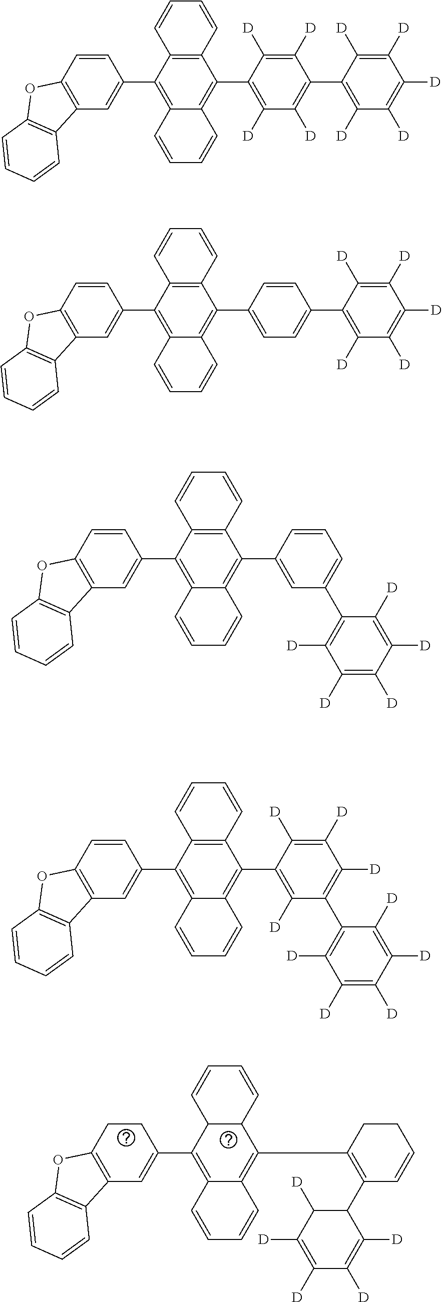

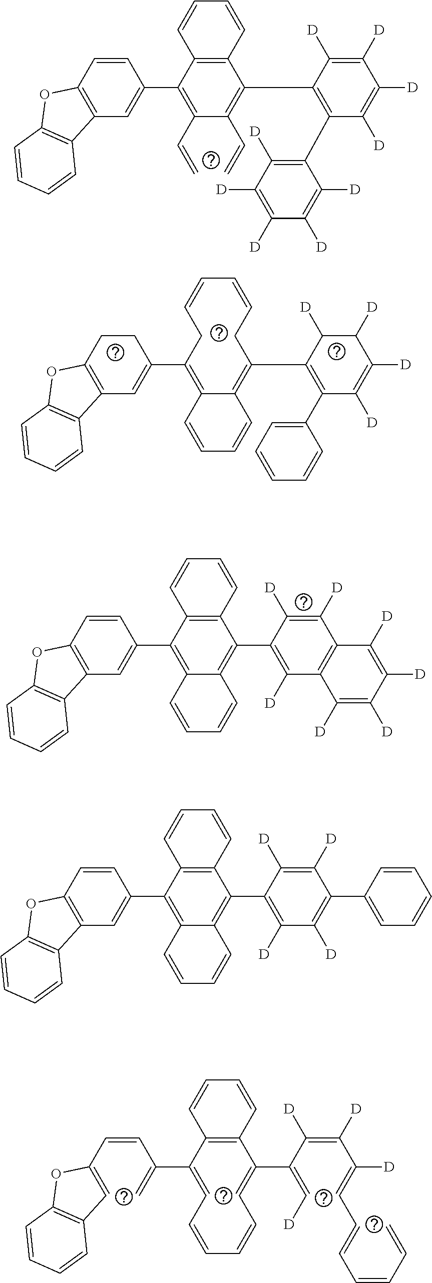

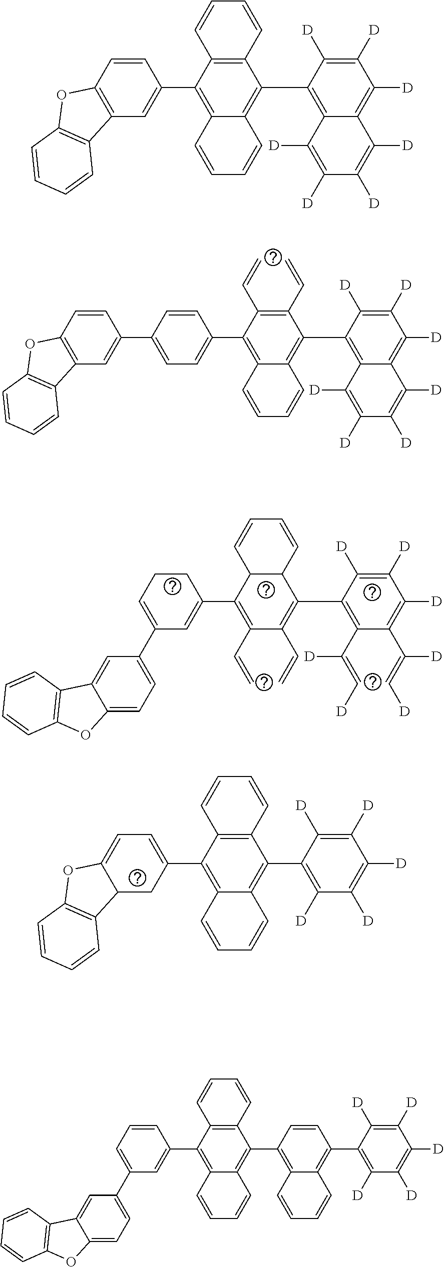

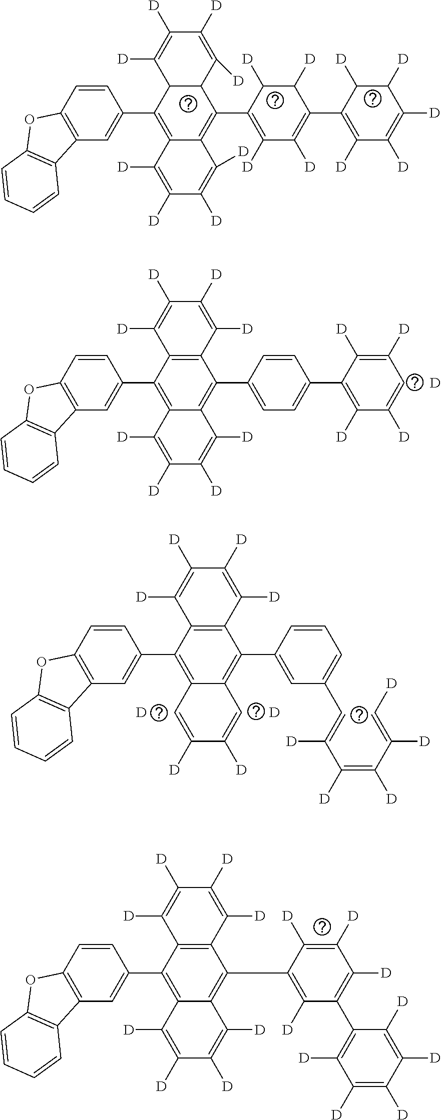

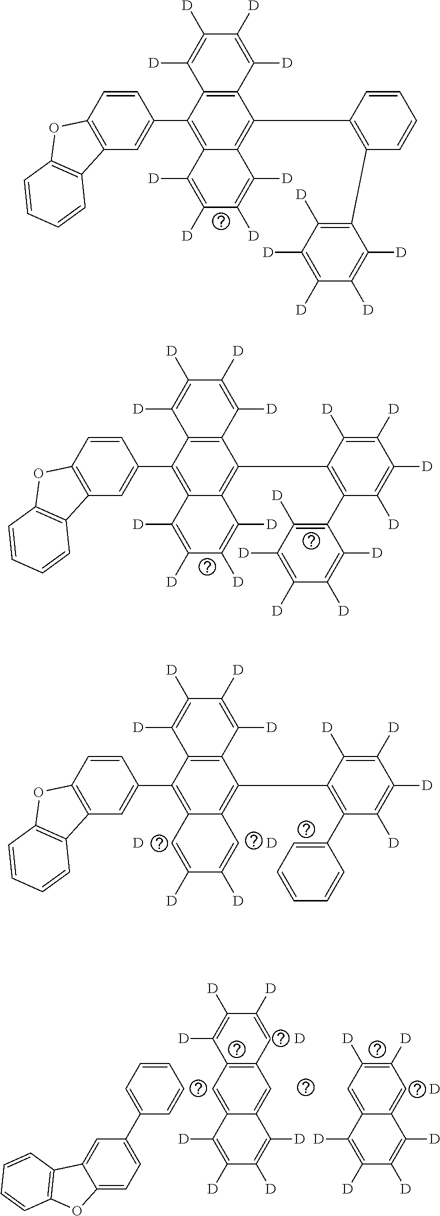

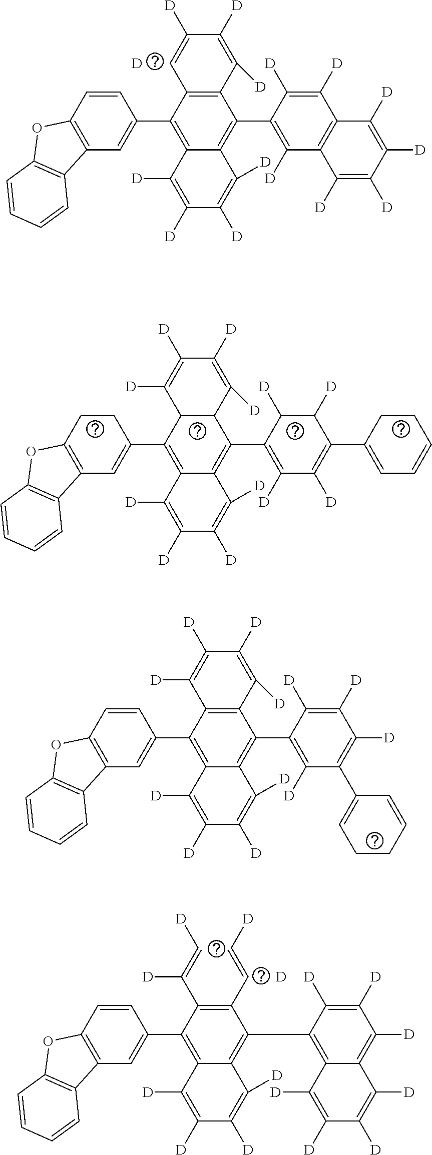

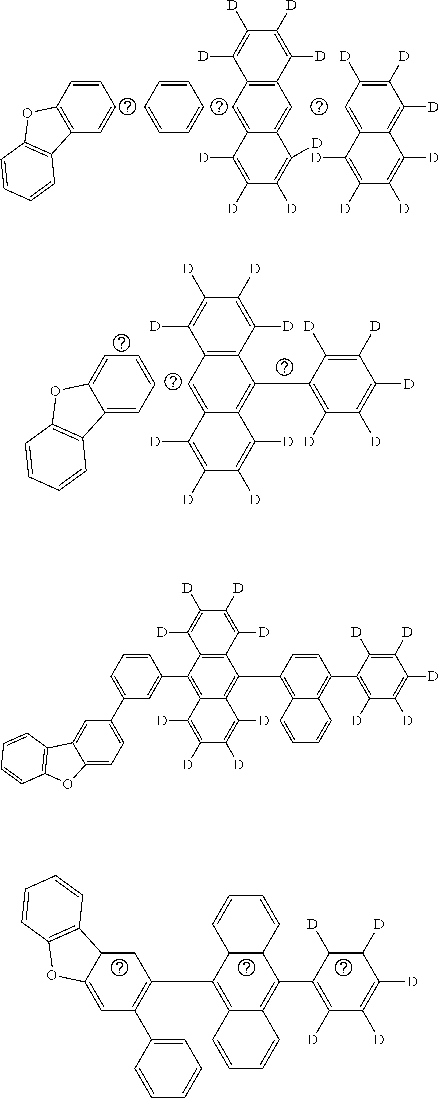

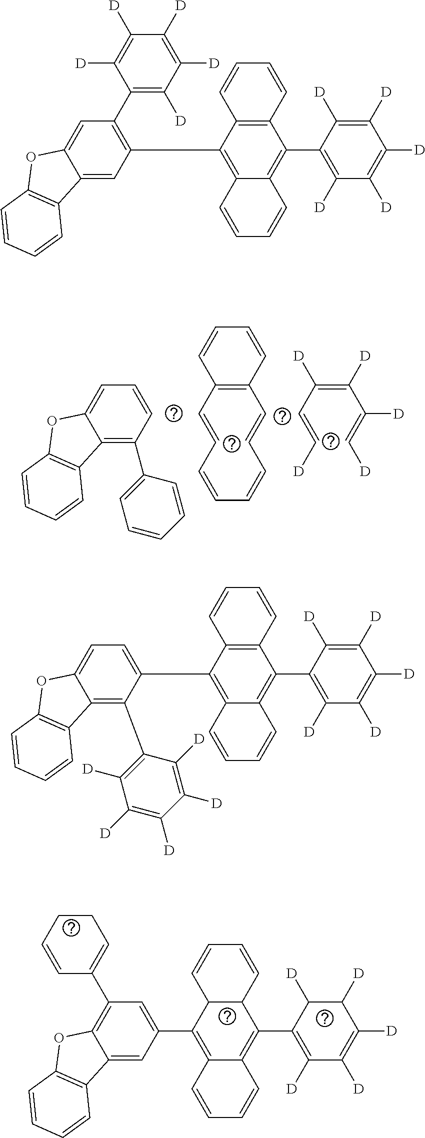

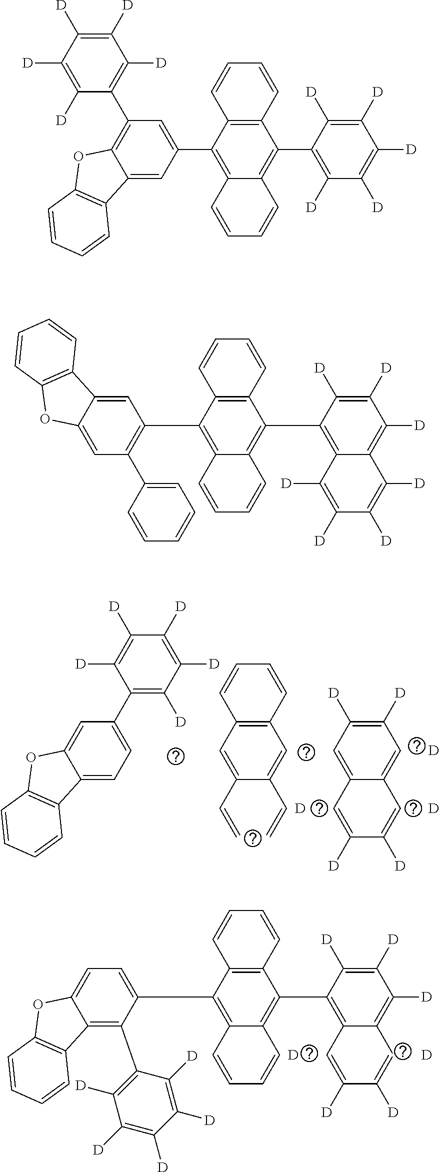

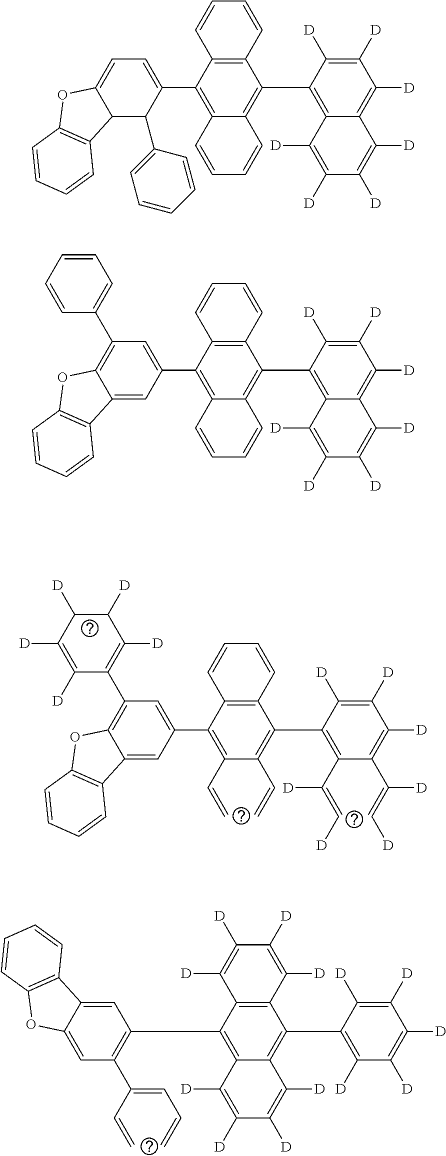

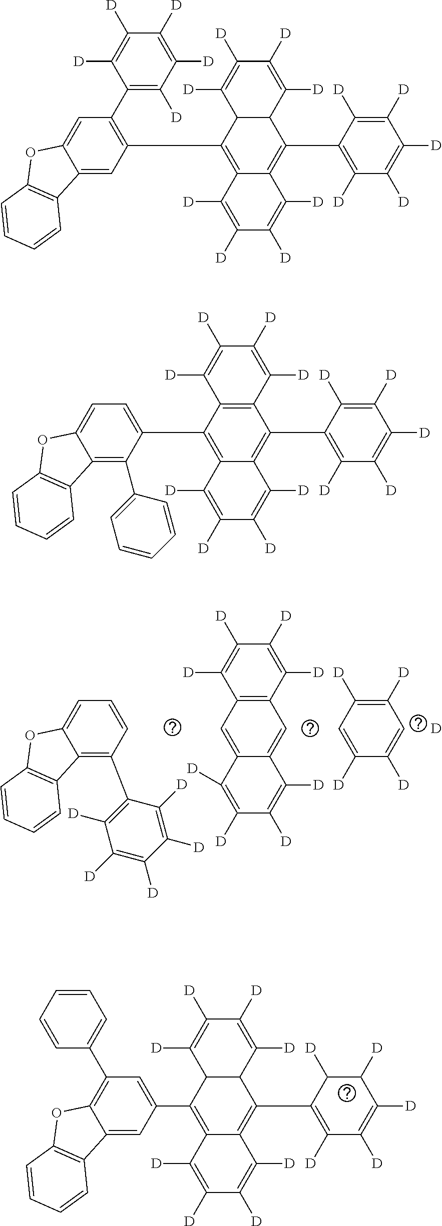

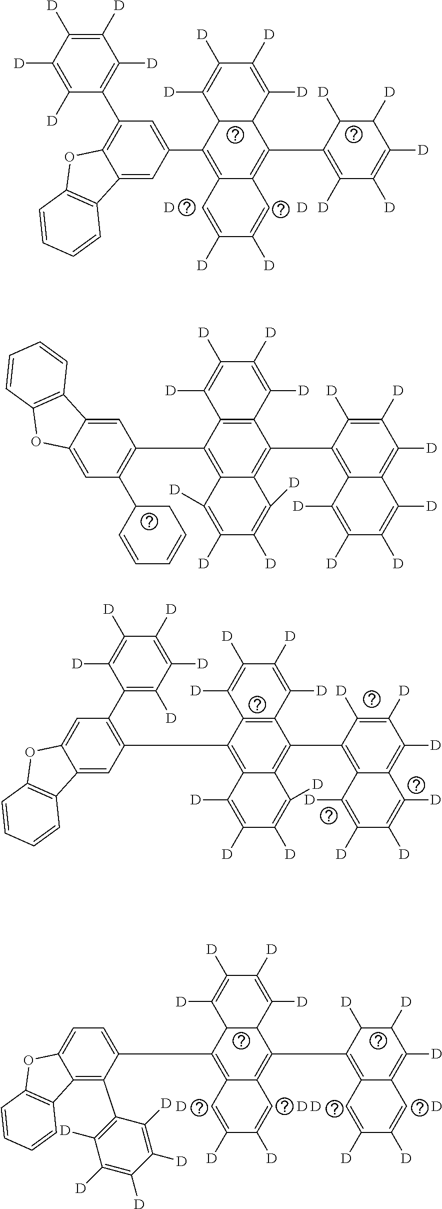

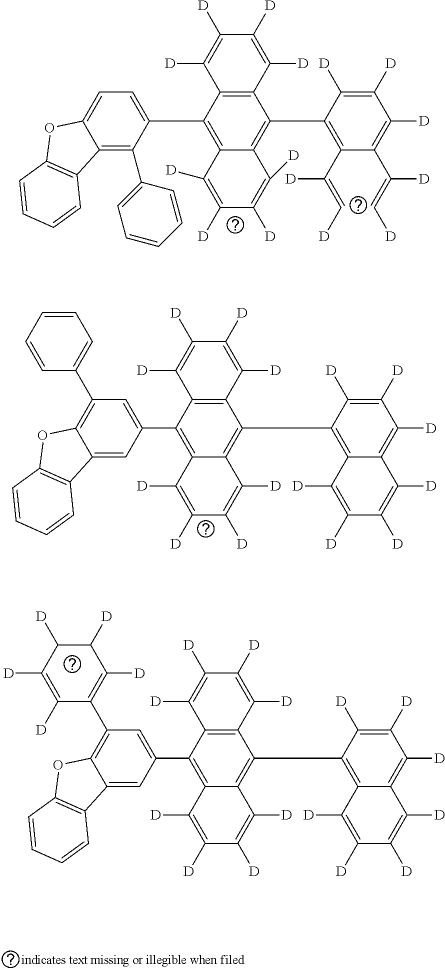

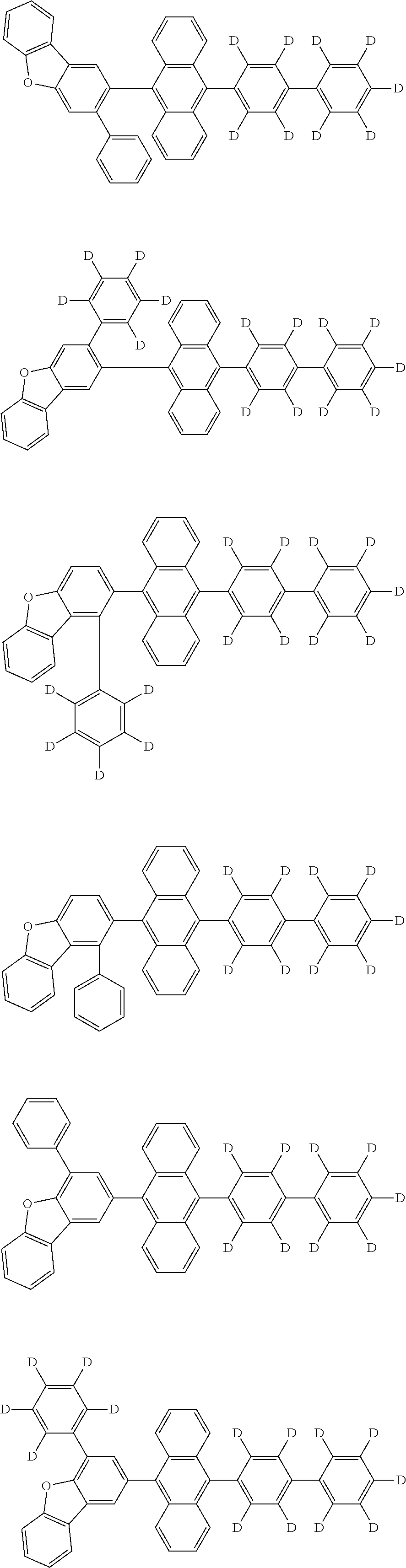

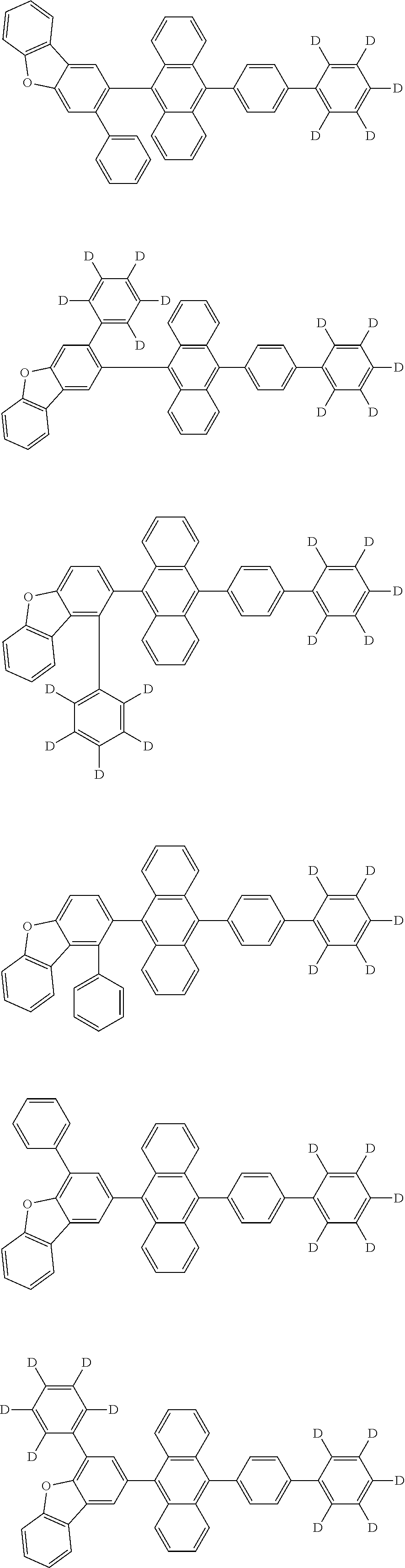

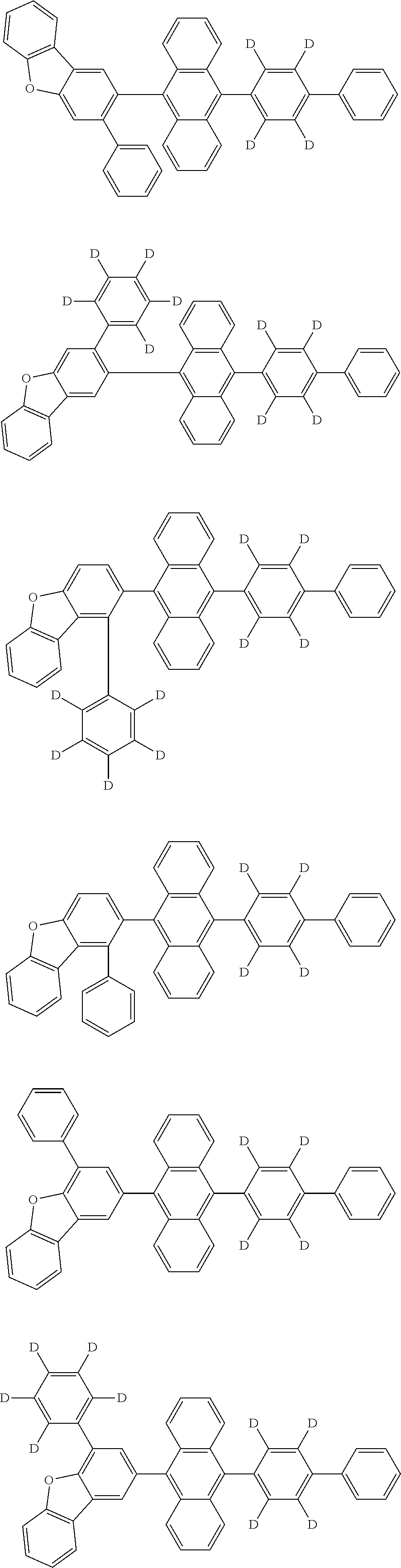

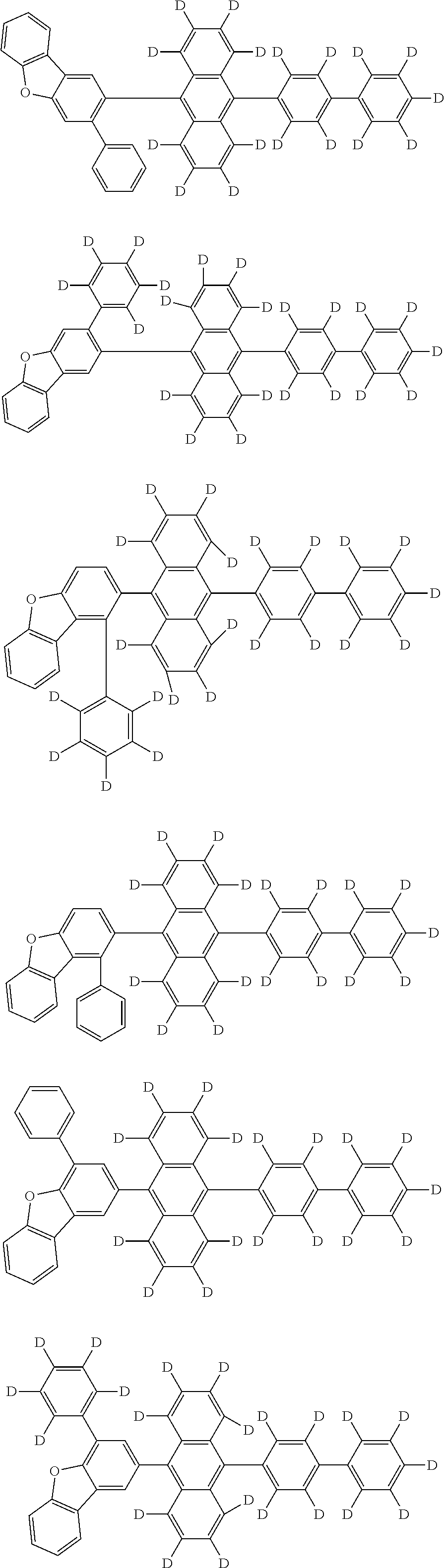

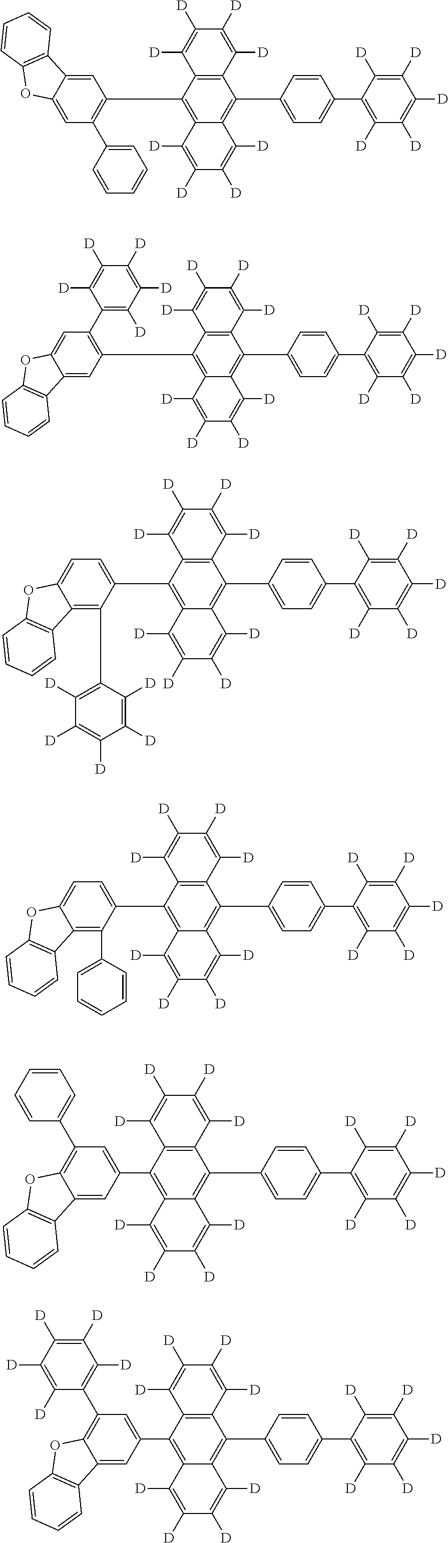

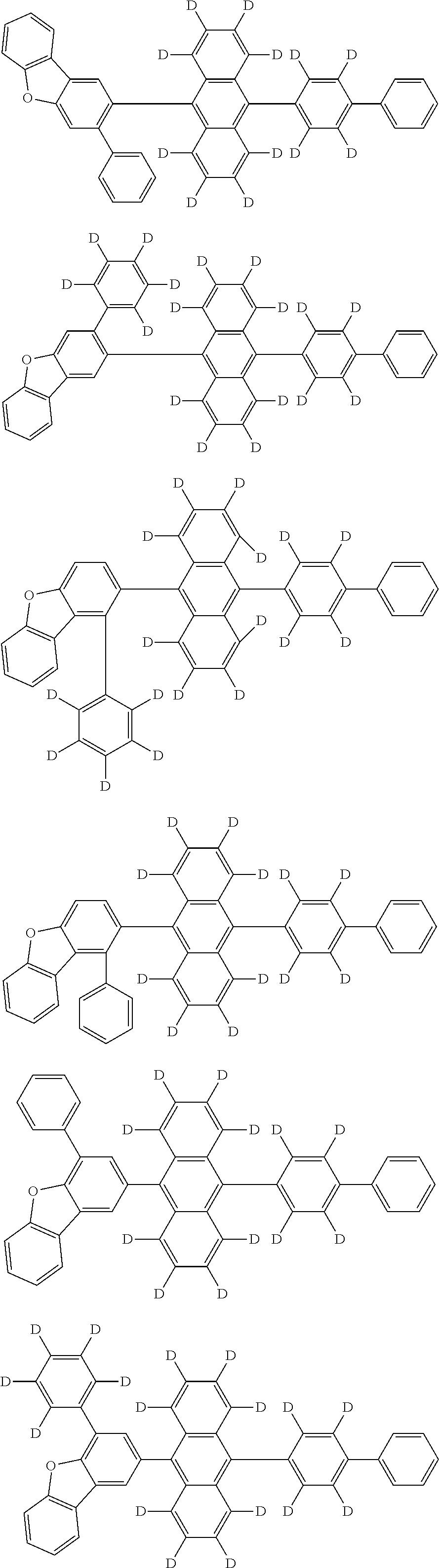

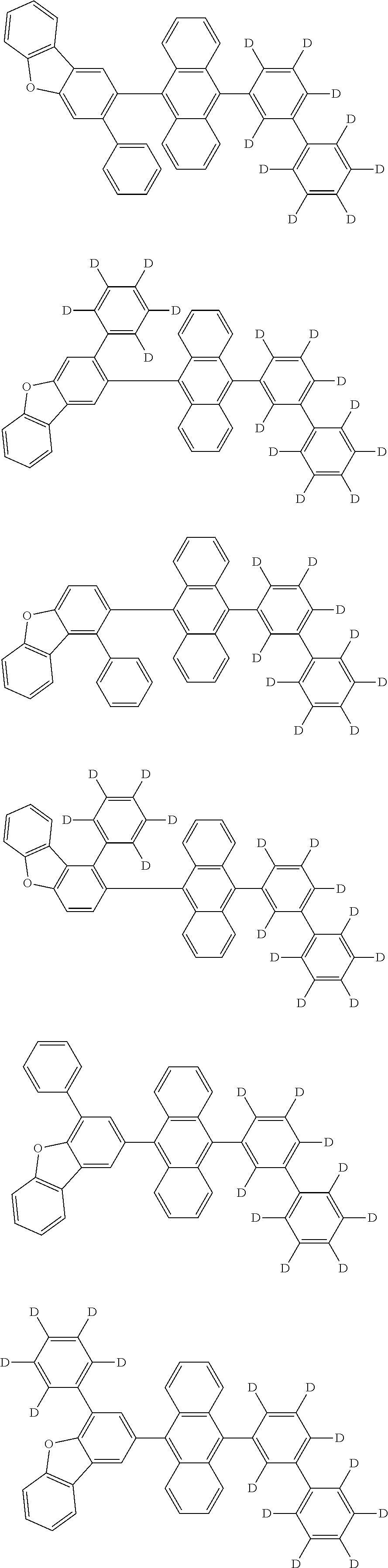

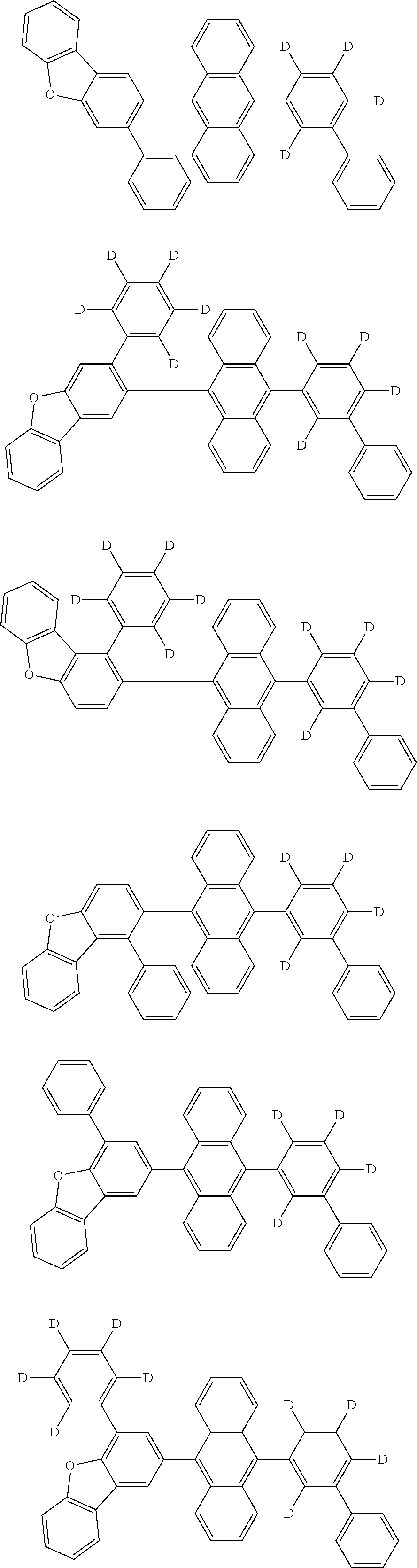

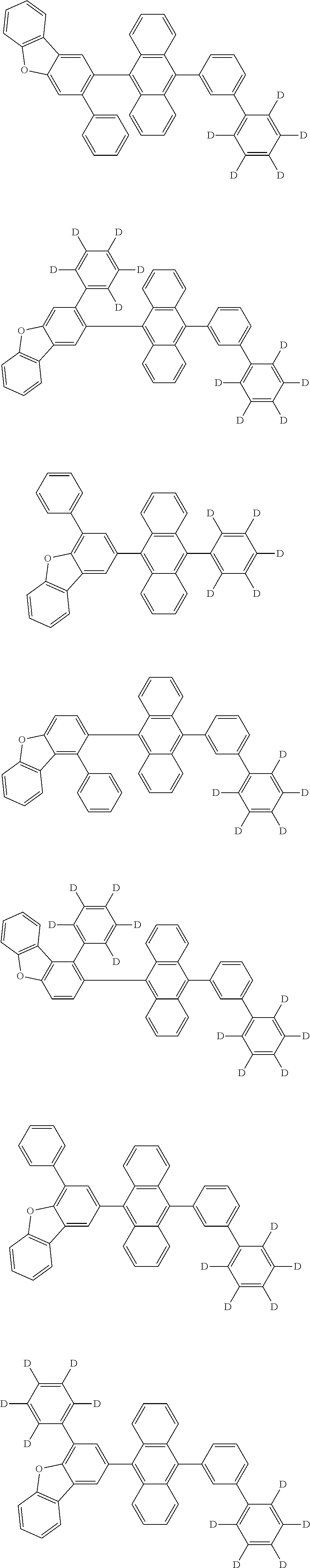

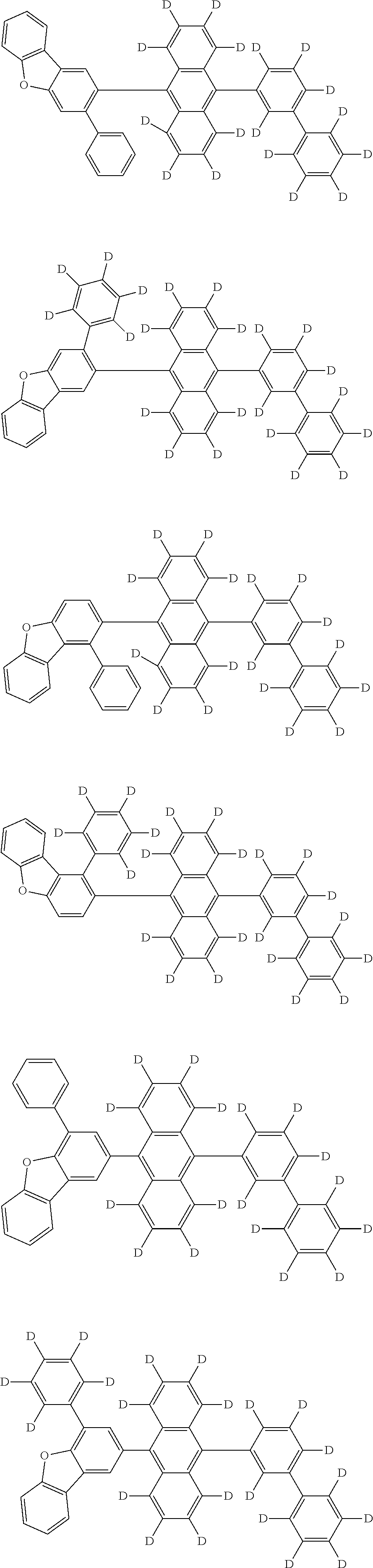

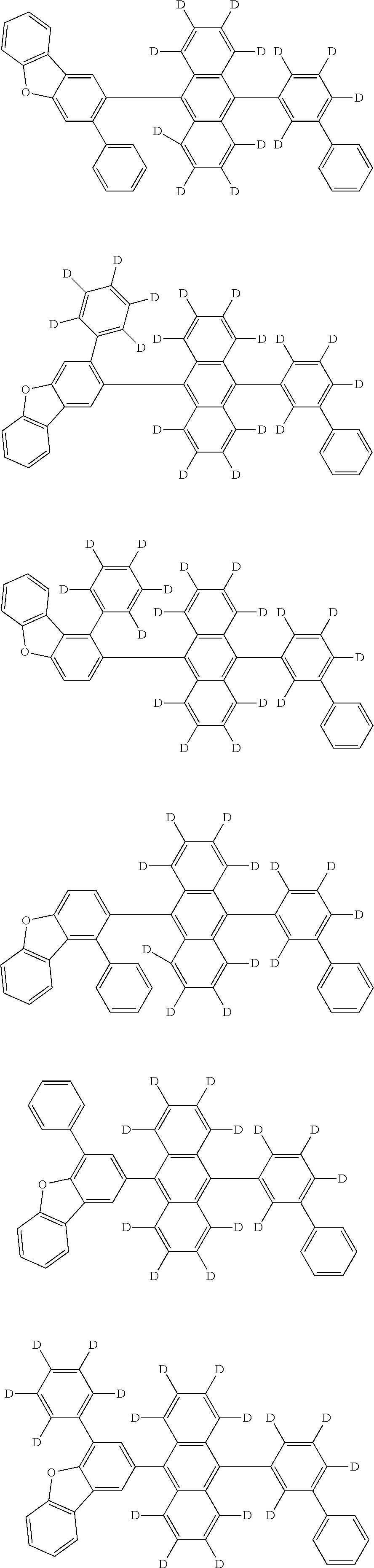

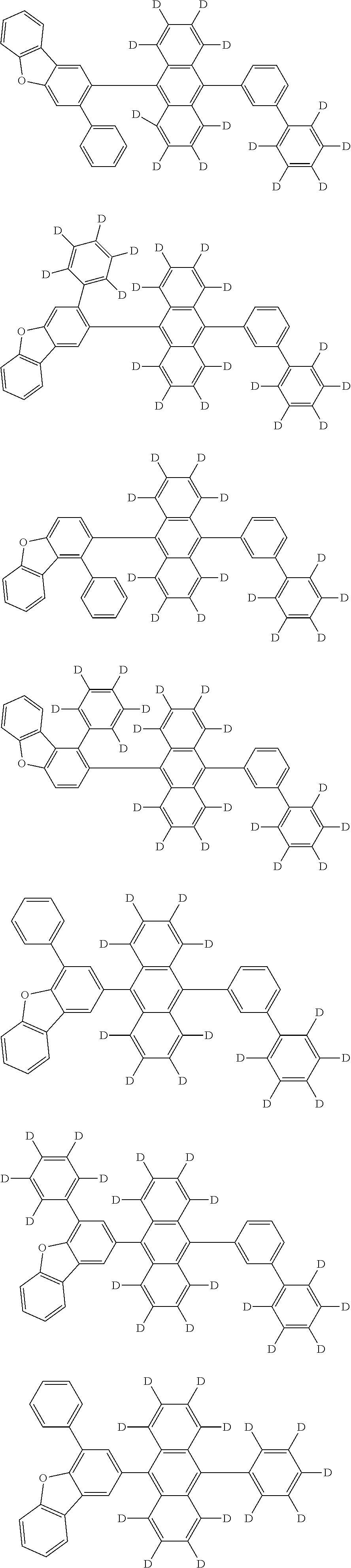

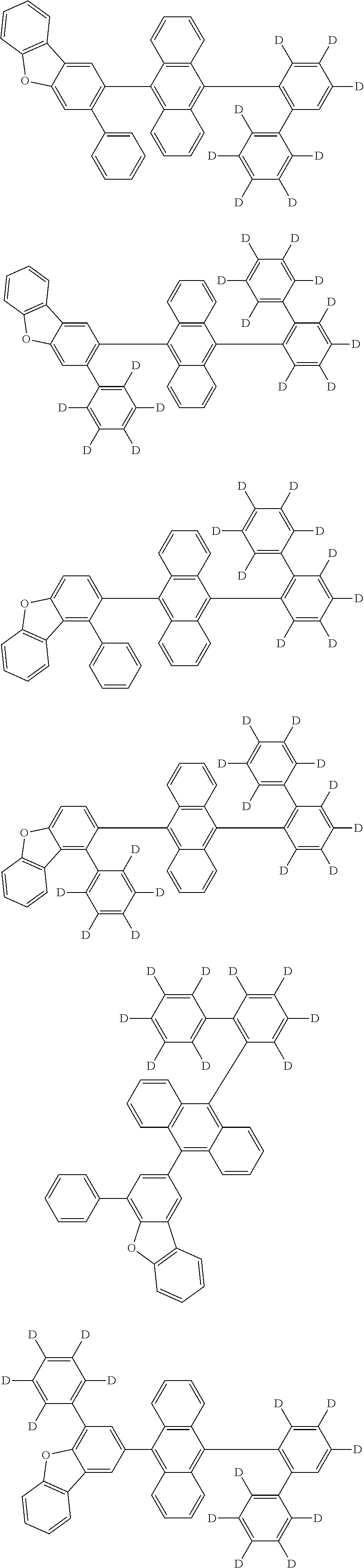

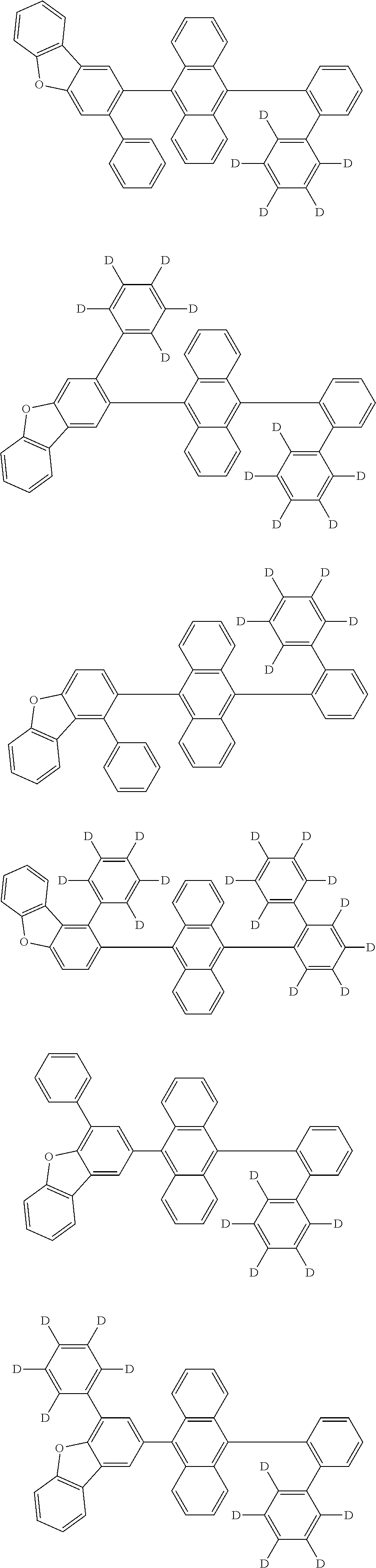

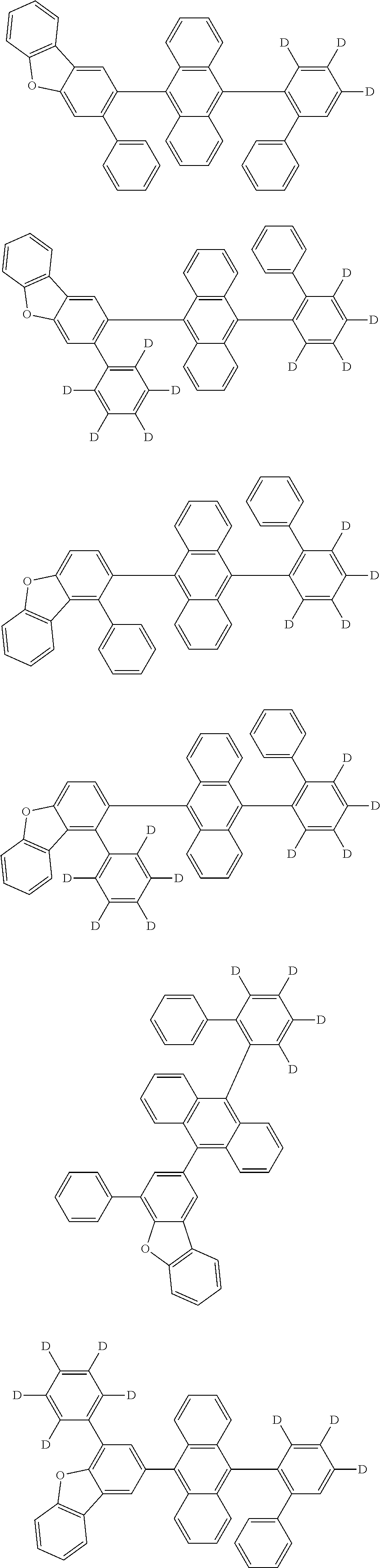

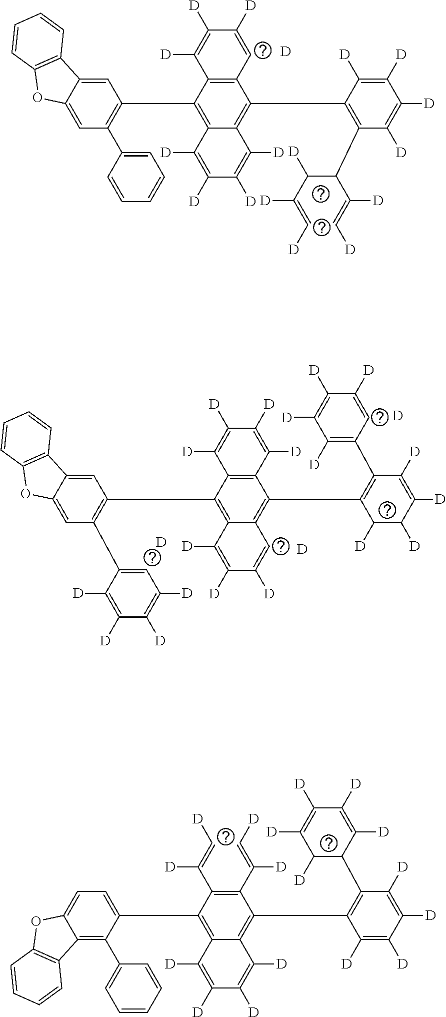

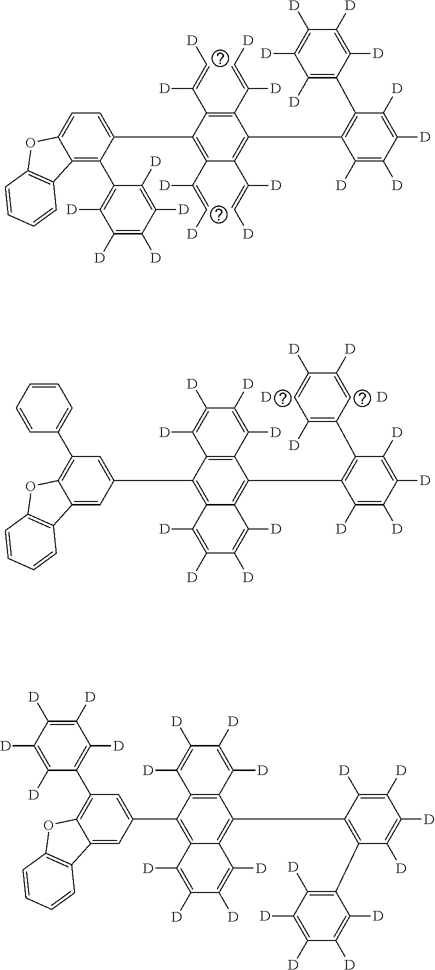

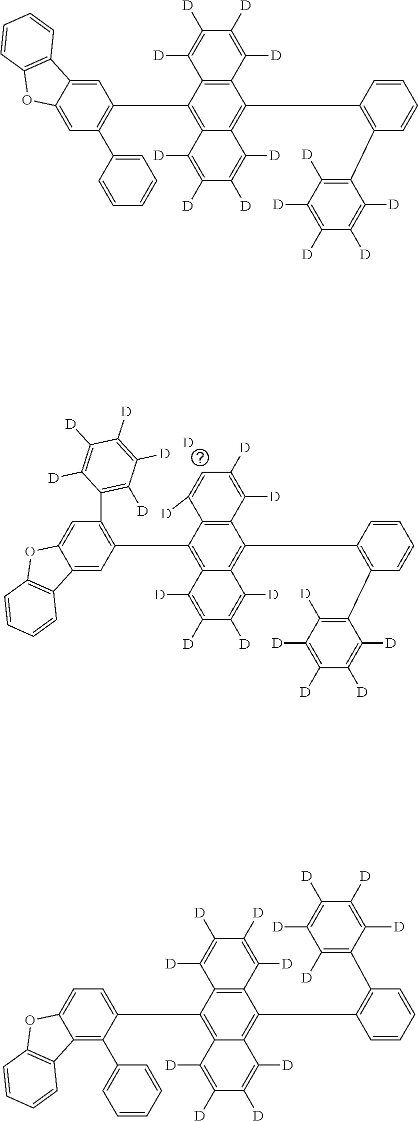

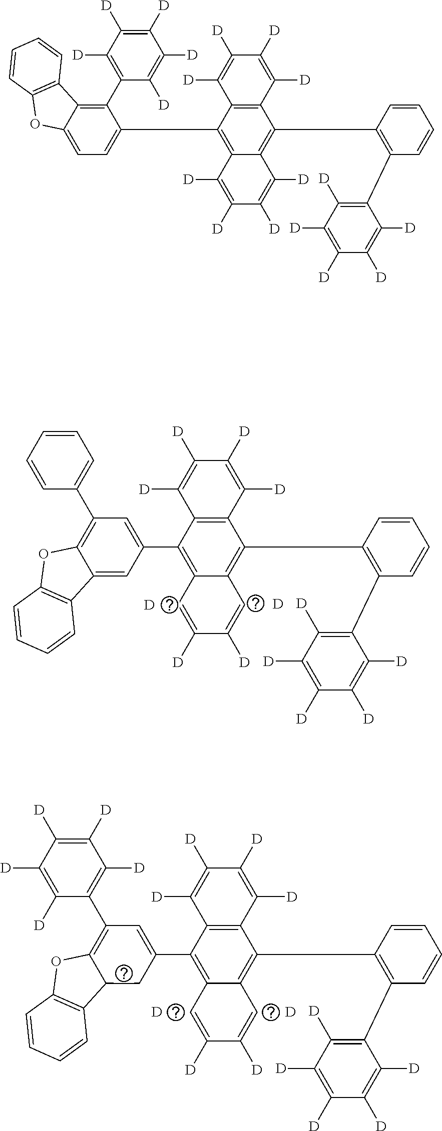

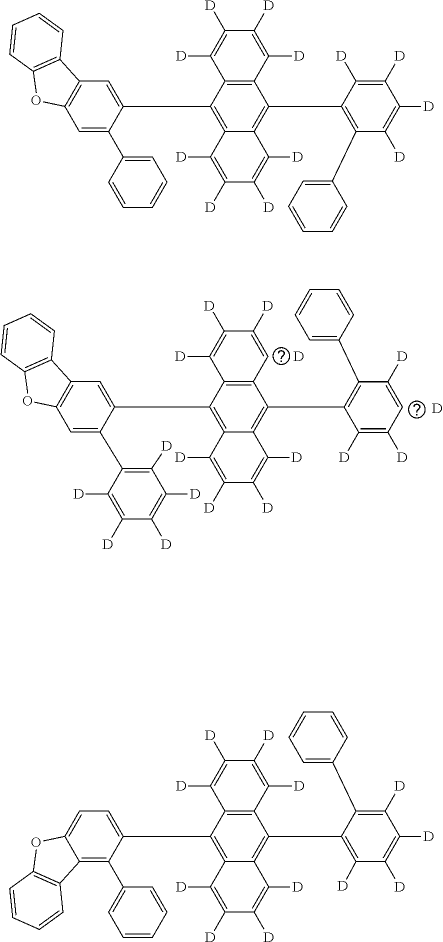

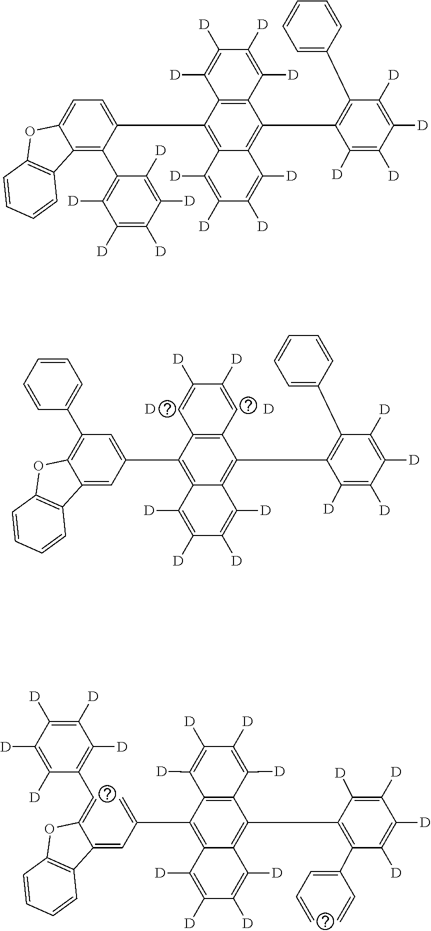

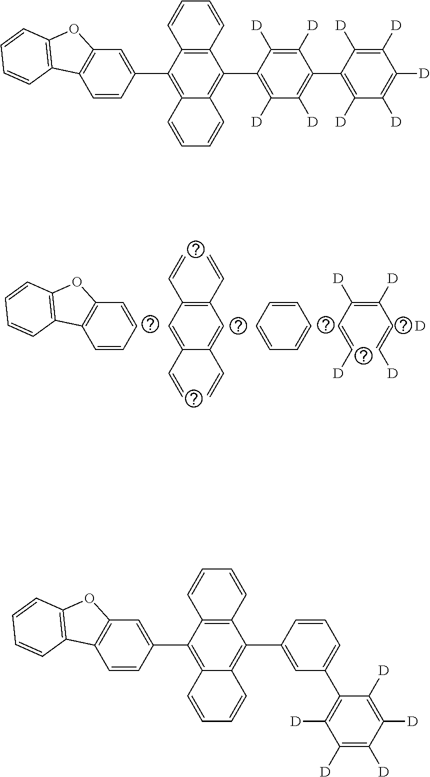

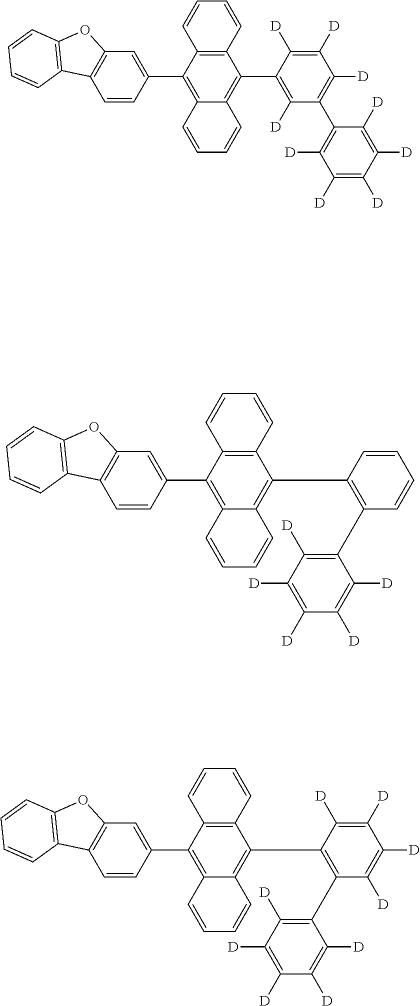 [SMILES: [2H]c1c([2H])c([2H])c(-c2c([2H])c([2H])c([2H])c(-c3c4ccccc4c(-c4ccc5c(c4)oc4ccccc45)c4ccccc34)c2[2H])c([2H])c1[2H] ; [2H]c1c([2H])c([2H])c(-c2ccccc2-c2c3ccccc3c(-c3ccc4c(c3)oc3ccccc34)c3ccccc23)c([2H])c1[2H] ; [2H]c1c([2H])c([2H])c(-c2c([2H])c([2H])c([2H])c([2H])c2-c2c3ccccc3c(-c3ccc4c(c3)oc3ccccc34)c3ccccc23)c([2H])c1[2H]]

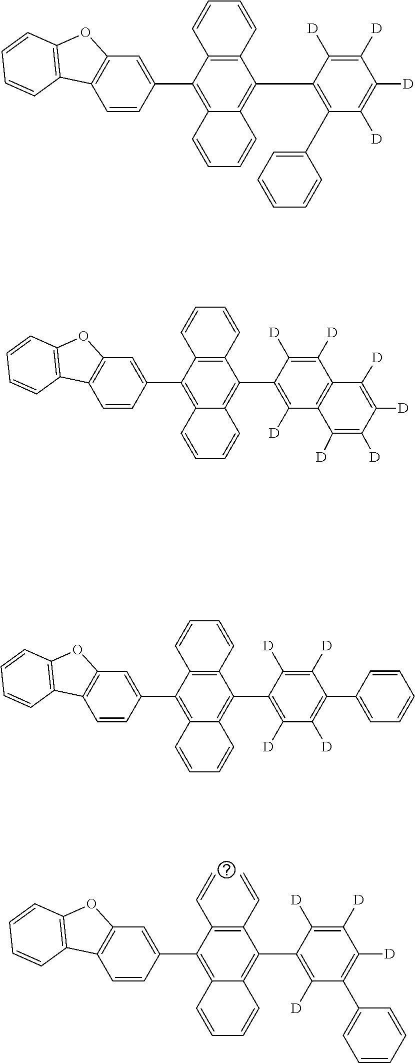 [SMILES: [2H]c1c([2H])c([2H])c(-c2c3ccccc3c(-c3ccc4c(c3)oc3ccccc34)c3ccccc23)c(-c2ccccc2)c1[2H] ; [2H]c1c([2H])c([2H])c2c([2H])c(-c3c4ccccc4c(-c4ccc5c(c4)oc4ccccc45)c4ccccc34)c([2H])c([2H])c2c1[2H] ; [2H]c1c([2H])c(-c2c3ccccc3c(-c3ccc4c(c3)oc3ccccc34)c3ccccc23)c([2H])c([2H])c1-c1ccccc1 ; [2H]c1c([2H])c(-c2ccccc2)c([2H])c(-c2c(C=C)c(C=C)c(-c3ccc4c(c3)oc3ccccc34)c3ccccc23)c1[2H]]

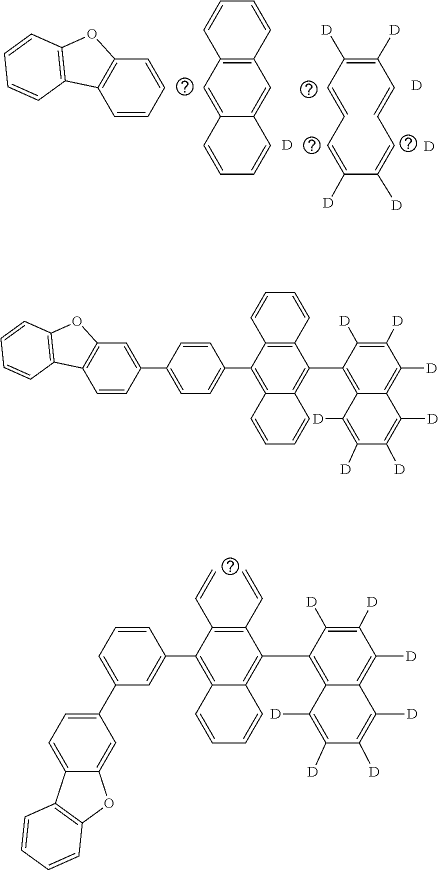

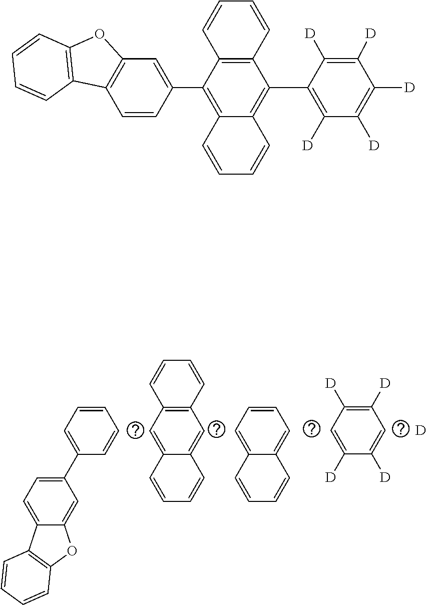

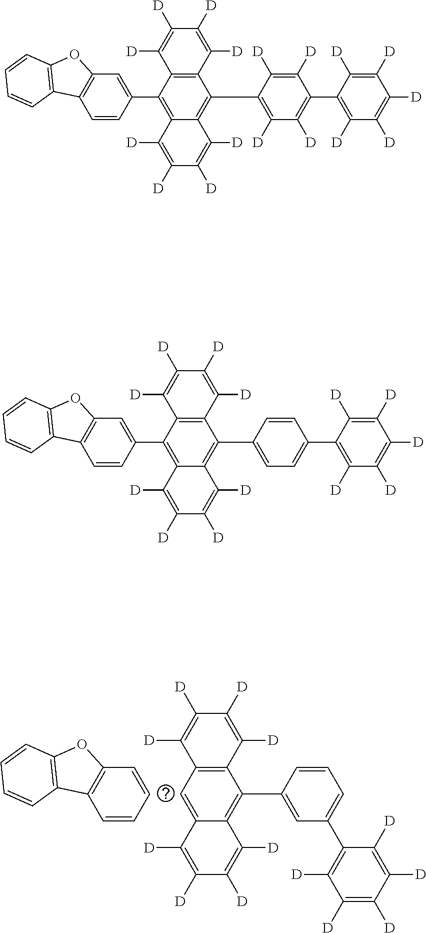

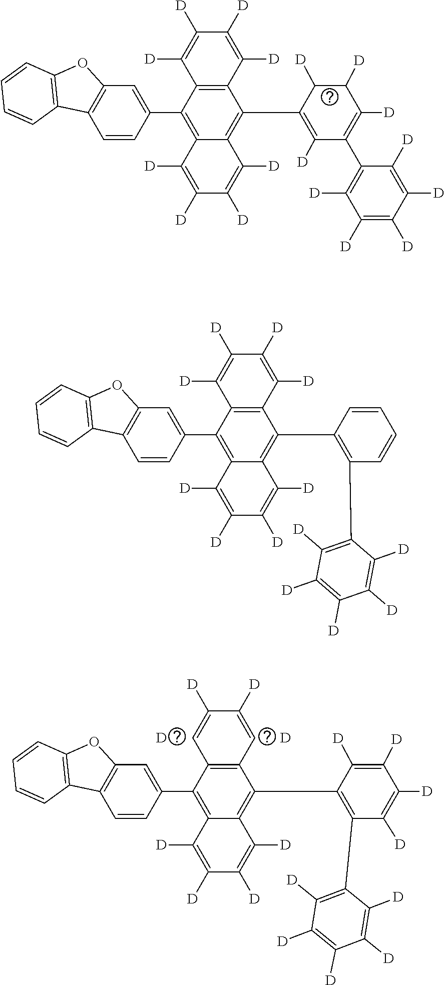

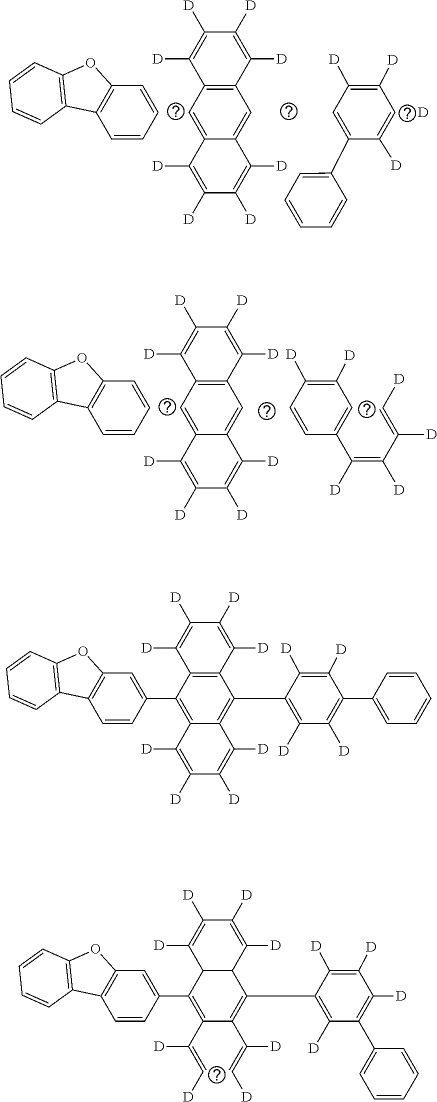

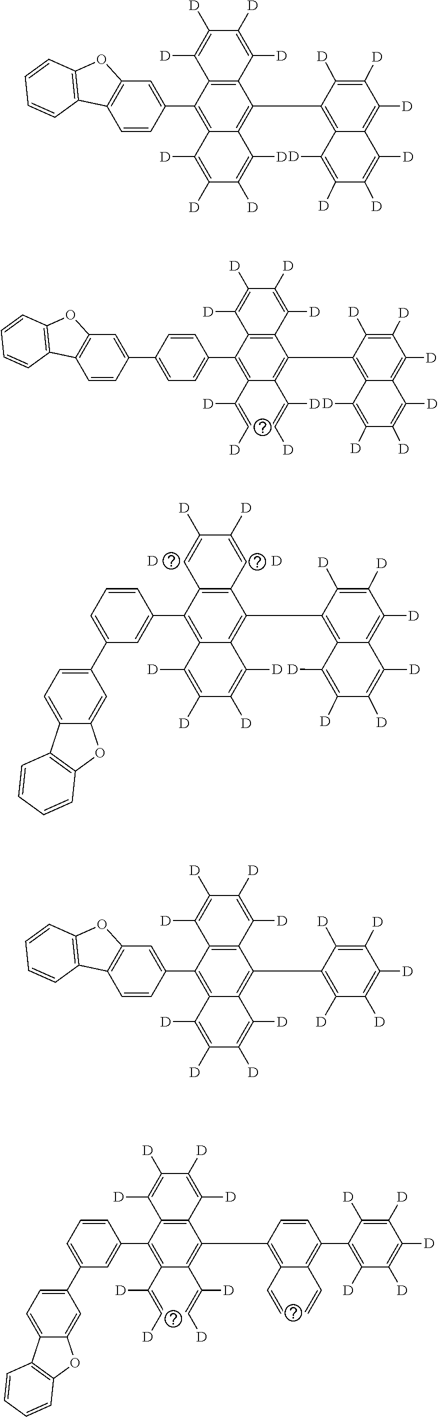

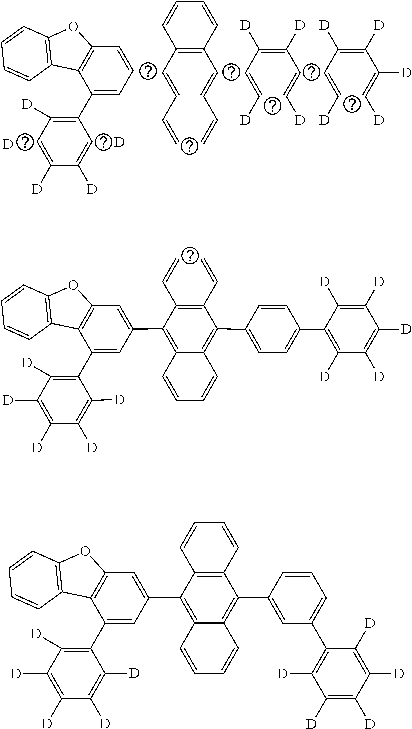

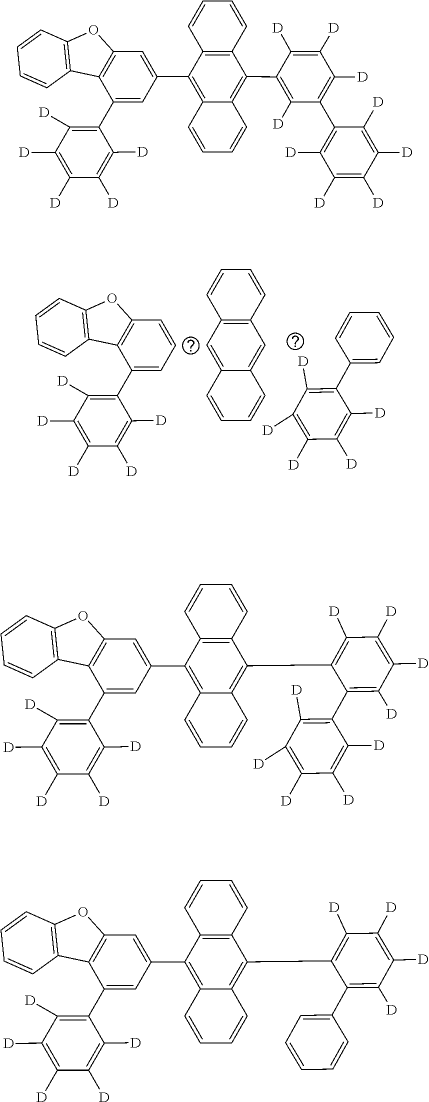

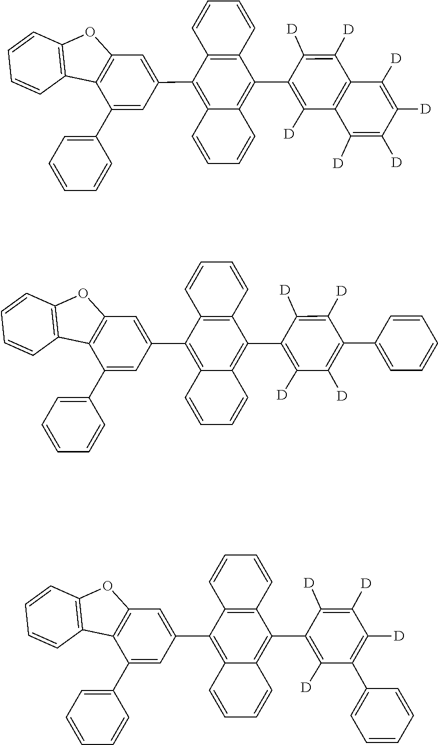 [SMILES: [2H]c1c([2H])c([2H])c2c([2H])c(-c3c4ccccc4c(-c4cc(-c5ccccc5)c5c(c4)oc4ccccc45)c4ccccc34)c([2H])c([2H])c2c1[2H] ; [2H]c1c([2H])c(-c2c3ccccc3c(-c3cc(-c4ccccc4)c4c(c3)oc3ccccc34)c3ccccc23)c([2H])c([2H])c1-c1ccccc1 ; [2H]c1c([2H])c(-c2ccccc2)c([2H])c(-c2c3ccccc3c(-c3cc(-c4ccccc4)c4c(c3)oc3ccccc34)c3ccccc23)c1[2H]]

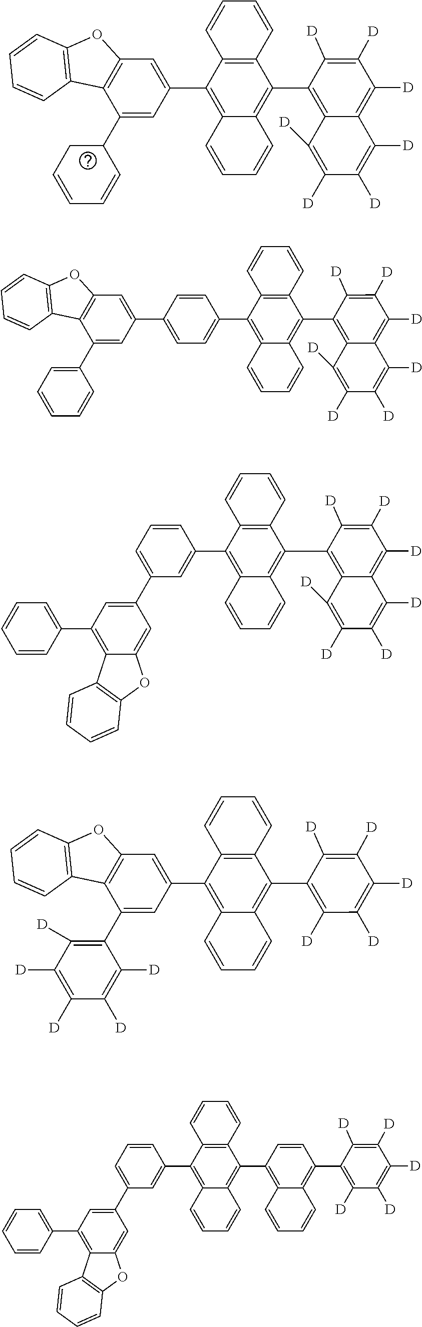

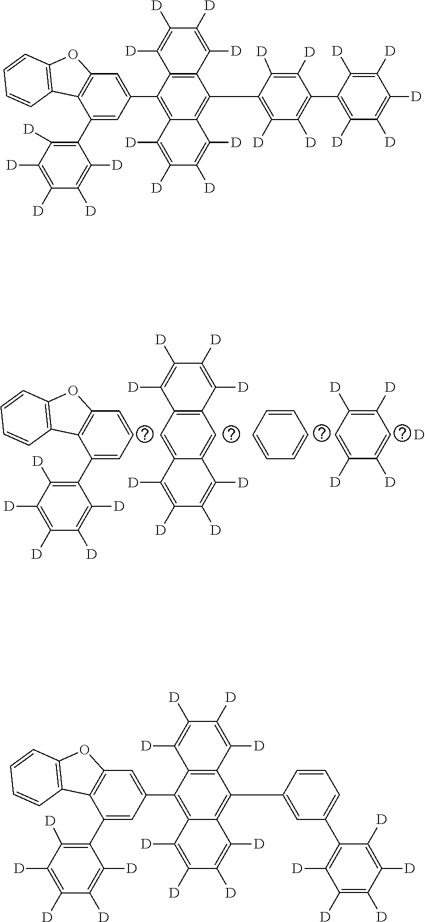

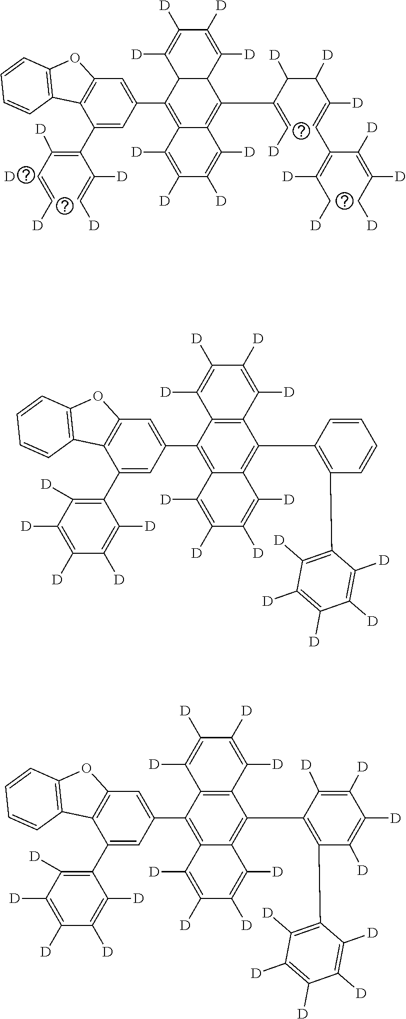

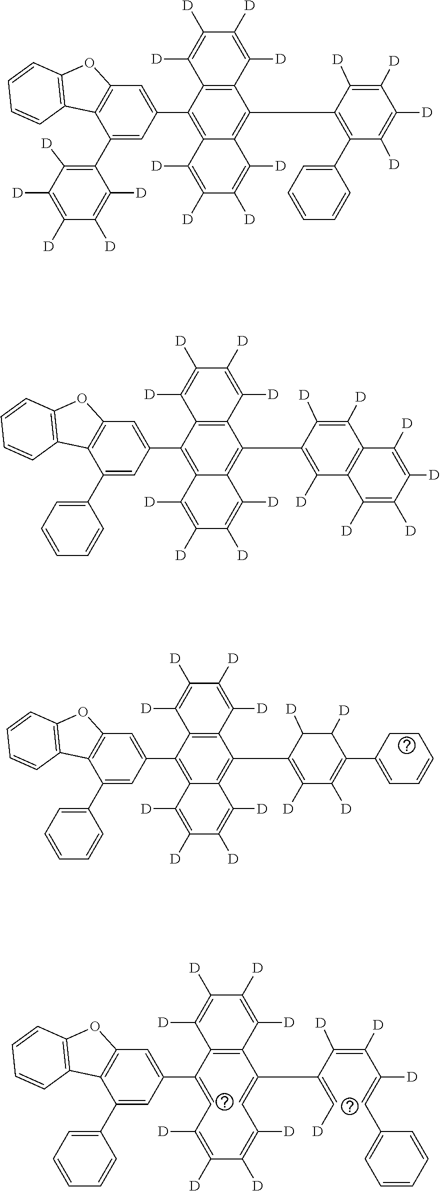

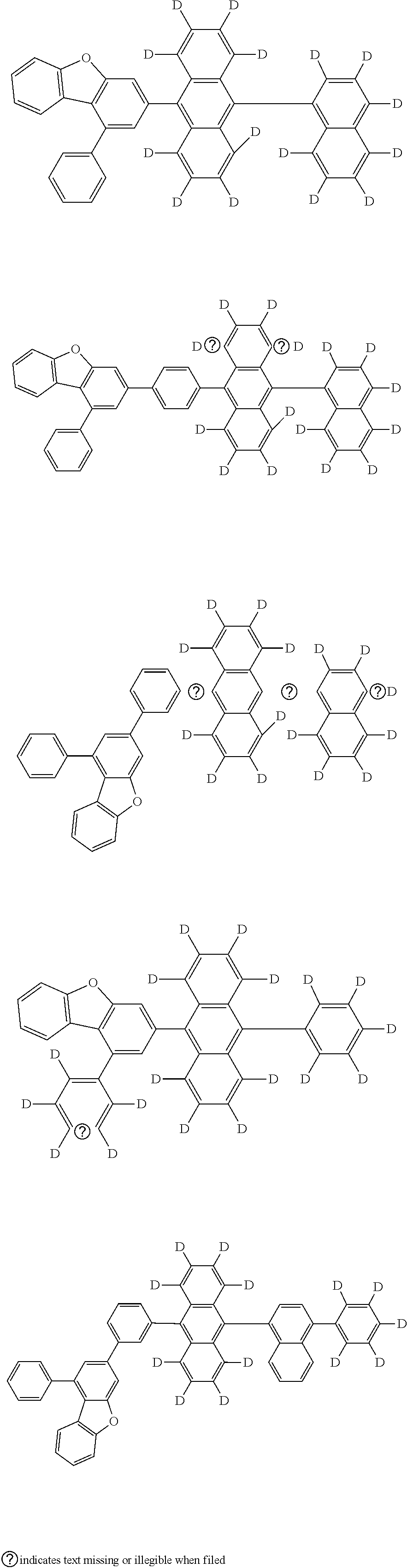

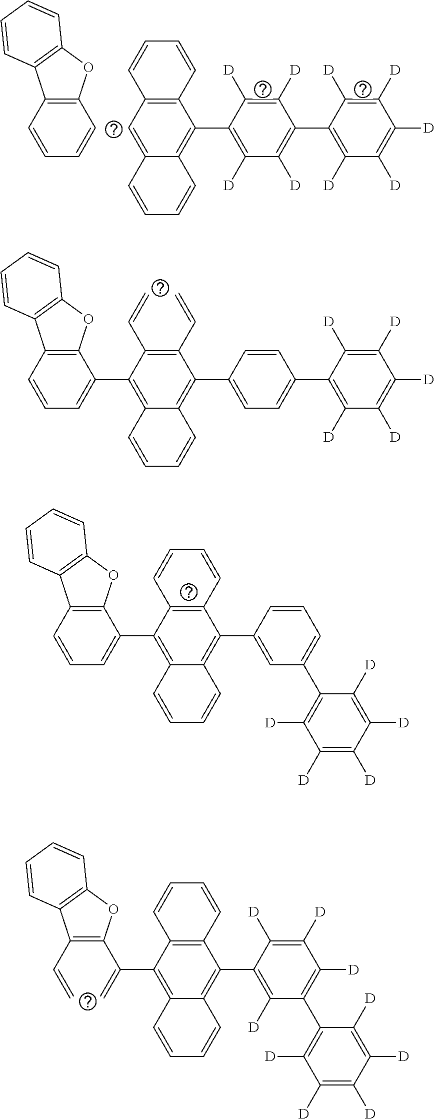

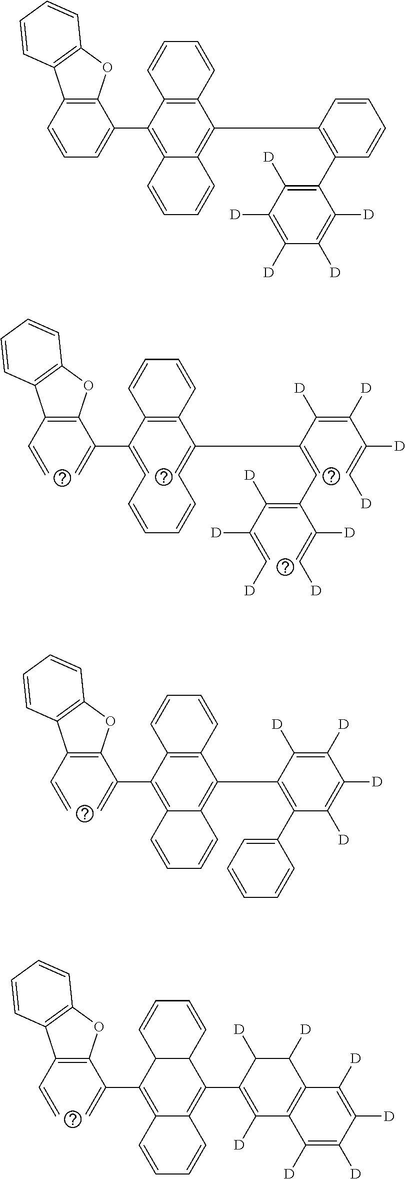 [SMILES: [2H]c1c([2H])c([2H])c(-c2ccccc2-c2c3ccccc3c(-c3cccc4c3oc3ccccc34)c3ccccc23)c([2H])c1[2H] ; [2H]\C=C([2H])\C([2H])=C([2H])/C(=C/C(=C([2H])/C([2H])=C/[2H])C(/[2H])=C/[2H])c1ccccccc(C(=C)c2oc3ccccc3c2C=C)c2ccccc12 ; [2H]c1c([2H])c([2H])c(-c2c3ccccc3c(C(=C)c3oc4ccccc4c3C=C)c3ccccc23)c(-c2ccccc2)c1[2H] ; [2H]C1=C(C2=c3ccccc3=C(C(=C)c3oc4ccccc4c3C=C)C3C=CC=CC23)C([2H])C([2H])c2c([2H])c([2H])c([2H])c([2H])c21]

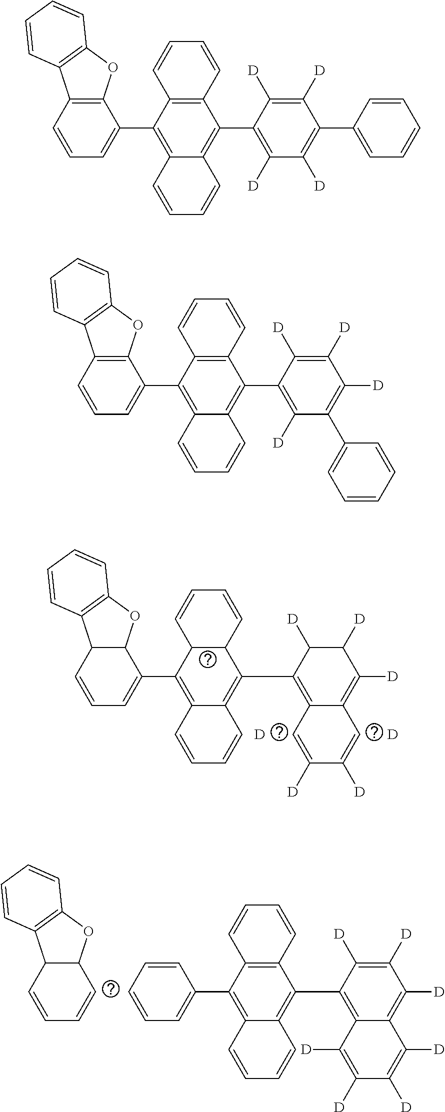

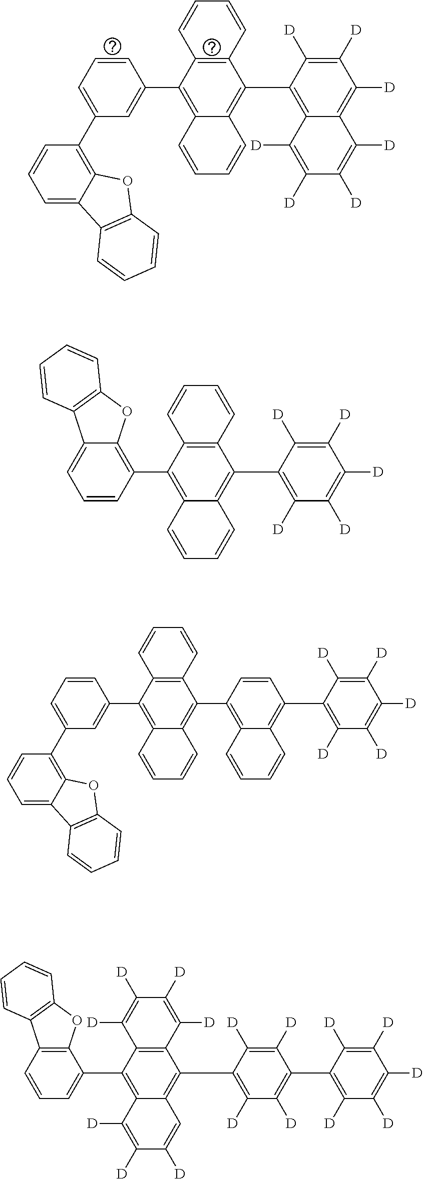 [SMILES: [2H]c1c([2H])c([2H])c2c(-c3c4ccccc4c(-c4cccc(-c5cccc6c5oc5ccccc56)c4)c4ccccc34)c([2H])c([2H])c([2H])c2c1[2H] ; [2H]c1c([2H])c([2H])c(-c2c3ccccc3c(-c3cccc4c3oc3ccccc34)c3ccccc23)c([2H])c1[2H] ; [2H]c1c([2H])c([2H])c(-c2ccc(-c3c4ccccc4c(-c4cccc(-c5cccc6c5oc5ccccc56)c4)c4ccccc34)c3ccccc23)c([2H])c1[2H] ; [2H]c1cc2c(-c3c([2H])c([2H])c(-c4c([2H])c([2H])c([2H])c([2H])c4[2H])c([2H])c3[2H])c3c([2H])c([2H])c([2H])c([2H])c3c(-c3cccc4c3oc3ccccc34)c2c([2H])c1[2H]]

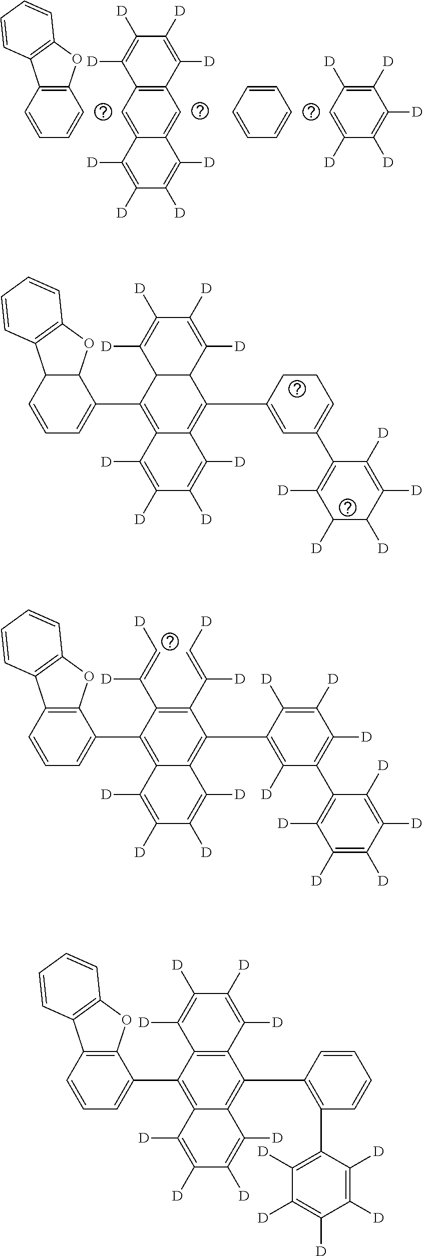

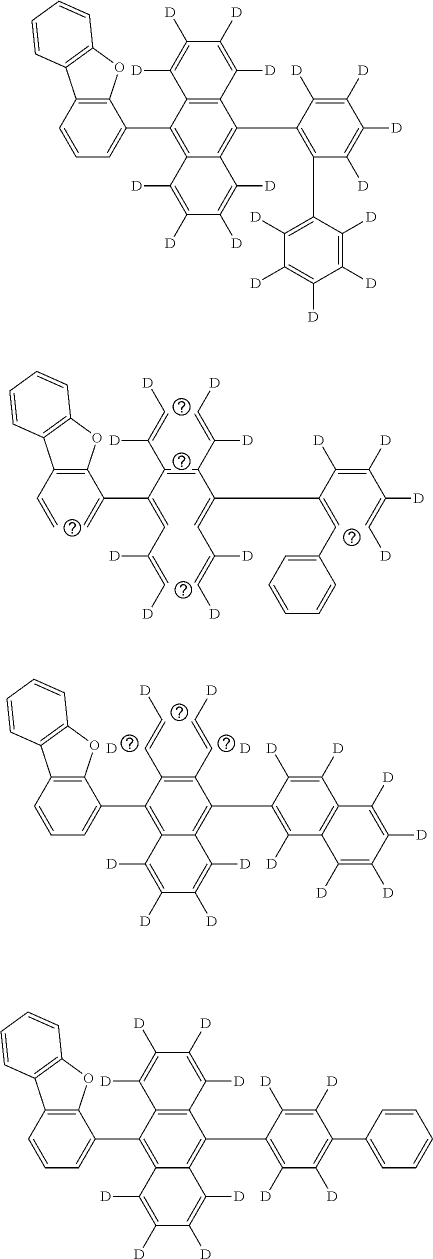

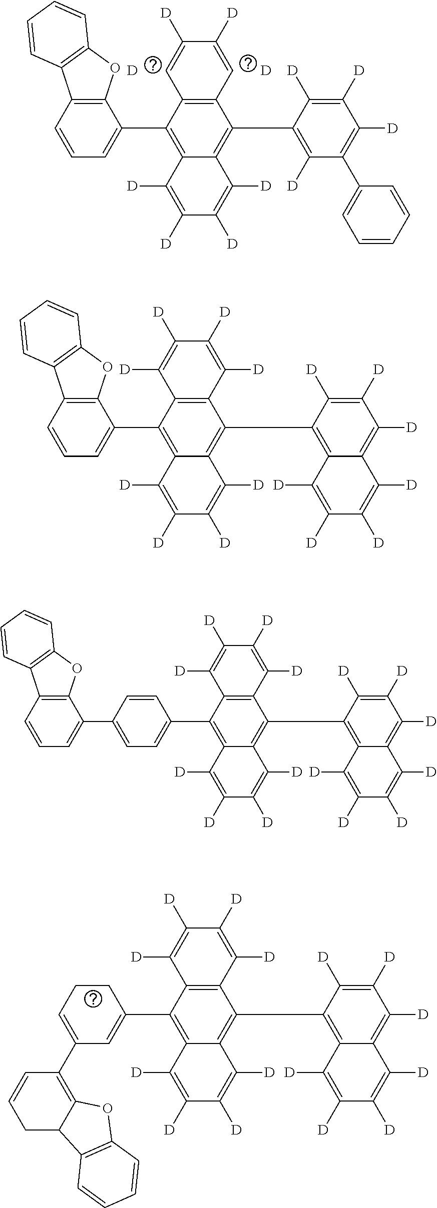

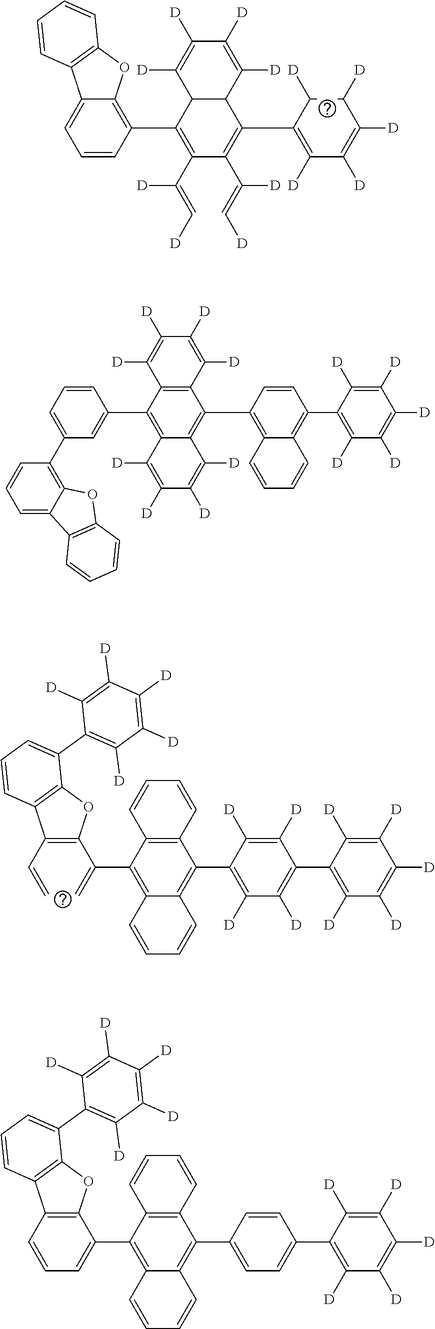

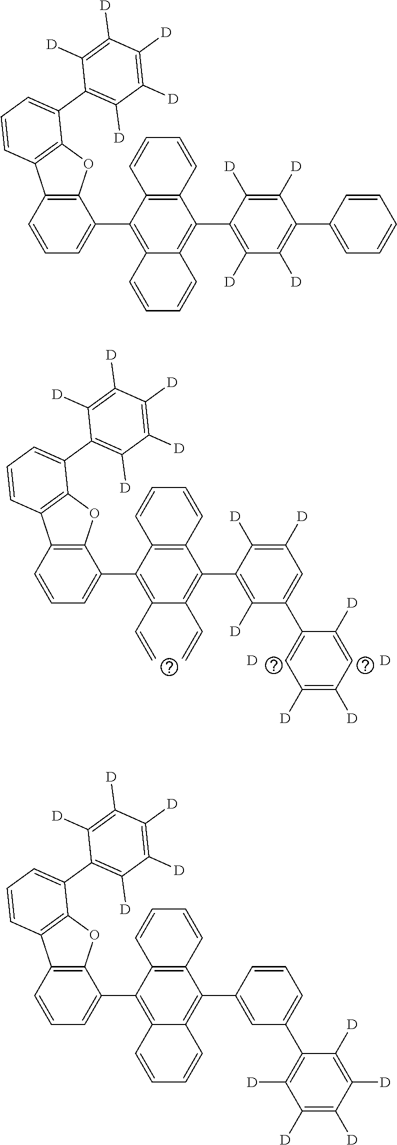

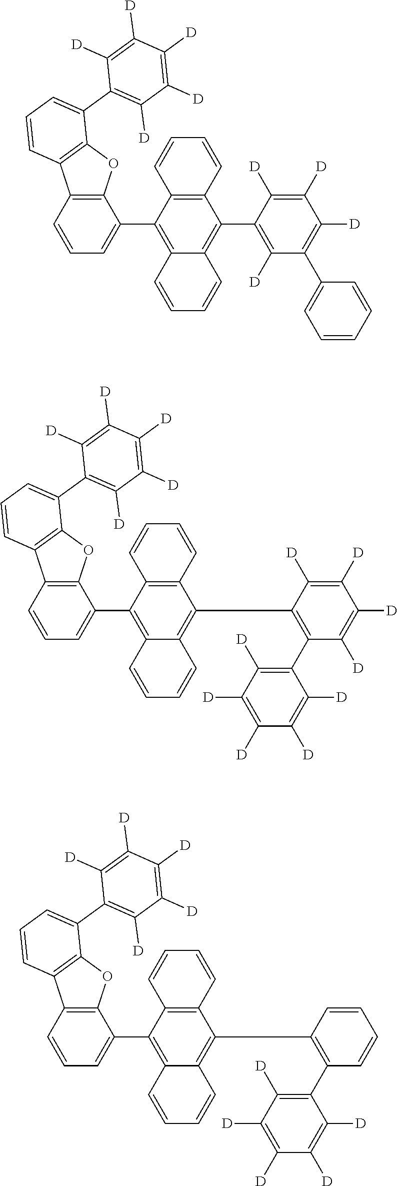 [SMILES: [2H]c1c([2H])c([2H])c(-c2cccc3c2oc2c(-c4c5ccccc5c(-c5c([2H])c([2H])c([2H])c(-c6ccccc6)c5[2H])c5ccccc45)cccc23)c([2H])c1[2H] ; [2H]c1c([2H])c([2H])c(-c2c([2H])c([2H])c([2H])c([2H])c2-c2c3ccccc3c(-c3cccc4c3oc3c(-c5c([2H])c([2H])c([2H])c([2H])c5[2H])cccc34)c3ccccc23)c([2H])c1[2H] ; [2H]c1c([2H])c([2H])c(-c2ccccc2-c2c3ccccc3c(-c3cccc4c3oc3c(-c5c([2H])c([2H])c([2H])c([2H])c5[2H])cccc34)c3ccccc23)c([2H])c1[2H]]

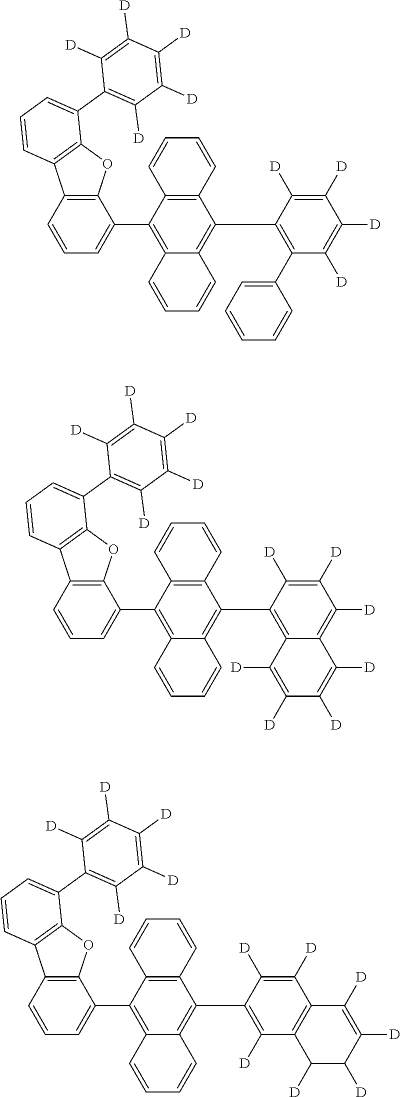 [SMILES: [2H]c1c([2H])c([2H])c(-c2cccc3c2oc2c(-c4c5ccccc5c(-c5c([2H])c([2H])c([2H])c([2H])c5-c5ccccc5)c5ccccc45)cccc23)c([2H])c1[2H] ; [2H]c1c([2H])c([2H])c(-c2cccc3c2oc2c(-c4c5ccccc5c(-c5c([2H])c([2H])c([2H])c6c([2H])c([2H])c([2H])c([2H])c56)c5ccccc45)cccc23)c([2H])c1[2H] ; [2H]C1=C([2H])C([2H])C([2H])c2c([2H])c(-c3c4ccccc4c(-c4cccc5c4oc4c(-c6c([2H])c([2H])c([2H])c([2H])c6[2H])cccc45)c4ccccc34)c([2H])c([2H])c21]

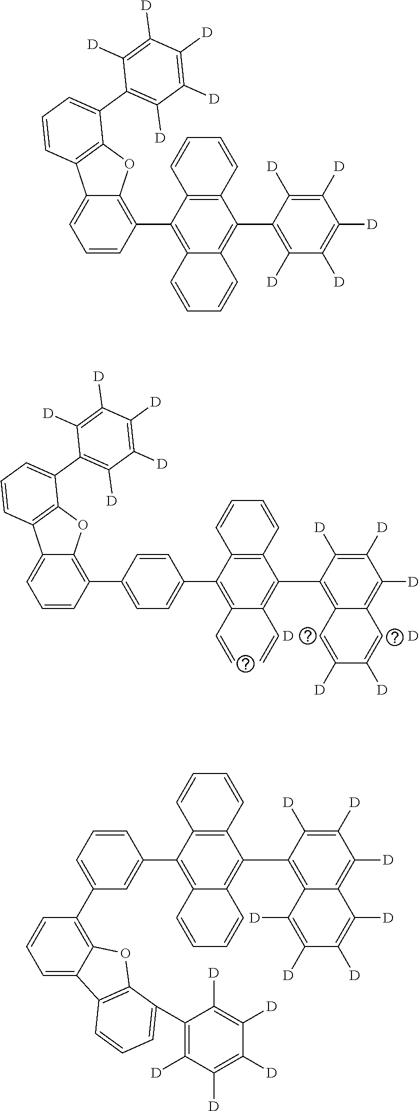

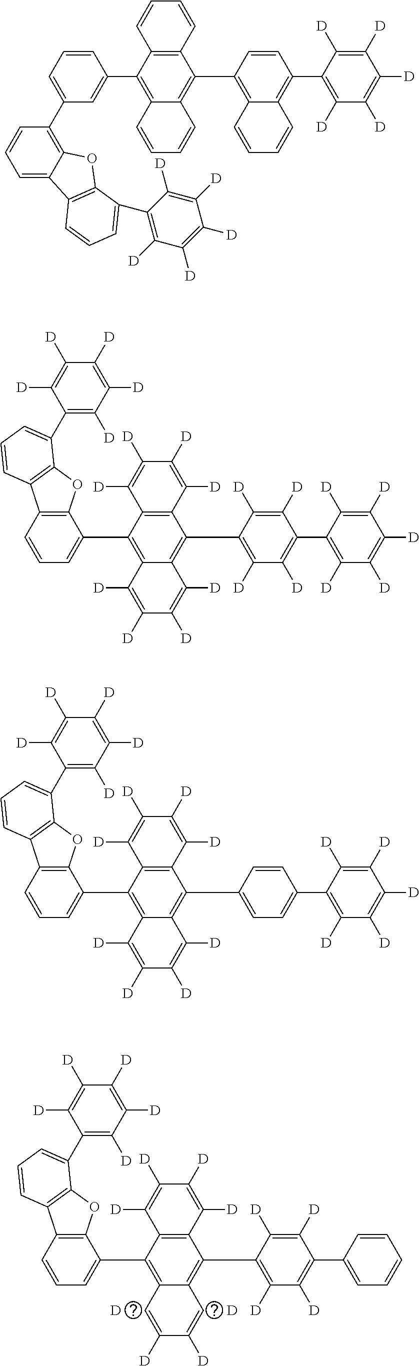

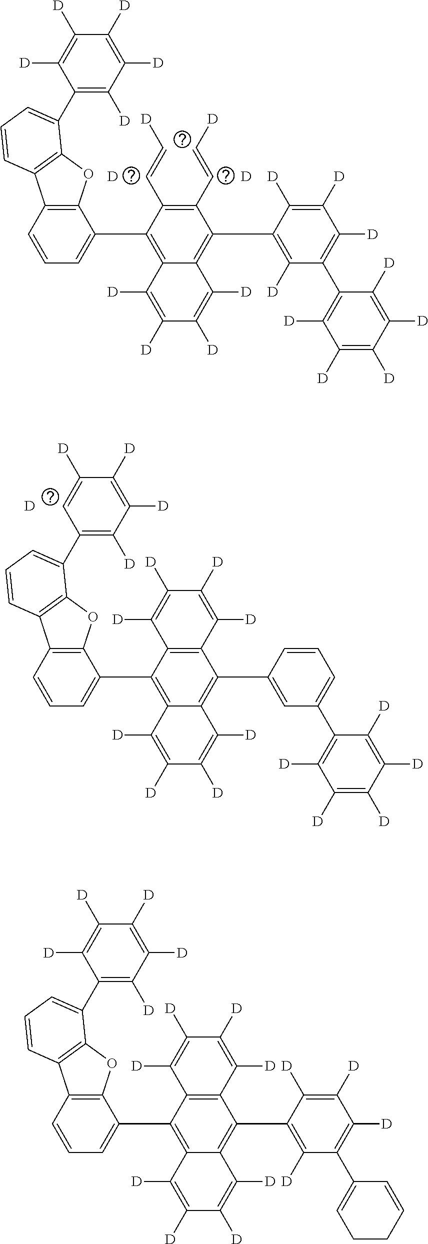

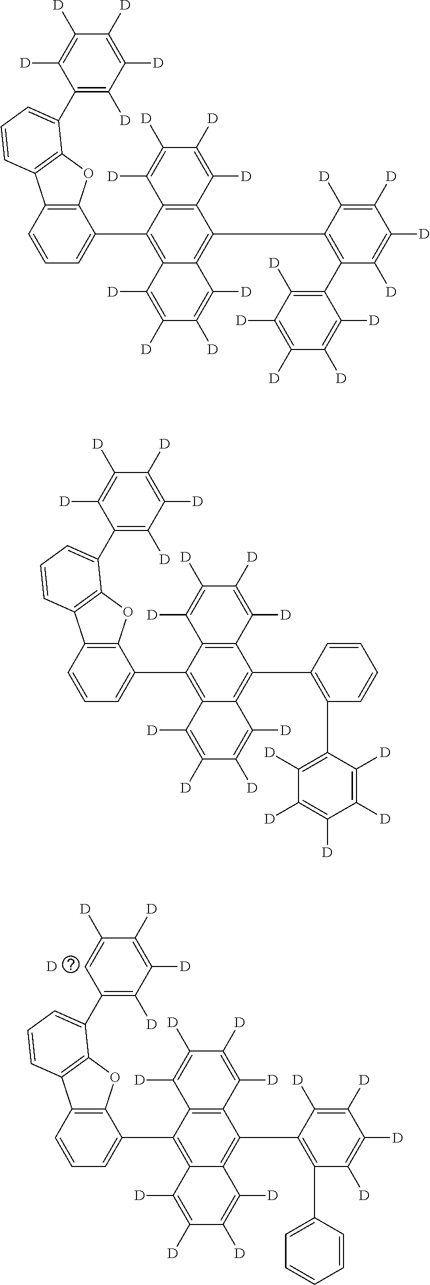

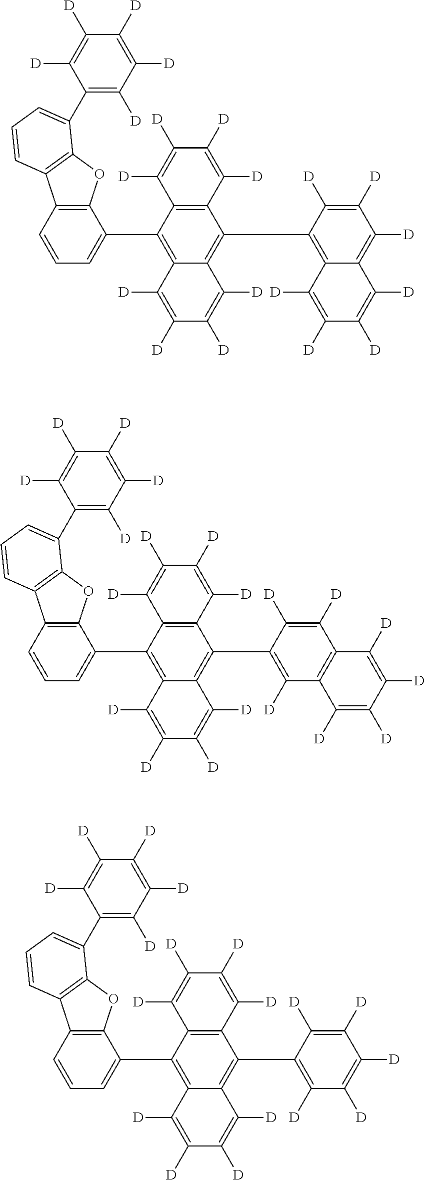

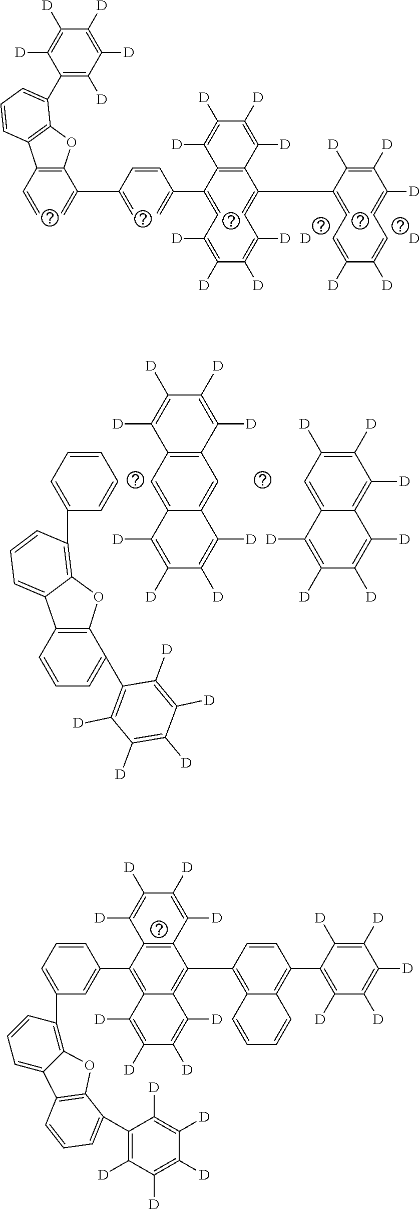

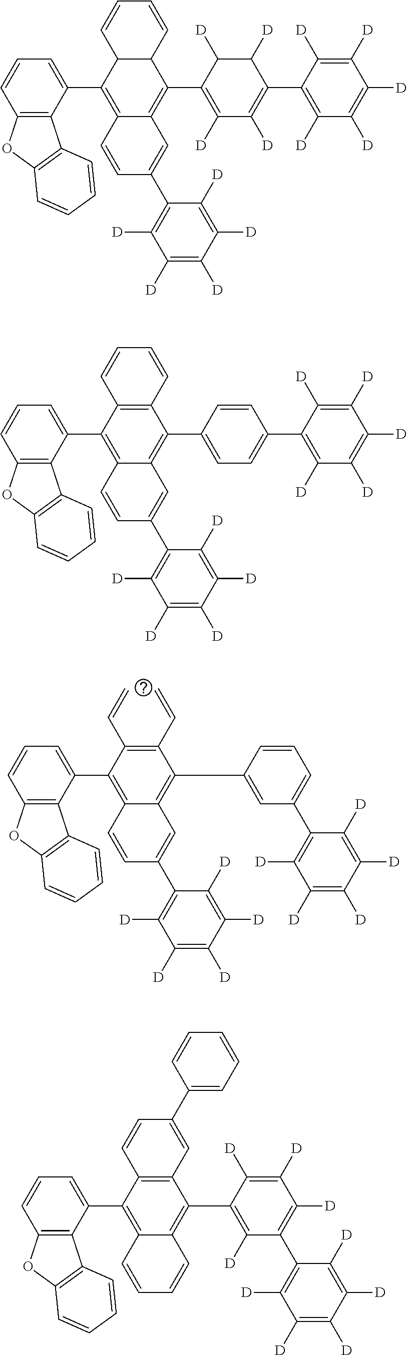

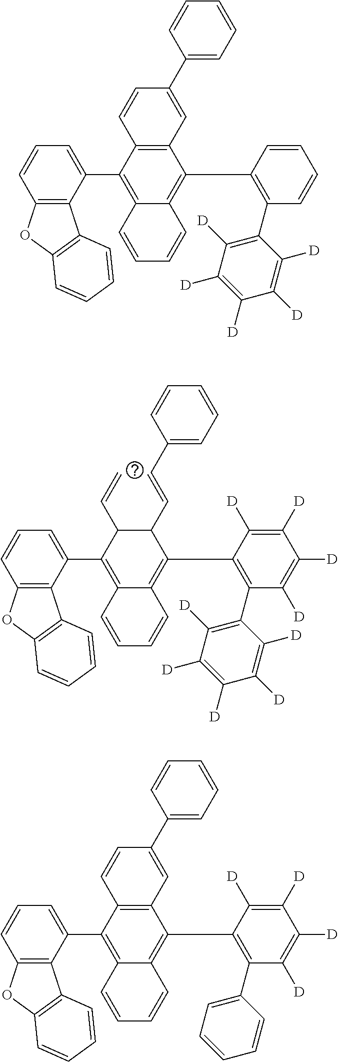

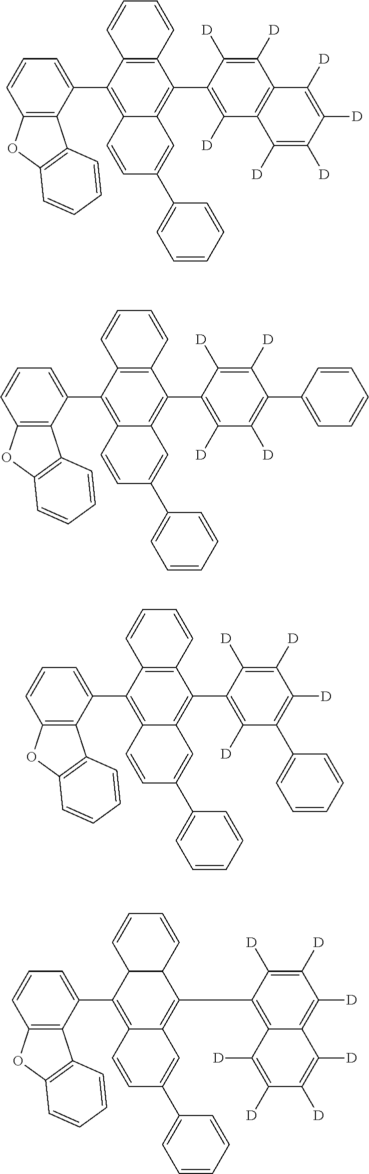

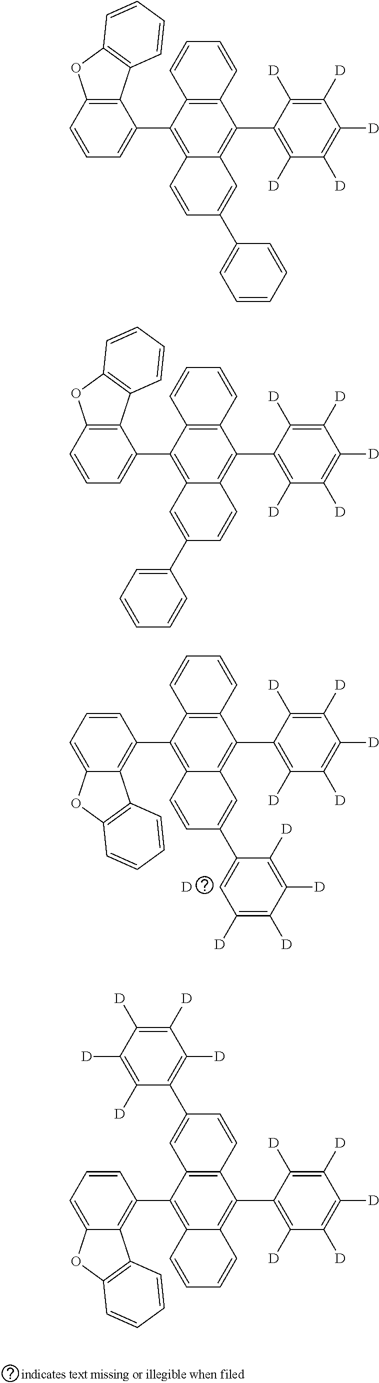

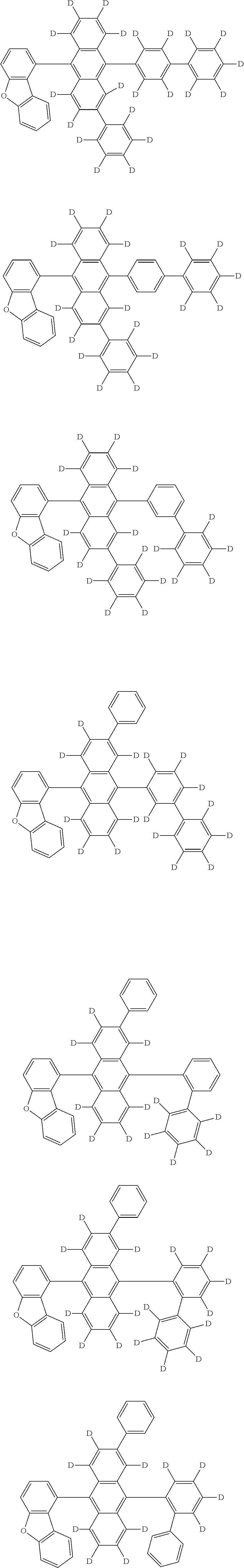

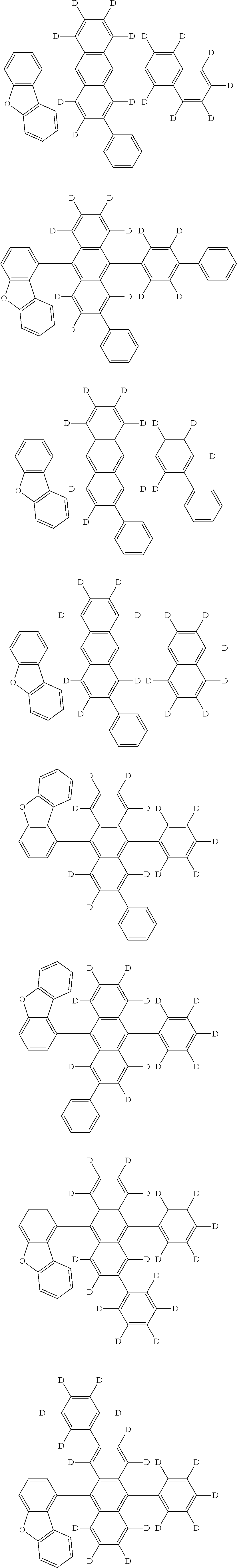

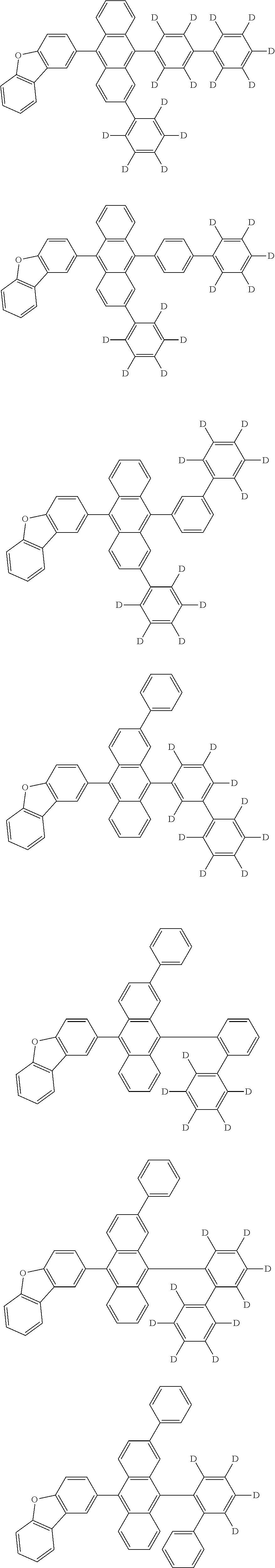

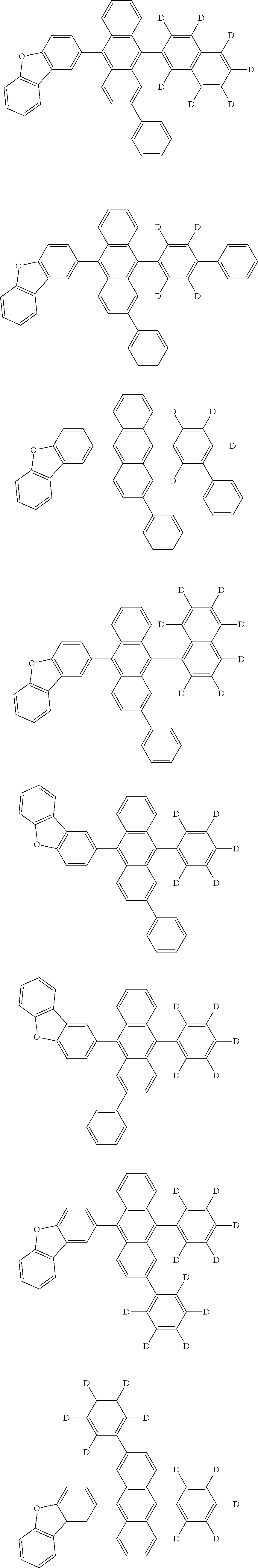

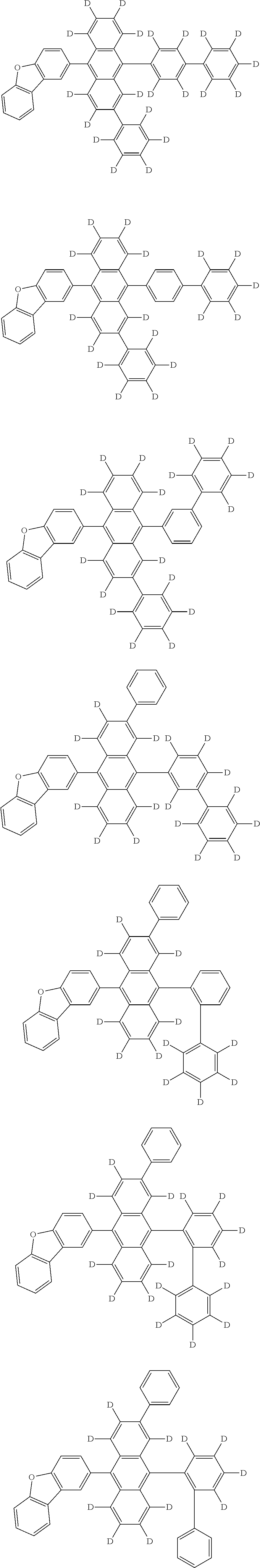

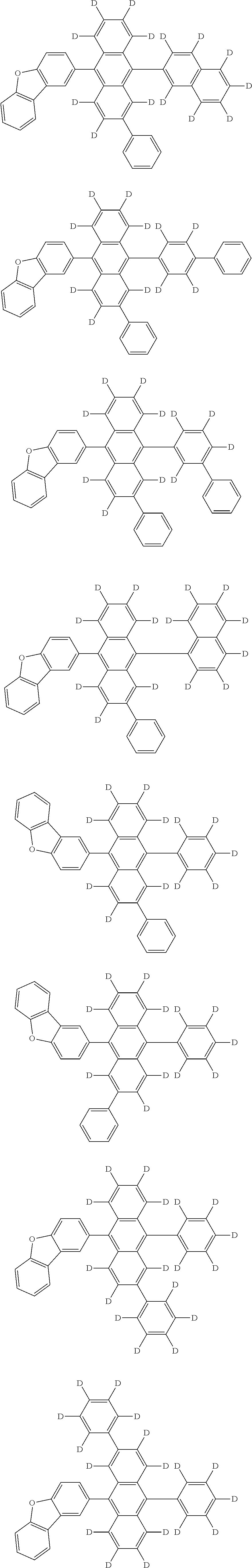

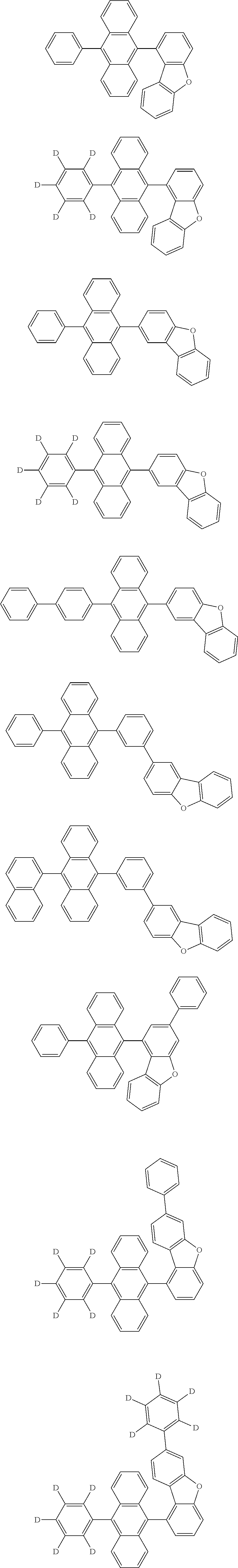

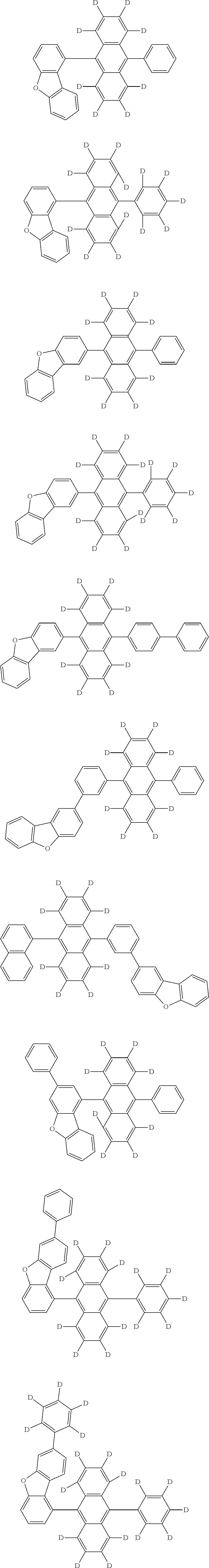

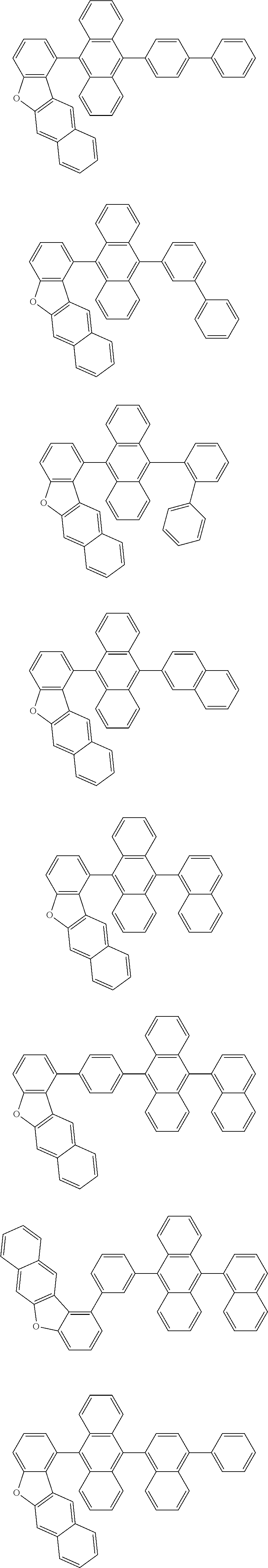

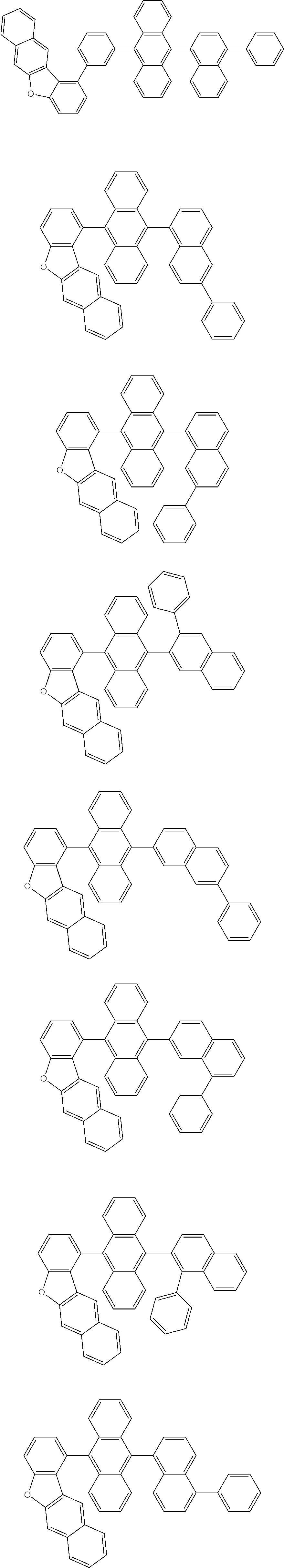

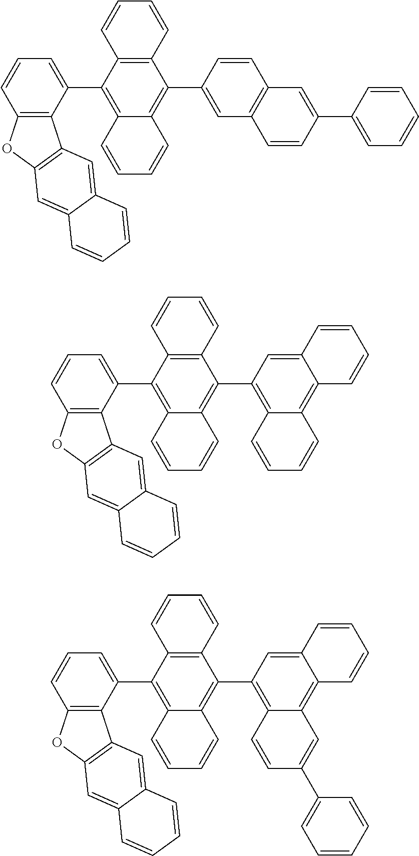

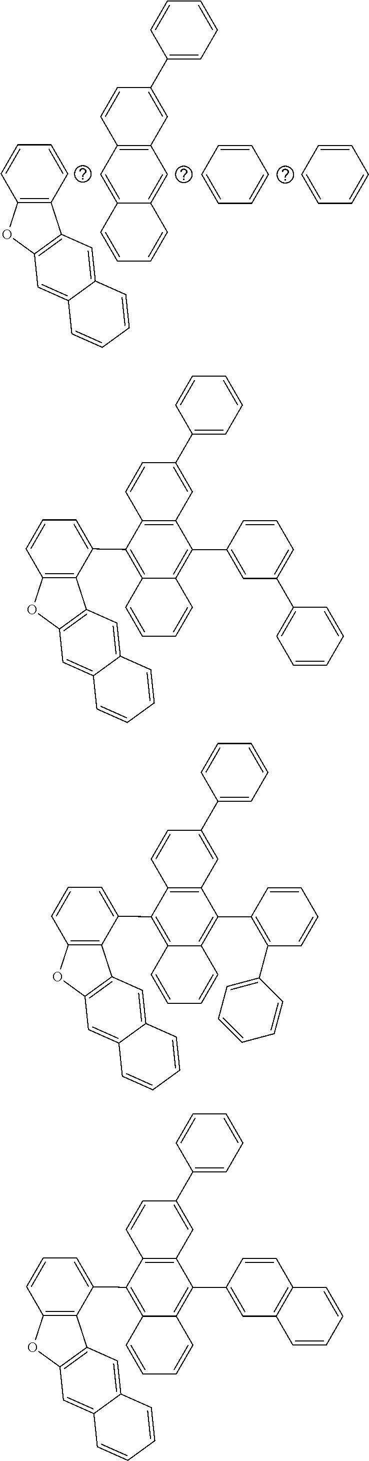

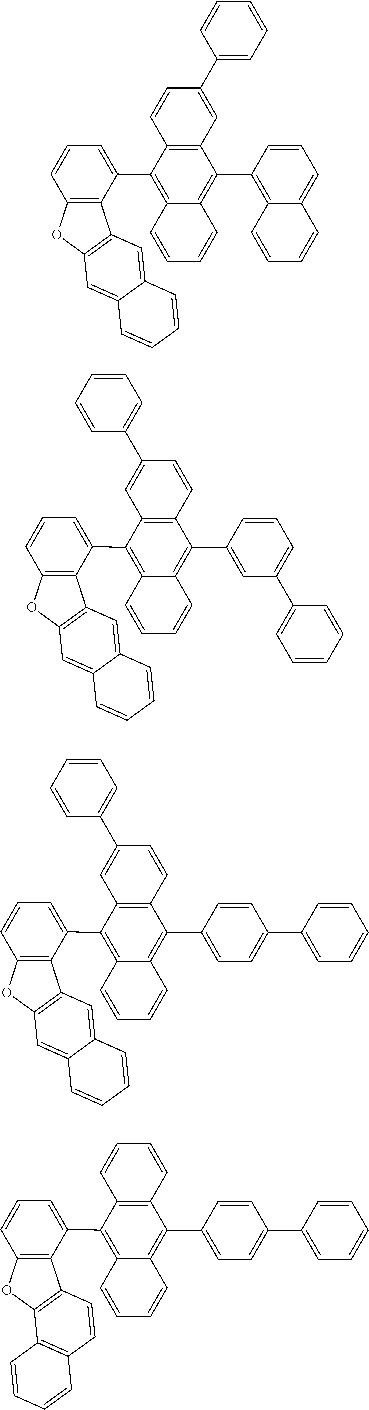

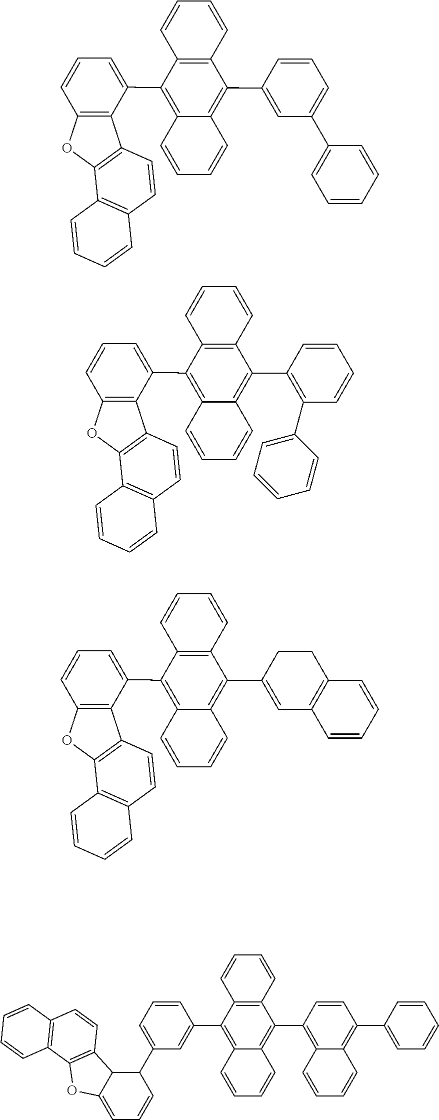 [SMILES: c1ccc(-c2cccc(-c3c4ccccc4c(-c4cccc5oc6c7ccccc7ccc6c45)c4ccccc34)c2)cc1 ; c1ccc(-c2ccccc2-c2c3ccccc3c(-c3cccc4oc5c6ccccc6ccc5c34)c3ccccc23)cc1 ; C1=C(c2c3ccccc3c(-c3cccc4oc5c6ccccc6ccc5c34)c3ccccc23)CCc2ccccc21 ; C1=CC(c2cccc(-c3c4ccccc4c(-c4ccc(-c5ccccc5)c5ccccc45)c4ccccc34)c2)C2C(=C1)Oc1c2ccc2ccccc12]

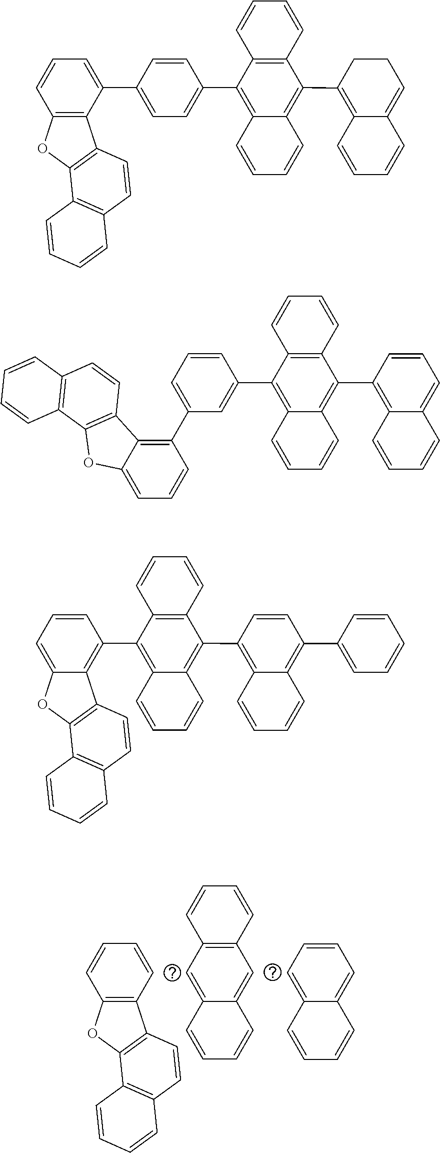

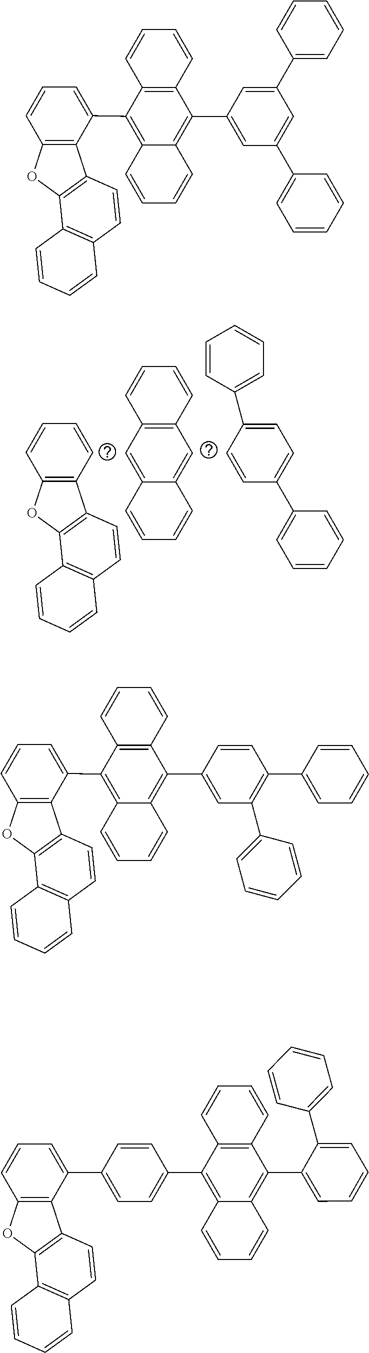

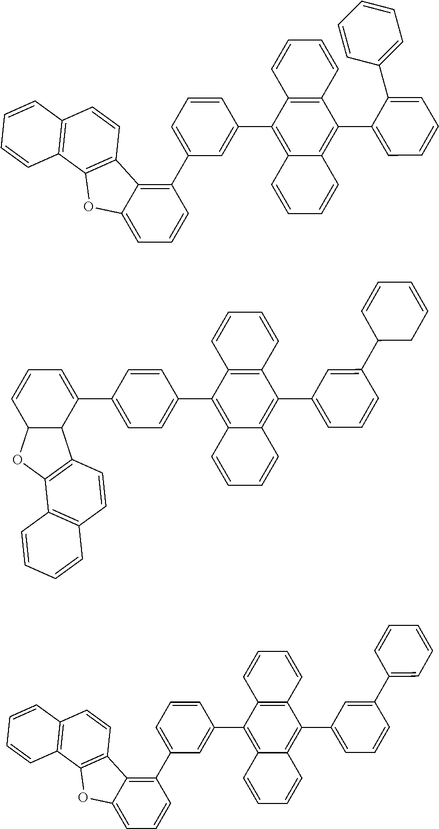 [SMILES: c1ccc(-c2ccccc2-c2c3ccccc3c(-c3cccc(-c4cccc5oc6c7ccccc7ccc6c45)c3)c3ccccc23)cc1 ; C1=CCC(c2cccc(-c3c4ccccc4c(-c4ccc(C5=CC=CC6Oc7c(ccc8ccccc78)C56)cc4)c4ccccc34)c2)C=C1 ; c1ccc(-c2cccc(-c3c4ccccc4c(-c4cccc(-c5cccc6oc7c8ccccc8ccc7c56)c4)c4ccccc34)c2)cc1]

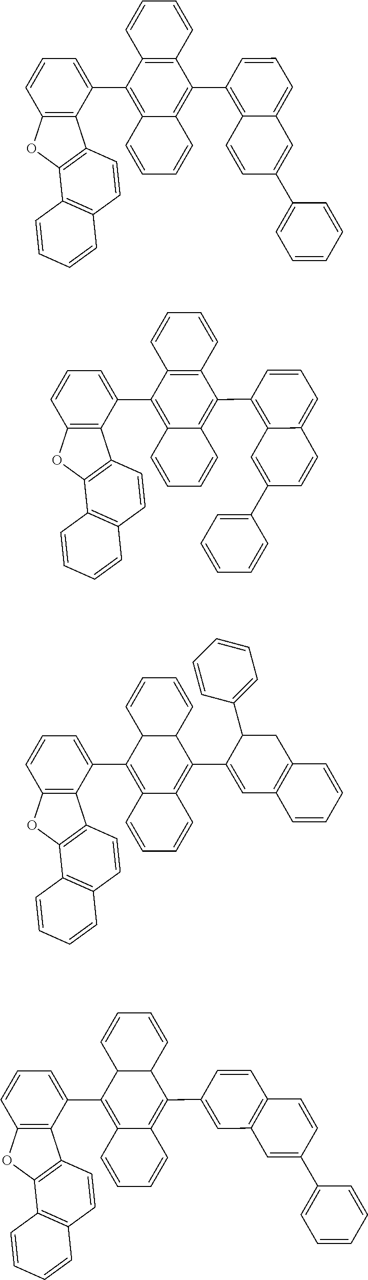 [SMILES: c1ccc(-c2ccc3c(-c4c5ccccc5c(-c5cccc6oc7c8ccccc8ccc7c56)c5ccccc45)cccc3c2)cc1 ; c1ccc(-c2ccc3cccc(-c4c5ccccc5c(-c5cccc6oc7c8ccccc8ccc7c56)c5ccccc45)c3c2)cc1 ; C1=CC2C(C3=Cc4ccccc4CC3c3ccccc3)=c3ccccc3=C(c3cccc4oc5c6ccccc6ccc5c34)C2C=C1 ; C1=CC2C(c3ccc4ccc(-c5ccccc5)cc4c3)=c3ccccc3=C(c3cccc4oc5c6ccccc6ccc5c34)C2C=C1]

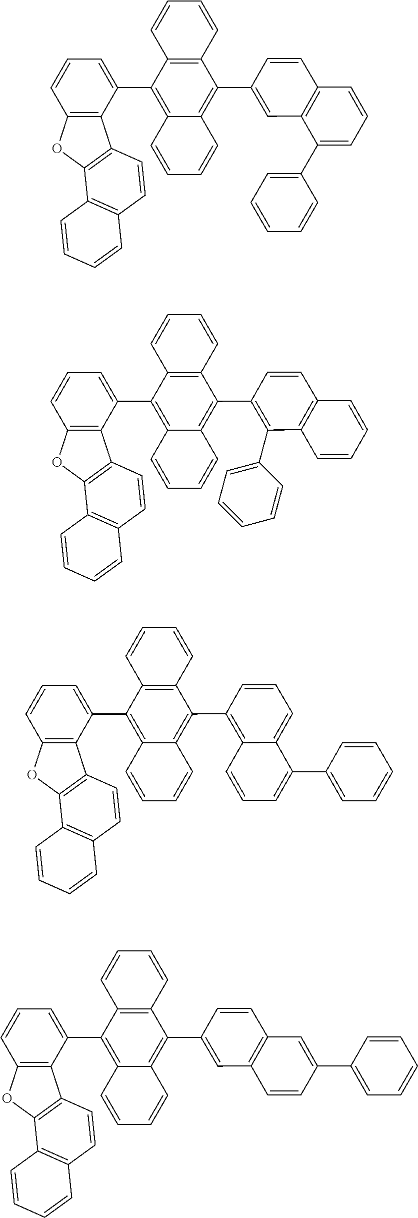 [SMILES: c1ccc(-c2cccc3ccc(-c4c5ccccc5c(-c5cccc6oc7c8ccccc8ccc7c56)c5ccccc45)cc23)cc1 ; c1ccc(-c2c(-c3c4ccccc4c(-c4cccc5oc6c7ccccc7ccc6c45)c4ccccc34)ccc3ccccc23)cc1 ; c1ccc(-c2cccc3c(-c4c5ccccc5c(-c5cccc6oc7c8ccccc8ccc7c56)c5ccccc45)cccc23)cc1 ; c1ccc(-c2ccc3cc(-c4c5ccccc5c(-c5cccc6oc7c8ccccc8ccc7c56)c5ccccc45)ccc3c2)cc1]

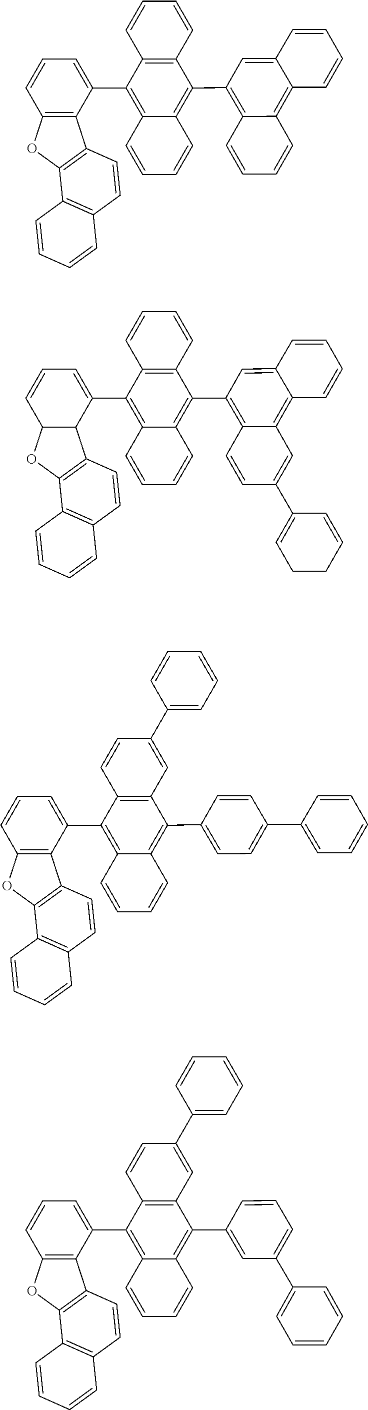 [SMILES: c1ccc2c(c1)cc(-c1c3ccccc3c(-c3cccc4oc5c6ccccc6ccc5c34)c3ccccc13)c1ccccc12 ; C1=CC2Oc3c(ccc4ccccc34)C2C(c2c3ccccc3c(-c3cc4ccccc4c4cc(C5=CCCC=C5)ccc34)c3ccccc23)=C1 ; c1ccc(-c2ccc(-c3c4ccccc4c(-c4cccc5oc6c7ccccc7ccc6c45)c4ccc(-c5ccccc5)cc34)cc2)cc1 ; c1ccc(-c2cccc(-c3c4ccccc4c(-c4cccc5oc6c7ccccc7ccc6c45)c4ccc(-c5ccccc5)cc34)c2)cc1]

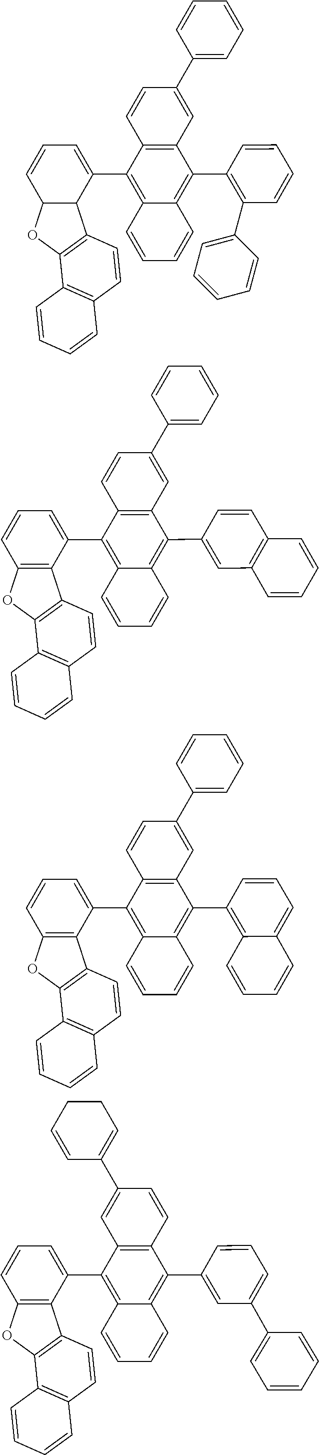

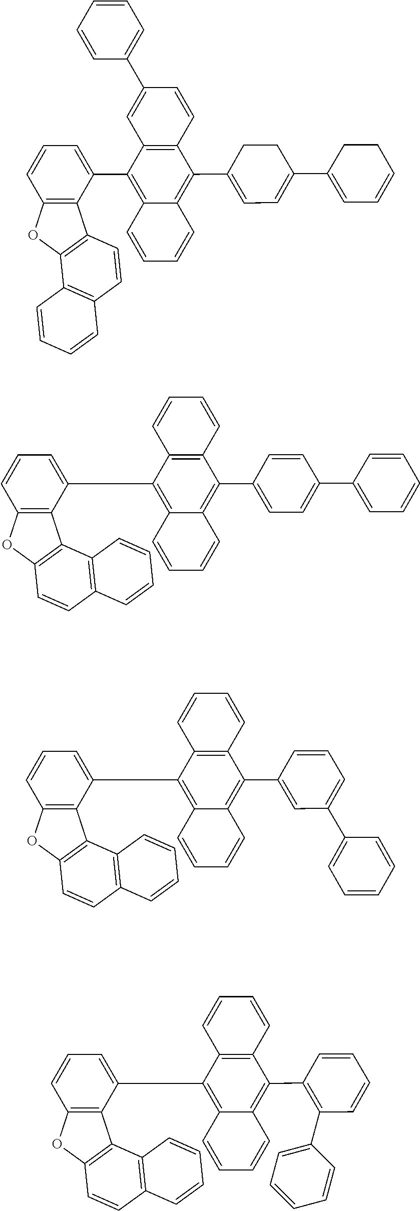 [SMILES: C1=CCCC(C2=CC=C(c3c4ccccc4c(-c4cccc5oc6c7ccccc7ccc6c45)c4cc(-c5ccccc5)ccc34)CC2)=C1 ; c1ccc(-c2ccc(-c3c4ccccc4c(-c4cccc5oc6ccc7ccccc7c6c45)c4ccccc34)cc2)cc1 ; c1ccc(-c2cccc(-c3c4ccccc4c(-c4cccc5oc6ccc7ccccc7c6c45)c4ccccc34)c2)cc1 ; c1ccc(-c2ccccc2-c2c3ccccc3c(-c3cccc4oc5ccc6ccccc6c5c34)c3ccccc23)cc1]

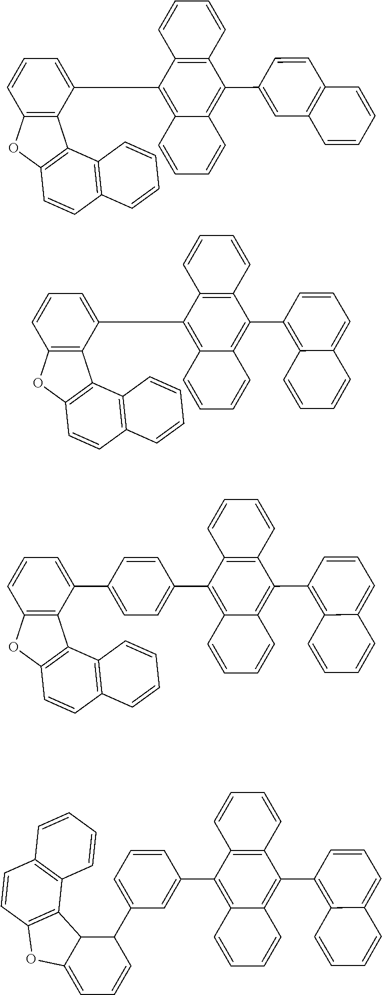 [SMILES: c1ccc2cc(-c3c4ccccc4c(-c4cccc5oc6ccc7ccccc7c6c45)c4ccccc34)ccc2c1 ; c1ccc2c(-c3c4ccccc4c(-c4cccc5oc6ccc7ccccc7c6c45)c4ccccc34)cccc2c1 ; c1ccc2c(-c3c4ccccc4c(-c4ccc(-c5cccc6oc7ccc8ccccc8c7c56)cc4)c4ccccc34)cccc2c1 ; C1=CC(c2cccc(-c3c4ccccc4c(-c4cccc5ccccc45)c4ccccc34)c2)C2C(=C1)Oc1ccc3ccccc3c12]

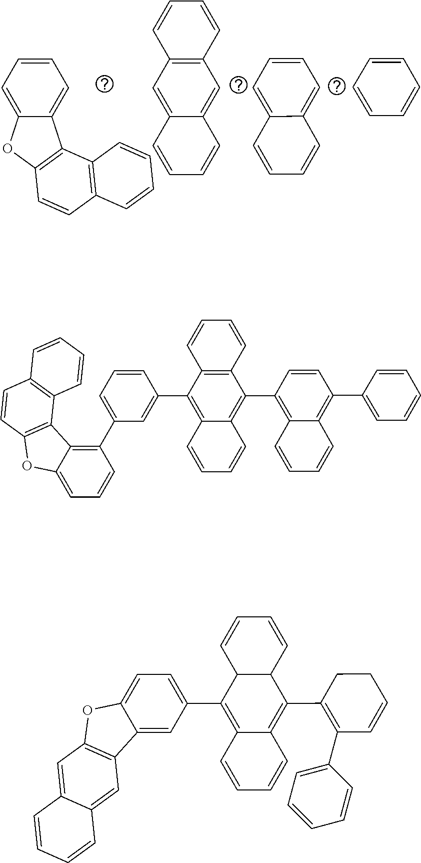

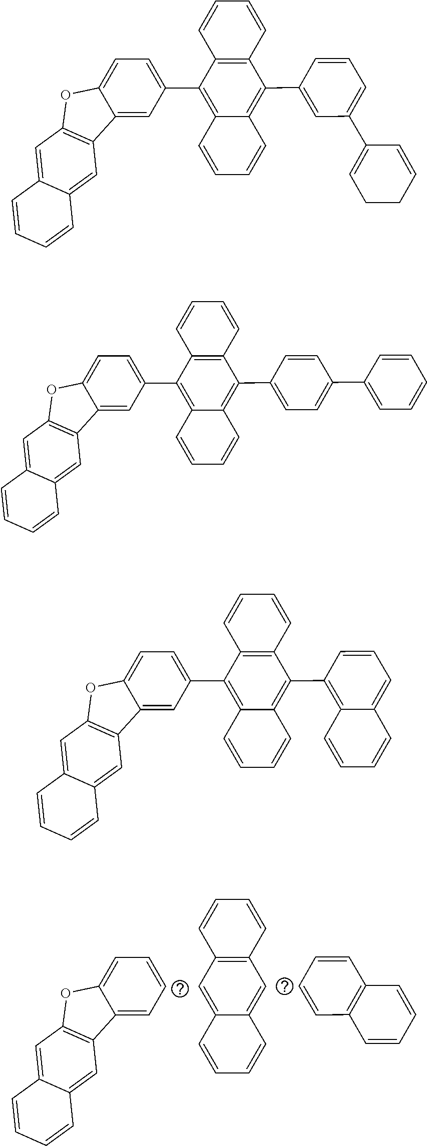

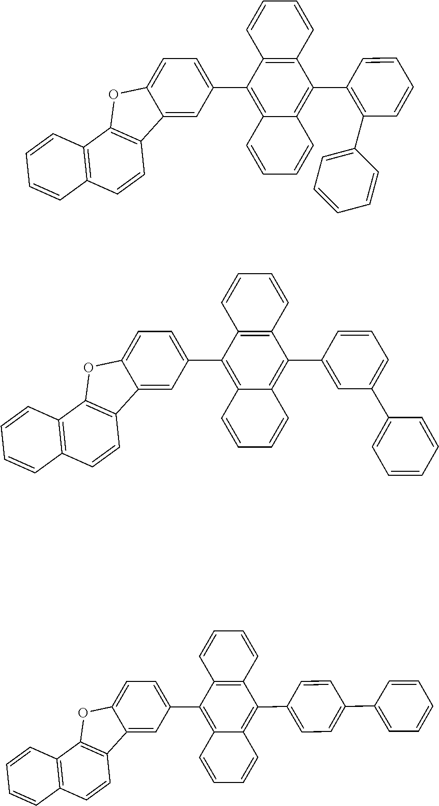 [SMILES: c1ccc(-c2ccccc2-c2c3ccccc3c(-c3ccc4oc5c6ccccc6ccc5c4c3)c3ccccc23)cc1 ; c1ccc(-c2cccc(-c3c4ccccc4c(-c4ccc5oc6c7ccccc7ccc6c5c4)c4ccccc34)c2)cc1 ; c1ccc(-c2ccc(-c3c4ccccc4c(-c4ccc5oc6c7ccccc7ccc6c5c4)c4ccccc34)cc2)cc1]

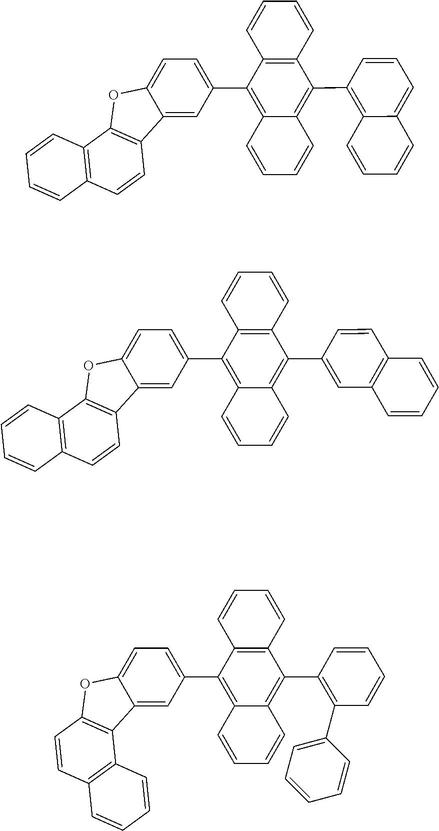 [SMILES: c1ccc2c(-c3c4ccccc4c(-c4ccc5oc6c7ccccc7ccc6c5c4)c4ccccc34)cccc2c1 ; c1ccc2cc(-c3c4ccccc4c(-c4ccc5oc6c7ccccc7ccc6c5c4)c4ccccc34)ccc2c1 ; c1ccc(-c2ccccc2-c2c3ccccc3c(-c3ccc4oc5ccc6ccccc6c5c4c3)c3ccccc23)cc1]

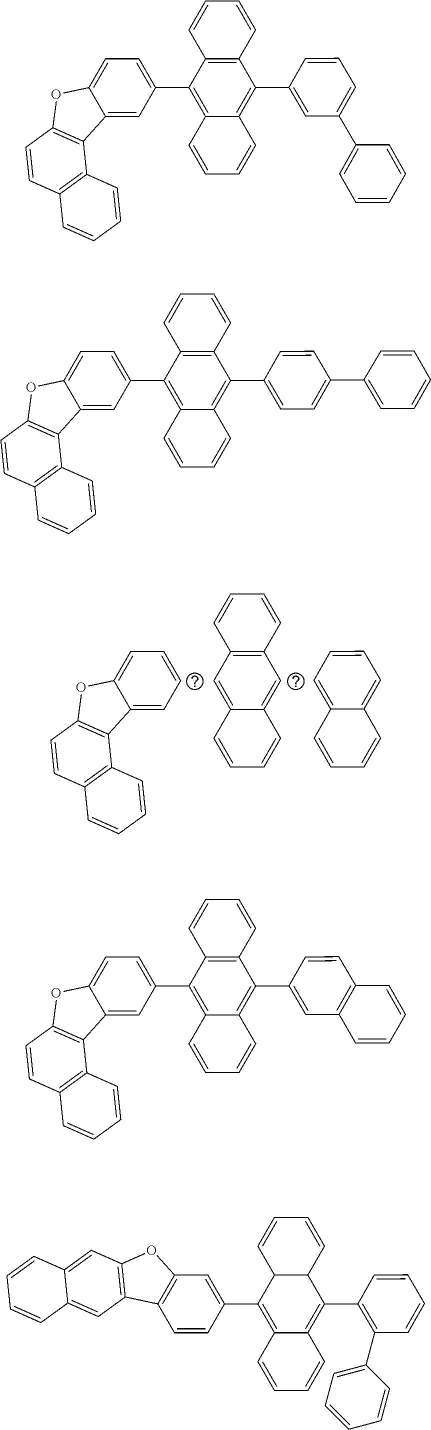

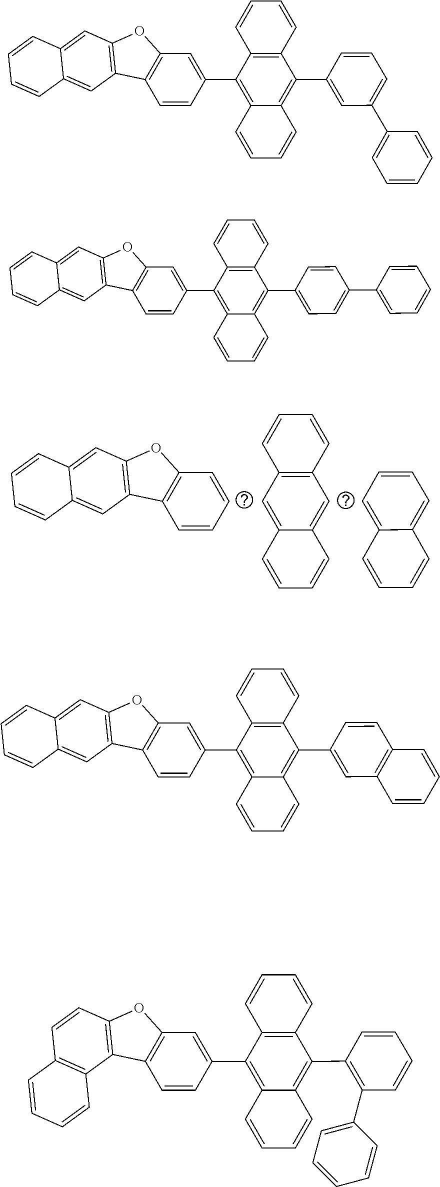

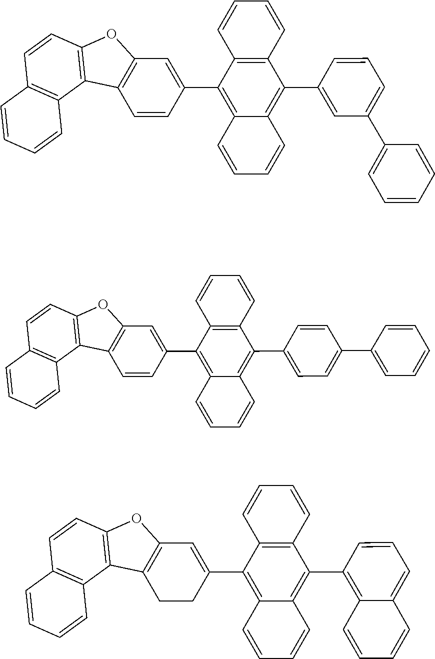 [SMILES: c1ccc(-c2cccc(-c3c4ccccc4c(-c4ccc5c(c4)oc4ccc6ccccc6c45)c4ccccc34)c2)cc1 ; c1ccc(-c2ccc(-c3c4ccccc4c(-c4ccc5c(c4)oc4ccc6ccccc6c45)c4ccccc34)cc2)cc1 ; C1=C(c2c3ccccc3c(-c3cccc4ccccc34)c3ccccc23)CCc2c1oc1ccc3ccccc3c21]

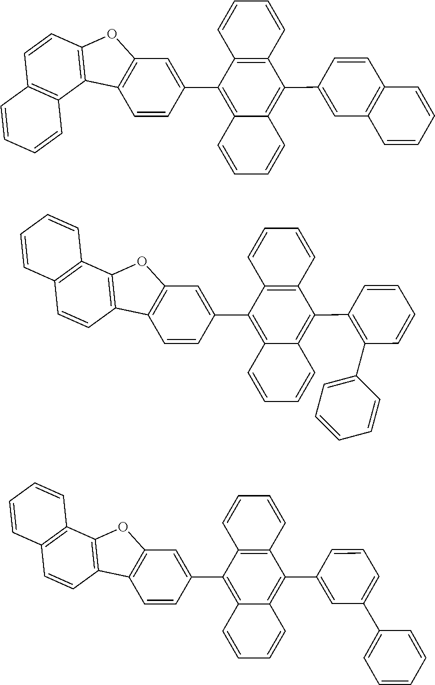 [SMILES: c1ccc2cc(-c3c4ccccc4c(-c4ccc5c(c4)oc4ccc6ccccc6c45)c4ccccc34)ccc2c1 ; c1ccc(-c2ccccc2-c2c3ccccc3c(-c3ccc4c(c3)oc3c5ccccc5ccc43)c3ccccc23)cc1 ; c1ccc(-c2cccc(-c3c4ccccc4c(-c4ccc5c(c4)oc4c6ccccc6ccc54)c4ccccc34)c2)cc1]

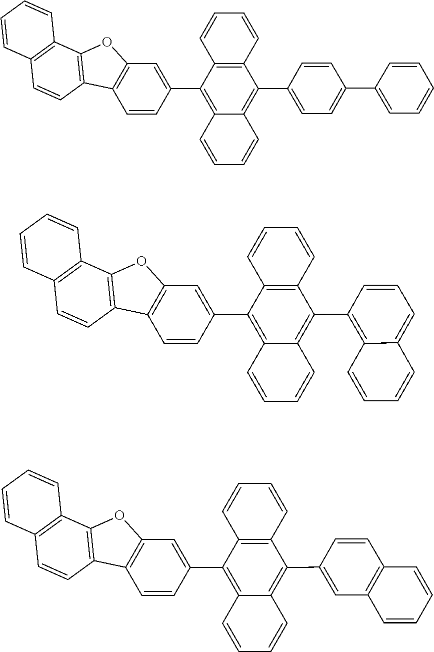 [SMILES: c1ccc(-c2ccc(-c3c4ccccc4c(-c4ccc5c(c4)oc4c6ccccc6ccc54)c4ccccc34)cc2)cc1 ; c1ccc2c(-c3c4ccccc4c(-c4ccc5c(c4)oc4c6ccccc6ccc54)c4ccccc34)cccc2c1 ; c1ccc2cc(-c3c4ccccc4c(-c4ccc5c(c4)oc4c6ccccc6ccc54)c4ccccc34)ccc2c1]

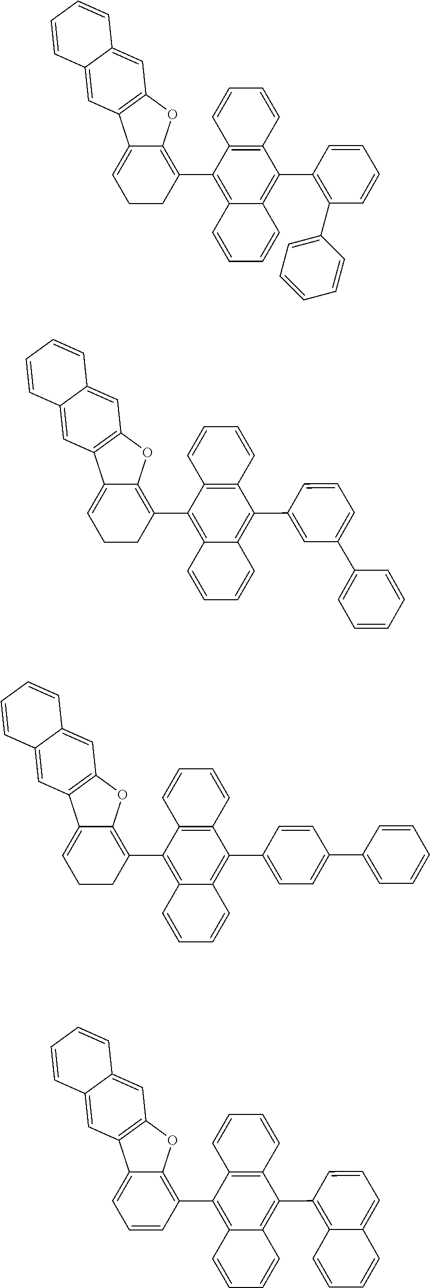 [SMILES: C1=c2c(oc3cc4ccccc4cc23)=C(c2c3ccccc3c(-c3ccccc3-c3ccccc3)c3ccccc23)CC1 ; C1=c2c(oc3cc4ccccc4cc23)=C(c2c3ccccc3c(-c3cccc(-c4ccccc4)c3)c3ccccc23)CC1 ; C1=c2c(oc3cc4ccccc4cc23)=C(c2c3ccccc3c(-c3ccc(-c4ccccc4)cc3)c3ccccc23)CC1 ; c1ccc2cc3c(cc2c1)oc1c(-c2c4ccccc4c(-c4cccc5ccccc45)c4ccccc24)cccc13]

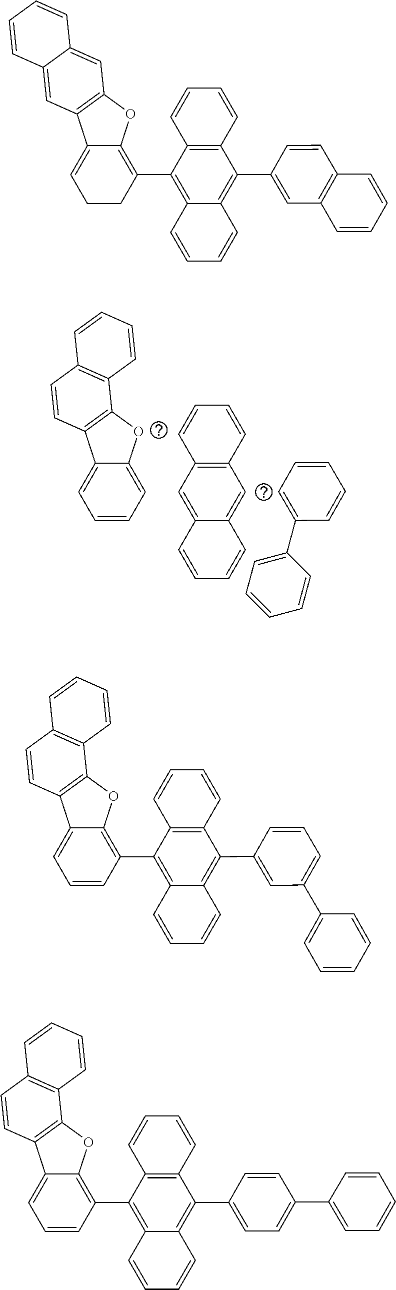

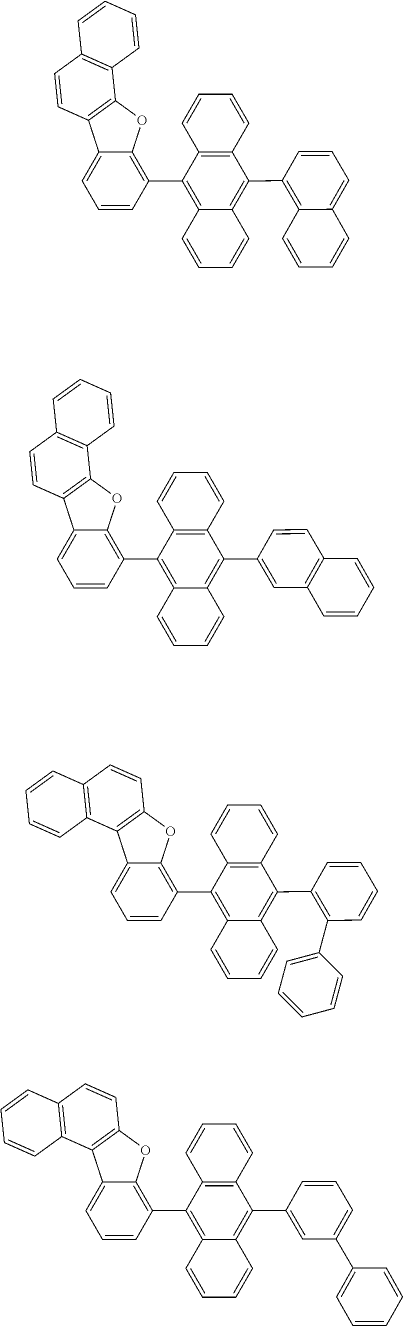 [SMILES: c1ccc2c(-c3c4ccccc4c(-c4cccc5c4oc4c6ccccc6ccc54)c4ccccc34)cccc2c1 ; c1ccc2cc(-c3c4ccccc4c(-c4cccc5c4oc4c6ccccc6ccc54)c4ccccc34)ccc2c1 ; c1ccc(-c2ccccc2-c2c3ccccc3c(-c3cccc4c3oc3ccc5ccccc5c34)c3ccccc23)cc1 ; c1ccc(-c2cccc(-c3c4ccccc4c(-c4cccc5c4oc4ccc6ccccc6c45)c4ccccc34)c2)cc1]

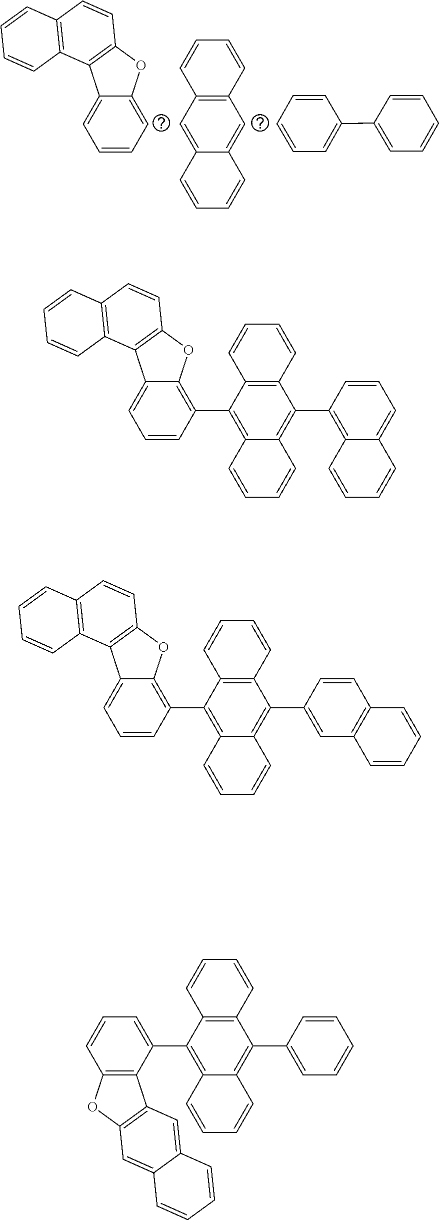

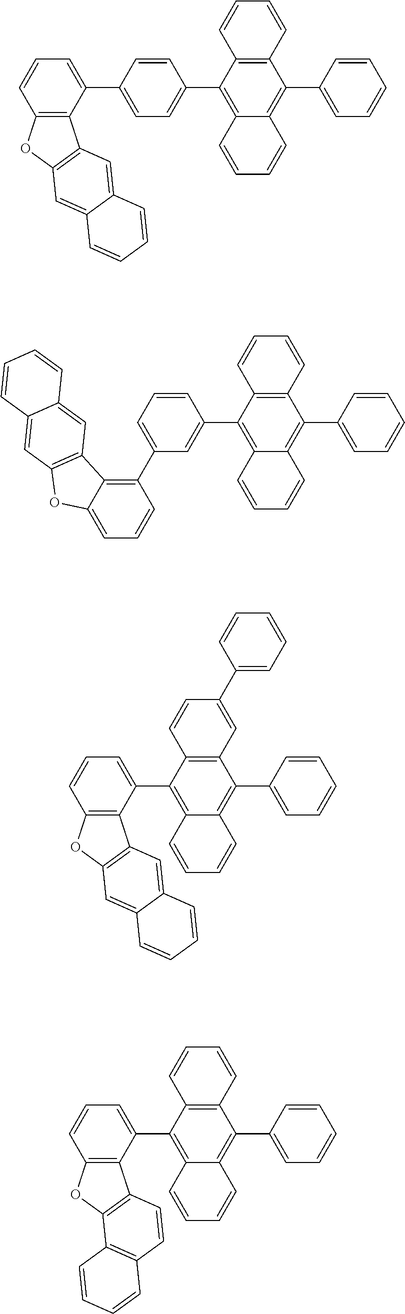 [SMILES: c1ccc(-c2c3ccccc3c(-c3ccc(-c4cccc5oc6cc7ccccc7cc6c45)cc3)c3ccccc23)cc1 ; c1ccc(-c2c3ccccc3c(-c3cccc(-c4cccc5oc6cc7ccccc7cc6c45)c3)c3ccccc23)cc1 ; c1ccc(-c2ccc3c(-c4cccc5oc6cc7ccccc7cc6c45)c4ccccc4c(-c4ccccc4)c3c2)cc1 ; c1ccc(-c2c3ccccc3c(-c3cccc4oc5c6ccccc6ccc5c34)c3ccccc23)cc1]

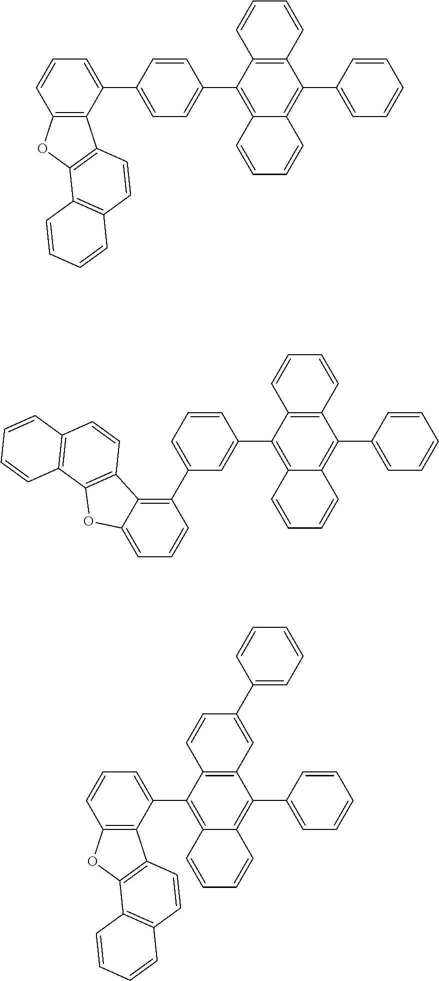 [SMILES: c1ccc(-c2c3ccccc3c(-c3ccc(-c4cccc5oc6c7ccccc7ccc6c45)cc3)c3ccccc23)cc1 ; c1ccc(-c2c3ccccc3c(-c3cccc(-c4cccc5oc6c7ccccc7ccc6c45)c3)c3ccccc23)cc1 ; c1ccc(-c2ccc3c(-c4cccc5oc6c7ccccc7ccc6c45)c4ccccc4c(-c4ccccc4)c3c2)cc1]

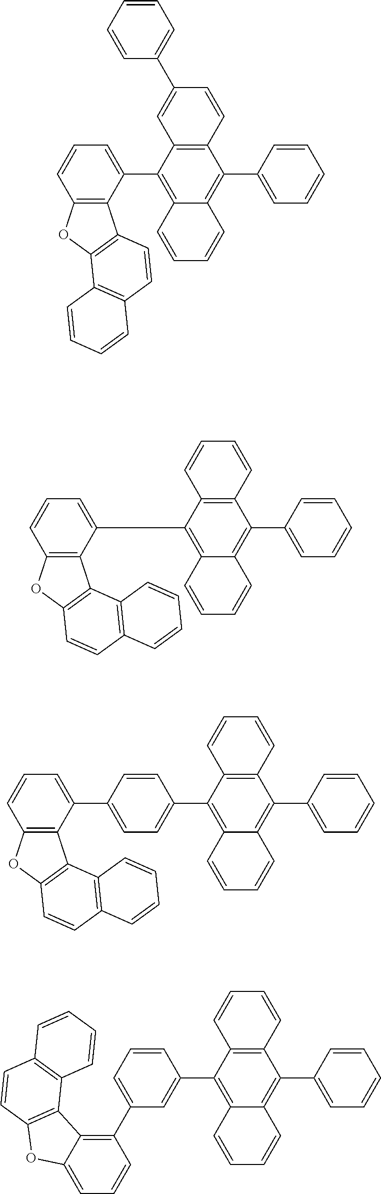 [SMILES: c1ccc(-c2ccc3c(-c4ccccc4)c4ccccc4c(-c4cccc5oc6c7ccccc7ccc6c45)c3c2)cc1 ; c1ccc(-c2c3ccccc3c(-c3cccc4oc5ccc6ccccc6c5c34)c3ccccc23)cc1 ; c1ccc(-c2c3ccccc3c(-c3ccc(-c4cccc5oc6ccc7ccccc7c6c45)cc3)c3ccccc23)cc1 ; c1ccc(-c2c3ccccc3c(-c3cccc(-c4cccc5oc6ccc7ccccc7c6c45)c3)c3ccccc23)cc1]

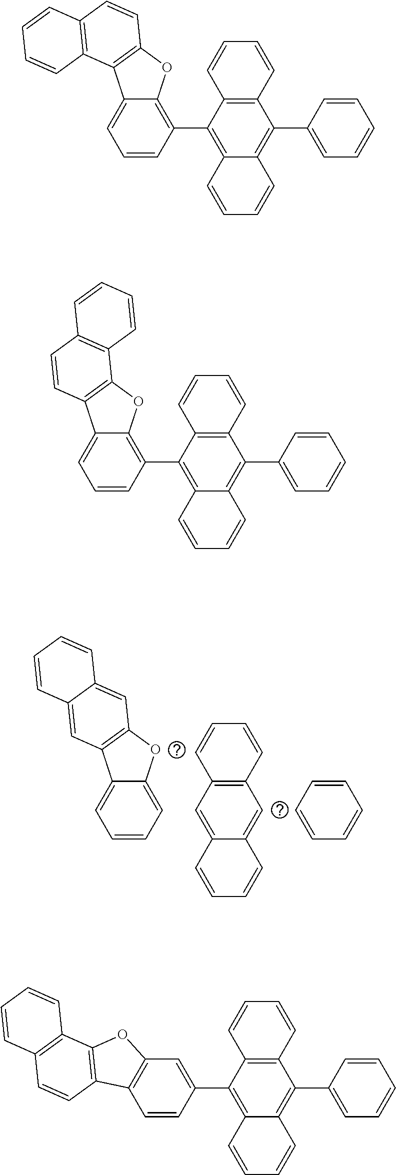

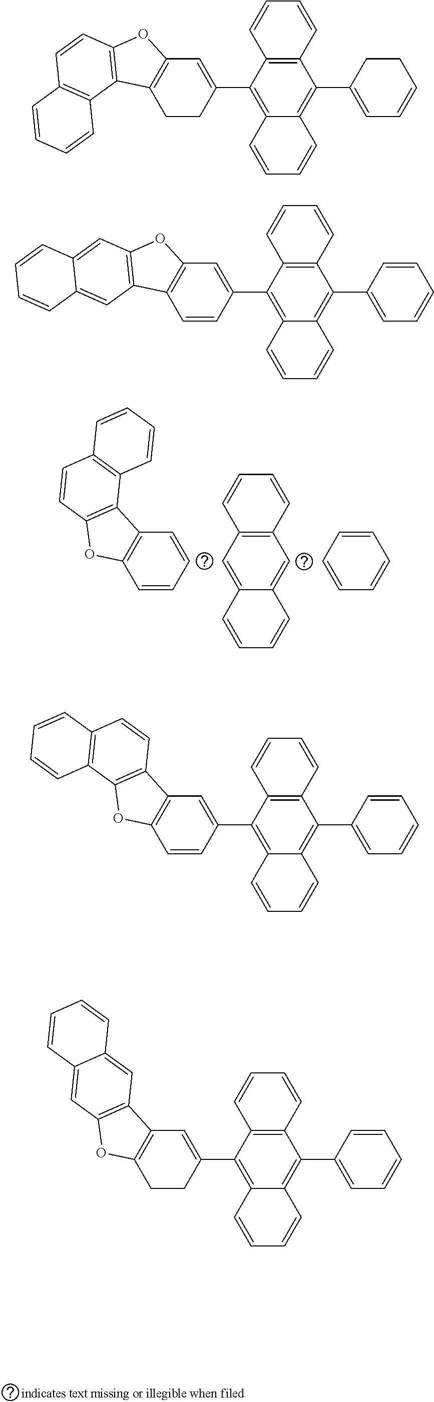

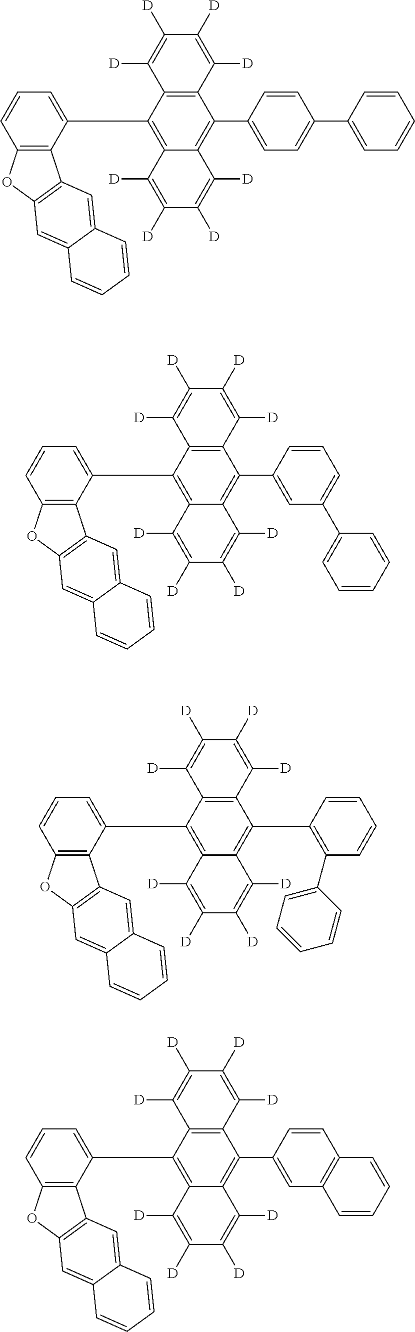 [SMILES: [2H]c1c([2H])c([2H])c2c(-c3cccc4oc5cc6ccccc6cc5c34)c3c([2H])c([2H])c([2H])c([2H])c3c(-c3ccc(-c4ccccc4)cc3)c2c1[2H] ; [2H]c1c([2H])c([2H])c2c(-c3cccc4oc5cc6ccccc6cc5c34)c3c([2H])c([2H])c([2H])c([2H])c3c(-c3cccc(-c4ccccc4)c3)c2c1[2H] ; [2H]c1c([2H])c([2H])c2c(-c3cccc4oc5cc6ccccc6cc5c34)c3c([2H])c([2H])c([2H])c([2H])c3c(-c3ccccc3-c3ccccc3)c2c1[2H] ; [2H]c1c([2H])c([2H])c2c(-c3cccc4oc5cc6ccccc6cc5c34)c3c([2H])c([2H])c([2H])c([2H])c3c(-c3ccc4ccccc4c3)c2c1[2H]]

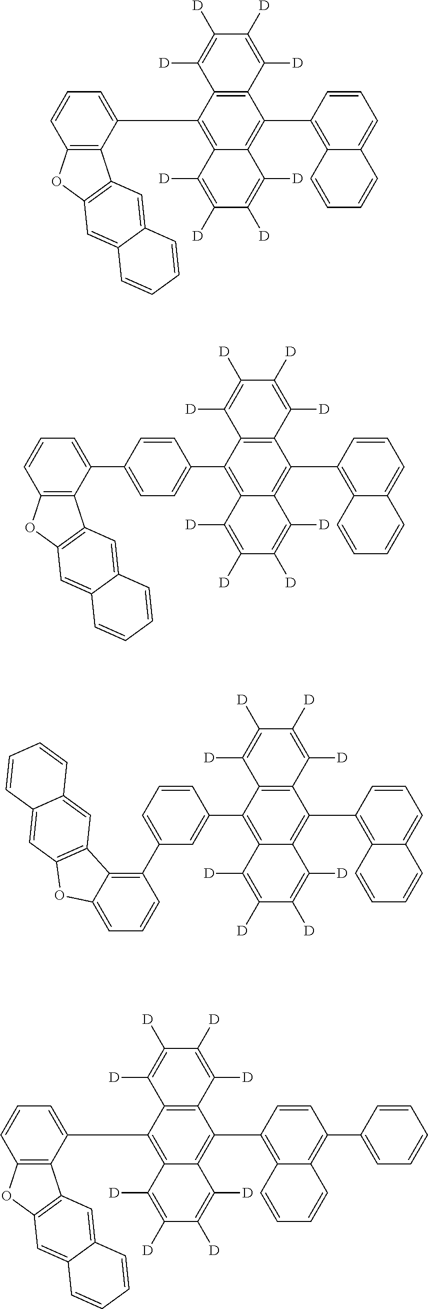 [SMILES: [2H]c1c([2H])c([2H])c2c(-c3cccc4oc5cc6ccccc6cc5c34)c3c([2H])c([2H])c([2H])c([2H])c3c(-c3cccc4ccccc34)c2c1[2H] ; [2H]c1c([2H])c([2H])c2c(-c3cccc4ccccc34)c3c([2H])c([2H])c([2H])c([2H])c3c(-c3ccc(-c4cccc5oc6cc7ccccc7cc6c45)cc3)c2c1[2H] ; [2H]c1c([2H])c([2H])c2c(-c3cccc4ccccc34)c3c([2H])c([2H])c([2H])c([2H])c3c(-c3cccc(-c4cccc5oc6cc7ccccc7cc6c45)c3)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccccc4)c4ccccc34)c2c1[2H]]

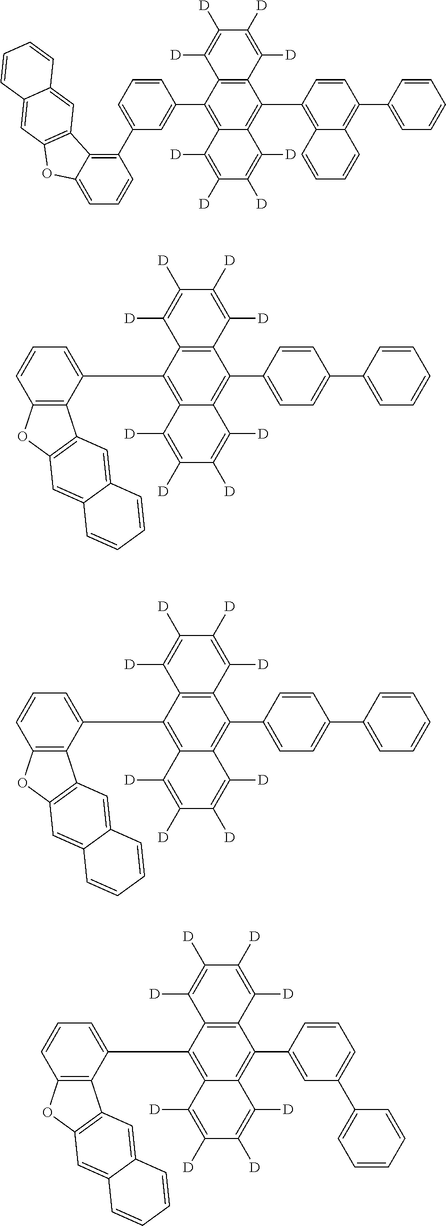 [SMILES: [2H]c1c([2H])c([2H])c2c(-c3ccc(-c4ccccc4)c4ccccc34)c3c([2H])c([2H])c([2H])c([2H])c3c(-c3cccc(-c4cccc5oc6cc7ccccc7cc6c45)c3)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccccc4)cc3)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccccc4)cc3)c2c1[2H] ; [2H]c1c([2H])c([2H])c2c(-c3cccc4oc5cc6ccccc6cc5c34)c3c([2H])c([2H])c([2H])c([2H])c3c(-c3cccc(-c4ccccc4)c3)c2c1[2H]]

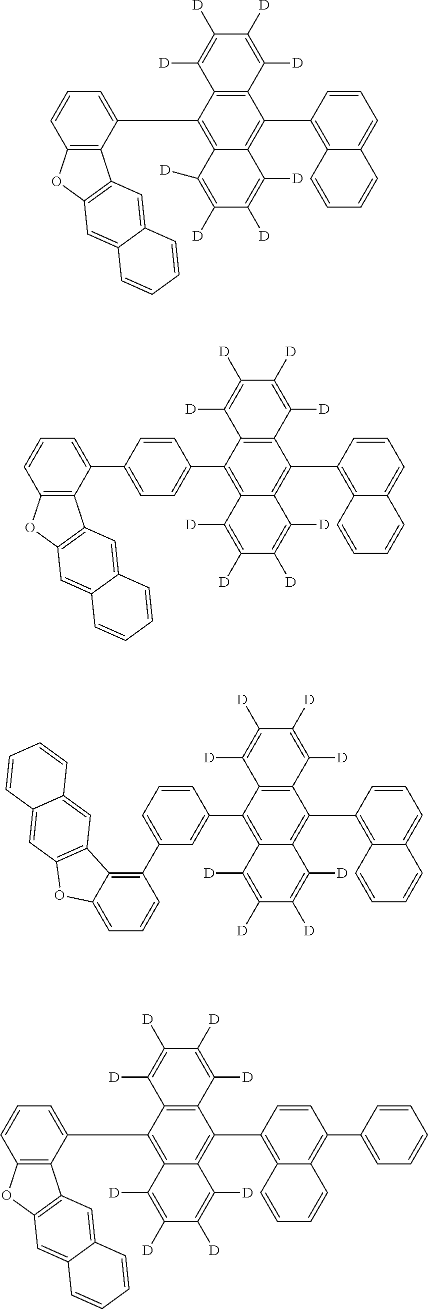 [SMILES: [2H]c1c([2H])c([2H])c2c(-c3cccc4oc5cc6ccccc6cc5c34)c3c([2H])c([2H])c([2H])c([2H])c3c(-c3cccc4ccccc34)c2c1[2H] ; [2H]c1c([2H])c([2H])c2c(-c3cccc4ccccc34)c3c([2H])c([2H])c([2H])c([2H])c3c(-c3ccc(-c4cccc5oc6cc7ccccc7cc6c45)cc3)c2c1[2H] ; [2H]c1c([2H])c([2H])c2c(-c3cccc4ccccc34)c3c([2H])c([2H])c([2H])c([2H])c3c(-c3cccc(-c4cccc5oc6cc7ccccc7cc6c45)c3)c2c1[2H] ; [2H]c1c([2H])c([2H])c2c(-c3cccc4oc5cc6ccccc6cc5c34)c3c([2H])c([2H])c([2H])c([2H])c3c(-c3ccc(-c4ccccc4)c4ccccc34)c2c1[2H]]

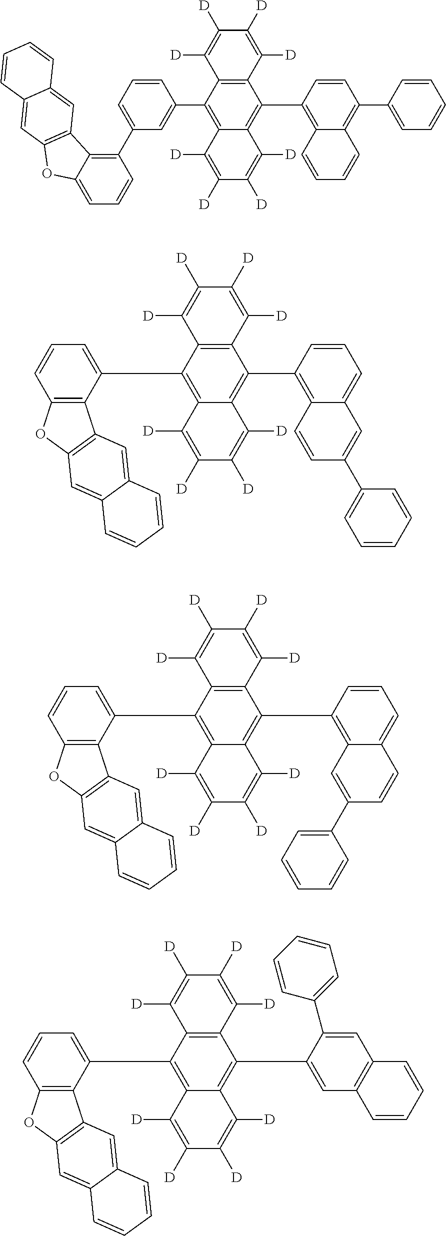 [SMILES: [2H]c1c([2H])c([2H])c2c(-c3ccc(-c4ccccc4)c4ccccc34)c3c([2H])c([2H])c([2H])c([2H])c3c(-c3cccc(-c4cccc5oc6cc7ccccc7cc6c45)c3)c2c1[2H] ; [2H]c1c([2H])c([2H])c2c(-c3cccc4oc5cc6ccccc6cc5c34)c3c([2H])c([2H])c([2H])c([2H])c3c(-c3cccc4cc(-c5ccccc5)ccc34)c2c1[2H] ; [2H]c1c([2H])c([2H])c2c(-c3cccc4oc5cc6ccccc6cc5c34)c3c([2H])c([2H])c([2H])c([2H])c3c(-c3cccc4ccc(-c5ccccc5)cc34)c2c1[2H] ; [2H]c1c([2H])c([2H])c2c(-c3cccc4oc5cc6ccccc6cc5c34)c3c([2H])c([2H])c([2H])c([2H])c3c(-c3cc4ccccc4cc3-c3ccccc3)c2c1[2H]]

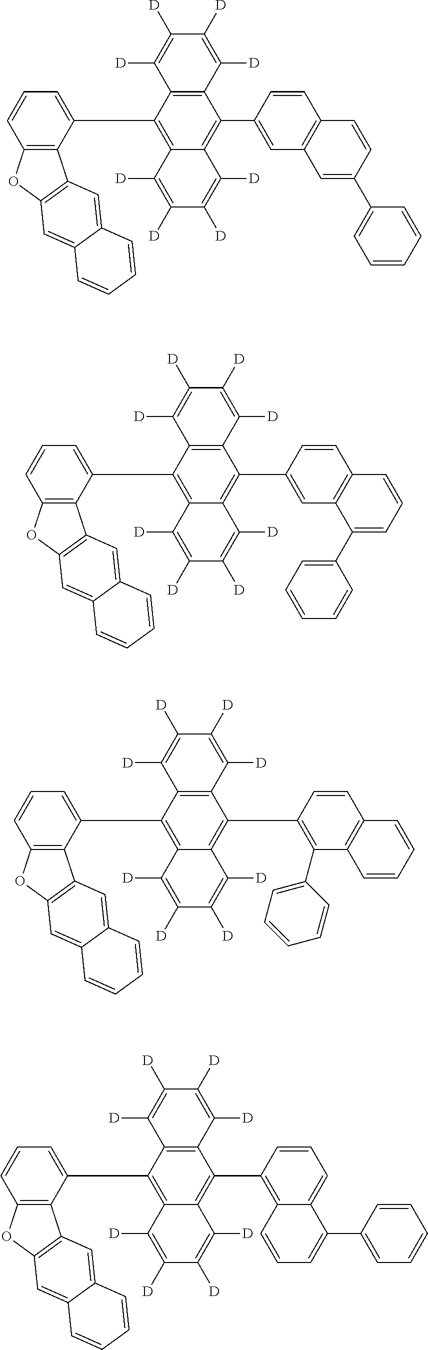 [SMILES: [2H]c1c([2H])c([2H])c2c(-c3cccc4oc5cc6ccccc6cc5c34)c3c([2H])c([2H])c([2H])c([2H])c3c(-c3ccc4ccc(-c5ccccc5)cc4c3)c2c1[2H] ; [2H]c1c([2H])c([2H])c2c(-c3cccc4oc5cc6ccccc6cc5c34)c3c([2H])c([2H])c([2H])c([2H])c3c(-c3ccc4cccc(-c5ccccc5)c4c3)c2c1[2H] ; [2H]c1c([2H])c([2H])c2c(-c3cccc4oc5cc6ccccc6cc5c34)c3c([2H])c([2H])c([2H])c([2H])c3c(-c3ccc4ccccc4c3-c3ccccc3)c2c1[2H] ; [2H]c1c([2H])c([2H])c2c(-c3cccc4oc5cc6ccccc6cc5c34)c3c([2H])c([2H])c([2H])c([2H])c3c(-c3cccc4c(-c5ccccc5)cccc34)c2c1[2H]]

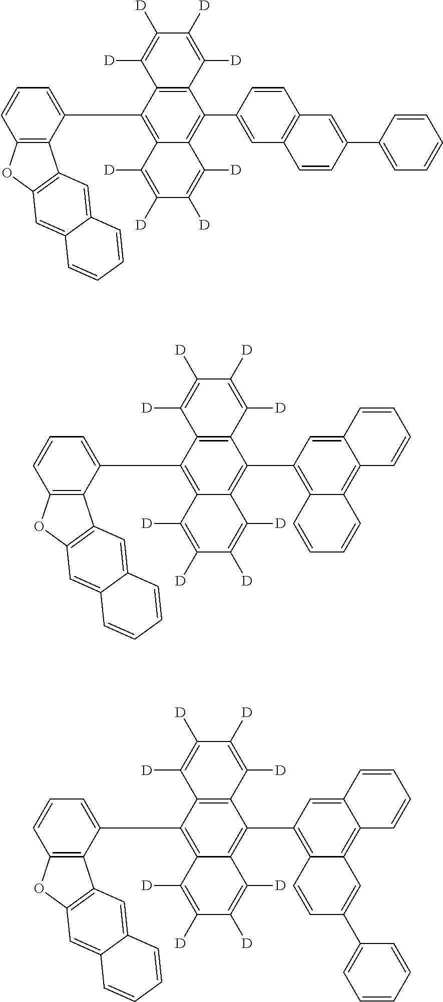 [SMILES: [2H]c1c([2H])c([2H])c2c(-c3cccc4oc5cc6ccccc6cc5c34)c3c([2H])c([2H])c([2H])c([2H])c3c(-c3ccc4cc(-c5ccccc5)ccc4c3)c2c1[2H] ; [2H]c1c([2H])c([2H])c2c(-c3cccc4oc5cc6ccccc6cc5c34)c3c([2H])c([2H])c([2H])c([2H])c3c(-c3cc4ccccc4c4ccccc34)c2c1[2H] ; [2H]c1c([2H])c([2H])c2c(-c3cccc4oc5cc6ccccc6cc5c34)c3c([2H])c([2H])c([2H])c([2H])c3c(-c3cc4ccccc4c4cc(-c5ccccc5)ccc34)c2c1[2H]]

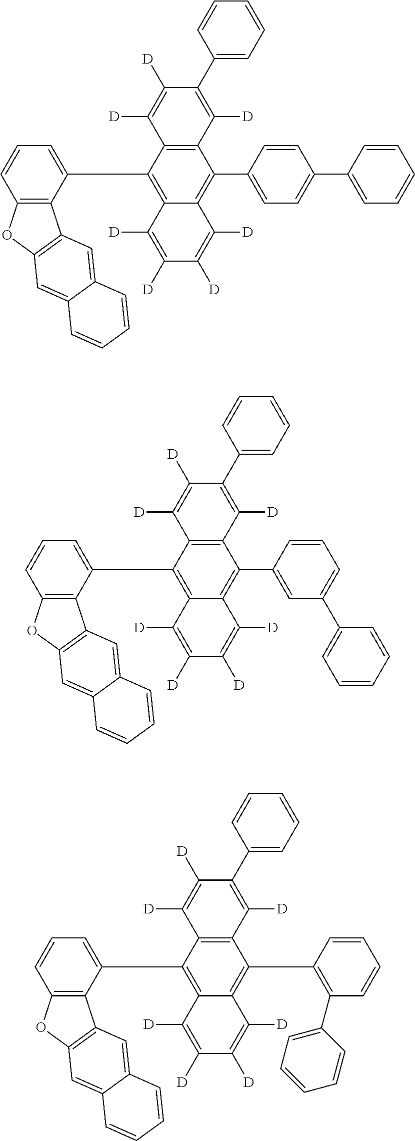 [SMILES: [2H]c1c([2H])c([2H])c2c(-c3cccc4oc5cc6ccccc6cc5c34)c3c([2H])c([2H])c(-c4ccccc4)c([2H])c3c(-c3ccc(-c4ccccc4)cc3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3cccc(-c4ccccc4)c3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3ccccc3-c3ccccc3)c2c1[2H]]

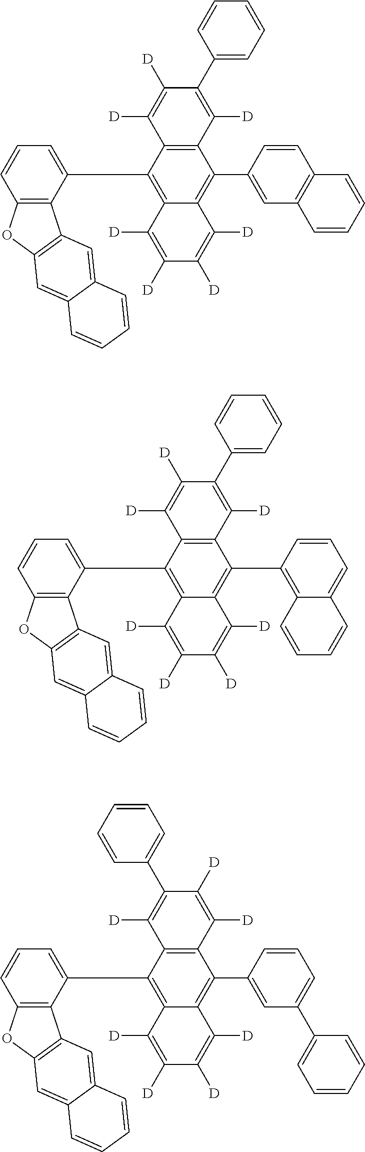 [SMILES: [2H]c1c([2H])c([2H])c2c(-c3cccc4oc5cc6ccccc6cc5c34)c3c([2H])c([2H])c(-c4ccccc4)c([2H])c3c(-c3ccc4ccccc4c3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3cccc4ccccc34)c2c1[2H] ; [2H]c1c([2H])c([2H])c2c(-c3cccc4oc5cc6ccccc6cc5c34)c3c([2H])c(-c4ccccc4)c([2H])c([2H])c3c(-c3cccc(-c4ccccc4)c3)c2c1[2H]]

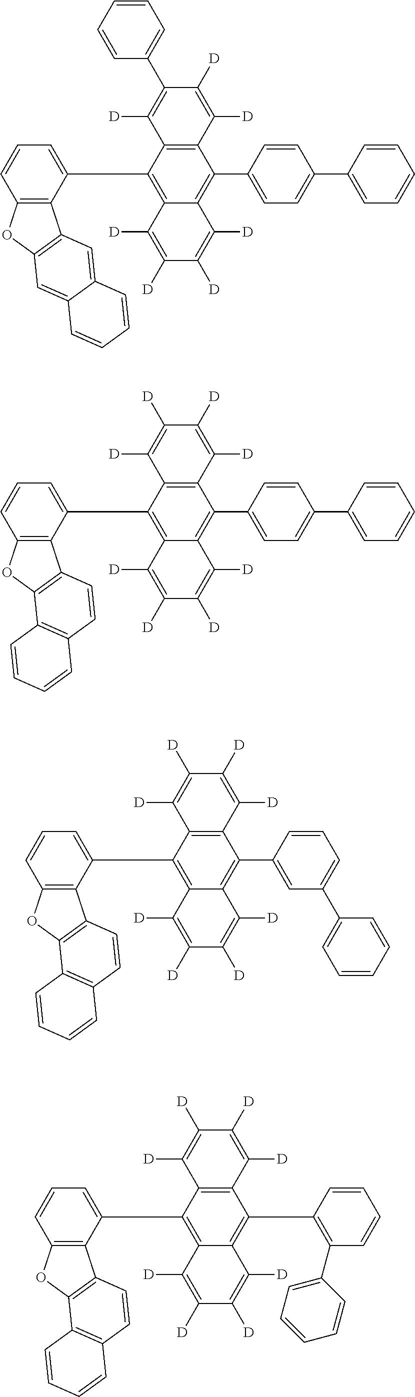 [SMILES: [2H]c1c([2H])c([2H])c2c(-c3cccc4oc5cc6ccccc6cc5c34)c3c([2H])c(-c4ccccc4)c([2H])c([2H])c3c(-c3ccc(-c4ccccc4)cc3)c2c1[2H] ; [2H]c1c([2H])c([2H])c2c(-c3cccc4oc5c6ccccc6ccc5c34)c3c([2H])c([2H])c([2H])c([2H])c3c(-c3ccc(-c4ccccc4)cc3)c2c1[2H] ; [2H]c1c([2H])c([2H])c2c(-c3cccc4oc5c6ccccc6ccc5c34)c3c([2H])c([2H])c([2H])c([2H])c3c(-c3cccc(-c4ccccc4)c3)c2c1[2H] ; [2H]c1c([2H])c([2H])c2c(-c3cccc4oc5c6ccccc6ccc5c34)c3c([2H])c([2H])c([2H])c([2H])c3c(-c3ccccc3-c3ccccc3)c2c1[2H]]

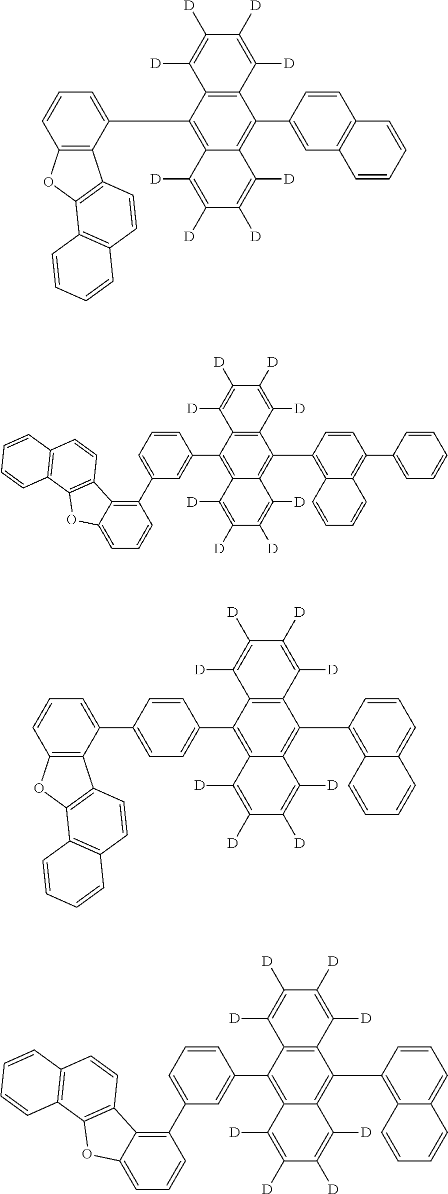 [SMILES: [2H]c1c([2H])c([2H])c2c(-c3cccc4oc5c6ccccc6ccc5c34)c3c([2H])c([2H])c([2H])c([2H])c3c(-c3ccc4ccccc4c3)c2c1[2H] ; [2H]c1c([2H])c([2H])c2c(-c3ccc(-c4ccccc4)c4ccccc34)c3c([2H])c([2H])c([2H])c([2H])c3c(-c3cccc(-c4cccc5oc6c7ccccc7ccc6c45)c3)c2c1[2H] ; [2H]c1c([2H])c([2H])c2c(-c3cccc4ccccc34)c3c([2H])c([2H])c([2H])c([2H])c3c(-c3ccc(-c4cccc5oc6c7ccccc7ccc6c45)cc3)c2c1[2H] ; [2H]c1c([2H])c([2H])c2c(-c3cccc4ccccc34)c3c([2H])c([2H])c([2H])c([2H])c3c(-c3cccc(-c4cccc5oc6c7ccccc7ccc6c45)c3)c2c1[2H]]

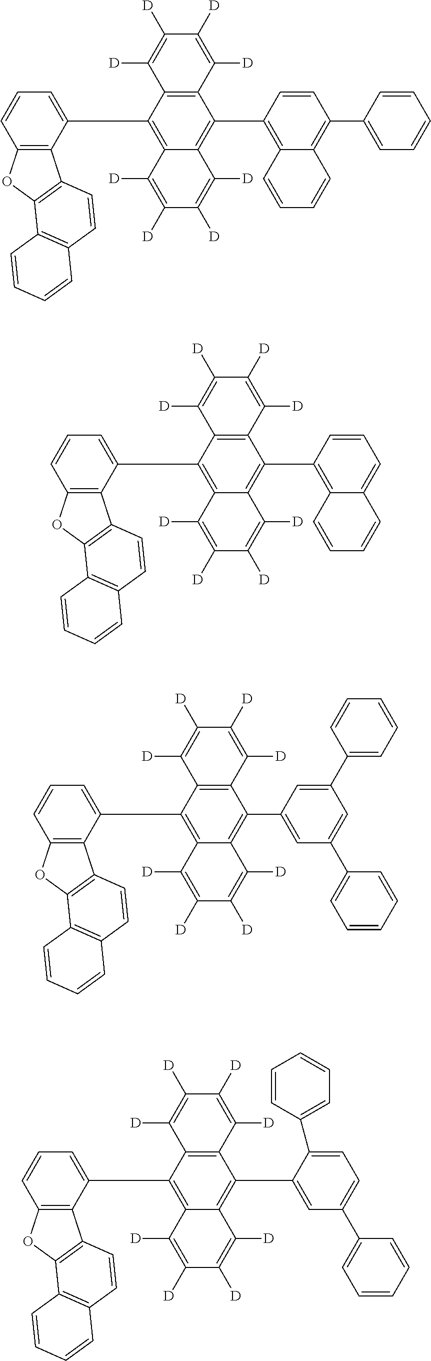 [SMILES: [2H]c1c([2H])c([2H])c2c(-c3cccc4oc5c6ccccc6ccc5c34)c3c([2H])c([2H])c([2H])c([2H])c3c(-c3ccc(-c4ccccc4)c4ccccc34)c2c1[2H] ; [2H]c1c([2H])c([2H])c2c(-c3cccc4oc5c6ccccc6ccc5c34)c3c([2H])c([2H])c([2H])c([2H])c3c(-c3cccc4ccccc34)c2c1[2H] ; [2H]c1c([2H])c([2H])c2c(-c3cccc4oc5c6ccccc6ccc5c34)c3c([2H])c([2H])c([2H])c([2H])c3c(-c3cc(-c4ccccc4)cc(-c4ccccc4)c3)c2c1[2H] ; [2H]c1c([2H])c([2H])c2c(-c3cccc4oc5c6ccccc6ccc5c34)c3c([2H])c([2H])c([2H])c([2H])c3c(-c3cc(-c4ccccc4)ccc3-c3ccccc3)c2c1[2H]]

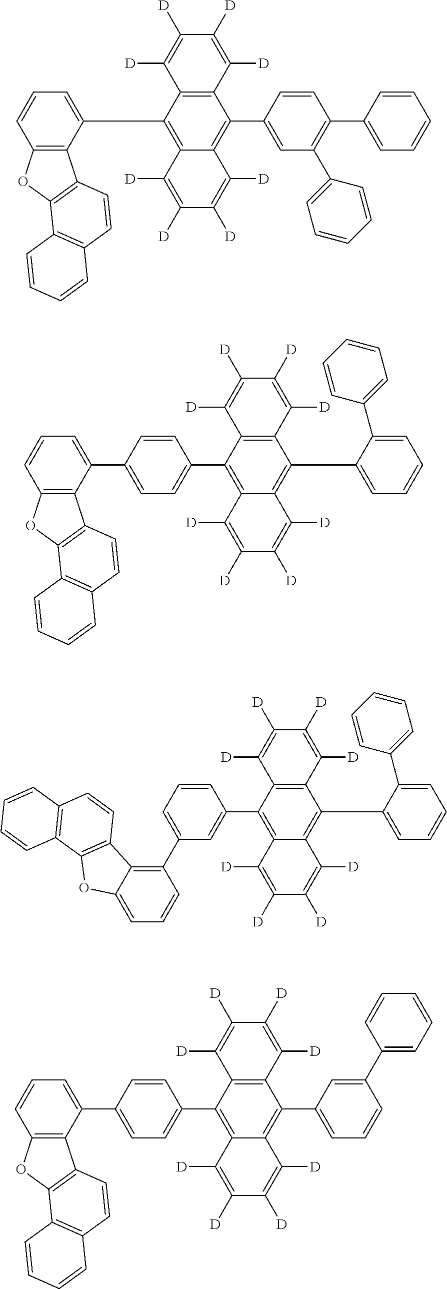

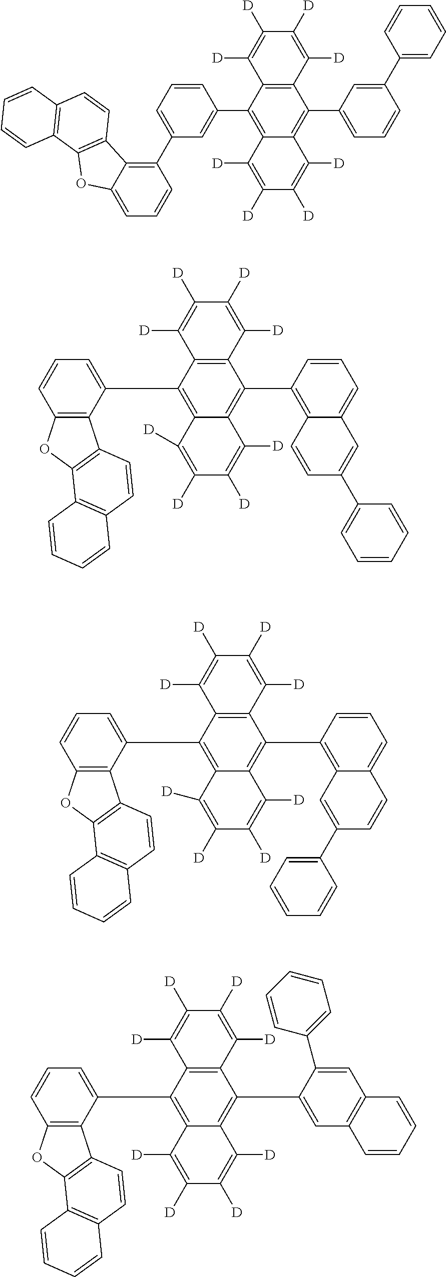 [SMILES: [2H]c1c([2H])c([2H])c2c(-c3cccc(-c4cccc5oc6c7ccccc7ccc6c45)c3)c3c([2H])c([2H])c([2H])c([2H])c3c(-c3cccc(-c4ccccc4)c3)c2c1[2H] ; [2H]c1c([2H])c([2H])c2c(-c3cccc4oc5c6ccccc6ccc5c34)c3c([2H])c([2H])c([2H])c([2H])c3c(-c3cccc4cc(-c5ccccc5)ccc34)c2c1[2H] ; [2H]c1c([2H])c([2H])c2c(-c3cccc4oc5c6ccccc6ccc5c34)c3c([2H])c([2H])c([2H])c([2H])c3c(-c3cccc4ccc(-c5ccccc5)cc34)c2c1[2H] ; [2H]c1c([2H])c([2H])c2c(-c3cccc4oc5c6ccccc6ccc5c34)c3c([2H])c([2H])c([2H])c([2H])c3c(-c3cc4ccccc4cc3-c3ccccc3)c2c1[2H]]

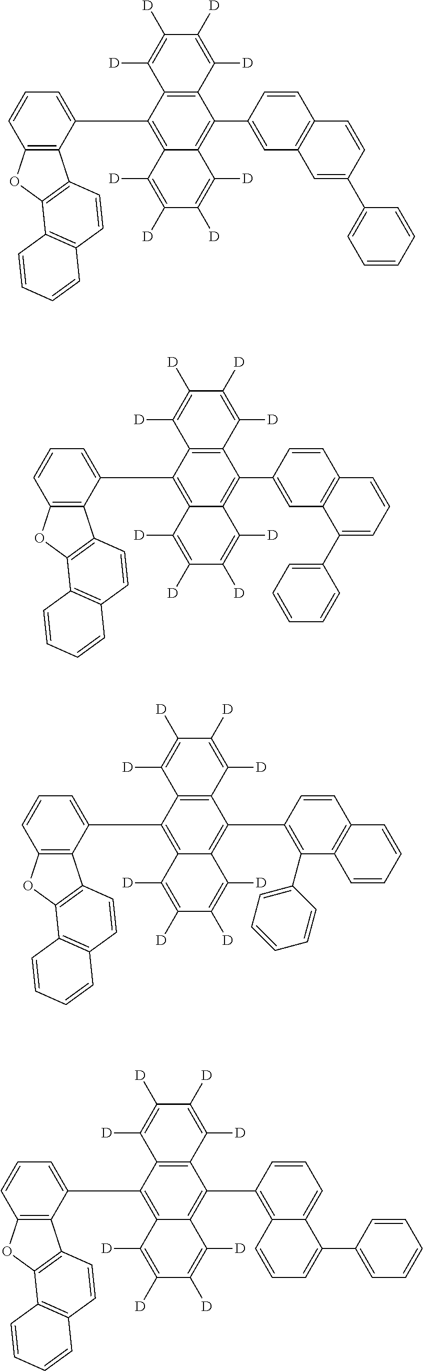 [SMILES: [2H]c1c([2H])c([2H])c2c(-c3cccc4oc5c6ccccc6ccc5c34)c3c([2H])c([2H])c([2H])c([2H])c3c(-c3ccc4ccc(-c5ccccc5)cc4c3)c2c1[2H] ; [2H]c1c([2H])c([2H])c2c(-c3cccc4oc5c6ccccc6ccc5c34)c3c([2H])c([2H])c([2H])c([2H])c3c(-c3ccc4cccc(-c5ccccc5)c4c3)c2c1[2H] ; [2H]c1c([2H])c([2H])c2c(-c3cccc4oc5c6ccccc6ccc5c34)c3c([2H])c([2H])c([2H])c([2H])c3c(-c3ccc4ccccc4c3-c3ccccc3)c2c1[2H] ; [2H]c1c([2H])c([2H])c2c(-c3cccc4oc5c6ccccc6ccc5c34)c3c([2H])c([2H])c([2H])c([2H])c3c(-c3cccc4c(-c5ccccc5)cccc34)c2c1[2H]]

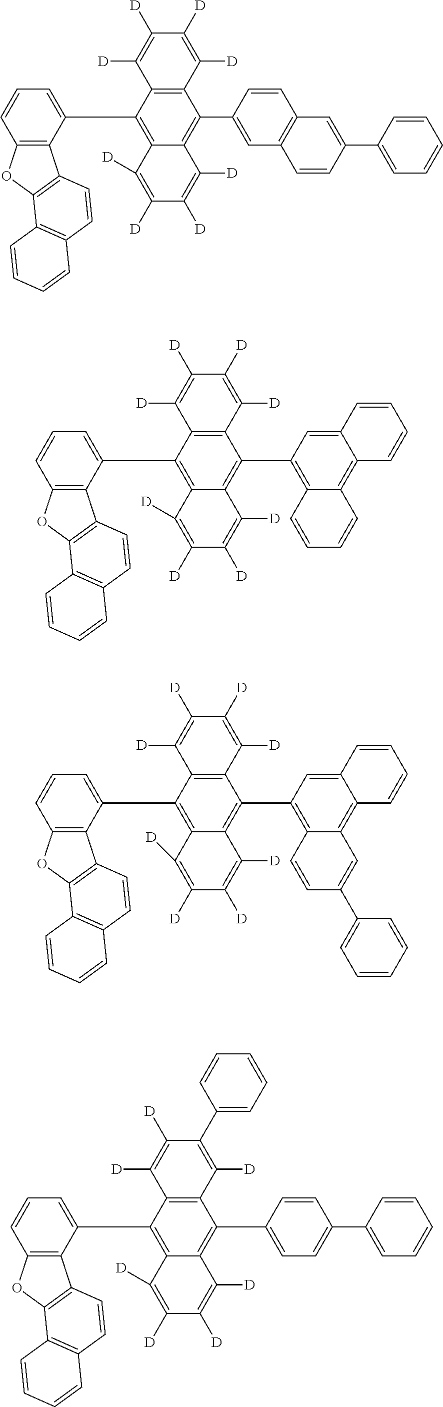 [SMILES: [2H]c1c([2H])c([2H])c2c(-c3cccc4oc5c6ccccc6ccc5c34)c3c([2H])c([2H])c([2H])c([2H])c3c(-c3ccc4cc(-c5ccccc5)ccc4c3)c2c1[2H] ; [2H]c1c([2H])c([2H])c2c(-c3cccc4oc5c6ccccc6ccc5c34)c3c([2H])c([2H])c([2H])c([2H])c3c(-c3cc4ccccc4c4ccccc34)c2c1[2H] ; [2H]c1c([2H])c([2H])c2c(-c3cccc4oc5c6ccccc6ccc5c34)c3c([2H])c([2H])c([2H])c([2H])c3c(-c3cc4ccccc4c4cc(-c5ccccc5)ccc34)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccc(-c4ccccc4)cc3)c2c1[2H]]

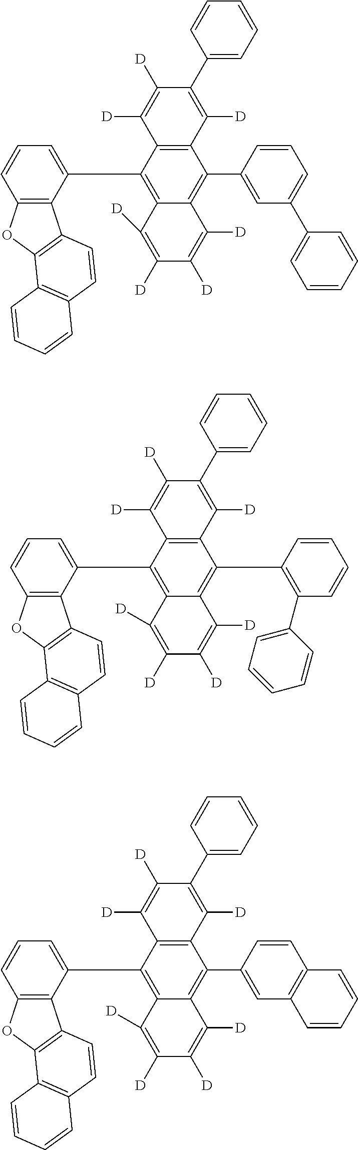 [SMILES: [2H]c1c([2H])c([2H])c2c(-c3cccc4oc5c6ccccc6ccc5c34)c3c([2H])c([2H])c(-c4ccccc4)c([2H])c3c(-c3cccc(-c4ccccc4)c3)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccccc3-c3ccccc3)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccc4ccccc4c3)c2c1[2H]]

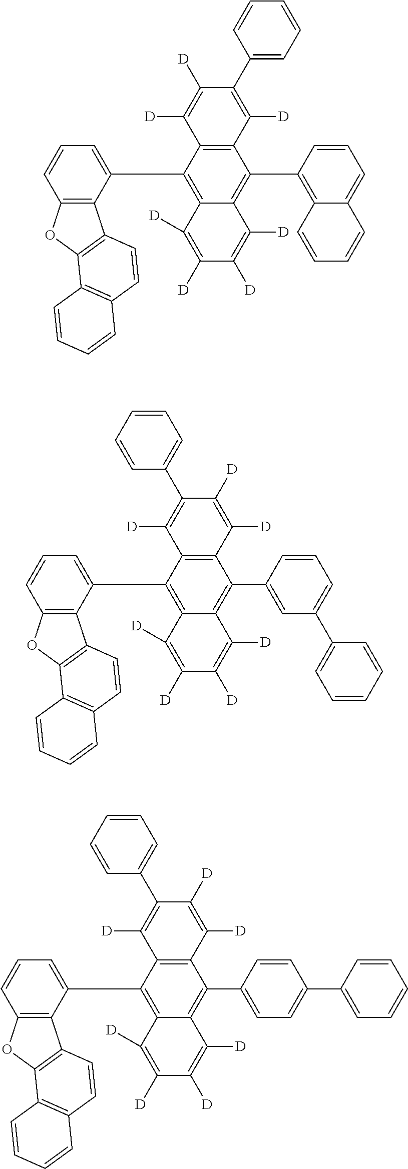 [SMILES: [2H]c1c([2H])c([2H])c2c(-c3cccc4oc5c6ccccc6ccc5c34)c3c([2H])c([2H])c(-c4ccccc4)c([2H])c3c(-c3cccc4ccccc34)c2c1[2H] ; [2H]c1c([2H])c([2H])c2c(-c3cccc4oc5c6ccccc6ccc5c34)c3c([2H])c(-c4ccccc4)c([2H])c([2H])c3c(-c3cccc(-c4ccccc4)c3)c2c1[2H] ; [2H]c1c([2H])c([2H])c2c(-c3cccc4oc5c6ccccc6ccc5c34)c3c([2H])c(-c4ccccc4)c([2H])c([2H])c3c(-c3ccc(-c4ccccc4)cc3)c2c1[2H]]

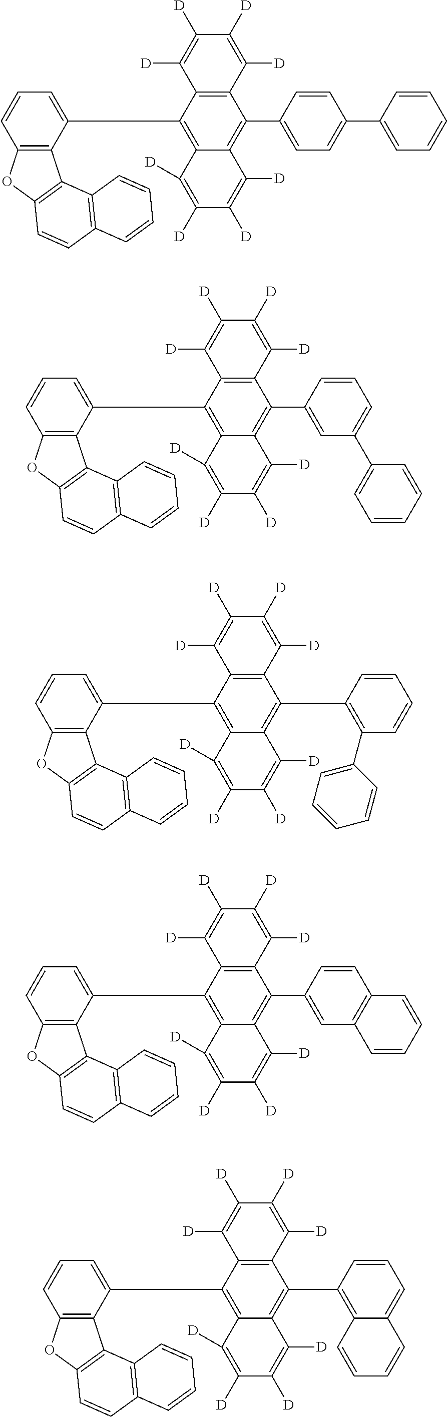

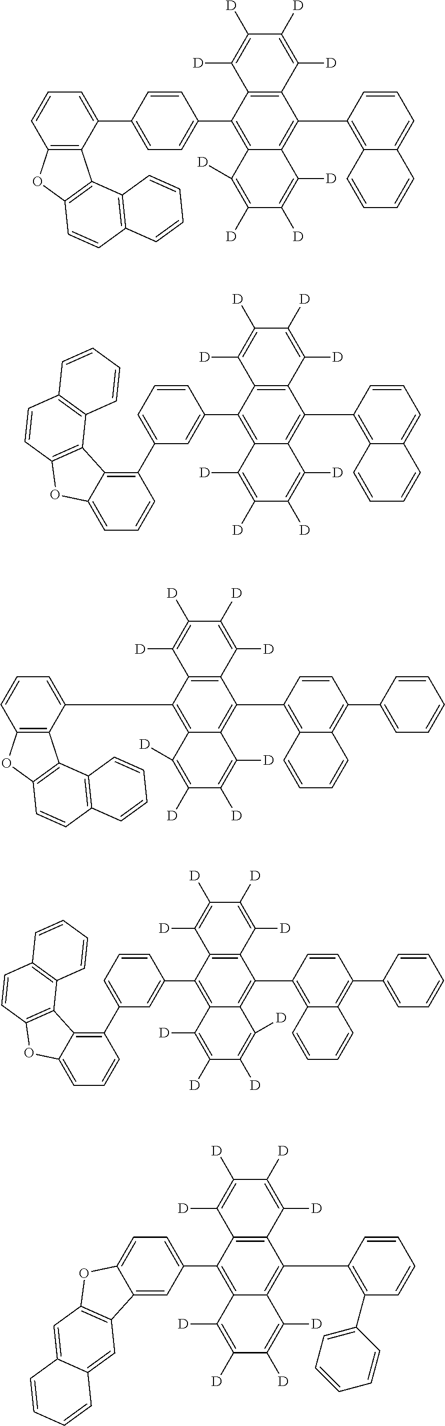

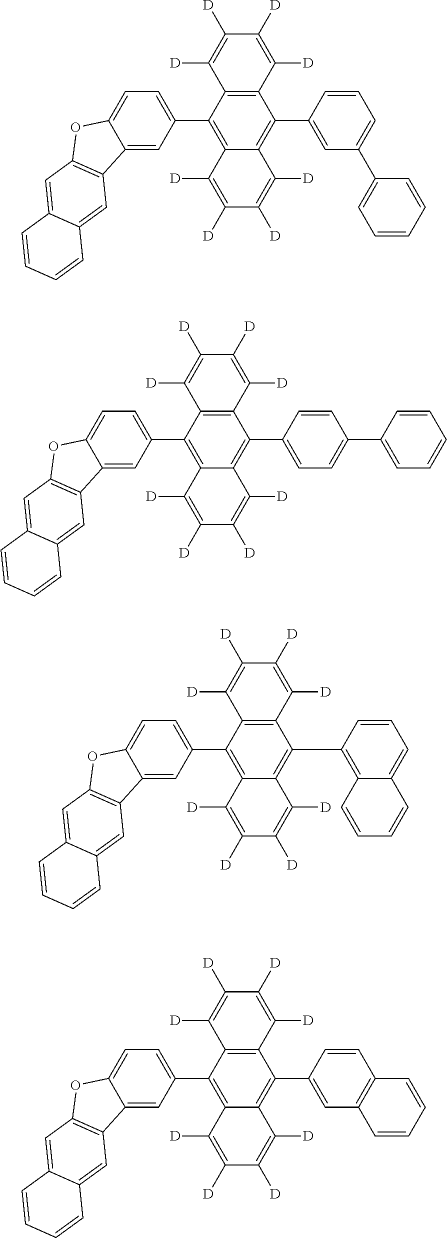 [SMILES: [2H]c1c([2H])c([2H])c2c(-c3ccc4oc5cc6ccccc6cc5c4c3)c3c([2H])c([2H])c([2H])c([2H])c3c(-c3cccc(-c4ccccc4)c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5cc6ccccc6cc5c4c3)c2c1[2H] ; [2H]c1c([2H])c([2H])c2c(-c3ccc4oc5cc6ccccc6cc5c4c3)c3c([2H])c([2H])c([2H])c([2H])c3c(-c3ccc4ccccc4c3)c2c1[2H]]

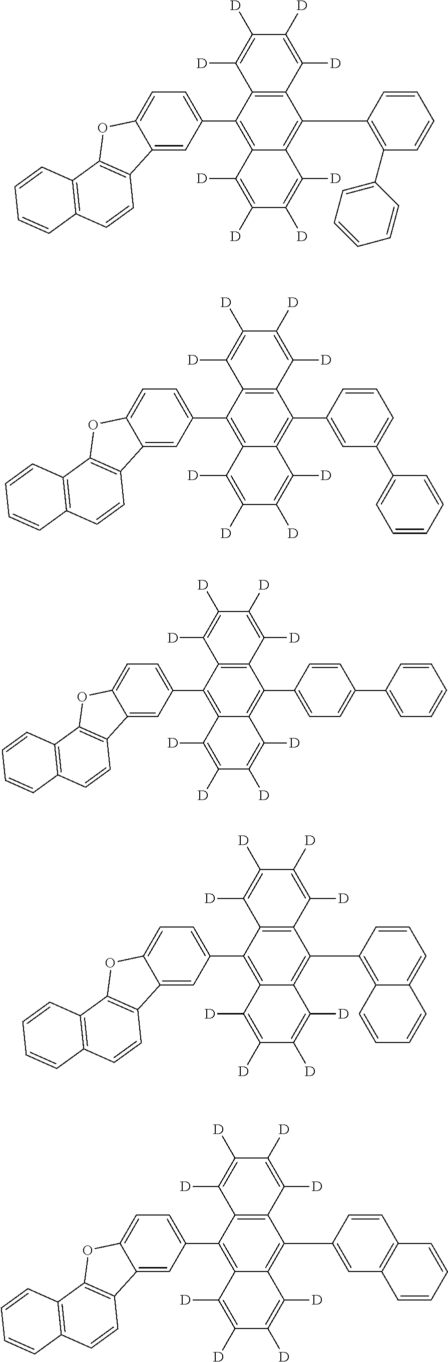

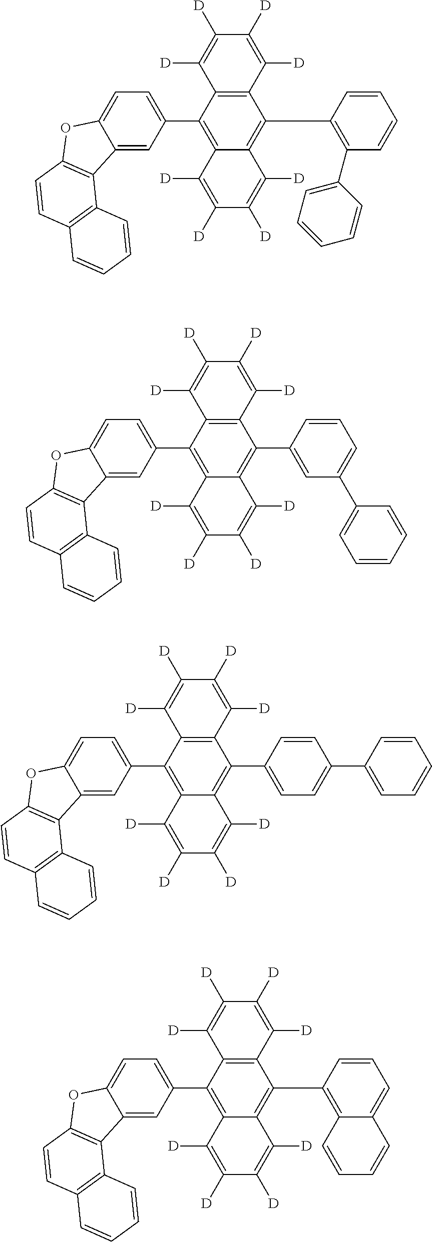 [SMILES: [2H]c1c([2H])c([2H])c2c(-c3ccccc3-c3ccccc3)c3c([2H])c([2H])c([2H])c([2H])c3c(-c3ccc4oc5ccc6ccccc6c5c4c3)c2c1[2H] ; [2H]c1c([2H])c([2H])c2c(-c3ccc4oc5ccc6ccccc6c5c4c3)c3c([2H])c([2H])c([2H])c([2H])c3c(-c3cccc(-c4ccccc4)c3)c2c1[2H] ; [2H]c1c([2H])c([2H])c2c(-c3ccc4oc5ccc6ccccc6c5c4c3)c3c([2H])c([2H])c([2H])c([2H])c3c(-c3ccc(-c4ccccc4)cc3)c2c1[2H] ; [2H]c1c([2H])c([2H])c2c(-c3cccc4ccccc34)c3c([2H])c([2H])c([2H])c([2H])c3c(-c3ccc4oc5ccc6ccccc6c5c4c3)c2c1[2H]]

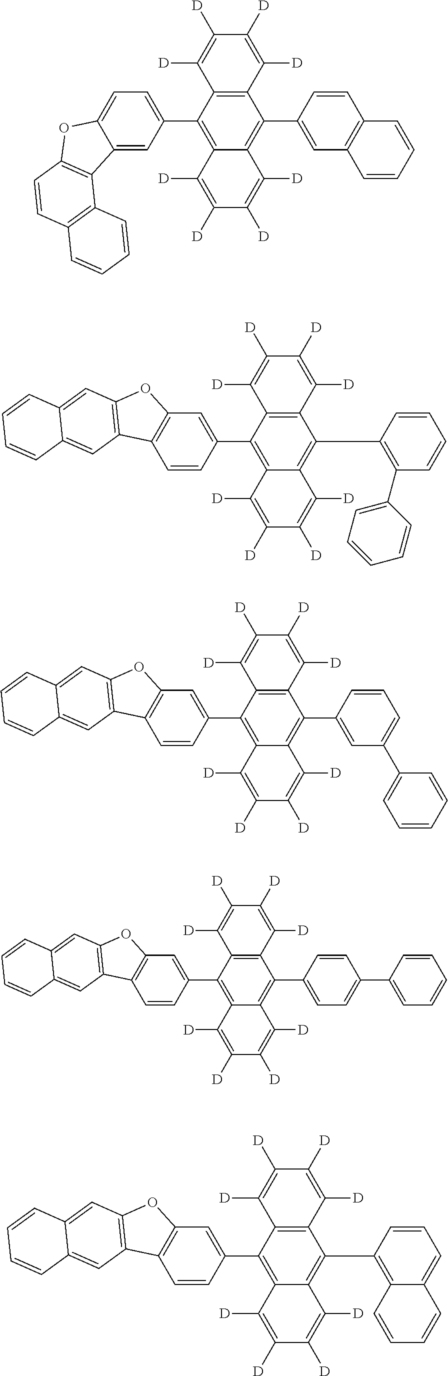

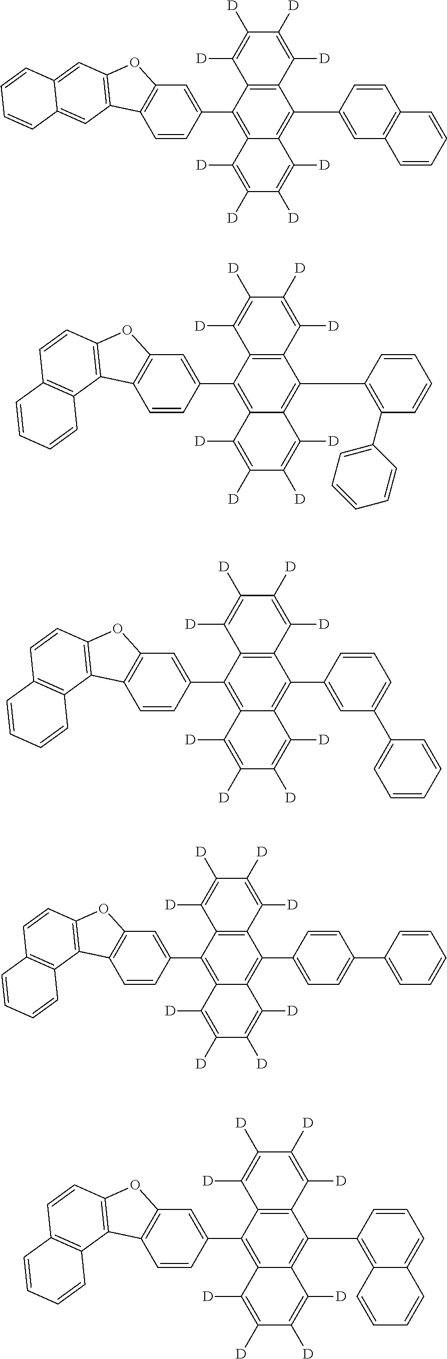

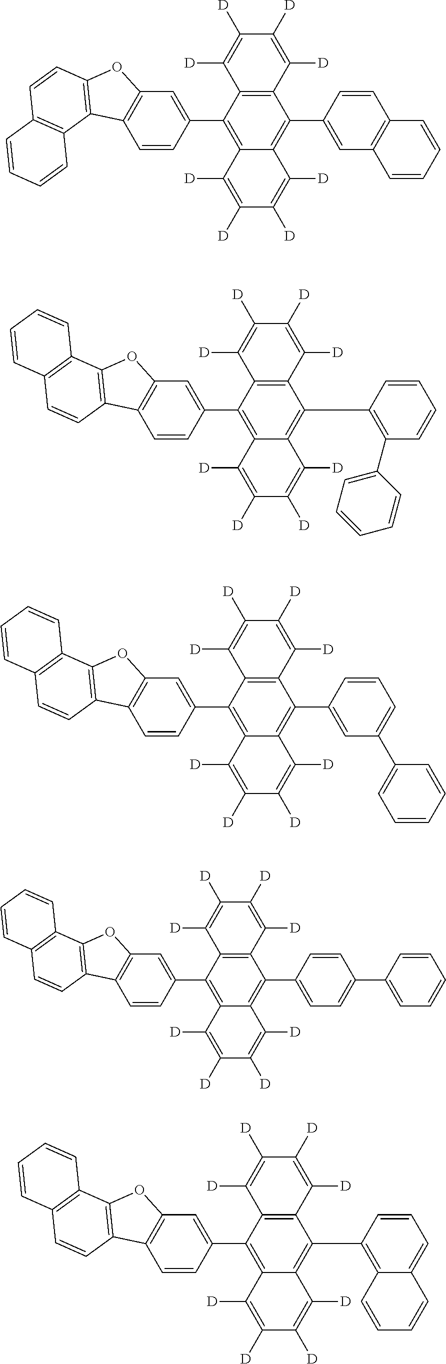

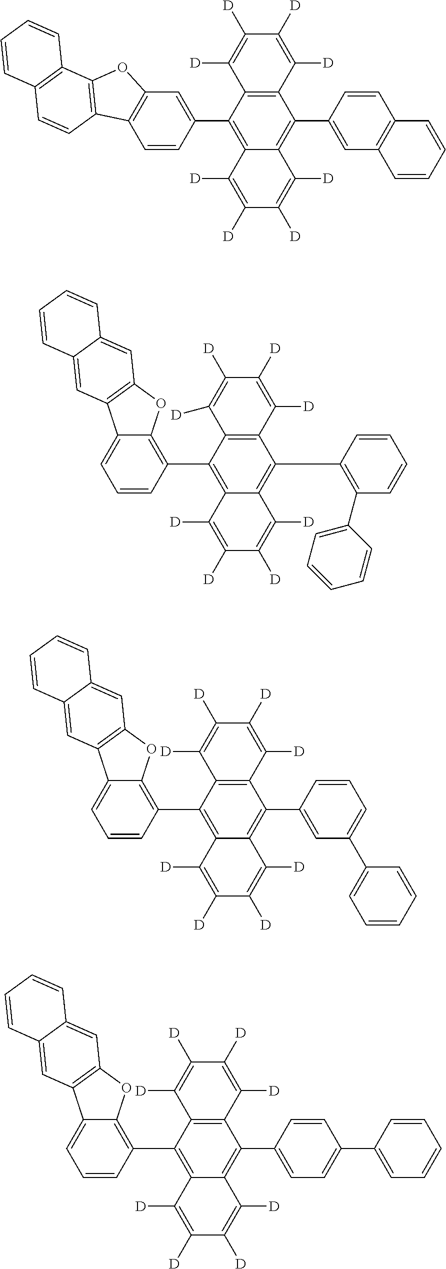 [SMILES: [2H]c1c([2H])c([2H])c2c(-c3ccc4c(c3)oc3c5ccccc5ccc43)c3c([2H])c([2H])c([2H])c([2H])c3c(-c3ccc4ccccc4c3)c2c1[2H] ; [2H]c1c([2H])c([2H])c2c(-c3cccc4c3oc3cc5ccccc5cc34)c3c([2H])c([2H])c([2H])c([2H])c3c(-c3ccccc3-c3ccccc3)c2c1[2H] ; [2H]c1c([2H])c([2H])c2c(-c3cccc4c3oc3cc5ccccc5cc34)c3c([2H])c([2H])c([2H])c([2H])c3c(-c3cccc(-c4ccccc4)c3)c2c1[2H] ; [2H]c1c([2H])c([2H])c2c(-c3cccc4c3oc3cc5ccccc5cc34)c3c([2H])c([2H])c([2H])c([2H])c3c(-c3ccc(-c4ccccc4)cc3)c2c1[2H]]

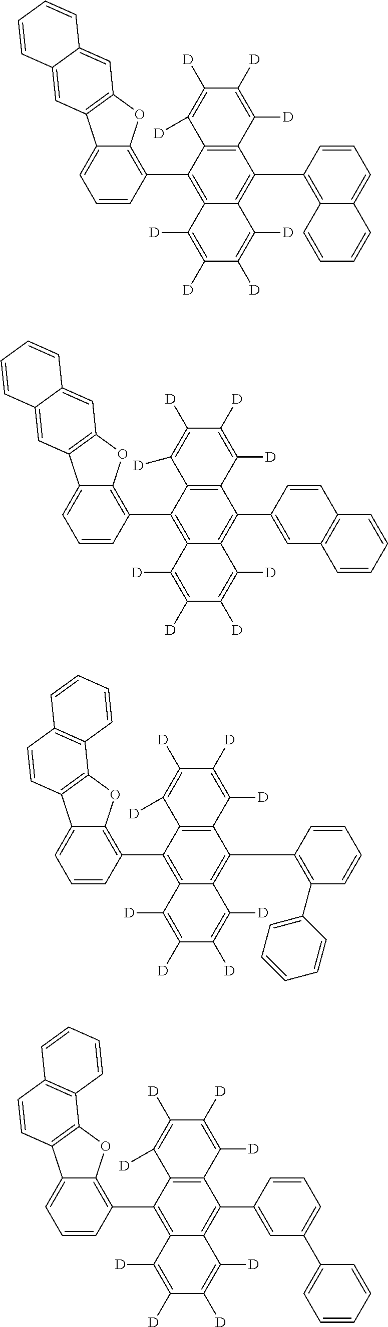 [SMILES: [2H]c1c([2H])c([2H])c2c(-c3cccc4c3oc3cc5ccccc5cc34)c3c([2H])c([2H])c([2H])c([2H])c3c(-c3cccc4ccccc34)c2c1[2H] ; [2H]c1c([2H])c([2H])c2c(-c3cccc4c3oc3cc5ccccc5cc34)c3c([2H])c([2H])c([2H])c([2H])c3c(-c3ccc4ccccc4c3)c2c1[2H] ; [2H]c1c([2H])c([2H])c2c(-c3cccc4c3oc3c5ccccc5ccc43)c3c([2H])c([2H])c([2H])c([2H])c3c(-c3ccccc3-c3ccccc3)c2c1[2H] ; [2H]c1c([2H])c([2H])c2c(-c3cccc4c3oc3c5ccccc5ccc43)c3c([2H])c([2H])c([2H])c([2H])c3c(-c3cccc(-c4ccccc4)c3)c2c1[2H]]

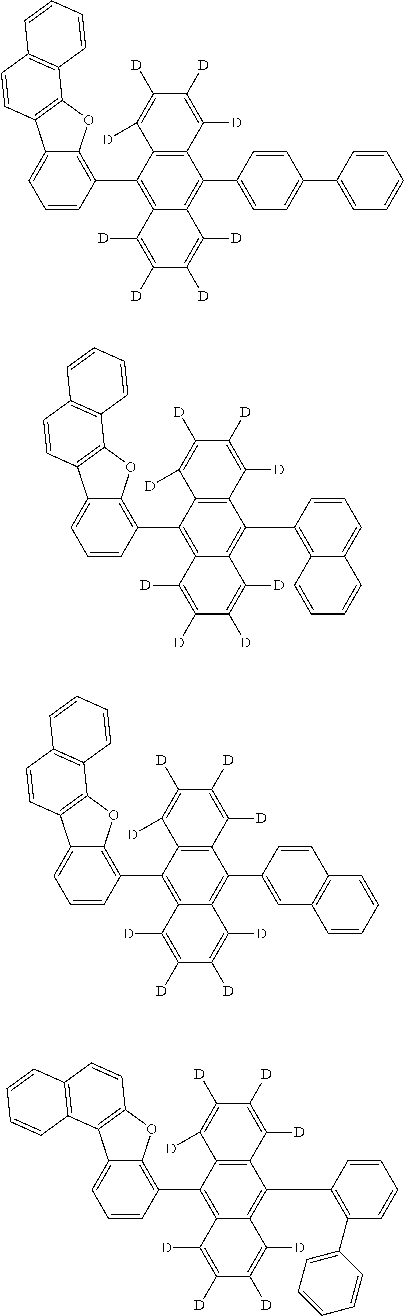 [SMILES: [2H]c1c([2H])c([2H])c2c(-c3cccc4c3oc3c5ccccc5ccc43)c3c([2H])c([2H])c([2H])c([2H])c3c(-c3ccc(-c4ccccc4)cc3)c2c1[2H] ; [2H]c1c([2H])c([2H])c2c(-c3cccc4c3oc3c5ccccc5ccc43)c3c([2H])c([2H])c([2H])c([2H])c3c(-c3cccc4ccccc34)c2c1[2H] ; [2H]c1c([2H])c([2H])c2c(-c3cccc4c3oc3c5ccccc5ccc43)c3c([2H])c([2H])c([2H])c([2H])c3c(-c3ccc4ccccc4c3)c2c1[2H] ; [2H]c1c([2H])c([2H])c2c(-c3cccc4c3oc3ccc5ccccc5c34)c3c([2H])c([2H])c([2H])c([2H])c3c(-c3ccccc3-c3ccccc3)c2c1[2H]]

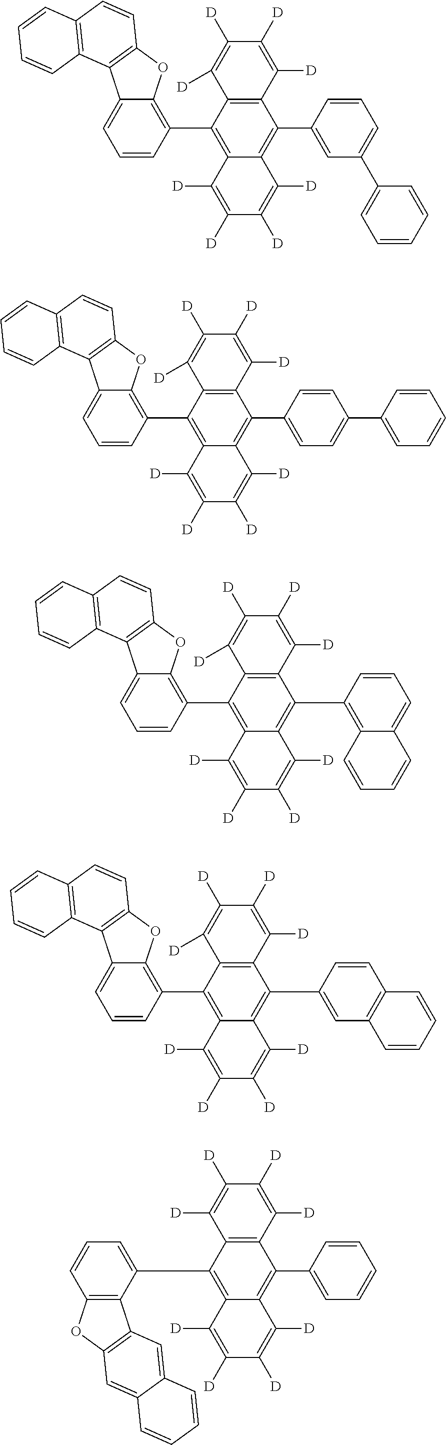

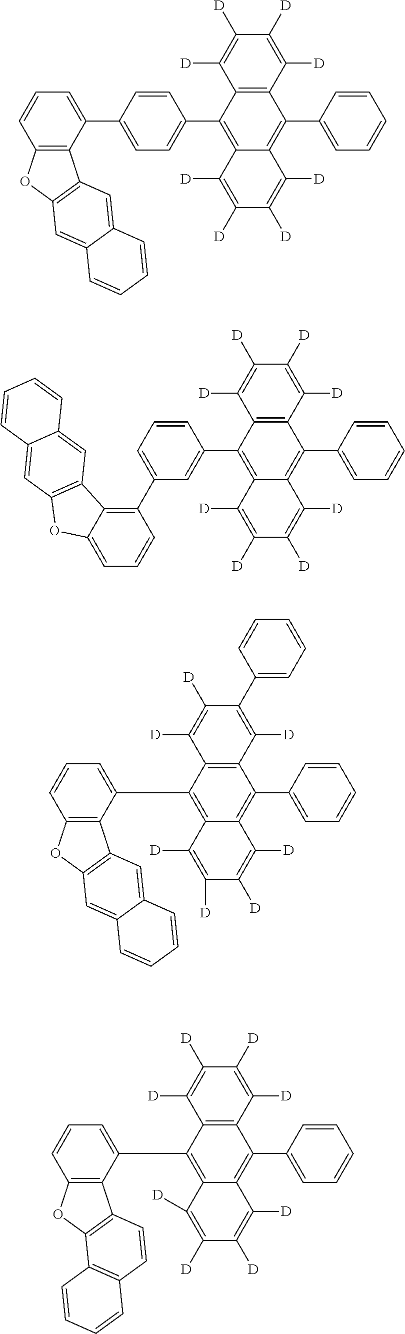 [SMILES: [2H]c1c([2H])c([2H])c2c(-c3ccc(-c4cccc5oc6cc7ccccc7cc6c45)cc3)c3c([2H])c([2H])c([2H])c([2H])c3c(-c3ccccc3)c2c1[2H] ; [2H]c1c([2H])c([2H])c2c(-c3cccc(-c4cccc5oc6cc7ccccc7cc6c45)c3)c3c([2H])c([2H])c([2H])c([2H])c3c(-c3ccccc3)c2c1[2H] ; [2H]c1c([2H])c([2H])c2c(-c3cccc4oc5cc6ccccc6cc5c34)c3c([2H])c([2H])c(-c4ccccc4)c([2H])c3c(-c3ccccc3)c2c1[2H] ; [2H]c1c([2H])c([2H])c2c(-c3cccc4oc5c6ccccc6ccc5c34)c3c([2H])c([2H])c([2H])c([2H])c3c(-c3ccccc3)c2c1[2H]]

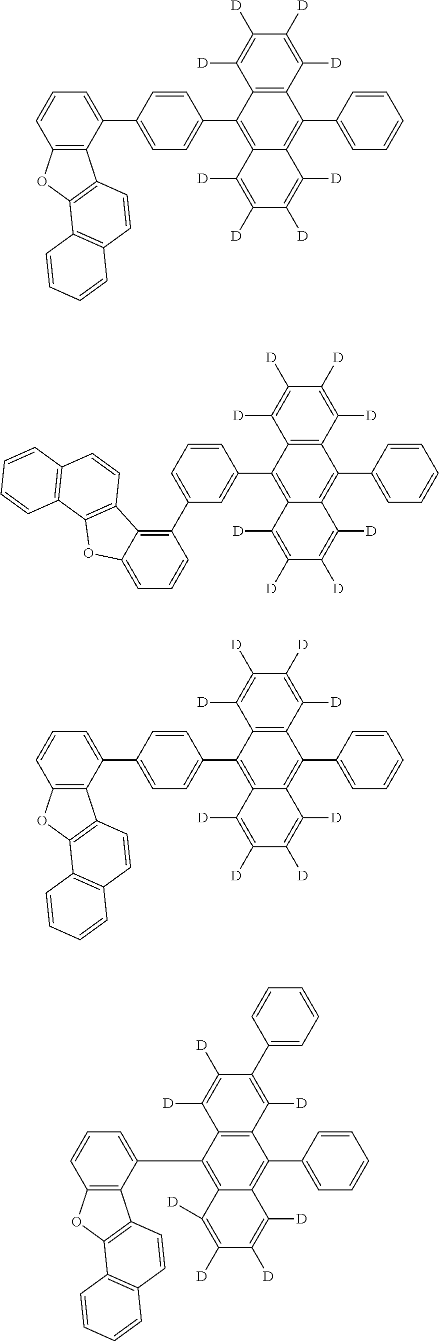 [SMILES: [2H]c1c([2H])c([2H])c2c(-c3ccc(-c4cccc5oc6c7ccccc7ccc6c45)cc3)c3c([2H])c([2H])c([2H])c([2H])c3c(-c3ccccc3)c2c1[2H] ; [2H]c1c([2H])c([2H])c2c(-c3cccc(-c4cccc5oc6c7ccccc7ccc6c45)c3)c3c([2H])c([2H])c([2H])c([2H])c3c(-c3ccccc3)c2c1[2H] ; [2H]c1c([2H])c([2H])c2c(-c3ccc(-c4cccc5oc6c7ccccc7ccc6c45)cc3)c3c([2H])c([2H])c([2H])c([2H])c3c(-c3ccccc3)c2c1[2H] ; [2H]c1c([2H])c([2H])c2c(-c3cccc4oc5c6ccccc6ccc5c34)c3c([2H])c([2H])c(-c4ccccc4)c([2H])c3c(-c3ccccc3)c2c1[2H]]

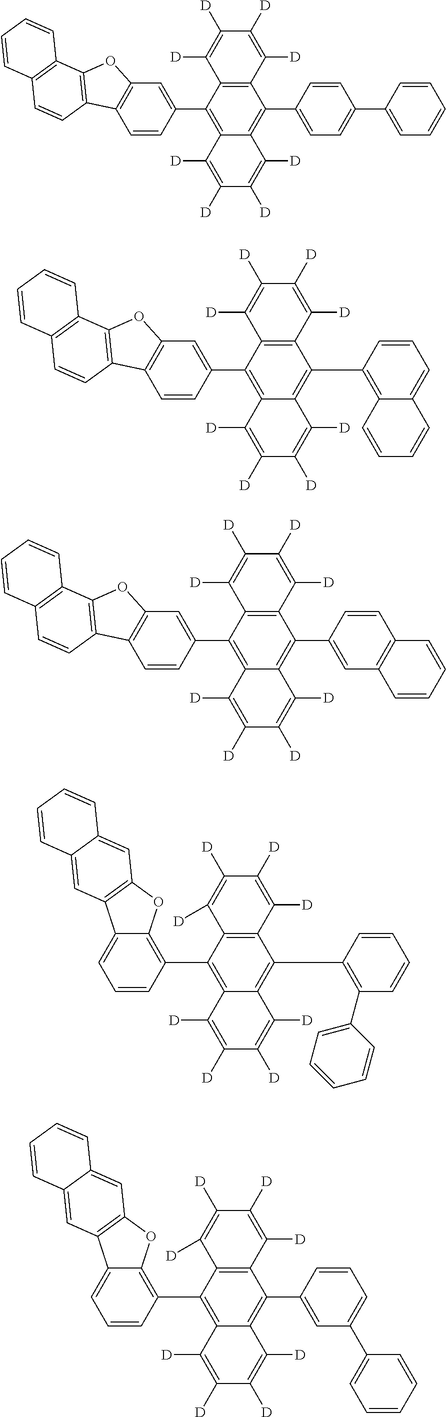

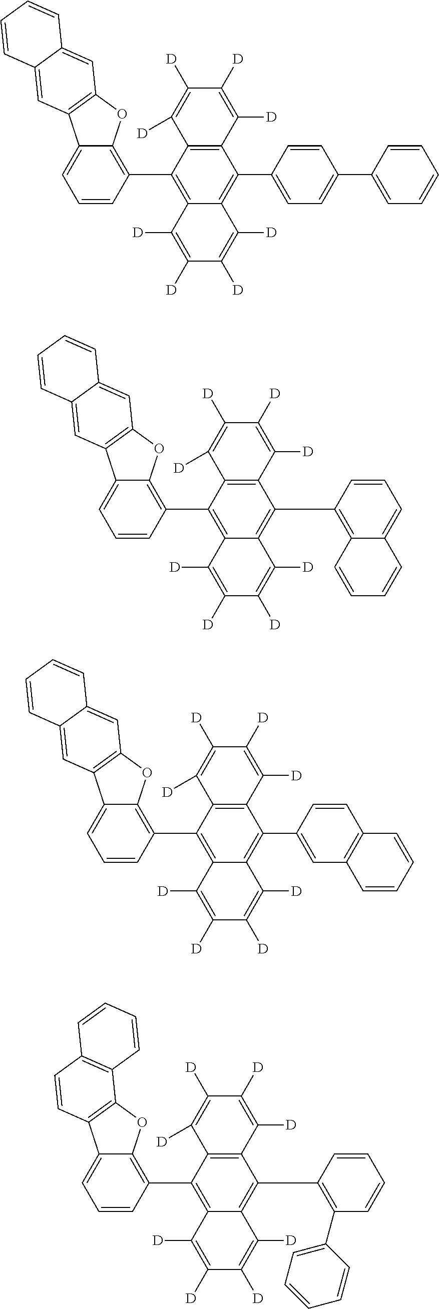 [SMILES: [2H]c1c([2H])c([2H])c2c(-c3cccc4c3oc3cc5ccccc5cc34)c3c([2H])c([2H])c([2H])c([2H])c3c(-c3ccc(-c4ccccc4)cc3)c2c1[2H] ; [2H]c1c([2H])c([2H])c2c(-c3cccc4c3oc3cc5ccccc5cc34)c3c([2H])c([2H])c([2H])c([2H])c3c(-c3cccc4ccccc34)c2c1[2H] ; [2H]c1c([2H])c([2H])c2c(-c3cccc4c3oc3cc5ccccc5cc34)c3c([2H])c([2H])c([2H])c([2H])c3c(-c3ccc4ccccc4c3)c2c1[2H] ; [2H]c1c([2H])c([2H])c2c(-c3cccc4c3oc3c5ccccc5ccc43)c3c([2H])c([2H])c([2H])c([2H])c3c(-c3ccccc3-c3ccccc3)c2c1[2H]]

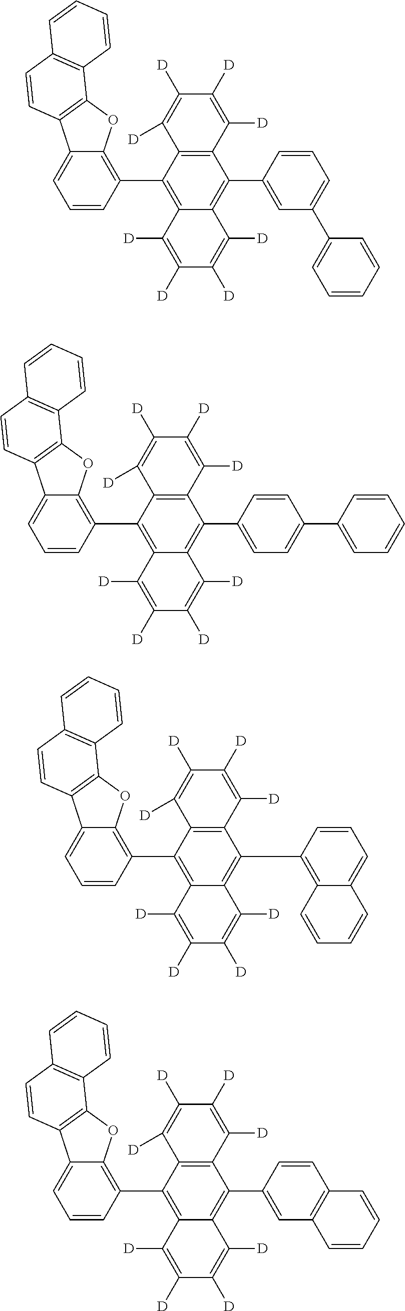 [SMILES: [2H]c1c([2H])c([2H])c2c(-c3cccc4c3oc3c5ccccc5ccc43)c3c([2H])c([2H])c([2H])c([2H])c3c(-c3cccc(-c4ccccc4)c3)c2c1[2H] ; [2H]c1c([2H])c([2H])c2c(-c3cccc4c3oc3c5ccccc5ccc43)c3c([2H])c([2H])c([2H])c([2H])c3c(-c3ccc(-c4ccccc4)cc3)c2c1[2H] ; [2H]c1c([2H])c([2H])c2c(-c3cccc4c3oc3c5ccccc5ccc43)c3c([2H])c([2H])c([2H])c([2H])c3c(-c3cccc4ccccc34)c2c1[2H] ; [2H]c1c([2H])c([2H])c2c(-c3cccc4c3oc3c5ccccc5ccc43)c3c([2H])c([2H])c([2H])c([2H])c3c(-c3ccc4ccccc4c3)c2c1[2H]]

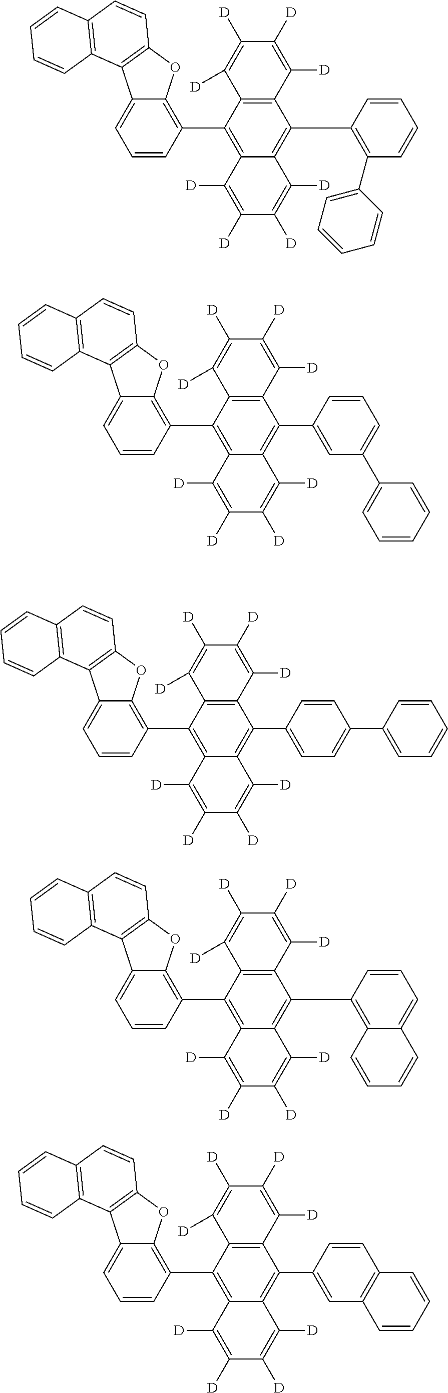

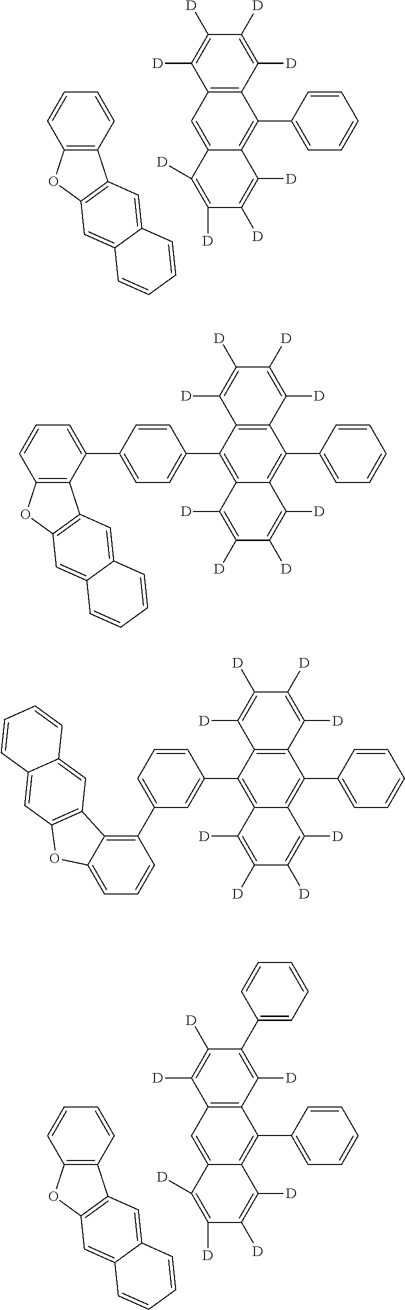

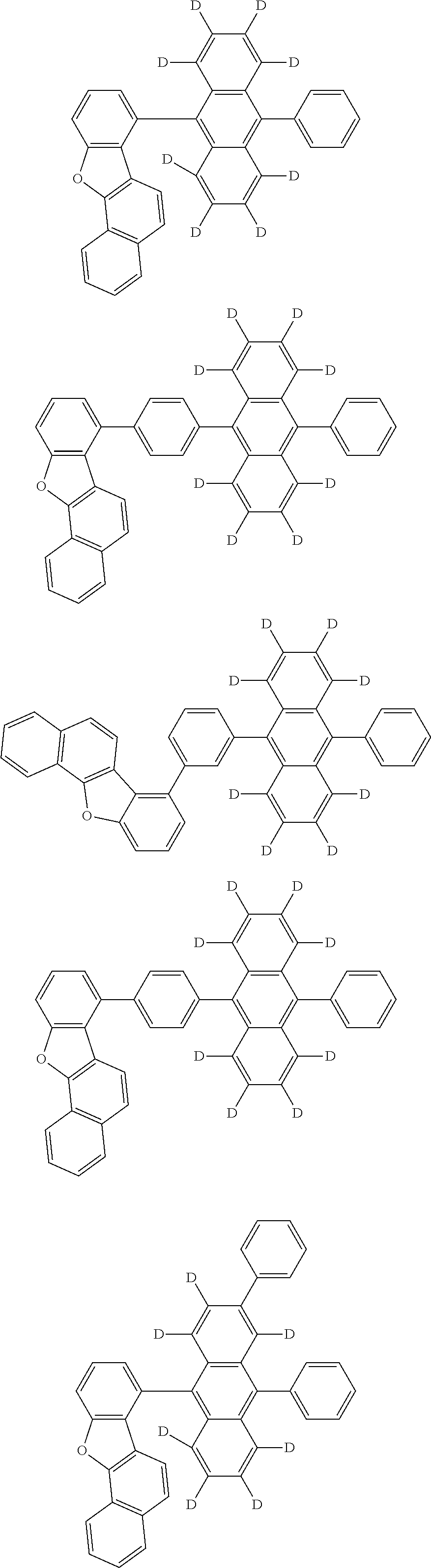

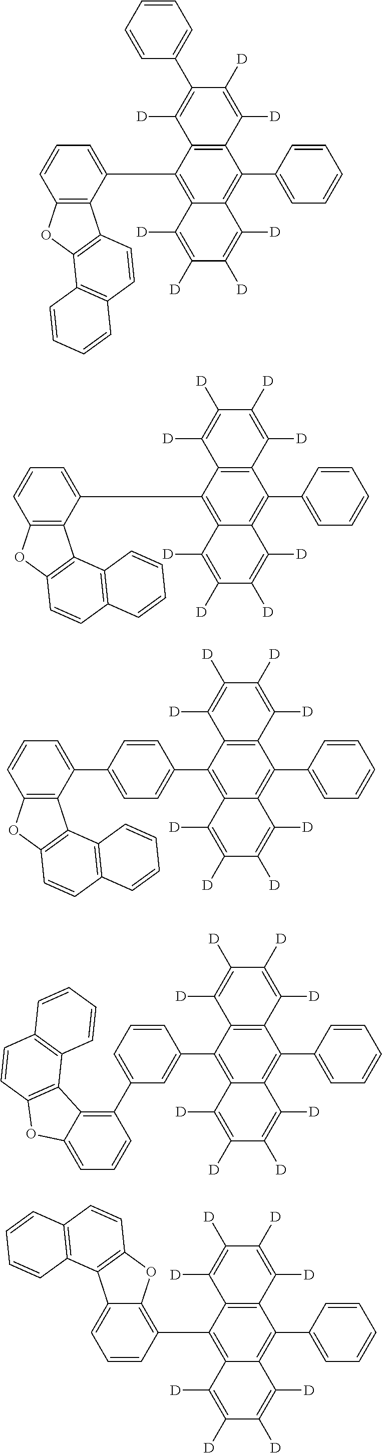

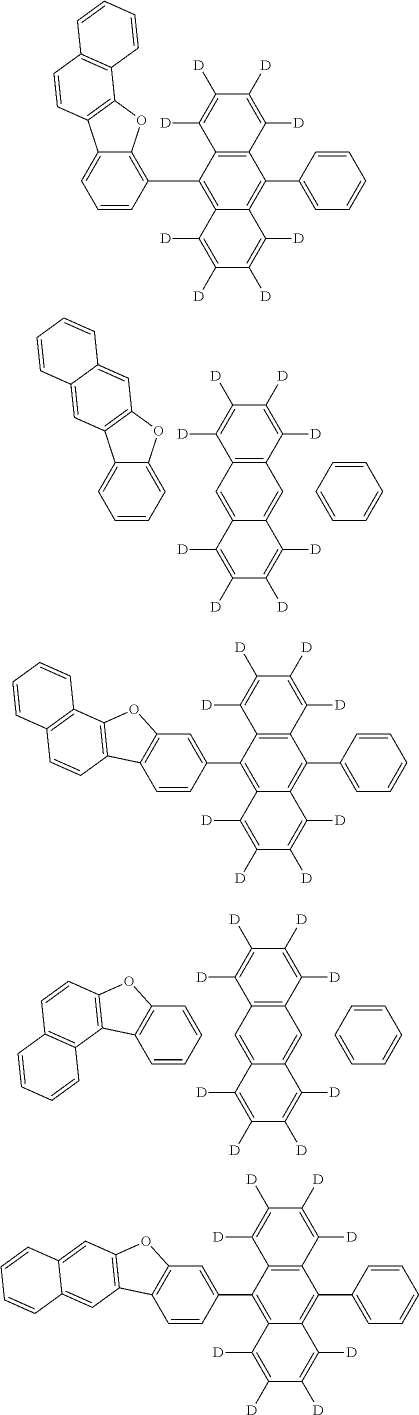

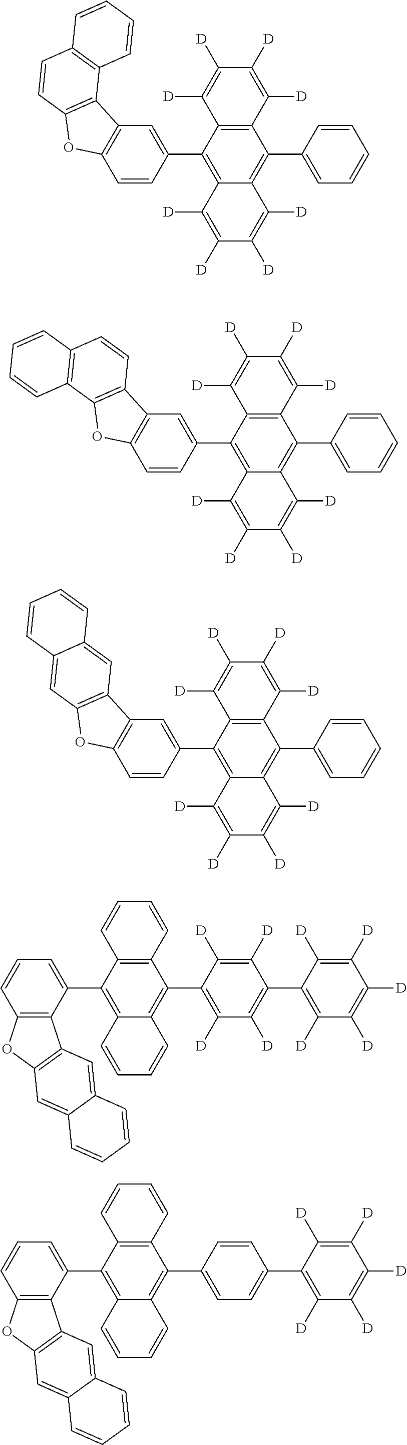

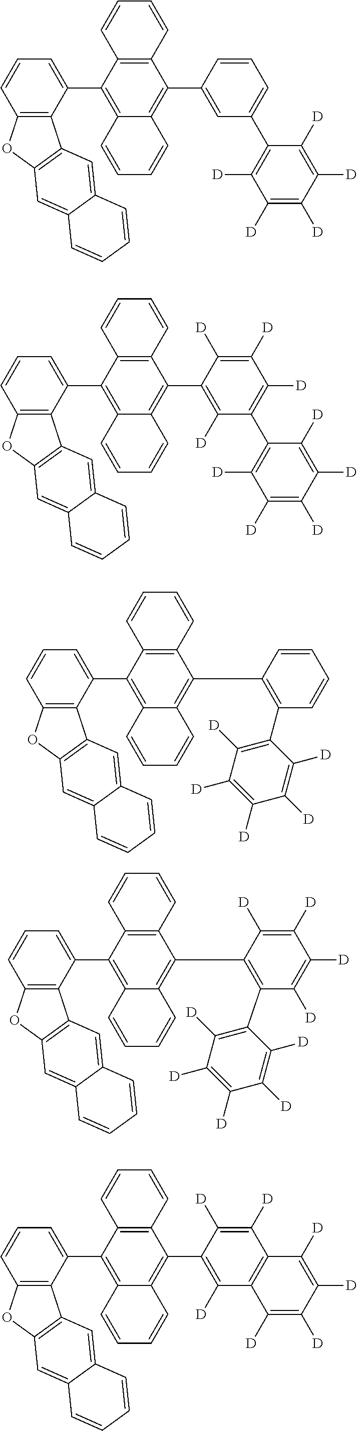

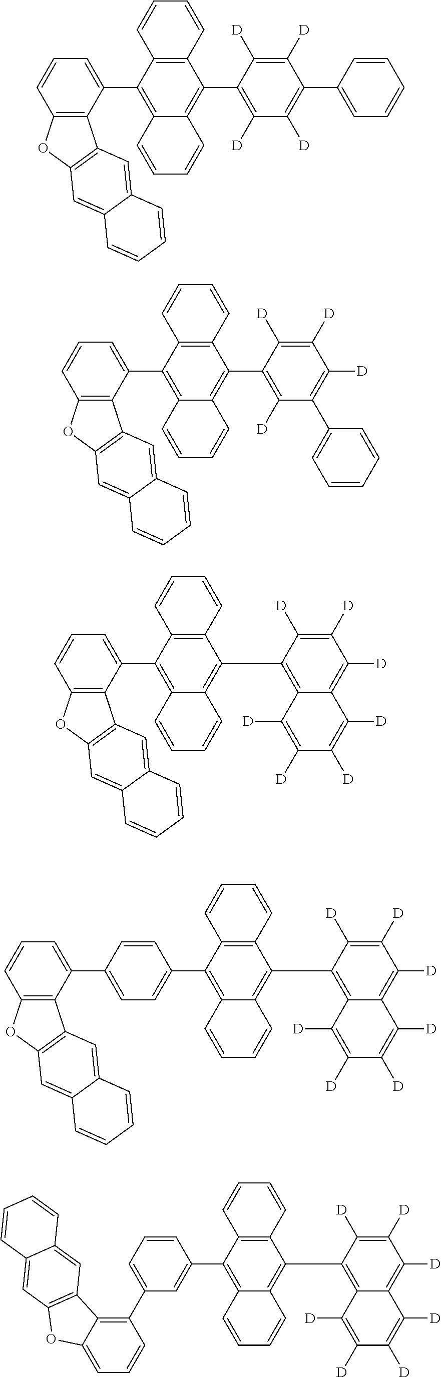

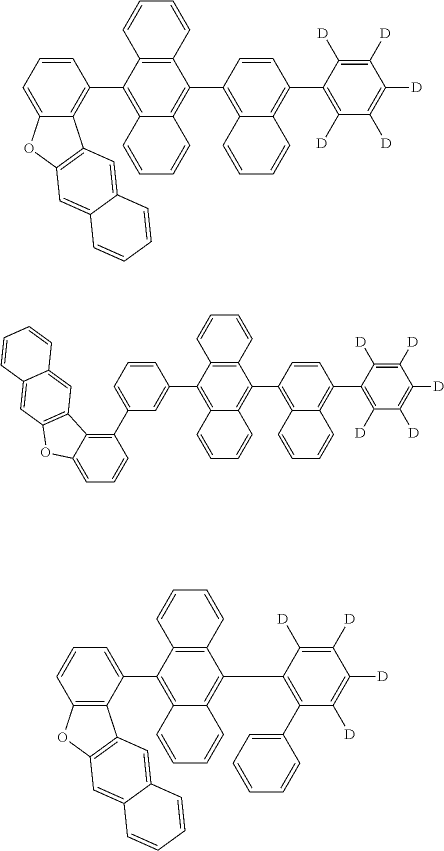 [SMILES: [2H]c1c([2H])c([2H])c(-c2ccc(-c3c4ccccc4c(-c4cccc5oc6cc7ccccc7cc6c45)c4ccccc34)c3ccccc23)c([2H])c1[2H] ; [2H]c1c([2H])c([2H])c(-c2ccc(-c3c4ccccc4c(-c4cccc(-c5cccc6oc7cc8ccccc8cc7c56)c4)c4ccccc34)c3ccccc23)c([2H])c1[2H] ; [2H]c1c([2H])c([2H])c(-c2c3ccccc3c(-c3cccc4oc5cc6ccccc6cc5c34)c3ccccc23)c(-c2ccccc2)c1[2H]]

1. A composition comprising a compound represented by the following formula (A1) and a compound represented by the following formula (B1) (provided that the compound represented by the formula (A1) and the compound represented by the formula (B1) are different compounds): 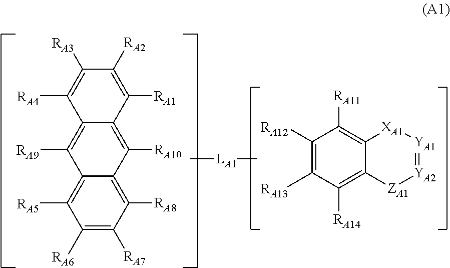
wherein in the formula (A1),
one of R_{A1} to R_{A10} is a single bond bonding with L_{A1};
one or more sets of the adjacent two or more of R_{A1} to R_{A10} which are not a single bond bonding with L_{A1} form a substituted or unsubstituted, saturated or unsaturated ring by bonding with each other, or do not form the ring;
one of R_{A1} to R_{A14} is a single bond bonding with L_{A1};
L_{A1} is a single bond, a substituted or unsubstituted arylene group having 6 to 50 ring carbon atoms, or a substituted or unsubstituted divalent heterocyclic group having 5 to 50 ring atoms;
X_{A1} is O or S;
Y_{A1} and Y_{A2} are independently a single bond, or C(Rₐ);
Z_{A1} is a single bond, or C(Rₐ)(R_{b});
one or more sets of the adjacent two or more of Rₐ, and R_{A11} to R_{A14} which are not a single bond bonding with L_{A1} form a substituted or unsubstituted, saturated or unsaturated ring by bonding with each other, or do not form the ring;
R_{b}, R_{A1} to R_{A10} and R_{A11} to R_{A14} which are not a single bond bonding with L_{A1} and which do not form the ring, and Rₐ which do not form the ring are independently a hydrogen atom, or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms;
when two or more of each of R₉₀₁ to R₉₀₇ are present, the two or more of each of R₉₀₁ to R₉₀₇ may be the same as or different from each other: 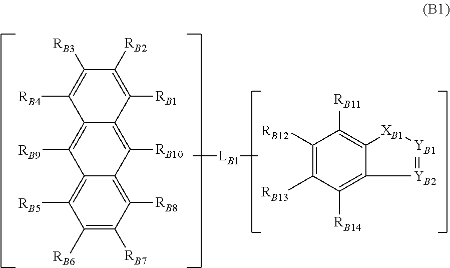
wherein in the formula (B1),
one of R_{B1} to R_{B10} is a single bond bonding with L_{B1};
one or more sets of the adjacent two or more of R_{B1} to R_{B10} which are not a single bond bonding with L_{B1} form a substituted or unsubstituted, saturated or unsaturated ring by bonding with each other, or do not form the ring;
X_{B1} is O, S, N(R_{c}), or C(R_{d})(Rₑ);
one of R_{B11} to R_{B14}, and R_{c} is a single bond bonding with L_{B1};
L_{B1} is a single bond, a substituted or unsubstituted arylene group having 6 to 50 ring carbon atoms, or a substituted or unsubstituted divalent heterocyclic group having 5 to 50 ring atoms;
Y_{B1} and Y_{B2} are independently a single bond, or C(R_{f});
R_{d} and Rₑ, and R_{f}'s form a substituted or unsubstituted, saturated or unsaturated ring by bonding with each other, or do not form the ring;
one or more sets of the adjacent two or more of R_{B11} to R_{B14} which are not a single bond bonding with L_{B1} form a substituted or unsubstituted, saturated or unsaturated ring by bonding with each other, or do not form the ring; and
R_{B1} to R_{B10} which do not form the ring and which are not a single bond bonding with L_{B1}, R_{B11} to R_{B14} which do not form the ring and which are not a single bond bonding with L^{B1}, R_{c} which are not a single bond bonding with L_{B1}, and R_{d}, Rₑ and R_{f} which do not form the ring are independently a hydrogen atom, or a substituent R; and the substituent R is the same as defined in the formula (A1).

2. The composition according to claim 1, wherein the compound represented by the formula (A1) is a compound represented by the following formula (A2): 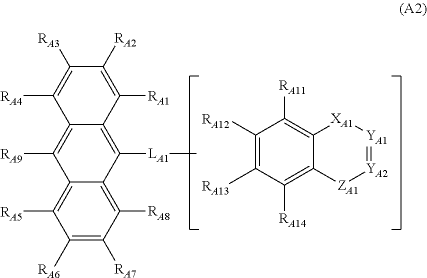
wherein in the formula (A2), R_{A1} to R_{A9}, R_{A11} to R_{A14}, L_{A1}, X_{A1}, Y_{A2}, Y_{A2}, and Z_{A1} are the same as defined in the formula (A1).

3. The composition according to claim 1, wherein the compound represented by the formula (A1) is a compound represented by the following formula (A2-1): 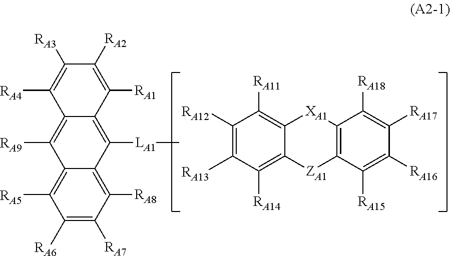
wherein in the formula (A2-1), R_{A1} to R_{A9}, R_{A11} to R_{A14}, L_{A1}, X_{A1}, and Z_{A1} are the same as defined in the formula (A1);
one or more sets of the adjacent two or more of Rₐ and R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

4. The composition according to claim 1, wherein the compound represented by the formula (A1) is a compound represented by the following formula (A3): 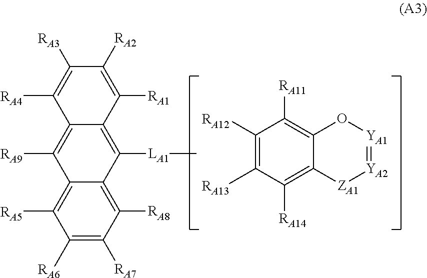
wherein in the formula (A3), R_{A1} to R_{A9}, R_{A11} to R_{A14}, L_{A1}, Y_{A1}, Y_{A2}, and Z_{A1} are the same as defined in the formula (A1).

5. The composition according to claim 1, wherein the compound represented by the formula (A1) is a compound represented by the following formula (A4): 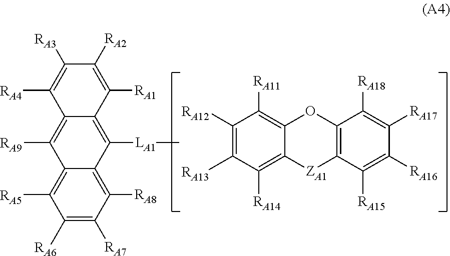
wherein in the formula (A4), R_{A1} to R_{A9}, R_{A11} to R_{A14}, L_{A1}, and Z_{A1} are the same as defined in the formula (A1);
one or more sets of the adjacent two or more of Rₐ, and R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

6. The composition according to claim 1, wherein the compound represented by the formula (A1) is a compound represented by the following formula (A5-1): 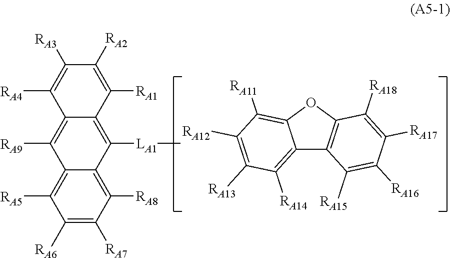
wherein in the formula (A5-1), R_{A1} to R_{A9}, R_{A11} to R_{A14}, and L_{A1} are the same as defined in the formula (A1);
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

7. The composition according to claim 1, wherein the compound represented by the formula (A1) is selected from the group consisting of a compound represented by the following formula (A5-1-1), and a compound represented by the following formula (A5-1-2): 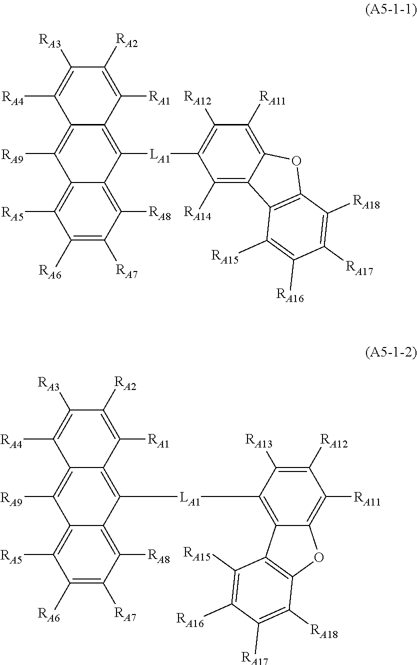
wherein in the formulas (A5-1-1) and (A5-1-2), R_{A1} to R_{A9}, R_{A11} to R_{A14}, and L_{A1} are the same as defined in the formula (A1);
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

8. The composition according to claim 1, wherein the compound represented by the formula (A1) is selected from the group consisting of a compound represented by the following formula (A5-1-1b), and a compound represented by the following formula (A5-1-2b): 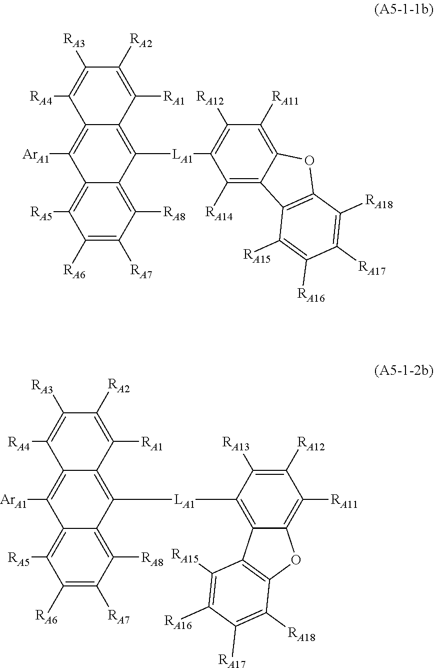
wherein in the formulas (A5-1-1b) and (A5-1-2b), R_{A1} to R_{A8}, R_{A11} to R_{A14}, and L_{A1} are the same as defined in the formula (A1);
Ar_{A1} is a substituted or unsubstituted aryl group having 6 to 50 ring carbon atoms;
one or more sets of the adjacent two or more of R_{A14} to R_{A18} form a substituted or unsubstituted, saturated or unsaturated ring by bonding with each other, or do not form the ring; and
R_{A15} to R_{A18} which do not form the ring are independently a hydrogen atom, or a substituent R; and the substituent R is the same as defined in the formula (A1).

9. The composition according to claim 1, wherein the compound represented by the formula (A1) is selected from the group consisting of a compound represented by the following formula (A6-1), a compound represented by the following formula (A6-2), and a compound represented by the following formula (A6-3): 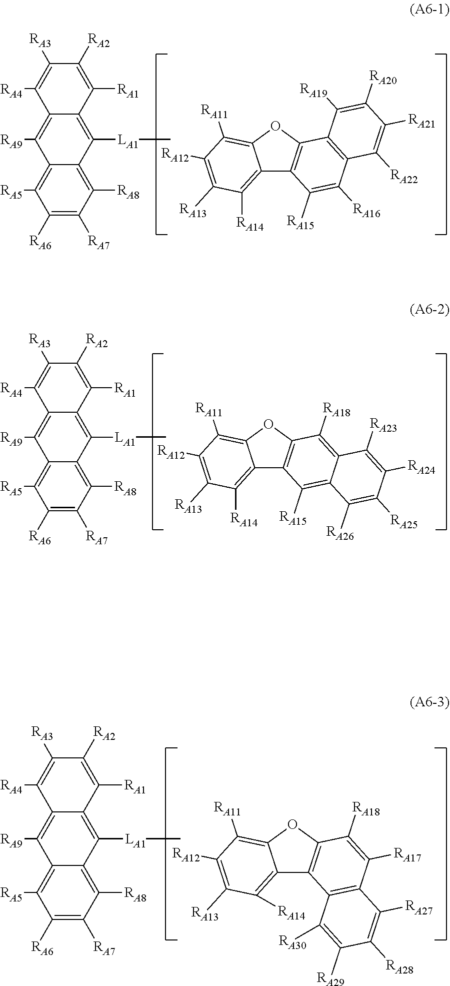
wherein in the formulas (A6-1) to (A6-3), R_{A1} to R_{A9}, R_{A11} to R_{A14}, and L_{A1} are the same as defined in the formula (A1);
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; and the substituent R is the same as defined in the formula (A1).

10. The composition according to claim 1, wherein the compound represented by the formula (A1) is selected from the group consisting of a compound represented by the following formula (A6-1a), a compound represented by the following formula (A6-2a), and a compound represented by the following formula (A6-3a): 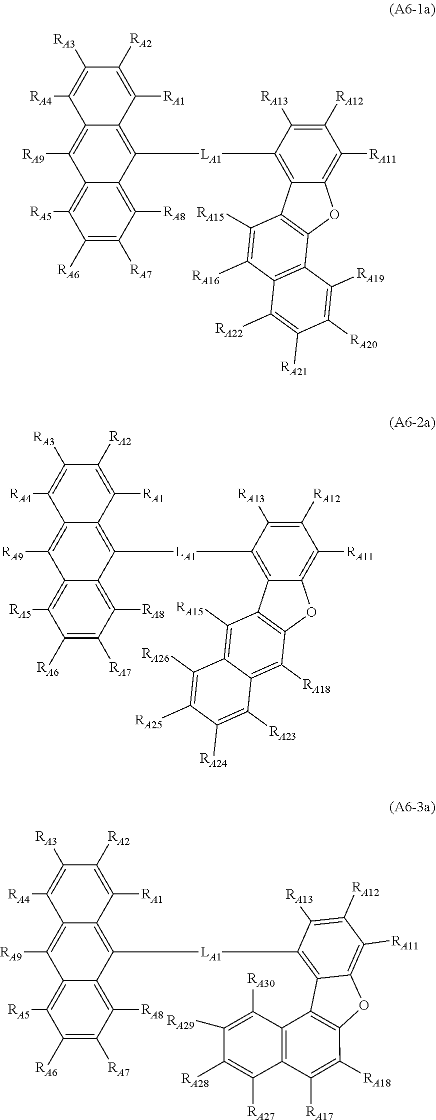
wherein in the formulas (A6-1a) to (A6-3a), R_{A1} to R_{A9}, R_{A11} to R_{A13}, and L_{A1} are the same as defined in the formula (A1);
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; and the substituent R is the same as defined in the formula (A1).

11. The composition according to claim 1, wherein the compound represented by the formula (A1) is selected from the group consisting of a compound represented by the following formula (A6-1b), a compound represented by the following formula (A6-2b), and a compound represented by the following formula (A6-3b): 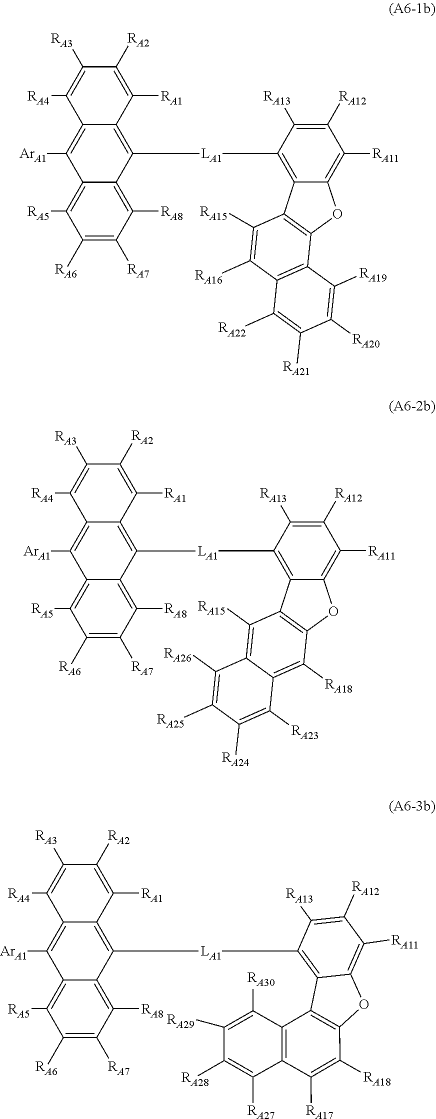
wherein in the formulas (A6-1b) to (A6-3b), R_{A1} to R_{A8}, R_{A11} to R_{A13}, and L_{A1} are the same as defined in the formula (A1);
Ar_{A1} is a substituted or unsubstituted aryl group having 6 to 50 ring carbon atoms;
one or more sets of the adjacent two or more of R_{A15} to R_{A30} form a substituted or unsubstituted, saturated or unsaturated ring by bonding with each other, or do not form the ring; and
R_{A15} to R_{A30} which do not form the ring are independently a hydrogen atom, or a substituent R; and the substituent R is the same as defined in the formula (A1).

12. The composition according to claim 1, wherein R_{A1} to R_{A8} in the formula (A1) are hydrogen atoms.

13. The composition according to claim 1, wherein R_{A11} to R_{A14} which are not a single bond bonding with L_{A1} and which do not form the ring in the formula (A1) are hydrogen atoms.

14. The composition according to claim 1, wherein L_{A1} in the formula (A1) is a single bond, or a substituted or unsubstituted arylene group having 6 to 50 ring carbon atoms.

15. The composition according to claim 1, wherein the compound represented by the formula (B1) is a compound represented by the following formula (B2): 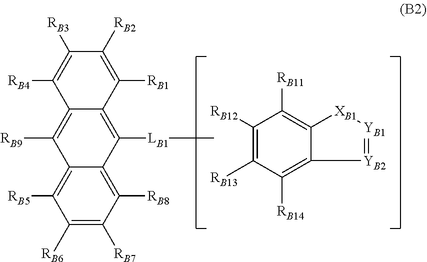
wherein in the formula (B2), R_{B1} to R_{B9}, R_{B11} to R_{B14}, L_{B1}, X_{B1}, Y_{B1}, and Y_{B2} are the same as defined in the formula (B1).

16. The composition according to claim 1, wherein the compound represented by the formula (B1) is selected from the group consisting of a compound represented by the following formula (B3-1), and a compound represented by the following formula (B3-2): 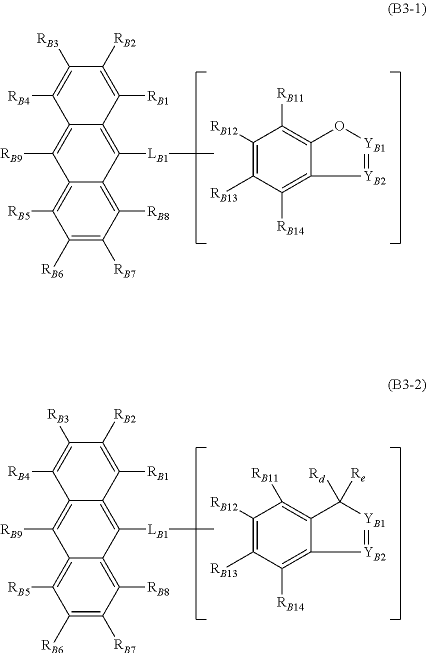
wherein in the formulas (B3-1) and (B3-2), R_{B1} to R_{B9}, R_{B11} to R_{B14}, L_{B1}, Y_{B1}, Y_{B2}, R_{d}, and Rₑ are the same as defined in the formula (B1).

17. The composition according to claim 1, wherein the compound represented by the formula (B1) is a compound represented by the following formula (B4): 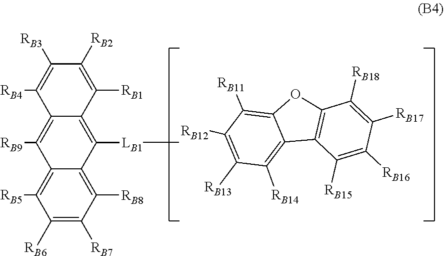
wherein in the formula (B4), R_{B1} to R_{B9}, R_{B11} to R_{B14}, and L_{B1} are the same as defined in the formula (B1);
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

18. The composition according to claim 1, wherein the compound represented by the formula (B1) is selected from the group consisting of a compound represented by the following formula (B4-1), and a compound represented by the following formula (B4-2): 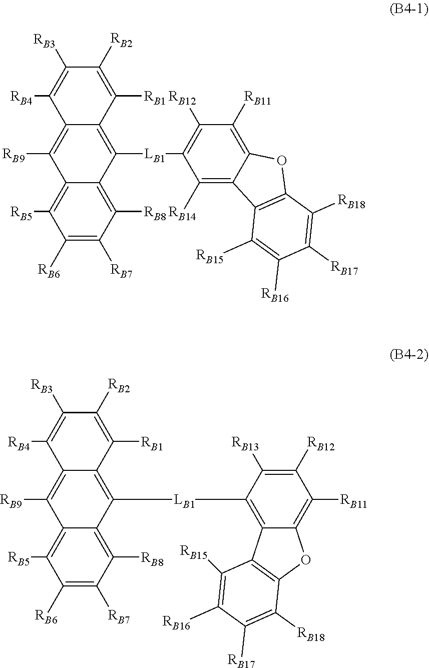
wherein in the formulas (B4-1) and (B4-2), R_{B1} to R_{B9}, R_{B11} to R_{B14}, and L_{B1} are the same as defined in the formula (B1);
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

19. The composition according to claim 1, wherein the compound represented by the formula (B1) is selected from the group consisting of a compound represented by the following formula (B4-1b), and a compound represented by the following formula (B4-2b): 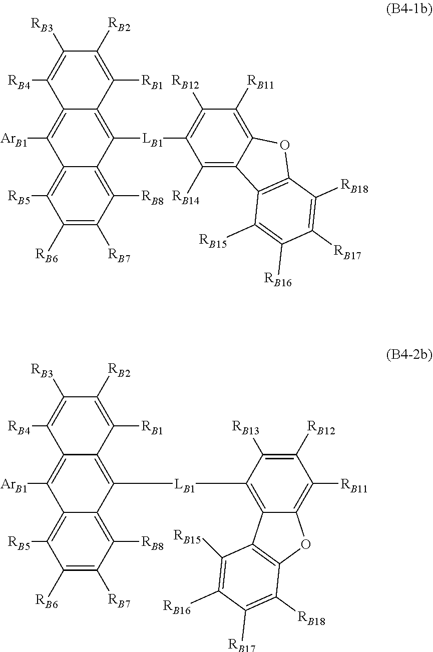
wherein in the formulas (B4-1b) and (B4-2b), R_{B1} to R_{B8}, R_{B11} to R_{B14}, and L_{B1} are the same as defined in the formula (B1);
Ar_{B1} is a substituted or unsubstituted aryl group having 6 to 50 ring carbon atoms;
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

20. The composition according to claim 1, wherein the compound represented by the formula (B1) is selected from the group consisting of a compound represented by the following formula (B5-1), a compound represented by the following formula (B5-2), and a compound represented by the following formula (B5-3): 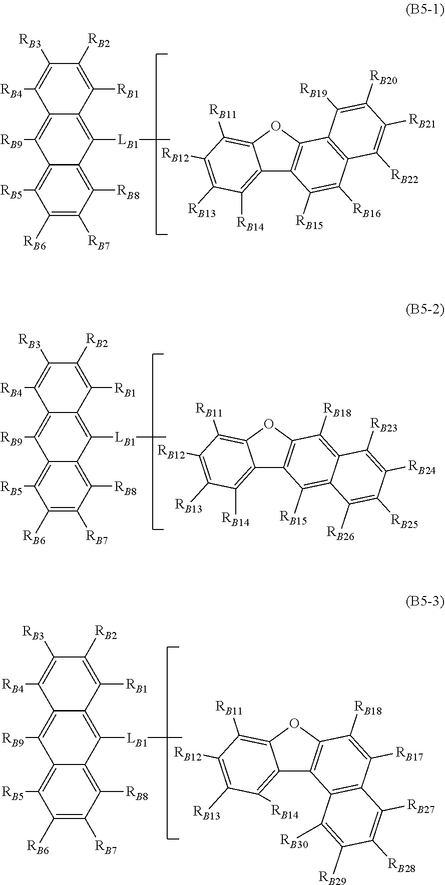
wherein in the formulas (B5-1) to (B5-3), R_{B1} to R_{B9}, R_{B11} to R_{B14}, and L_{B1} are the same as defined in the formula (B1);
one or more sets of the adjacent two or more of R_{B15} to R_{B30} form a substituted or unsubstituted, saturated or unsaturated ring by bonding with each other, or do not form the ring; and
R_{B15} to R_{B30} which do not form the ring are independently a hydrogen atom, or a substituent R; and the substituent R is the same as defined in the formula (B1).

21. The composition according to claim 1, wherein the compound represented by the formula (B1) is selected from the group consisting of a compound represented by the following formula (B5-1a), a compound represented by the following formula (B5-2a), and a compound represented by the following formula (B5-3a): 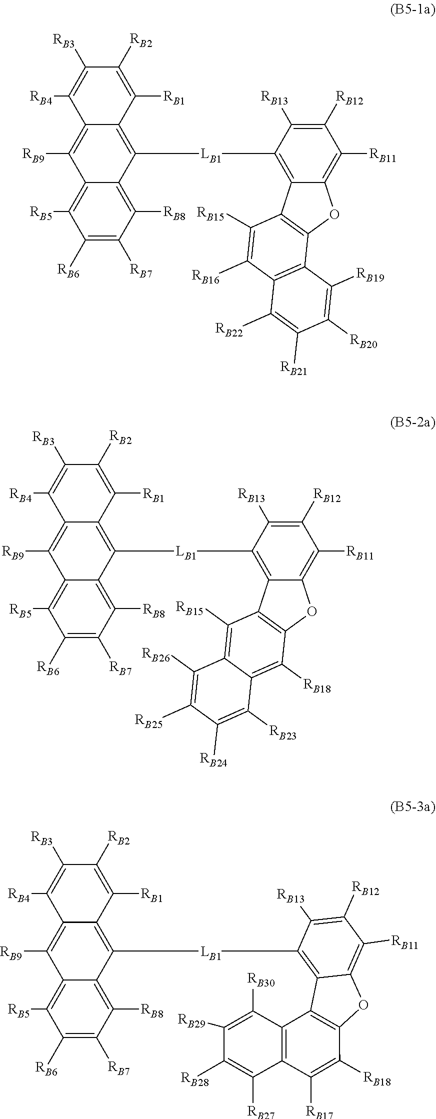
wherein in the formulas (B5-1a) to (B5-3a), R_{B1} to R_{B9}, R_{B11} to R_{B13}, and L_{B1} are the same as defined in the formula (B1);
one or more sets of the adjacent two or more of R_{B15} to R_{B30} form a substituted or unsubstituted, saturated or unsaturated ring by bonding with each other, or do not form the ring; and
R_{B15} to R₃₀ which do not form the ring are independently a hydrogen atom, or a substituent R; and the substituent R is the same as defined in the formula (B1).

22. The composition according to claim 1, wherein the compound represented by the formula (B1) is selected from the group consisting of a compound represented by the following formula (B5-1b), a compound represented by the following formula (B5-2b), and a compound represented by the following formula (B5-3b): 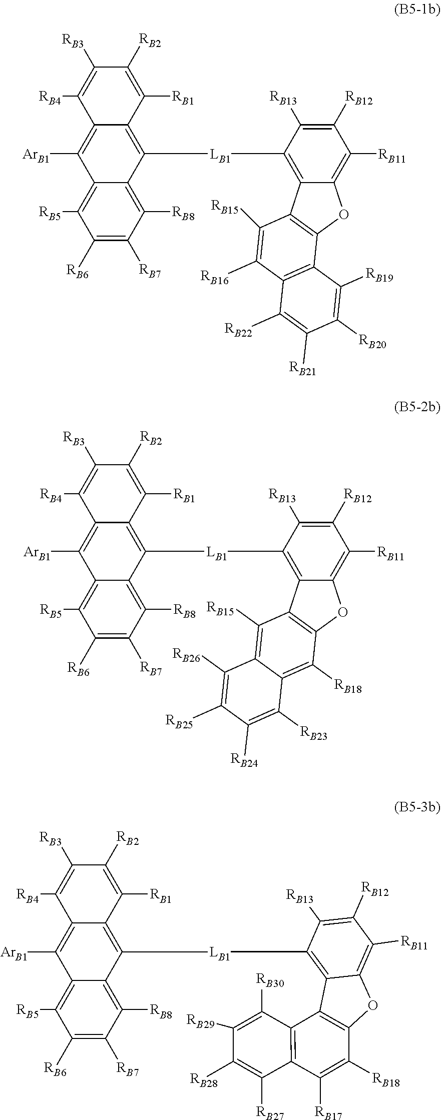
wherein in the formulas (B5-1b) to (B5-3b), R_{B1} to R_{B8}, R_{B11} to R_{B13}, and L_{B1} are the same as defined in the formula (B1);
one or more sets of the adjacent two or more of R_{B15} to R_{B30} form a substituted or unsubstituted, saturated or unsaturated ring by bonding with each other, or do not form the ring;
R_{B15} to R_{B30} which do not form the ring are independently a hydrogen atom, or a substituent R; the substituent R is the same as defined in the formula (B1); and
Ar_{B1} is a substituted or unsubstituted aryl group having 6 to 50 ring carbon atoms.

23. The composition according to claim 1, wherein the compound represented by the formula (B1) is a compound represented by the following formula (B7): 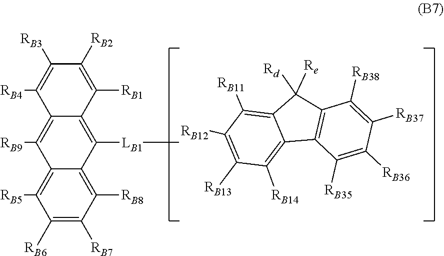
wherein in the formula (B7), R_{B1} to R_{B9}, R_{B11} to R_{B14}, L_{B1}, R_{d}, and Rₑ are the same as defined in the formula (B1);
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

24. The composition according to claim 1, wherein the compound represented by the formula (B1) is selected from the group consisting of a compound represented by the following formula (B7-1a), a compound represented by the following formula (B7-2a), and a compound represented by the following formula (B7-3a): 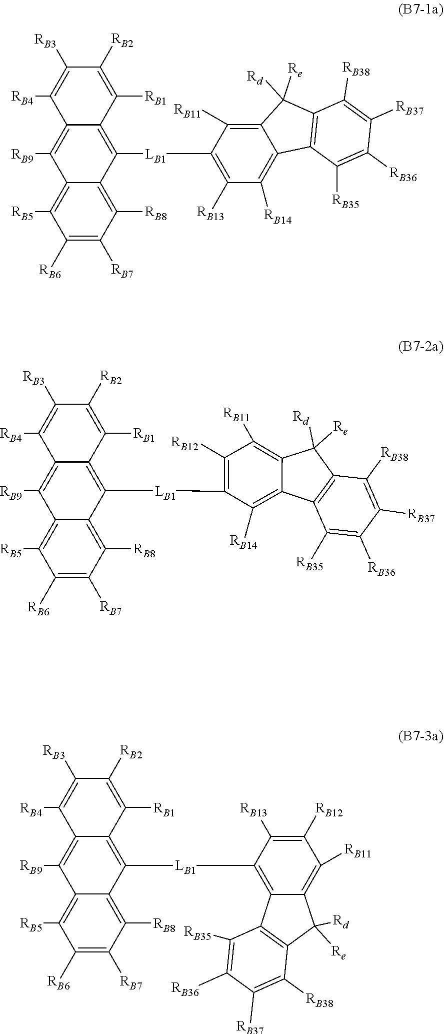
wherein in the formulas (B7-1a) to (B7-3a), R_{B1} to R_{B9}, R_{B11} to R_{B14}, L_{B1}, R_{d}, and Rₑ are the same as defined in the formula (B1);
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

25. The composition according to claim 1, wherein the compound represented by the formula (B1) is selected from the group consisting of a compound represented by the following formula (B7-1b), a compound represented by the following formula (B7-2b), and a compound represented by the following formula (B7-3b): 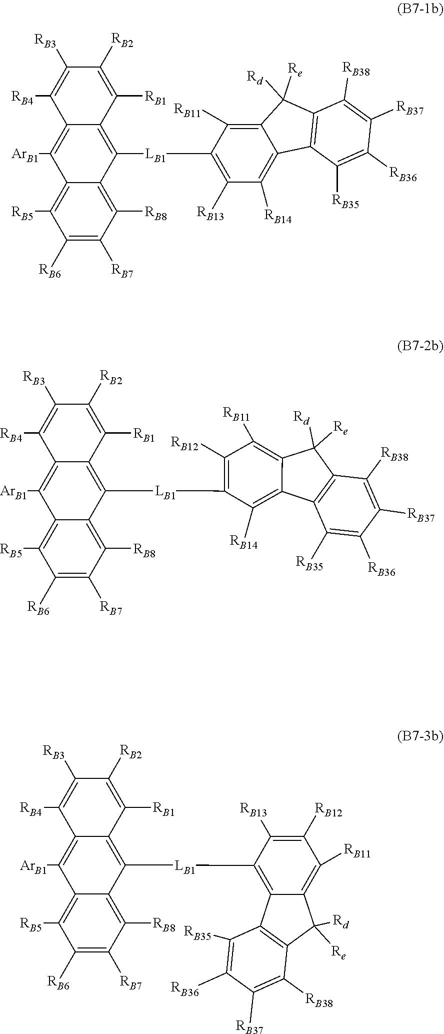
wherein in the formulas (B7-1b) to (B7-3b), R_{B1} to R_{B8}, R_{B11} to R_{B14}, L_{B1}, R_{d}, and Rₑ are the same as defined in the formula (B1);
Ar_{B1} is a substituted or unsubstituted aryl group having 6 to 50 ring carbon atoms;
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

26. The composition according to claim 1, wherein R_{B1} to R_{B8} in the formula (B1) are hydrogen atoms.

27. The composition according to claim 1, wherein R_{B11} to R_{B14} which are not a single bond bonding with L_{B1} and which do not form the ring in the formula (B1) are hydrogen atoms.

28. The composition according to claim 1, wherein L_{B1} in the formula (B1) is a single bond, or a substituted or unsubstituted arylene group having 6 to 50 ring carbon atoms.

29. The composition according to claim 1, wherein the compound represented by the formula (A1) is a compound represented by the following formula (A5-1), and
the compound represented by the formula (B1) is a compound represented by the following formula (B4) (provided that the compound represented by the formula (A5-1) and the compound represented by the formula (B4) are different compounds): 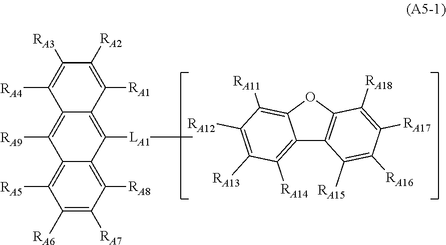
wherein in the formula (A5-1), R_{A1} to R_{A9}, R_{A11} to R_{A14}, and L_{A1} are the same as defined in the formula (A1);
one or more sets of the adjacent two or more of R_{A14} to R_{A18} form a substituted or unsubstituted, saturated or unsaturated ring by bonding with each other, or do not form the ring;
R_{A15} to R_{A18} which do not form the ring are independently a hydrogen atom, or a substituent R; the substituent R is the same as defined in the formula (A1): 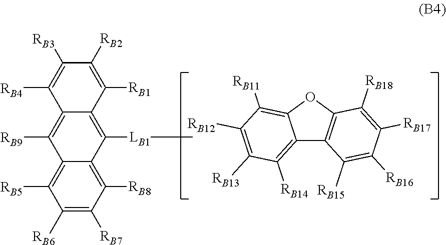
wherein in the formula (B4), R_{B1} to R_{B9}, R_{B11} to R_{B14}, and L_{B1} are the same as defined in the formula (B1);
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

30. The composition according to claim 1, wherein the compound represented by the formula (A1) is a compound selected from the group consisting of a compound represented by the following formula (A5-1-1b), and a compound represented by the following formula (A5-1-2b), and
the compound represented by the formula (B1) is selected from the group consisting of a compound represented by the following formula (B4-1b), and a compound represented by the following formula (B4-2b): 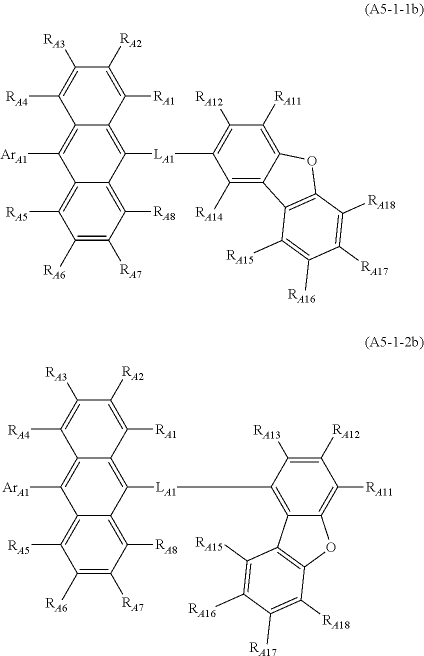
wherein in the formulas (A5-1-1b) and (A5-1-2b), R_{A1} to R_{A8}, R_{A11} to R_{A14}, and L_{A1} are the same as defined in the formula (A1);
Ar_{A1} is a substituted or unsubstituted aryl group having 6 to 50 ring carbon atoms;
one or more sets of the adjacent two or more of R_{A14} to R_{A18} form a substituted or unsubstituted, saturated or unsaturated ring by bonding with each other, or do not form the ring;
R_{A15} to R_{A18} which do not form the ring are independently a hydrogen atom, or a substituent R; the substituent R is the same as defined in the formula (A1): 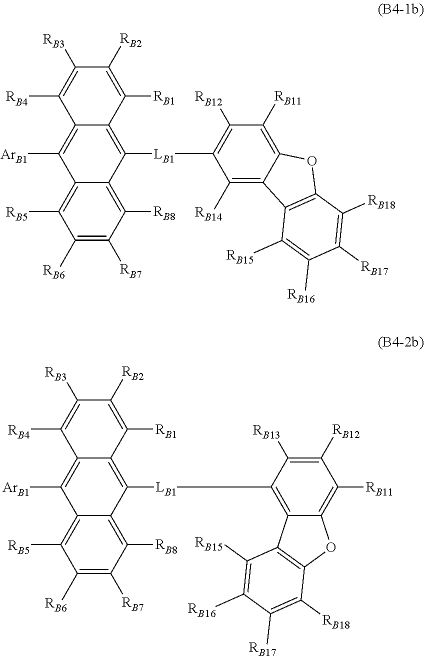
wherein in the formulas (B4-1b) and (B4-2b), R_{B1} to R_{B8}, R_{B11} to R_{B14}, and L_{B1} are the same as defined in the formula (B1);
Ar_{B1} is a substituted or unsubstituted aryl group having 6 to 50 ring carbon atoms;
one or more sets of the adjacent two or more of R_{B14} to R_{B18} form a substituted or unsubstituted, saturated or unsaturated ring by bonding with each other, or do not form the ring; and
R_{B15} to R_{B18} which do not form the ring are independently a hydrogen atom, or a substituent R; and the substituent R is the same as defined in the formula (B1).

31. The composition according to claim 1, wherein the compound represented by the formula (A1) is selected from the group consisting of a compound represented by the following formula (A6-1b), a compound represented by the following formula (A6-2b), and a compound represented by the following formula (A6-3b), and
the compound represented by the formula (B1) is selected from the group consisting of a compound represented by the following formula (B5-1b), a compound represented by the following formula (B5-2b), and a compound represented by the following formula (B5-3b): 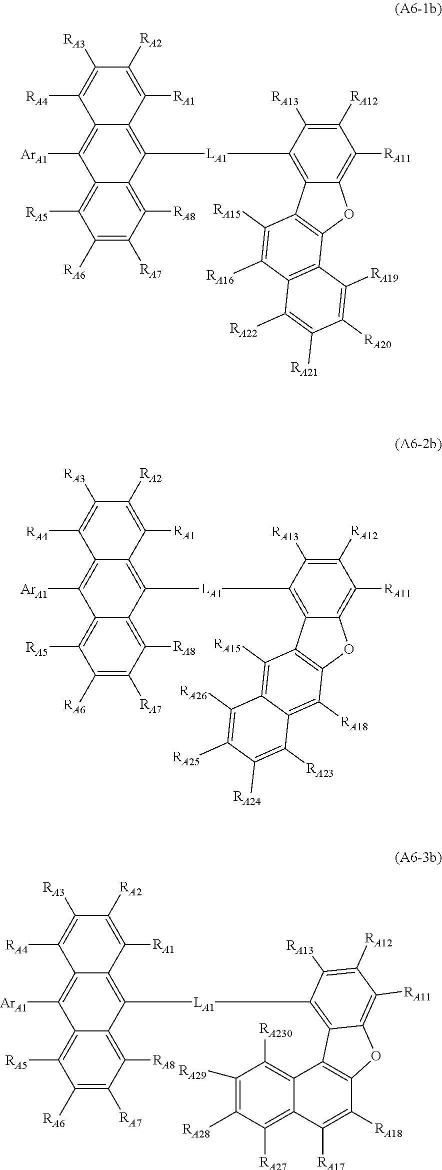
wherein in the formulas (A6-1b) to (A6-3b), R_{A1} to R_{A8}, R_{A11} to R_{A13}, and L_{A1} are the same as defined in the formula (A1);
Ar_{A1} is a substituted or unsubstituted aryl group having 6 to 50 ring carbon atoms;
one or more sets of the adjacent two or more of R_{A15} to R₃₀ form a substituted or unsubstituted, saturated or unsaturated ring by bonding with each other, or do not form the ring;
R_{A15} to R_{A30} which do not form the ring are independently a hydrogen atom, or a substituent R; the substituent R is the same as defined in the formula (A1): 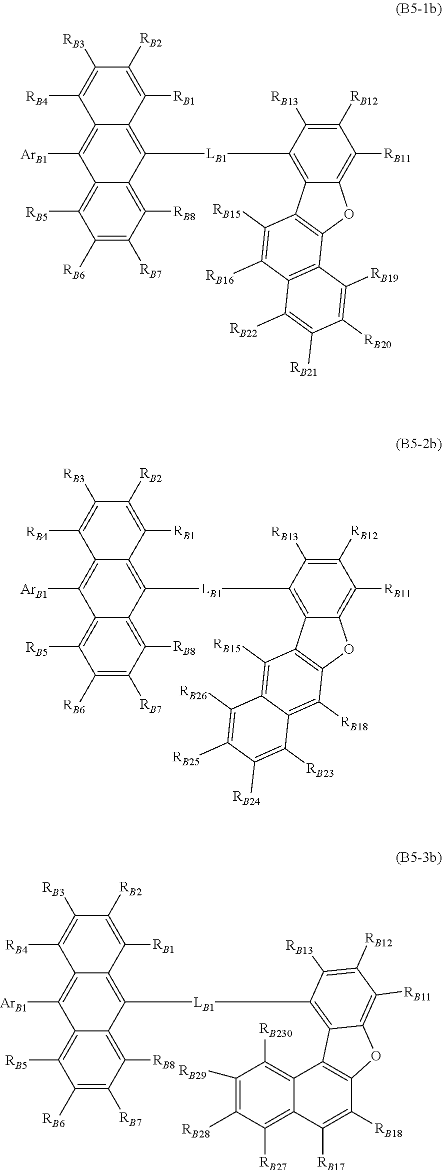
wherein in the formulas (B5-1b) to (B5-3b), R_{B1} to R_{B8}, R_{B11} to R_{B13}, and L_{B1} are the same as defined in the formula (B1);
one or more sets of the adjacent two or more of R_{B15} to R_{B30} form a substituted or unsubstituted, saturated or unsaturated ring by bonding with each other, or do not form the ring;
R_{B15} to R_{B30} which do not form the ring are independently a hydrogen atom, or a substituent R; the substituent R is the same as defined in the formula (B1); and
Ar_{B1} is a substituted or unsubstituted aryl group having 6 to 50 ring carbon atoms.

32. The composition according to claim 1, wherein the compound represented by the formula (A1) is a compound represented by the following formula (A5-1), and
the compound represented by the formula (B1) is a compound represented by the following formula (B7): 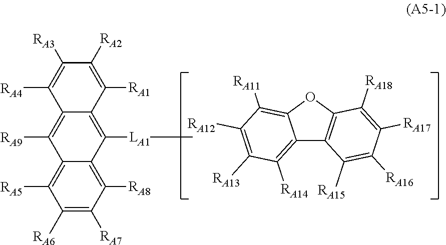
wherein in the formula (A5-1), R_{A1} to R_{A9}, R_{A11} to R_{A14}, and L_{A1} are the same as defined in the formula (A1);
one or more sets of the adjacent two or more of R_{A14} to R_{A18} form a substituted or unsubstituted, saturated or unsaturated ring by bonding with each other, or do not form the ring;
R_{A15} to R_{A18} which do not form the ring are independently a hydrogen atom, or a substituent R; the substituent R is the same as defined in the formula (A1): 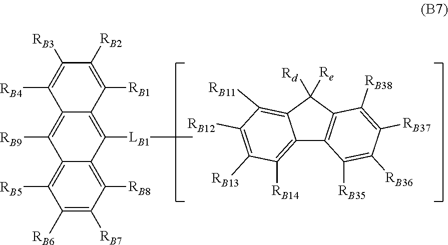
wherein in the formula (B7), R_{B1} to R_{B9}, R_{B11} to R_{B14}, L_{B1}, R_{d}, and Rₑ are the same as defined in the formula (B1);
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

33. The composition according to claim 1, wherein the compound represented by the formula (A1) is selected from the group consisting of a compound represented by the following formula (A6-1b), a compound represented by the following formula (A6-2b), and a compound represented by the following formula (A6-3b), and
the compound represented by the formula (B1) is selected from the group consisting of a compound represented by the following formula (B7-1b), a compound represented by the following formula (B7-2b), and a compound represented by the following formula (B7-3b): 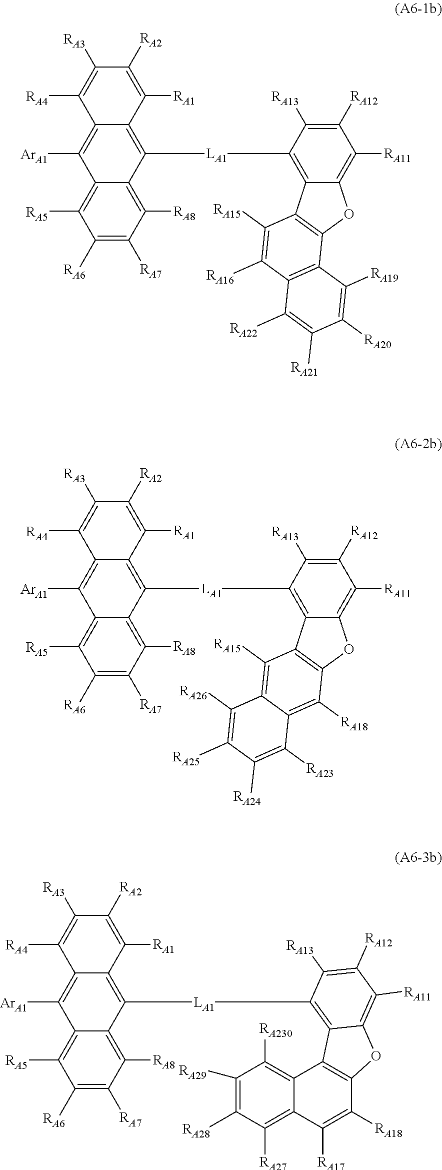
wherein in the formulas (A6-1b) to (A6-3b), R_{A1} to R_{A8}, R_{A11} to R_{A13}, and L_{A1} are the same as defined in the formula (A1);
Ar_{A1} is a substituted or unsubstituted aryl group having 6 to 50 ring carbon atoms;
one or more sets of the adjacent two or more of R_{A15} to R_{A30} form a substituted or unsubstituted, saturated or unsaturated ring by bonding with each other, or do not form the ring;
R_{A15} to R_{A30} which do not form the ring are independently a hydrogen atom, or a substituent R; the substituent R is the same as defined in the formula (A1): 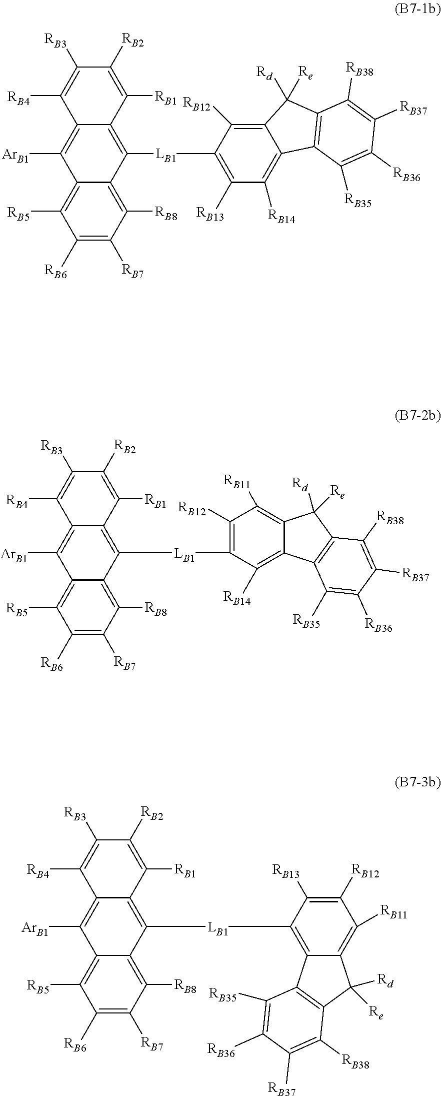
wherein in the formulas (B7-1b) to (B7-3b), R_{B1} to R_{B8}, R_{B11} to R_{B14}, L_{B1}, R_{d}, and Rₑ are the same as defined in the formula (B1);
Ar_{B1} is a substituted or unsubstituted aryl group having 6 to 50 ring carbon atoms;
one or more sets of the adjacent two or more of R_{B35} to R_{B38} form a substituted or unsubstituted, saturated or unsaturated ring by bonding with each other, or do not form the ring; and
R_{B35} to R_{B38} which do not form the ring are independently a hydrogen atom, or a substituent R; and the substituent R is the same as defined in the formula (B1).

34. The composition according to claim 1, wherein the compound represented by the formula (A1) is selected from the group consisting of a compound represented by the following formula (A5-2-1), and a compound represented by the following formula (A5-2-2), and
the compound represented by the formula (B1) is selected from the group consisting of a compound represented by the following formula (B5-1), a compound represented by the following formula (B5-2), and a compound represented by the following formula (B5-3): 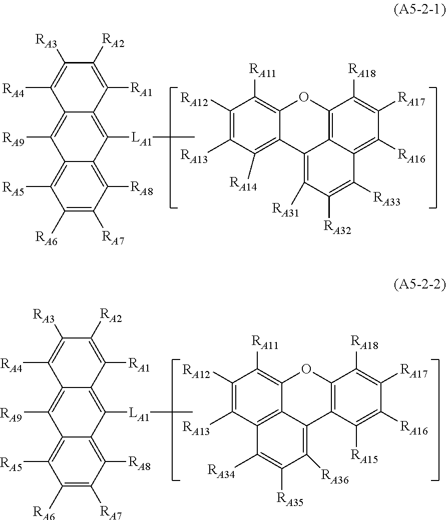
wherein in the formulas (A5-2-1) and (A5-2-2), R_{A1} to R_{A9}, R_{A11} to R_{A14}, and L_{A1} are the same as defined in the formula (A1);
one or more sets of the adjacent two or more of R_{A13} to R_{A18}, and R_{A31} to R_{A36} form a substituted or unsubstituted, saturated or unsaturated ring by bonding with each other, or do not form the ring;
R_{A15} to R_{A18} and R_{A31} to R_{A36}, which do not form the ring are independently a hydrogen atom, or a substituent R; the substituent R is the same as defined in the formula (A1): 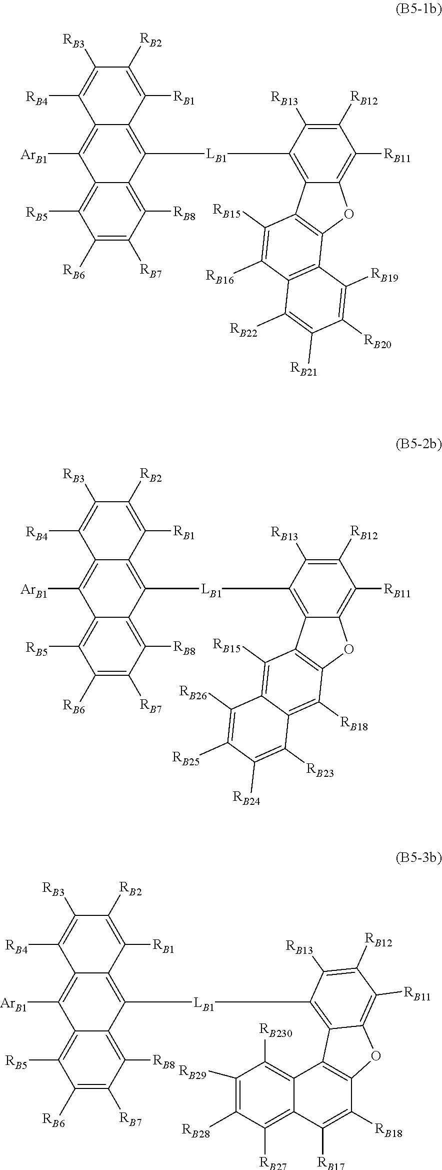
wherein in the formulas (B5-1) to (B5-3), R_{B1} to R_{B9}, R_{B11} to R_{B14}, and L_{B1} are the same as defined in the formula (B1);
one or more sets of the adjacent two or more of R_{B15} to R_{B30} form a substituted or unsubstituted, saturated or unsaturated ring by bonding with each other, or do not form the ring; and
R_{B15} to R_{B30} which do not form the ring are independently a hydrogen atom, or a substituent R; and the substituent R is the same as defined in the formula (B1).

35. The composition according to claim 1, wherein the substituent in the case of “substituted or unsubstituted” in the formulas (A1) and (B1) is selected from the group consisting of
an unsubstituted alkyl group having 1 to 50 carbon atoms,
an unsubstituted alkenyl group having 2 to 50 carbon atoms,
an unsubstituted alkynyl group having 2 to 50 carbon atoms,
an unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇)
(wherein R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other),
a halogen atom, a cyano group, a nitro group,
an unsubstituted aryl group having 6 to 50 ring carbon atoms, and
an unsubstituted monovalent heterocyclic group having 5 to 50 ring atoms.

36. A powder composed of the composition according to claim 1.

37. An organic electroluminescence device comprising the composition according to claim 1.

38. A method for fabricating an organic electroluminescence device, comprising: forming an emitting layer by depositing the powder according to claim 36 in a single deposition source.

39. An organic electroluminescence device comprising
a cathode,
an anode, and
at least one emitting layer arranged between the cathode and the anode,
wherein the at least one emitting layer comprises a compound represented by the following formula (A1) and a compound represented by the following formula (B1) (provided that the compound represented by the formula (A1) and the compound represented by the formula (B1) are different compounds): 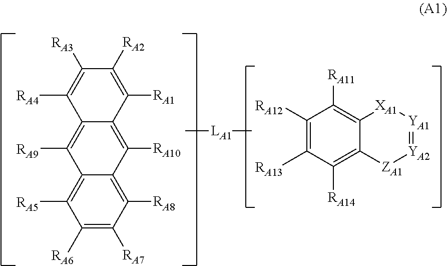
wherein in the formula (A1),
one of R_{A1} to R_{A10} is a single bond bonding with L_{A1};
one or more sets of the adjacent two or more of R_{A1} to R_{A10} which are not a single bond bonding with L_{A1} form a substituted or unsubstituted, saturated or unsaturated ring by bonding with each other, or do not form the ring;
one of R_{A11} to R_{A14} is a single bond bonding with L_{A1};
L_{A1} is a single bond, a substituted or unsubstituted arylene group having 6 to 50 ring carbon atoms, or a substituted or unsubstituted divalent heterocyclic group having 5 to 50 ring atoms;
X_{A1} is O or S;
Y_{A1} and Y_{A2} are independently a single bond, or C(Rₐ);
Z_{A1} is a single bond, or C(Rₐ)(R_{b});
one or more sets of the adjacent two or more of Rₐ, and R_{A11} to R_{A14} which are not a single bond bonding with L_{A1} form a substituted or unsubstituted, saturated or unsaturated ring by bonding with each other, or do not form the ring;
R_{b}, R_{A1} to R_{A10} and R_{A11} to R_{A14} which are not a single bond bonding with L_{A1} and which do not form the ring, and Rₐ which do not form the ring are independently a hydrogen atom, or a substituent R;
the substituent R is selected from the group consisting of
a substituted or unsubstituted alkyl group having 1 to 50 carbon atoms,
a substituted or unsubstituted alkenyl group having 2 to 50 carbon atoms,
a substituted or unsubstituted alkynyl group having 2 to 50 carbon atoms,
a substituted or unsubstituted cycloalkyl group having 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group having 6 to 50 ring carbon atoms, and
a substituted or unsubstituted monovalent heterocyclic group having 5 to 50 ring atoms;
R₉₀₁ to R₉₀₇ are independently
a hydrogen atom,
a substituted or unsubstituted alkyl group having 1 to 50 carbon atoms,
a substituted or unsubstituted cycloalkyl group having 3 to 50 ring carbon atoms,
a substituted or unsubstituted aryl group having 6 to 50 ring carbon atoms, or
a substituted or unsubstituted monovalent heterocyclic group having 5 to 50 ring atoms; when two or more of each of R₉₀₁ to R₉₀₇ are present, the two or more of each of R₉₀₁ to R₉₀₇ may be the same as or different from each other: 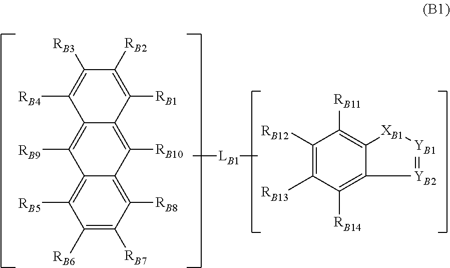
wherein in the formula (B1),
one of R_{B1} to R_{B10} is a single bond bonding with L_{B1};
one or more sets of the adjacent two or more of R_{B1} to R_{B10} which are not a single bond bonding with L_{B1} form a substituted or unsubstituted, saturated or unsaturated ring by bonding with each other, or do not form the ring;
X_{B1} is O, S, N(R_{c}), or C(R_{d})(Rₑ);
one of R_{B11} to R_{B14}, and R_{c} is a single bond bonding with L_{B1};
L_{B1} is a single bond, a substituted or unsubstituted arylene group having 6 to 50 ring carbon atoms, or a substituted or unsubstituted divalent heterocyclic group having 5 to 50 ring atoms;
Y_{B1} and Y_{B2} are independently a single bond, or C(R_{f});
R_{d} and Rₑ, and R_{f}'s form a substituted or unsubstituted, saturated or unsaturated ring by bonding with each other, or do not form the ring;
one or more sets of the adjacent two or more of R_{B11} to R_{B14} which are not a single bond bonding with L_{B1} form a substituted or unsubstituted, saturated or unsaturated ring by bonding with each other, or do not form the ring; and
R_{B1} to R_{B10} which do not form the ring and which are not a single bond bonding with L_{B1}, R^{B11} to R_{B14} which do not form the ring and which are not a single bond bonding with L_{B1}, R_{c} which are not a single bond bonding with L_{B1}, and R_{d}, Rₑ and R_{f} which do not form the ring are independently a hydrogen atom, or a substituent R; and the substituent R is the same as defined in the formula (A1).

40. The organic electroluminescence device according to claim 39, wherein both of the compound represented by the formula (A1), and the compound represented by the formula (B1) are a host material in the emitting layer.

41. An electronic apparatus comprising the organic electroluminescence device according to claim 39.
